(19) United States
(12) Patent Application Publication
(10) Pub. No.: US 2022/0049226 A1
(43) Pub. Date: Feb. 17, 2022

(54) METHODS OF TREATING SENSITIZED PATIENTS WITH HYPOIMMUNOGENIC CELLS, AND ASSOCIATED METHODS AND COMPOSITIONS

(71) Applicant: Sana Biotechnology, Inc., Seattle, WA (US)

(72) Inventors: Sonja Schrepfer, San Mateo, CA (US); Steve Harr, Seattle, WA (US); Charles E. Murry, Seattle, WA (US)

(21) Appl. No.: 17/401,174

(22) Filed: Aug. 12, 2021

Related U.S. Application Data
(60) Provisional application No. 63/065,342, filed on Aug. 13, 2020.
(60) Provisional application No. 63/136,137, filed on Jan. 11, 2021.
(60) Provisional application No. 63/151,628, filed on Feb. 19, 2021.
(60) Provisional application No. 63/175,030, filed on Apr. 14, 2021.

(51) Int. Cl. C12N 5/071; C07K 14/74; C07K 14/705; A61K 35/39; A61K 35/17; C12N 5/0783

(52) U.S. Cl. CPC C12N 5/0676; C07K 14/70539; C12N 5/0636; A61K 35/39; A61K 35/17; C07K 14/70596

(57) ABSTRACT

Disclosed herein are hypoimmunogenic cells for administering to a sensitized patient. In some instances, the patient is sensitized from a previous pregnancy or a previous transplant. In some embodiments, the cells exogenously express CD47 proteins and exhibit reduced expression of MHC class I proteins, MHC class II proteins, or both.

### CROSS REFERENCE TO RELATED APPLICATIONS

This application claims priority under 35 U.S.C. § 119(e) to U.S. Provisional Application Nos. 63/065,342 filed Aug. 13, 2020; 63/136,137 filed Jan. 11, 2021; 63/151,628 filed Feb. 19, 2021; and 63/175,030 filed Apr. 14, 2021, the disclosures of which are herein incorporated by reference in their entireties.

### SUMMARY

Sensitization to antigens (e.g., donor alloantigens) is a problem facing clinical transplantation therapies. For example, the propensity for the transplant recipient's immune system to reject allogeneic material greatly reduces the potential efficacy of therapeutics and diminishes the possible positive effects surrounding such treatments. Fortunately, there is substantial evidence in both animal models and human patients that hypoimmunogenic cell or tissue transplantation is a scientifically feasible and clinically promising approach to the treatment of numerous disorders and conditions.

As such, there remains a need for novel approaches, compositions and methods for producing cell-based therapies that avoid detection by the recipient's immune system.

Sensitization to antigens (e.g., donor alloantigens) is a problem facing clinical transplantation therapies. For example, the propensity for the transplant recipient's immune system to reject allogeneic material greatly reduces the potential efficacy of therapeutics and diminishes the possible positive effects surrounding such treatments. Fortunately, there is substantial evidence in both animal models and human patients that hypoimmunogenic cell or tissue transplantation is a scientifically feasible and clinically promising approach to the treatment of numerous disorders and conditions.

As such, there remains a need for novel approaches, compositions and methods for producing cell-based therapies that avoid detection by the recipient's immune system.

In some aspects, provided is a method of treating a patient in need thereof comprising administering a population of hypoimmunogenic cells, wherein the hypoimmunogenic cells comprise a first exogenous polynucleotide encoding CD47 and (I) one or more of: (a) reduced expression of major histocompatibility complex (MHC) class I and/or class II human leukocyte antigens; (b) reduced expression of MHC class I and class II human leukocyte antigens; (c) reduced expression of beta-2-microglobulin (B2M) and/or MHC class II transactivator (CIITA); and/or (d) reduced expression of B2M and CIITA; wherein the reduced expression is due to a modification and the reduced expression is relative to a cell of the same cell type that does not comprise the modification; (II) wherein: the patient is a sensitized patient, wherein the patient: (i) is sensitized against one or more alloantigens; (ii) is sensitized against one or more autologous antigens; (iii) is sensitized from a previous transplant; (iv) is sensitized from a previous pregnancy; (v) received a previous treatment for a condition or disease; and/or (vi) is a tissue or organ transplant patient, and the hypoimmunogenic cells are administered prior to, concurrent with, and/or after administering the tissue or organ transplant.

In some aspects, provided is a method of treating a patient in need thereof comprising administering a population of pancreatic islet cells, wherein the pancreatic islet cells comprise a first exogenous polynucleotide encoding CD47 and (I) one or more of: (a) reduced expression of major histocompatibility complex (MHC) class I and/or class II human leukocyte antigens; (b) reduced expression of MHC class I and class II human leukocyte antigens; (c) reduced expression of beta-2-microglobulin (B2M) and/or MHC class II transactivator (CIITA); and/or (d) reduced expression of B2M and CIITA; wherein the reduced expression is due to a modification and the reduced expression is relative to a cell of the same cell type that does not comprise the modification; (II) wherein: (a) the patient is not a sensitized patient; or (b) the patient is a sensitized patient, wherein the patient: (i) is sensitized against one or more alloantigens; (ii) is sensitized against one or more autologous antigens; (iii) is sensitized from a previous transplant; (iv) is sensitized from a previous pregnancy; (v) received a previous treatment for a condition or disease; and/or (vi) is a tissue or organ patient, and the pancreatic islet cells are administered prior to administering the tissue or organ transplant.

In some aspects, provided is a method of treating a patient in need thereof comprising administering a population of cardiac progenitor cells, wherein the cardiac progenitor cells comprise a first exogenous polynucleotide encoding CD47 and (I) one or more of: (a) reduced expression of major histocompatibility complex (MHC) class I and/or class II human leukocyte antigens; (b) reduced expression of MHC class I and class II human leukocyte antigens; (c) reduced expression of beta-2-microglobulin (B2M) and/or MHC class II transactivator (CIITA); and/or (d) reduced expression of B2M and CIITA; wherein the reduced expression is due to a modification and the reduced expression is relative to a cell of the same cell type that does not comprise the modification; (II) wherein: (a) the patient is not a sensitized patient; or (b) the patient is a sensitized patient, wherein the patient: (i) is sensitized against one or more alloantigens; (ii) is sensitized against one or more autologous antigens; (iii) is sensitized from a previous transplant; (iv) is sensitized from a previous pregnancy; (v) received a previous treatment for a condition or disease; and/or (vi) is a tissue or organ patient, and the cardiac muscle cells are administered prior to administering the tissue or organ transplant.

In some aspects, provided is a method of treating a patient in need thereof comprising administering a population of glial progenitor cells, wherein the glial progenitor cells comprise a first exogenous polynucleotide encoding CD47 and (I) one or more of: (a) reduced expression of major histocompatibility complex (MHC) class I and/or class II human leukocyte antigens; (b) reduced expression of MHC class I and class II human leukocyte antigens; (c) reduced expression of beta-2-microglobulin (B2M) and/or MHC class II transactivator (CIITA); and/or (d) reduced expression of B2M and CIITA; wherein the reduced expression is due to a modification and the reduced expression is relative to a cell of the same cell type that does not comprise the modification; (II) wherein: (a) the patient is not a sensitized patient; or (b) the patient is a sensitized patient, wherein the patient: (i) is sensitized against one or more alloantigens; (ii) is sensitized against one or more autologous antigens; (iii) is sensitized from a previous transplant; (iv) is sensitized from a previous pregnancy; (v) received a previous treatment for a condition or disease; and/or (vi) is a tissue or organ patient, and the glial progenitor cells are administered prior to administering the tissue or organ transplant.

In some embodiments, the patient is a sensitized patient and wherein the patient exhibits memory B cells and/or memory T cells reactive against the one or more alloantigens or one or more autologous antigens. In some embodiments, the one or more alloantigens comprise human leukocyte antigens.

In some embodiments, the patient is a sensitized patient who is sensitized from a previous transplant, wherein: (a) the previous transplant is selected from the group consisting of a cell transplant, a blood transfusion, a tissue transplant, and an organ transplant, optionally the previous transplant is an allogeneic transplant; or (b) the previous transplant is a transplant selected from the group consisting of a chimera of human origin, a modified non-human autologous cell, a modified autologous cell, an autologous tissue, and an autologous organ, optionally the previous transplant is an autologous transplant.

In some embodiments, the patient is a sensitized patient who is sensitized from a previous pregnancy and wherein the patient had previously exhibited alloimmunization in pregnancy, optionally wherein the alloimmunization in pregnancy is hemolytic disease of the fetus and newborn (HDFN), neonatal alloimmune neutropenia (NAN) or fetal and neonatal alloimmune thrombocytopenia (FNAIT).

In some embodiments, the patient is a sensitized patient who is sensitized from a previous treatment for a condition or disease, wherein the condition or disease is different from or the same as the disease or condition for which the patient is being treated as described herein.

In some embodiments, the patient received a previous treatment for a condition or disease, wherein the previous treatment did not comprise the population of cells, and wherein: (a) the population of cells is administered for the treatment of the same condition or disease as the previous treatment; (b) the population of cells exhibits an enhanced therapeutic effect for the treatment of the condition or disease in the patient as compared to the previous treatment; (c) the population of cells exhibits a longer therapeutic effect for the treatment of the condition or disease in the patient as compared to the previous treatment; (d) the previous treatment was therapeutically effective; (e) the previous treatment was therapeutically ineffective; (f) the patient developed an immune reaction against the previous treatment; and/or (g) the population of cells is administered for the treatment of a different condition or disease as the previous treatment.

In some embodiments, the previous treatment comprises administering a population of therapeutic cells comprising a suicide gene or a safety switch system, and the immune reaction occurs in response to activation of the suicide gene or the safety switch system.

In some embodiments, the previous treatment comprises a mechanically assisted treatment, optionally wherein the mechanically assisted treatment comprises hemodialysis or a ventricle assist device.

In some embodiments, the previous treatment comprises an allogeneic CAR-T cell based therapy or an autologous CAR-T cell based therapy, wherein the autologous CAR-T cell based therapy is selected from the group consisting of brexucabtagene autoleucel, axicabtagene ciloleucel, idecabtagene vicleucel, lisocabtagene maraleucel, tisagenlecleucel, Descartes-08 or Descartes-11 from Cartesian Therapeutics, CTL110 from Novartis, P-BMCA-101 from Poseida Therapeutics, AUTO4 from Autolus Limited, UCARTCS from Cellectis, PBCAR19B or PBCAR269A from Precision Biosciences, FT819 from Fate Therapeutics, and CYAD-211 from Clyad Oncology.

In some embodiments, the patient has an allergy, optionally wherein the allergy is an allergy selected from the group consisting of a hay fever, a food allergy, an insect allergy, a drug allergy, and atopic dermatitis.

In some embodiments, the cells further comprise one or more exogenous polypeptides selected from the group consisting of DUX4, CD24, CD46, CD55, CD59, CD200, PD-L1, HLA-E, HLA-G, IDO1, FasL, IL-35, IL-39, CCL21, CCL22, Mfge8, Serpin B9, and a combination thereof.

In some embodiments, the cells further comprise reduced expression levels of CD142, relative to a cell of the same cell type that does not comprise a modification. In some embodiments, the cells further comprise reduced expression levels of CD46, relative to a cell of the same cell type that does not comprise a modification. In some embodiments, the cells further comprise reduced expression levels of CD59, relative to a cell of the same cell type that does not comprise a modification.

In some embodiments, the cells are differentiated from stem cells. In some embodiments, the stem cells are mesenchymal stem cells. In some embodiments, the stem cells are embryonic stem cells. In some embodiments, the stem cells are pluripotent stem cells, optionally wherein the pluripotent stem cells are induced pluripotent stem cells. In some embodiments, the cells are selected from the group consisting of cardiac cells, cardiac progenitor cells, neural cells, glial progenitor cells, endothelial cells, T cells, B cells, pancreatic islet cells, retinal pigmented epithelium cells, hepatocytes, thyroid cells, skin cells, blood cells, plasma cells, platelets, renal cells, epithelial cells, chimeric antigen receptor (CAR) T cells, NK cells, and CAR-NK cells. In some embodiments, the cells are derived from primary cells. In some embodiments, the primary cells are primary T cells, primary beta cells, or primary retinal pigment epithelial cells. In some embodiments, the cells derived from primary T cells are derived from a pool of T cells comprising primary T cells from one or more subjects different from the patient.

In some embodiments, the cells comprise a second exogenous polynucleotide encoding a chimeric antigen receptor (CAR). In some embodiments, the antigen binding domain of the CAR binds to CD19, CD22, or BCMA.

In some embodiments, the CAR is a CD19-specific CAR such that the cell is a CD19 CAR T cell. In some embodiments, the CAR is a CD22-specific CAR such that the cell is a CD22 CART cell. In some embodiments, the cell comprises a CD19-specific CAR and a CD22-specific CAR such that the cell is a CD19/CD22 CAR T cell. In some embodiments, the CD19-specific CAR and the CD22-specific CAR are encoded by a single bicistronic polynucleotide. In some embodiments, the CD19-specific CAR and the CD22-specific CAR are encoded by two separate polynucleotides.

In some embodiments, the first and/or second exogenous polynucleotide is inserted into a genomic locus comprising a safe harbor locus, a target locus, a B2M gene locus, a CIITA gene locus, a TRAC gene locus, or a TRB gene locus.

In some embodiments, the first and second genomic loci are the same. In some embodiments, the first and second genomic loci are different. In some embodiments, the cells each further comprise a third exogenous polynucleotide inserted into a third genomic locus. In some embodiments, the third genomic locus is the same as the first or second genomic loci. In some embodiments, the third genomic locus is different from the first and/or second genomic loci.

In some embodiments, the safe harbor locus is selected from the group consisting of: a CCR5 gene locus, a PPP1R12C (also known as AAVS1) gene, a ROSA26 gene locus, and a CLYBL gene locus. T In some embodiments, the target locus is selected from the group consisting of: a CXCR4 gene locus, an albumin gene locus, a SHS231 locus, a CD142 gene locus, a MICA gene locus, a MICB gene locus, a LRP1 gene locus, a HMGB1 gene locus, an ABO gene locus, a RHD gene locus, a FUT1 gene locus, and a KDM5D gene locus.

In some embodiments, the insertion into the CCR5 gene locus is in exon 1-3, intron 1-2 or another coding sequence (CDS) of the CCR5 gene. In some embodiments, the insertion into the PPP1R12C gene locus is intron 1 or intron 2 of the PPP1R12C gene. In some embodiments, the insertion into the CLYBL gene locus is intron 2 of the CLYBL gene. In some embodiments, the insertion into the ROSA26 gene locus is intron 1 of the ROSA26 gene. In some embodiments, the insertion into the insertion into the safe harbor locus is a SHS231 locus. In some embodiments, the insertion into the CD142 gene locus is in exon 2 or another CDS of the CD142 gene. In some embodiments, the insertion into the MICA gene locus is in a CDS of the MICA gene. In some embodiments, the insertion into the MICB gene locus is in a CDS of the MICB gene. In some embodiments, the insertion into the B2M gene locus is in exon 2 or another CDS of the B2M gene. In some embodiments, the insertion into the CIITA gene locus is in exon 3 or another CDS of the CIITA gene. In some embodiments, the insertion into the TRAC gene locus is in exon 2 or another CDS of the TRAC gene. In some embodiments, the insertion into the TRB gene locus is in a CDS of the TRB gene.

In some embodiments, the cells derived from primary T cells comprise reduced expression of one or more of: an endogenous T cell receptor; cytotoxic T-lymphocyte-associated protein 4 (CTLA4); programmed cell death (PD1); and programmed cell death ligand 1 (PD-L1), wherein the reduced expression is due to a modification and the reduced expression is relative to a cell of the same cell type that does not comprise the modification. In some embodiments, the cells derived from primary T cells comprised reduced expression of TRAC.

In some embodiments, the cells are T cells derived from induced pluripotent stem cells that comprise reduced expression of one or more of: an endogenous T cell receptor; cytotoxic T-lymphocyte-associated protein 4 (CTLA4); programmed cell death (PD1); and programmed cell death ligand 1 (PD-L1). In some embodiments, the cells are T cells derived from induced pluripotent stem cells that comprise reduced expression of TRAC and TRB.

In some embodiments, the exogenous polynucleotide is operably linked to a promoter. In some embodiments, the promoter is a CAG and/or an EF1a promoter.

In some embodiments, the population of cells is administered at least 1 day or more after the patient is sensitized against one or more alloantigens, or at least 1 day or more after the patient had received the allogeneic transplant. In some embodiments, the population of cells is administered at least 1 week or more after the patient is sensitized against one or more alloantigens, or at least 1 week or more after the patient had received the allogeneic transplant.

In some embodiments, the population of cells is administered at least 1 month or more after the patient is sensitized against one or more alloantigens, at least 1 month or more after the patient had received the allogeneic transplant.

In some embodiments, the patient exhibits no immune response upon administration of the population of cells. In some embodiments, the no immune response upon administration of the population of cells is selected from the group consisting of no systemic immune response, no adaptive immune response, no innate immune response, no T cell response, no B cell response, and no systemic acute cellular immune response.

In some embodiments, the patient exhibits one or more of: (a) no systemic TH1 activation upon administering the population of cells; (b) no immune activation of peripheral blood mononuclear cells (PBMCs) upon administering the population of cells; (c) no donor specific IgG antibodies against the population of cells upon administering the population of cells; (d) no IgM and IgG antibody production against the population of cells upon administering the population of cells; and (e) no cytotoxic T cell killing of the population of cells upon administering the population of cells.

In some embodiments, the patient is not administered an immunosuppressive agent at least 3 days or more before or after the administration of the population of cells.

In some embodiments, the method comprises a dosing regimen comprising: a first administration comprising a therapeutically effective amount of the population of cells; a recovery period; and a second administration comprising a therapeutically effective amount of the population of cells. In some embodiments, the recovery period comprises at least 1 month or more. In some embodiments, the recovery period comprises at least 2 months or more.

In some embodiments, the second administration is initiated when the cells from the first administration are no longer detectable in the patient, optionally wherein the cells are no longer detectable due to elimination resulting from a suicide gene or a safety switch system.

In some embodiments, the hypoimmunogenic cells are eliminated by a suicide gene or a safety switch system, and wherein the second administration is initiated when the cells from the first administration are no longer detectable in the patient.

In some embodiments, the method further comprises administering the dosing regimen at least twice. In some embodiments, the population of cells is administered for treatment of a cellular deficiency or as a cellular therapy for the treatment of a condition or disease in a tissue or organ selected from the group consisting of heart, lung, kidney, liver, pancreas, intestine, stomach, cornea, bone marrow, blood vessel, heart valve, brain, spinal cord, and bone.

In some embodiments of the method: (a) the cellular deficiency is associated with a neurodegenerative disease or the cellular therapy is for the treatment of a neurodegenerative disease; (b) the cellular deficiency is associated with a liver disease or the cellular therapy is for the treatment of liver disease; (c) the cellular deficiency is associated with a corneal disease or the cellular therapy is for the treatment of corneal disease; (d) the cellular deficiency is associated with a cardiovascular condition or disease or the cellular therapy is for the treatment of a cardiovascular condition or disease; (e) the cellular deficiency is associated with diabetes or the cellular therapy is for the treatment of diabetes; (f) the cellular deficiency is associated with a vascular condition or disease or the cellular therapy is for the treatment of a vascular condition or disease; (g) the cellular deficiency is associated with autoimmune thyroiditis or the cellular therapy is for the treatment of autoimmune thyroiditis; or (h) the cellular deficiency is associated with a kidney disease or the cellular therapy is for the treatment of a kidney disease.

In some embodiments of the method: (a) the neurodegenerative disease is selected from the group consisting of leukodystrophy, Huntington's disease, Parkinson's disease, multiple sclerosis, transverse myelitis, and Pelizaeus-Merzbacher disease (PMD); (b) the liver disease comprises cirrhosis of the liver; (c) the corneal disease is Fuchs dystrophy or congenital hereditary endothelial dystrophy; or (d) the cardiovascular disease is myocardial infarction or congestive heart failure.

In some embodiments, the population of cells comprises: (a) cells selected from the group consisting of glial progenitor cells, oligodendrocytes, astrocytes, and dopaminergic neurons, optionally wherein the dopaminergic neurons are selected from the group consisting of neural stem cells, neural progenitor cells, immature dopaminergic neurons, and mature dopaminergic neurons; (b) hepatocytes or hepatic progenitor cells; (c) corneal endothelial progenitor cells or corneal endothelial cells; (d) cardiomyocytes or cardiac progenitor cells; (e) pancreatic islet cells, including pancreatic beta islet cells, optionally wherein the pancreatic islet cells are selected from the group consisting of a pancreatic islet progenitor cell, an immature pancreatic islet cell, and a mature pancreatic islet cell; (f) endothelial cells; (g) thyroid progenitor cells; or (h) renal precursor cells or renal cells.

In some embodiments, the population of cells is administered for the treatment of cancer. In some embodiments, the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

In some embodiments, the patient is receiving a tissue or organ transplant, optionally wherein the tissue or organ transplant or partial organ transplant is selected from the group consisting of a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, a bone transplant, a partial lung transplant, a partial kidney transplant, a partial liver transplant, a partial pancreas transplant, a partial intestine transplant, and a partial cornea transplant.

In some embodiments, the tissue or organ transplant is an allograft transplant. In some embodiments, the tissue or organ transplant is an autograft transplant.

In some embodiments, the population of cells is administered for the treatment of a cellular deficiency in a tissue or organ and the tissue or organ transplant is for the replacement of the same tissue or organ. In some embodiments, the population of cells is administered for the treatment of a cellular deficiency in a tissue or organ and the tissue or organ transplant is for the replacement of a different tissue or organ. In some embodiments, the organ transplant is a kidney transplant and the population of cells is a population of pancreatic beta islet cells. In some embodiments, the patient has diabetes. In some embodiments, the organ transplant is a heart transplant and the population of cells is a population of pacemaker cells. In some embodiments, the organ transplant is a pancreas transplant and the population of cells is a population of beta islet cells. In some embodiments, the organ transplant is a partial liver transplant and the population of cells is a population of hepatocytes or hepatic progenitor cells.

In some aspect, provided here is use of a population of hypoimmunogenic cells for treatment of a disorder in a patient, wherein the hypoimmunogenic cells comprises a first exogenous polynucleotide encoding CD47 and (I) one or more of: (a) reduced expression of major histocompatibility complex (MHC) class I and/or class II human leukocyte antigens; (b) reduced expression of MHC class I and class II human leukocyte antigens; (c) reduced expression of beta-2-microglobulin (B2M) and/or MHC class II transactivator (CIITA); and/or (d) reduced expression of B2M and CIITA; wherein the reduced expression is due to a modification and the reduced expression is relative to a cell of the same cell type that does not comprise the modification; (II) wherein: (a) the patient is not a sensitized patient; or (b) the patient is a sensitized patient.

In some aspect, provided here is use of a population of pancreatic islet cells for treatment of a disorder in a patient, wherein the pancreatic islet cells comprises a first exogenous polynucleotide encoding CD47 and (I) one or more of: (a) reduced expression of major histocompatibility complex (MHC) class I and/or class II human leukocyte antigens; (b) reduced expression of MHC class I and class II human leukocyte antigens; (c) reduced expression of beta-2-microglobulin (B2M) and/or MHC class II transactivator (CIITA); and/or (d) reduced expression of B2M and CIITA; wherein the reduced expression is due to a modification and the reduced expression is relative to a cell of the same cell type that does not comprise the modification; (II) wherein: (a) the patient is not a sensitized patient; or (b) the patient is a sensitized patient.

In some aspect, provided here is use of a population of cardiac muscle cells for treatment of a disorder in a patient, wherein the cardiac muscle cells comprises a first exogenous polynucleotide encoding CD47 and (I) one or more of: (a) reduced expression of major histocompatibility complex (MHC) class I and/or class II human leukocyte antigens; (b) reduced expression of MHC class I and class II human leukocyte antigens; (c) reduced expression of beta-2-microglobulin (B2M) and/or MHC class II transactivator (CIITA); and/or (d) reduced expression of B2M and CIITA; wherein the reduced expression is due to a modification and the reduced expression is relative to a cell of the same cell type that does not comprise the modification; (II) wherein: (a) the patient is not a sensitized patient; or (b) the patient is a sensitized patient.

In some aspect, provided here is use of a population of glial progenitor cells for treatment of a disorder in a patient, wherein the glial progenitor cells comprises a first exogenous polynucleotide encoding CD47 and (I) one or more of: (a) reduced expression of major histocompatibility complex (MHC) class I and/or class II human leukocyte antigens; (b) reduced expression of MHC class I and class II human leukocyte antigens; (c) reduced expression of beta-2-microglobulin (B2M) and/or MHC class II transactivator (CIITA); and/or (d) reduced expression of B2M and CIITA; wherein the reduced expression is due to a modification and the reduced expression is relative to a cell of the same cell type that does not comprise the modification; (II) wherein: (a) the patient is not a sensitized patient; or (b) the patient is a sensitized patient.

In some embodiments, the patient is a sensitized patient and wherein the patient exhibits memory B cells and/or memory T cells reactive against the one or more alloantigens or one or more autologous antigens. In some embodiments, the one or more alloantigens comprise human leukocyte antigens.

In some embodiments, the patient is a sensitized patient who is sensitized from a previous transplant, wherein: the previous transplant is selected from the group consisting of a cell transplant, a blood transfusion, a tissue transplant, and an organ transplant, optionally the previous transplant is an allogeneic transplant; or the previous transplant is a transplant selected from the group consisting of a chimera of human origin, a modified non-human autologous cell, a modified autologous cell, an autologous tissue, and an autologous organ, optionally the previous transplant is an autologous transplant.

In some embodiments, the patient is a sensitized patient who is sensitized from a previous pregnancy and wherein the patient had previously exhibited alloimmunization in pregnancy, optionally wherein the alloimmunization in pregnancy is hemolytic disease of the fetus and newborn (HDFN), neonatal alloimmune neutropenia (NAN) or fetal and neonatal alloimmune thrombocytopenia (FNAIT).

In some embodiments, the patient is a sensitized patient who is sensitized from a previous treatment for a condition or disease. In some embodiments, the patient received a previous treatment for a condition or disease, wherein the previous treatment did not comprise the population of cells, and wherein: (a) the population of cells is administered for the treatment of the same condition or disease as the previous treatment; (b) the population of cells exhibits an enhanced therapeutic effect for the treatment of the condition or disease in the patient as compared to the previous treatment; (c) the population of cells exhibits a longer therapeutic effect for the treatment of the condition or disease in the patient as compared to the previous treatment; (d) the previous treatment was therapeutically effective; (e) the previous treatment was therapeutically ineffective; (f) the patient developed an immune reaction against the previous treatment; and/or (g) the population of cells is administered for the treatment of a different condition or disease as the previous treatment.

In some embodiments, the previous treatment comprises administering a population of therapeutic cells comprising a suicide gene or a safety switch system, and the immune reaction occurs in response to activation of the suicide gene or the safety switch system.

In some embodiments, the previous treatment comprises a mechanically assisted treatment, optionally wherein the mechanically assisted treatment comprises hemodialysis or a ventricle assist device.

In some embodiments, the patient has an allergy, optionally wherein the allergy is an allergy selected from the group consisting of a hay fever, a food allergy, an insect allergy, a drug allergy, and atopic dermatitis.

In some embodiments, the cells further comprise one or more exogenous polypeptides selected from the group consisting of DUX4, CD24, CD46, CD55, CD59, CD200, PD-L1, HLA-E, HLA-G, IDO1, FasL, IL-35, IL-39, CCL21, CCL22, Mfge8, Serpin B9, and a combination thereof.

In some embodiments, the cells further comprise reduced expression levels of CD142, relative to a cell of the same cell type that does not comprise a modification. In some embodiments, the cells further comprise reduced expression levels of CD46, relative to a cell of the same cell type that does not comprise a modification. In some embodiments, the cells further comprise reduced expression levels of CD59, relative to a cell of the same cell type that does not comprise a modification.

In some embodiments, the cells are differentiated from stem cells. In some embodiments, the stem cells are mesenchymal stem cells. In some embodiments, the stem cells are embryonic stem cells. In some embodiments, the stem cells are pluripotent stem cells, optionally wherein the pluripotent stem cells are induced pluripotent stem cells.

In some embodiments, the cells are selected from the group consisting of cardiac cells, neural cells, endothelial cells, T cells, B cells, pancreatic islet cells, retinal pigmented epithelium cells, hepatocytes, thyroid cells, skin cells, blood cells, plasma cells, platelets, renal cells, epithelial cells, chimeric antigen receptor (CAR) T cells, NK cells, and CAR-NK cells. In some embodiments, the cells are derived from primary cells. In some embodiments, the primary cells are primary T cells, primary beta cells, or primary retinal pigment epithelial cells. In some embodiments, the cells derived from primary T cells are derived from a pool of T cells comprising primary T cells from one or more subjects different from the patient.

In some embodiments, the cells comprise a second exogenous polynucleotide encoding a chimeric antigen receptor (CAR). In some embodiments, the antigen binding domain of the CAR binds to CD19, CD22, or BCMA. In some embodiments, the CAR is a CD19-specific CAR such that the cell is a CD19 CART cell. In some embodiments, the CAR is a CD22-specific CAR such that the cell is a CD22 CAR T cell. In some embodiments, the cell comprises a CD19-specific CAR and a CD22-specific CAR such that the cell is a CD19/CD22 CAR T cell. In some embodiments, the CD19-specific CAR and the CD22-specific CAR are encoded by a single bicistronic polynucleotide. In some embodiments, the CD19-specific CAR and the CD22-specific CAR are encoded by two separate polynucleotides.

In some embodiments, the first and/or second exogenous polynucleotide is inserted into a genomic locus comprising a safe harbor locus, a target locus, a B2M gene locus, a CIITA gene locus, a TRAC gene locus, or a TRB gene locus.

In some embodiments, the first and second genomic loci are the same. In some embodiments, the first and second genomic loci are different. In some embodiments, the cells each further comprise a third exogenous polynucleotide inserted into a third genomic locus. In some embodiments, the third genomic locus is the same as the first or second genomic loci. In some embodiments, the third genomic locus is different from the first and/or second genomic loci.

In some embodiments, the safe harbor locus is selected from the group consisting of: a CCR5 gene locus, a PPP1R12C (also known as AAVS1) gene, and a CLYBL gene locus.

In some embodiments, the target locus is selected from the group consisting of: a CXCR4 gene locus, an albumin gene locus, a SHS231 locus, a ROSA26 gene locus, a CD142 gene locus, a MICA gene locus, a MICB gene locus, a LRP1 gene locus, a HMGB1 gene locus, an ABO gene locus, a RHD gene locus, a FUT1 gene locus, and a KDM5D gene locus.

In some embodiments, the insertion into the CCR5 gene locus is in exon 1-3, intron 1-2 or another coding sequence (CDS) of the CCR5 gene. In some embodiments, the insertion into the PPP1R12C gene locus is intron 1 or intron 2 of the PPP1R12C gene. In some embodiments, the insertion into the CLYBL gene locus is intron 2 of the CLYBL gene. In some embodiments, the insertion into the ROSA26 gene locus is intron 1 of the ROSA26 gene. In some embodiments, the insertion into the safe harbor locus is a SHS231 locus. In some embodiments, the insertion into the CD142 gene locus is in exon 2 or another CDS of the CD142 gene. In some embodiments, the insertion into the MICA gene locus is in a CDS of the MICA gene. In some embodiments, the insertion into the MICB gene locus is in a CDS of the MICB gene. In some embodiments, the insertion into the B2M gene locus is in exon 2 or another CDS of the B2M gene. In some embodiments, the insertion into the CIITA gene locus is in exon 3 or another CDS of the CIITA gene. In some embodiments, the insertion into the TRAC gene locus is in exon 2 or another CDS of the TRAC gene. In some embodiments, the insertion into the TRB gene locus is in a CDS of the TRB gene.

In some embodiments, the cells derived from primary T cells comprise reduced expression of one or more of: (a) an endogenous T cell receptor; (b) cytotoxic T-lymphocyte-associated protein 4 (CTLA4); (c) programmed cell death (PD1); and (d) programmed cell death ligand 1 (PD-L1), wherein the reduced expression is due to a modification and the reduced expression is relative to a cell of the same cell type that does not comprise the modification. In some embodiments, the cells derived from primary T cells comprise reduced expression of TRAC.

In some embodiments, the cells are T cells derived from induced pluripotent stem cells that comprise reduced expression of one or more of: (a) an endogenous T cell receptor; (b) cytotoxic T-lymphocyte-associated protein 4 (CTLA4); (c) programmed cell death (PD1); and (d) programmed cell death ligand 1 (PD-L1), wherein the reduced expression is due to a modification and the reduced expression is relative to a cell of the same cell type that does not comprise the modification. In some embodiments, the cells are T cells derived from induced pluripotent stem cells that comprise reduced expression of TRAC and TRB.

In some embodiments, the exogenous polynucleotide is operably linked to a promoter. In some embodiments, the promoter is a CAG and/or an EF1a promoter.

In some embodiments, the population of cells is administered at least 1 day or more after the patient is sensitized against one or more alloantigens, or at least 1 day or more after the patient had received the allogeneic transplant. In some embodiments, the population of cells is administered at least 1 week or more after the patient is sensitized against one or more alloantigens, or at least 1 week or more after the patient had received the allogeneic transplant. In some embodiments, the population of cells is administered at least 1 month or more after the patient is sensitized against one or more alloantigens, at least 1 month or more after the patient had received the allogeneic transplant.

In some embodiments, the patient exhibits no immune response upon administration of the population of cells. In some embodiments, the no immune response upon administration of the population of cells is selected from the group consisting of no systemic immune response, no adaptive immune response, no innate immune response, no T cell response, no B cell response, and no systemic acute cellular immune response.

In some embodiments, the patient exhibits one or more of: (a) no systemic TH1 activation upon administering the population of cells; (b) no immune activation of peripheral blood mononuclear cells (PBMCs) upon administering the population of cells; (c) no donor specific IgG antibodies against the population of cells upon administering the population of cells; (d) no IgM and IgG antibody production against the population of cells upon administering the population of cells; and (e) no cytotoxic T cell killing of the population of cells upon administering the population of cells.

In some embodiments, the patient is not administered an immunosuppressive agent at least 3 days or more before or after the administration of the population of cells.

In some embodiments, the method comprises a dosing regimen comprising: (a) a first administration comprising a therapeutically effective amount of the population of cells; (b) a recovery period; and (c) a second administration comprising a therapeutically effective amount of the population of cells. In some embodiments, the recovery period comprises at least 1 month or more. In some embodiments, the recovery period comprises at least 2 months or more. In some embodiments, the second administration is initiated when the cells from the first administration are no longer detectable in the patient.

In some embodiments, the hypoimmunogenic cells are eliminated by a suicide gene or a safety switch system, and wherein the second administration is initiated when the cells from the first administration are no longer detectable in the patient.

In some embodiments, the use of the cells further comprises administering the dosing regimen at least twice.

In some embodiments, the population of cells is administered for treatment of a cellular deficiency or as a cellular therapy for the treatment of a condition or disease in a tissue or organ selected from the group consisting of heart, lung, kidney, liver, pancreas, intestine, stomach, cornea, bone marrow, blood vessel, heart valve, brain, spinal cord, and bone.

In some embodiments, (a) the cellular deficiency is associated with a neurodegenerative disease or the cellular therapy is for the treatment of a neurodegenerative disease; (b) the cellular deficiency is associated with a liver disease or the cellular therapy is for the treatment of liver disease; (c) the cellular deficiency is associated with a corneal disease or the cellular therapy is for the treatment of corneal disease; (d) the cellular deficiency is associated with a cardiovascular condition or disease or the cellular therapy is for the treatment of a cardiovascular condition or disease; (e) the cellular deficiency is associated with diabetes or the cellular therapy is for the treatment of diabetes; (f) the cellular deficiency is associated with a vascular condition or disease or the cellular therapy is for the treatment of a vascular condition or disease; (g) the cellular deficiency is associated with autoimmune thyroiditis or the cellular therapy is for the treatment of autoimmune thyroiditis; or (h) the cellular deficiency is associated with a kidney disease or the cellular therapy is for the treatment of a kidney disease.

In some embodiments, (a) the neurodegenerative disease is selected from the group consisting of leukodystrophy, Huntington's disease, Parkinson's disease, multiple sclerosis, transverse myelitis, and Pelizaeus-Merzbacher disease (PMD); (b) the liver disease comprises cirrhosis of the liver; (c) the corneal disease is Fuchs dystrophy or congenital hereditary endothelial dystrophy; or (d) the cardiovascular disease is myocardial infarction or congestive heart failure.

In some embodiments, the population of cells comprises: (a) cells selected from the group consisting of glial progenitor cells, (b) oligodendrocytes, astrocytes, and dopaminergic neurons, optionally wherein the dopaminergic neurons are selected from the group consisting of neural stem cells, neural progenitor cells, immature dopaminergic neurons, and mature dopaminergic neurons; (c) hepatocytes or hepatic progenitor cells; (d) corneal endothelial progenitor cells or corneal endothelial cells; (e) cardiomyocytes or cardiac progenitor cells; (f) pancreatic islet cells, including pancreatic beta islet cells, optionally wherein the pancreatic islet cells are selected from the group consisting of a pancreatic islet progenitor cell, an immature pancreatic islet cell, and a mature pancreatic islet cell; (g) endothelial cells; (h) thyroid progenitor cells; or (i) renal precursor cells or renal cells.

In some embodiments, the population of cells is administered for the treatment of cancer. In some embodiments, the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

In some embodiments, the patient is receiving a tissue or organ transplant, optionally wherein the tissue or organ transplant or partial organ transplant is selected from the group consisting of a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, a bone transplant, a partial lung transplant, a partial kidney transplant, a partial liver transplant, a partial pancreas transplant, a partial intestine transplant, and a partial cornea transplant.

In some embodiments, the tissue or organ transplant is an allograft transplant. In some embodiments, the tissue or organ transplant is an autograft transplant.

In some embodiments, the population of cells is administered for the treatment of a cellular deficiency in a tissue or organ and the tissue or organ transplant is for the replacement of the same tissue or organ. In some embodiments, the population of cells is administered for the treatment of a cellular deficiency in a tissue or organ and the tissue or organ transplant is for the replacement of a different tissue or organ. In some embodiments, the organ transplant is a kidney transplant and the population of cells is a population of renal precursor cells or renal cells. In some embodiments, the patient has diabetes. In some embodiments, the organ transplant is a heart transplant and the population of cells is a population of cardiac progenitor cells or pacemaker cells. In some embodiments, the organ transplant is a pancreas transplant and the population of cells is a population of pancreatic beta islet cells. In some embodiments, the organ transplant is a partial liver transplant and the population of cells is a population of hepatocytes or hepatic progenitor cells.

In some aspects, provided herein is a method of treating a patient in need thereof comprising administering a population of hypoimmunogenic cells, wherein the hypoimmunogenic cells comprise a first exogenous polynucleotide encoding CD47, a second exogenous polynucleotide encoding a CAR and (I) one or more of: (a) reduced expression of major histocompatibility complex (MHC) class I and/or class II human leukocyte antigens; (b) reduced expression of MHC class I and class II human leukocyte antigens; (c) reduced expression of beta-2-microglobulin (B2M) and/or MHC class II transactivator (CIITA); and/or (d) reduced expression of B2M and CIITA; wherein the reduced expression is due to a modification and the reduced expression is relative to a cell of the same cell type that does not comprise the modification; (II) wherein: (a) the patient is not a sensitized patient; or (b) the patient is a sensitized patient, wherein the patient: (i) is sensitized against one or more alloantigens; (ii) is sensitized against one or more autologous antigens; (iii) is sensitized from a previous transplant; (iv) is sensitized from a previous pregnancy; (v) received a previous treatment for a condition or disease; and/or (vi) is a tissue or organ patient, and the hypoimmunogenic cells are administered prior to administering the tissue or organ transplant.

In some embodiments, the patient is a sensitized patient and wherein the patient exhibits memory B cells and/or memory T cells reactive against the one or more alloantigens or one or more autologous antigens. In some embodiments, the one or more alloantigens comprise human leukocyte antigens.

In some embodiments, the patient is a sensitized patient who is sensitized from a previous transplant, wherein: the previous transplant is selected from the group consisting of a cell transplant, a blood transfusion, a tissue transplant, and an organ transplant, optionally the previous transplant is an allogeneic transplant; or the previous transplant is a transplant selected from the group consisting of a chimera of human origin, a modified non-human autologous cell, a modified autologous cell, an autologous tissue, and an autologous organ, optionally the previous transplant is an autologous transplant.

In some embodiments, the patient is a sensitized patient who is sensitized from a previous pregnancy and wherein the patient had previously exhibited alloimmunization in pregnancy, optionally wherein the alloimmunization in pregnancy is hemolytic disease of the fetus and newborn (HDFN), neonatal alloimmune neutropenia (NAN) or fetal and neonatal alloimmune thrombocytopenia (FNAIT).

In some embodiments, the patient is a sensitized patient who is sensitized from a previous treatment for a condition or disease. In some embodiments, the patient received a previous treatment for a condition or disease, wherein the previous treatment did not comprise the population of cells, and wherein: (a) the population of cells is administered for the treatment of the same condition or disease as the previous treatment; (b) the population of cells exhibits an enhanced therapeutic effect for the treatment of the condition or disease in the patient as compared to the previous treatment; (c) the population of cells exhibits a longer therapeutic effect for the treatment of the condition or disease in the patient as compared to the previous treatment; (d) the previous treatment was therapeutically effective; (e) the previous treatment was therapeutically ineffective; (f) the patient developed an immune reaction against the previous treatment; and/or (g) the population of cells is administered for the treatment of a different condition or disease as the previous treatment.

In some embodiments, the previous treatment comprises administering a population of therapeutic cells comprising a suicide gene or a safety switch system, and the immune reaction occurs in response to activation of the suicide gene or the safety switch system.

In some embodiments, the previous treatment comprises a mechanically assisted treatment, optionally wherein the mechanically assisted treatment comprises hemodialysis or a ventricle assist device.

In some embodiments, the patient has an allergy, optionally wherein the allergy is an allergy selected from the group consisting of a hay fever, a food allergy, an insect allergy, a drug allergy, and atopic dermatitis.

In some embodiments, the cells further comprise one or more exogenous polypeptides selected from the group consisting of DUX4, CD24, CD46, CD55, CD59, CD200, PD-L1, HLA-E, HLA-G, IDO1, FasL, IL-35, IL-39, CCL21, CCL22, Mfge8, Serpin B9, and a combination thereof.

In some embodiments, the cells further comprise reduced expression levels of CD142, relative to a cell of the same cell type that does not comprise a modification. In some embodiments, the cells further comprise reduced expression levels of CD46, relative to a cell of the same cell type that does not comprise a modification. In some embodiments, the cells further comprise reduced expression levels of CD59, relative to a cell of the same cell type that does not comprise a modification.

In some embodiments, the cells are differentiated from stem cells. In some embodiments, the stem cells are mesenchymal stem cells. In some embodiments, the stem cells are embryonic stem cells. In some embodiments, the stem cells are pluripotent stem cells, optionally wherein the pluripotent stem cells are induced pluripotent stem cells. In some embodiments, the cells are CAR T cells or CAR-NK cells. In some embodiments, the cells are derived from primary T cells. In some embodiments, the cells are derived from a pool of T cells comprising primary T cells from one or more subjects different from the patient.

In some embodiments, the antigen binding domain of the CAR binds to CD19, CD22, or BCMA. In some embodiments, the CAR is a CD19-specific CAR such that the cell is a CD19 CAR T cell. In some embodiments, the CAR is a CD22-specific CAR such that the cell is a CD22 CART cell. In some embodiments, the cell comprises a CD19-specific CAR and a CD22-specific CAR such that the cell is a CD19/CD22 CART cell. In some embodiments, the CD19-specific CAR and the CD22-specific CAR are encoded by a single bicistronic polynucleotide.

In some embodiments, the CD19-specific CAR and the CD22-specific CAR are encoded by two separate polynucleotides

In some embodiments, the first and/or second exogenous polynucleotide is inserted into a genomic locus comprising a safe harbor locus, a target locus, a B2M gene locus, a CIITA gene locus, a TRAC gene locus, or a TRB gene locus. In some embodiments, the first and second genomic loci are the same. In some embodiments, the first and second genomic loci are different.

In some embodiments, the cells each further comprise a third exogenous polynucleotide inserted into a third genomic locus. In some embodiments, the third genomic locus is the same as the first or second genomic loci. In some embodiments, the third genomic locus is different from the first and/or second genomic loci.

In some embodiments, the safe harbor locus is selected from the group consisting of: a CCR5 gene locus, a PPP1R12C (also known as AAVS1) gene, and a CLYBL gene locus. In some embodiments, the target locus is selected from the group consisting of: a CXCR4 gene locus, an albumin gene locus, a SHS231 locus, a ROSA26 gene locus, a CD142 gene locus, a MICA gene locus, a MICB gene locus, a LRP1 gene locus, a HMGB1 gene locus, an ABO gene locus, a RHD gene locus, a FUT1 gene locus, and a KDM5D gene locus.

In some embodiments, the insertion into the CCR5 gene locus is in exon 1-3, intron 1-2 or another coding sequence (CDS) of the CCR5 gene. In some embodiments, the insertion into the PPP1R12C gene locus is intron 1 or intron 2 of the PPP1R12C gene. In some embodiments, the insertion into the CLYBL gene locus is intron 2 of the CLYBL gene. In some embodiments, the insertion into the ROSA26 gene locus is intron 1 of the ROSA26 gene. In some embodiments, the insertion into the insertion into the safe harbor locus is a SHS231 locus. In some embodiments, the insertion into the CD142 gene locus is in exon 2 or another CDS of the CD142 gene. In some embodiments, the insertion into the MICA gene locus is in a CDS of the MICA gene. In some embodiments, the insertion into the MICB gene locus is in a CDS of the MICB gene. In some embodiments, the insertion into the B2M gene locus is in exon 2 or another CDS of the B2M gene. In some embodiments, the insertion into the CIITA gene locus is in exon 3 or another CDS of the CIITA gene. In some embodiments, the insertion into the TRAC gene locus is in exon 2 or another CDS of the TRAC gene. In some embodiments, the insertion into the TRB gene locus is in a CDS of the TRB gene.

In some embodiments, the cells derived from primary T cells comprise reduced expression of one or more of: an endogenous T cell receptor; cytotoxic T-lymphocyte-associated protein 4 (CTLA4); programmed cell death (PD1); and programmed cell death ligand 1 (PD-L1), wherein the reduced expression is due to a modification and the reduced expression is relative to a cell of the same cell type that does not comprise the modification. In some embodiments, the cells derived from primary T cells comprised reduced expression of TRAC.

In some embodiments, the cells are T cells derived from induced pluripotent stem cells that comprise reduced expression of one or more of: an endogenous T cell receptor; cytotoxic T-lymphocyte-associated protein 4 (CTLA4); programmed cell death (PD1); and programmed cell death ligand 1 (PD-L1), wherein the reduced expression is due to a modification and the reduced expression is relative to a cell of the same cell type that does not comprise the modification. In some embodiments, the cells are T cells derived from induced pluripotent stem cells that comprise reduced expression of TRAC and TRB.

In some embodiments, the exogenous polynucleotide is operably linked to a promoter. In some embodiments, the promoter is a CAG and/or an EF1a promoter.

In some embodiments, the population of cells is administered at least 1 day or more after the patient is sensitized against one or more alloantigens, or at least 1 day or more after the patient had received the allogeneic transplant. In some embodiments, the population of cells is administered at least 1 week or more after the patient is sensitized against one or more alloantigens, or at least 1 week or more after the patient had received the allogeneic transplant. In some embodiments, the population of cells is administered at least 1 month or more after the patient is sensitized against one or more alloantigens, at least 1 month or more after the patient had received the allogeneic transplant.

In some embodiments, the patient exhibits no immune response upon administration of the population of cells. In some embodiments, the no immune response upon administration of the population of cells is selected from the group consisting of no systemic immune response, no adaptive immune response, no innate immune response, no T cell response, no B cell response, and no systemic acute cellular immune response.

In some embodiments, the patient exhibits one or more of: (i) no systemic TH1 activation upon administering the population of cells; (ii) no immune activation of peripheral blood mononuclear cells (PBMCs) upon administering the population of cells; (iii) no donor specific IgG antibodies against the population of cells upon administering the population of cells; (iv) no IgM and IgG antibody production against the population of cells upon administering the population of cells; and (v) no cytotoxic T cell killing of the population of cells upon administering the population of cells.

In some embodiments, the patient is not administered an immunosuppressive agent at least 3 days or more before or after the administration of the population of cells.

In some embodiments, the method comprises a dosing regimen comprising: a first administration comprising a therapeutically effective amount of the population of cells; a recovery period; and a second administration comprising a therapeutically effective amount of the population of cells. In some embodiments, the recovery period comprises at least 1 month or more. In some embodiments, the recovery period comprises at least 2 months or more.

In some embodiments, the second administration is initiated when the cells from the first administration are no longer detectable in the patient.

In some embodiments, the hypoimmunogenic cells are eliminated by a suicide gene or a safety switch system, and wherein the second administration is initiated when the cells from the first administration are no longer detectable in the patient.

In some embodiments, the method further comprises administering the dosing regimen at least twice.

In some embodiments, the population of cells is administered for the treatment of cancer. In some embodiments, the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

In one aspect, provided is use of a population of hypoimmunogenic cells for treatment of a disorder in a patient, wherein the hypoimmunogenic cells comprises a first exogenous polynucleotide encoding CD47, a second exogenous polynucleotide encoding a CAR and

(I) one or more of: (a) reduced expression of major histocompatibility complex (MHC) class I and/or class II human leukocyte antigens; (b) reduced expression of MHC class I and class II human leukocyte antigens; (c) reduced expression of beta-2-microglobulin (B2M) and/or MHC class II transactivator (CIITA); and/or (d) reduced expression of B2M and CIITA; wherein the reduced expression is due to a modification and the reduced expression is relative to a cell of the same cell type that does not comprise the modification;
(II) wherein: the patient is not a sensitized patient; or the patient is a sensitized patient.

In some embodiments, the patient is a sensitized patient and wherein the patient exhibits memory B cells and/or memory T cells reactive against the one or more alloantigens or one or more autologous antigens.

In some embodiments, the one or more alloantigens comprise human leukocyte antigens.

In some embodiments, the patient is a sensitized patient who is sensitized from a previous transplant, wherein: the previous transplant is selected from the group consisting of a cell transplant, a blood transfusion, a tissue transplant, and an organ transplant, optionally the previous transplant is an allogeneic transplant; or the previous transplant is a transplant selected from the group consisting of a chimera of human origin, a modified non-human autologous cell, a modified autologous cell, an autologous tissue, and an autologous organ, optionally the previous transplant is an autologous transplant.

In some embodiments, the patient is a sensitized patient who is sensitized from a previous pregnancy and wherein the patient had previously exhibited alloimmunization in pregnancy, optionally wherein the alloimmunization in pregnancy is hemolytic disease of the fetus and newborn (HDFN), neonatal alloimmune neutropenia (NAN) or fetal and neonatal alloimmune thrombocytopenia (FNAIT).

In some embodiments, the patient is a sensitized patient who is sensitized from a previous treatment for a condition or disease. In some embodiments, the patient received a previous treatment for a condition or disease, wherein the previous treatment did not comprise the population of cells, and wherein: (a) the population of cells is administered for the treatment of the same condition or disease as the previous treatment; (b) the population of cells exhibits an enhanced therapeutic effect for the treatment of the condition or disease in the patient as compared to the previous treatment; (c) the population of cells exhibits a longer therapeutic effect for the treatment of the condition or disease in the patient as compared to the previous treatment; (d) the previous treatment was therapeutically effective; (e) the previous treatment was therapeutically ineffective; (f) the patient developed an immune reaction against the previous treatment; and/or (g) the population of cells is administered for the treatment of a different condition or disease as the previous treatment. In some embodiments, the previous treatment comprises administering a population of therapeutic cells comprising a suicide gene or a safety switch system, and the immune reaction occurs in response to activation of the suicide gene or the safety switch system. In some embodiments, the previous treatment comprises a mechanically assisted treatment, optionally wherein the mechanically assisted treatment comprises hemodialysis or a ventricle assist device.

In some embodiments, the patient has an allergy, optionally wherein the allergy is an allergy selected from the group consisting of a hay fever, a food allergy, an insect allergy, a drug allergy, and atopic dermatitis.

In some embodiments, the cells further comprise one or more exogenous polypeptides selected from the group consisting of DUX4, CD24, CD46, CD55, CD59, CD200, PD-L1, HLA-E, HLA-G, IDO1, FasL, IL-35, IL-39, CCL21, CCL22, Mfge8, Serpin B9, and a combination thereof.

In some embodiments, the cells further comprise reduced expression levels of CD142 relative to a cell of the same cell type that does not comprise a modification. In some embodiments, the cells further comprise reduced expression levels of CD46 relative to a cell of the same cell type that does not comprise a modification. In some embodiments, the cells further comprise reduced expression levels of CD59 relative to a cell of the same cell type that does not comprise a modification.

In some embodiments, the cells are differentiated from stem cells. In some embodiments, the stem cells are mesenchymal stem cells. In some embodiments, the stem cells are embryonic stem cells. In some embodiments, the stem cells are pluripotent stem cells, optionally wherein the pluripotent stem cells are induced pluripotent stem cells. In some embodiments, the cells are CAR T cells or CAR-NK cells. the cells are differentiated from stem cells. In some embodiments cells are derived from primary T cells. In some embodiments, the cells are derived from a pool of T cells comprising primary T cells from one or more subjects different from the patient.

In some embodiments, the antigen binding domain of the CAR binds to CD19, CD22, or BCMA. In some embodiments, the CAR is a CD19-specific CAR such that the cell is a CD19 CAR T cell. In some embodiments, the CAR is a CD22-specific CAR such that the cell is a CD22 CAR T cell. In some embodiments, the cell comprises a CD19-specific CAR and a CD22-specific CAR such that the cell is a CD19/CD22 CAR T cell. In some embodiments, the CD19-specific CAR and the CD22-specific CAR are encoded by a single bicistronic polynucleotide. In some embodiments, the CD19-specific CAR and the CD22-specific CAR are encoded by two separate polynucleotides

In some embodiments, the first and/or second exogenous polynucleotide is inserted into a genomic locus comprising a safe harbor locus, a target locus, a B2M gene locus, a CIITA gene locus, a TRAC gene locus, or a TRB gene locus.

In some embodiments, the first and second genomic loci are the same. In some embodiments, the first and second genomic loci are different. In some embodiments, the cells each further comprise a third exogenous polynucleotide inserted into a third genomic locus. In some embodiments, the third genomic locus is the same as the first or second genomic loci. In some embodiments, the third genomic locus is different from the first and/or second genomic loci.

In some embodiments, the safe harbor locus is selected from the group consisting of: a CCR5 gene locus, a PPP1R12C (also known as AAVS1) gene, and a CLYBL gene locus. In some embodiments, the target locus is selected from the group consisting of: a CXCR4 gene locus, an albumin gene locus, a SHS231 locus, a ROSA26 gene locus, a CD142 gene locus, a MICA gene locus, a MICB gene locus, a LRP1 gene locus, a HMGB1 gene locus, an ABO gene locus, a RHD gene locus, a FUT1 gene locus, and a KDM5D gene locus. In some embodiments, the insertion into the CCR5 gene locus is in exon 1-3, intron 1-2 or another coding sequence (CDS) of the CCR5 gene. In some embodiments, the insertion into the PPP1R12C gene locus is intron 1 or intron 2 of the PPP1R12C gene. In some embodiments, the insertion into the CLYBL gene locus is intron 2 of the CLYBL gene.

In some embodiments, the insertion into the ROSA26 gene locus is intron 1 of the ROSA26 gene. In some embodiments, the insertion into the insertion into the safe harbor locus is a SHS231 locus. In some embodiments, the insertion into the CD142 gene locus is in exon 2 or another CDS of the CD142 gene. In some embodiments, the insertion into the MICA gene locus is in a CDS of the MICA gene. In some embodiments, the insertion into the MICB gene locus is in a CDS of the MICB gene. In some embodiments, the insertion into the B2M gene locus is in exon 2 or another CDS of the B2M gene. In some embodiments, the insertion into the CIITA gene locus is in exon 3 or another CDS of the CIITA gene. In some embodiments, the insertion into the TRAC gene locus is in exon 2 or another CDS of the TRAC gene. In some embodiments, the insertion into the TRB gene locus is in a CDS of the TRB gene.

In some embodiments, the cells derived from primary T cells comprise reduced expression of one or more of: (a) an endogenous T cell receptor; (b) cytotoxic T-lymphocyte-associated protein 4 (CTLA4); (c) programmed cell death (PD1); and (d) programmed cell death ligand 1 (PD-L1), wherein the reduced expression is due to a modification and the reduced expression is relative to a cell of the same cell type that does not comprise the modification.

In some embodiments, the cells derived from primary T cells comprised reduced expression of TRAC.

In some embodiments, the cells are T cells derived from induced pluripotent stem cells that comprise reduced expression of one or more of: (a) an endogenous T cell receptor; (b) cytotoxic T-lymphocyte-associated protein 4 (CTLA4); (c) programmed cell death (PD1); and (d) programmed cell death ligand 1 (PD-L1), wherein the reduced expression is due to a modification and the reduced expression is relative to a cell of the same cell type that does not comprise the modification. In some embodiments, the cells are T cells derived from induced pluripotent stem cells that comprise reduced expression of TRAC and TRB.

In some embodiments, the exogenous polynucleotide is operably linked to a promoter. In some embodiments, the promoter is a CAG and/or an EF1a promoter.

In some embodiments, the population of cells is administered at least 1 day or more after the patient is sensitized against one or more alloantigens, or at least 1 day or more after the patient had received the allogeneic transplant. In some embodiments, the population of cells is administered at least 1 week or more after the patient is sensitized against one or more alloantigens, or at least 1 week or more after the patient had received the allogeneic transplant. In some embodiments, the population of cells is administered at least 1 month or more after the patient is sensitized against one or more alloantigens, at least 1 month or more after the patient had received the allogeneic transplant.

In some embodiments, the patient exhibits no immune response upon administration of the population of cells. In some embodiments, the no immune response upon administration of the population of cells is selected from the group consisting of no systemic immune response, no adaptive immune response, no innate immune response, no T cell response, no B cell response, and no systemic acute cellular immune response. In some embodiments, the patient exhibits one or more of: (a) no systemic TH1 activation upon administering the population of cells; (b) no immune activation of peripheral blood mononuclear cells (PBMCs) upon administering the population of cells; (c) no donor specific IgG antibodies against the population of cells upon administering the population of cells; (d) no IgM and IgG antibody production against the population of cells upon administering the population of cells; and (e) no cytotoxic T cell killing of the population of cells upon administering the population of cells.

In some embodiments, the patient is not administered an immunosuppressive agent at least 3 days or more before or after the administration of the population of cells.

In some embodiments, the method comprises a dosing regimen comprising: (a) a first administration comprising a therapeutically effective amount of the population of cells; (b) a recovery period; and (c) a second administration comprising a therapeutically effective amount of the population of cells. In some embodiments, the recovery period comprises at least 1 month or more. In some embodiments, the recovery period comprises at least 2 months or more. In some embodiments, the second administration is initiated when the cells from the first administration are no longer detectable in the patient. In some embodiments, the hypoimmunogenic cells are eliminated by a suicide gene or a safety switch system, and wherein the second administration is initiated when the cells from the first administration are no longer detectable in the patient. In some embodiments, the use of the cells provided herein further comprises administering the dosing regimen at least twice.

In some embodiments, the population of cells is administered for the treatment of cancer. In some embodiments, the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

In some embodiments of the use or the method described, the previous treatment comprises an allogeneic CAR-T cell based therapy or an autologous CAR-T cell based therapy, wherein the autologous CAR-T cell based therapy is selected from the group consisting of brexucabtagene autoleucel, axicabtagene ciloleucel, idecabtagene vicleucel, lisocabtagene maraleucel, tisagenlecleucel, Descartes-08 or Descartes-11 from Cartesian Therapeutics, CTL110 from Novartis, P-BMCA-101 from Poseida Therapeutics, AUTO4 from Autolus Limited, UCARTCS from Cellectis, PBCAR19B or PBCAR269A from Precision Biosciences, FT819 from Fate Therapeutics, and CYAD-211 from Clyad Oncology.

### BRIEF DESCRIPTION OF THE DRAWINGS

FIGS. 1A-1F are a set of representative ELISPOT quantitations from serum of NHPs crossover administered wild-type human (FIGS. 1A, 1B, 1D and 1F) and HIP (FIGS. 1A, 1C, 1D and 1E) iPSCs. FIGS. 1A-1C show results of the study group receiving wild-type human iPSCs (wt^{xeno}) at first injection, wt^{xeno} at second injection, and human HIP iPSCs (HIP^{xeno}) at third injection. FIGS. 1D-1F show results of the study group receiving HIP^{xeno} at first injection, HIP^{xeno} at second injection and wt^{xeno} at third injection. All assays run after receiving wt^{xeno} injection and HIP^{xeno} injection are shown as the bars with horizontal lines and the bars with vertical lines, respectively. Blood was drawn for analysis at various time points, for example, at pre-treatment (“pre-Tx”), day 7, day 13, day 75, and thereafter of cell administration including at crossover injection (“pre-Tx”) and at days 7, 13, and 75 thereafter. Day signifiers in brackets below indicate time that the blood was drawn relative to first injection (first row), second injection (second row) and third injection (third row), as shown in FIGS. 1A-7C, 8C and 8E.

FIGS. 2A and 2B are a set of representative graphs showing donor-specific IgG antibody binding in serum of NHPs crossover administered wild-type (FIG. 2A) or HIP (FIGS. 2A and 2B) human iPSCs. FIGS. 2A and 2B show results of the study group receiving wt^{xeno} at first injection, wt^{xeno} at second injection, and HIP^{xeno} at third injection. All assays run against wt^{xeno} and HIP^{xeno} are shown as circles with horizontal lines and circles with vertical lines, respectively in FIG. 2A. FIG. 2B shows IgG DSA levels after receiving the HIP^{xeno} injection.

FIGS. 3A and 3B are a set of representative graphs showing donor-specific IgG antibody binding in serum of NHPs crossover administered wild-type (FIGS. 3A and 3B) or HIP (FIG. 3A) human iPSCs. FIGS. 3A and 3B show results of the study group receiving HIP^{xeno} at first injection, HIP^{xeno} at second injection and wt^{xeno} at third injection. All assays run against wt^{xeno} and HIP^{xeno} are shown as circles with horizontal lines and circles with vertical lines, respectively in FIG. 2A. FIG. 3B shows IgG DSA levels after receiving the wt^{xeno} injection.

FIGS. 4A-4C are a set of representative graphs showing total IgM antibodies in serum of NHPs crossover administered wild-type (FIGS. 4A and 4B) or HIP (FIGS. 4A and 4C) human iPSCs. FIGS. 4A-4C show results of the study group receiving human HIP iPSCs) (HIP^{xeno}) at first injection, HIP^{xeno} at second injection and wt^{xeno} at third injection. FIG. 4B shows total IgM antibody levels after receiving wt^{xeno} injection and FIG. 4C shows total IgM antibody levels after receiving HIP^{xeno} at the second injection.

FIGS. 5A-5C are a set of representative graphs showing total IgM antibodies in serum of NHPs crossover administered wild-type (FIGS. 5A and 5B) or HIP (FIGS. 5A and 5C) human iPSCs. FIGS. 5A-5C show results of the study group receiving wt^{xeno} at first injection, wt^{xeno} at second injection and HIP^{xeno} at third injection. FIG. 5B shows total IgM antibody levels after receiving wt^{xeno} at second injection and FIG. 5C shows total IgM antibody levels after receiving HIP^{xeno} at third injection.

FIGS. 6A-6C are a set of representative graphs showing total IgG antibodies in serum of NHPs crossover administered wild-type (FIGS. 6A and 6B) or HIP (FIGS. 6A and 6C) human iPSCs. FIGS. 6A-6C show results of the study group receiving HIP^{xeno} at first injection, HIP^{xeno} at second injection and wt^{xeno} at third injection. FIG. 6B shows total IgG antibody levels after receiving wt^{xeno} at third injection and FIG. 6C shows total IgG antibody levels after receiving HIP^{xeno} at second injection.

FIGS. 7A-7C are a set of representative graphs showing total IgG antibodies in serum of NHPs crossover administered wild-type (FIGS. 7A and 7B) or HIP (FIGS. 7A and 7C) human iPSCs. FIGS. 7A-7C show results of the study group receiving HIP^{xeno} at first injection, wt^{xeno} at second injection and HIP^{xeno} at third injection. FIG. 7B shows total IgG antibody levels after receiving wt^{xeno} at second injection and FIG. 7C shows total IgG antibody levels after receiving HIP^{xeno} at third injection.

FIGS. 8A-8E are a set of representative graphs showing an absence of natural killer (NK) cell-mediated killing of HIP human iPSCs into the wild-type NHPs. FIGS. 8A-8C show NK cell-mediated killing in the study group receiving HIP^{xeno} at first injection, HIP^{xeno} at second injection and wt^{xeno} at third injection. The absence of NK cell-killing of human HIP iPSCs at the first injection phase (FIG. 8A) and the second injection phase (FIG. 8B) is depicted in real-time cellular biosensor data graphs. FIGS. 8D and 8E show NK cell-mediated killing in the study group receiving wt^{xeno} at first injection, wt^{xeno} at second injection and HIP^{xeno} at third injection. The absence of NK cell-killing of human HIP iPSCs at the third injection phase (FIG. 8D) is depicted in real-time cellular biosensor data graph. Percent target cell killing is shown on the left y-axis (mean±s.d.), killing speed on the right y-axis (killing t_{1/2}⁻¹, mean±s.e.m.; shown as open triangles). Assays run after receiving wt^{xeno} and HIP^{xeno} injection are shown as circles with horizontal lines and circles with vertical lines, respectively.

FIG. 9A shows representative BLI images of transplanted HIP rhesus iPSCs in the left leg of an allogeneic NHP recipient. BLI signals over time and the percent of the BLI signal over time relative to the level at day 0 or pre-transplantation are shown below the BLI images in FIGS. 9A, 10, 11, 12A-12B and 13C. FIG. 9B shows an immunohistological image of tissue from the injection site at 6 weeks after transplantation. The image shows SMA-positive vessels and luciferase-positive cells which indicate the transplanted HIP rhesus iPSCs and progeny thereof.

FIG. 10 shows representative BLI images of transplanted wildtype rhesus iPSCs in the left leg of an allogeneic NHP recipient (top row) and transplanted HIP rhesus iPSCs in the right leg of the same recipient which has been sensitized for 5 weeks following transplant of the wildtype rhesus iPSCs (bottom row).

FIG. 11 shows representative BLI images of transplanted wildtype rhesus iPSCs in the left leg of another allogeneic NHP recipient (top row) and transplanted HIP rhesus iPSCs in the right leg of the same recipient which has been sensitized for 5 weeks following transplant of the wildtype rhesus iPSCs (bottom row).

FIGS. 12A and 12B show representative BLI images of an allogeneic NHP recipient from a crossover study of HIP rhesus iPSCs to wildtype rhesus iPSCs. The top row shows images of the transplanted HIP rhesus iPSCs and progeny thereof in the left leg of an allogeneic NHP recipient and the bottom row shows transplanted wildtype rhesus iPSCs in the right leg of the same recipient. Also depicted in the bottom right are images of transplanted HIP rhesus iPSCs and progeny thereof in the left leg of an allogeneic NHP recipient at 8 weeks and 9 weeks after the initial HIP iPSC transplantation.

FIG. 13A shows representative BLI signals over time for representative allogeneic NHP recipients of transplanted wildtype rhesus iPSCs initially in the left leg of an allogeneic NHP recipient and transplanted HIP rhesus iPSCs in the right leg of the same recipient upon crossover injection. FIG. 13B shows representative BLI signals over time for representative allogeneic NHP recipients of transplanted HIP rhesus iPSCs initially in the left leg of an allogeneic NHP recipient and transplanted wildtype rhesus iPSCs in the right leg of the same recipient upon crossover injection. FIG. 13C shows representative BLI images of an allogeneic NHP recipient of HIP rhesus iPSCs administered in the first injection into the left leg from day 0 to week 9.

FIGS. 14A-14G show characterization of human wt and HIP iPSCs before xenogeneic transplantation into NHP recipients. FIGS. 14A and 14B show the morphology of wt^{xeno} (FIG. 14A) and HIP^{xeno} (FIG. 14B) cultures. Surface expression of HLA class I and class II and CD47 on wt^{xeno} (FIG. 14C) and HIP^{xeno} (FIG. 14D) was assessed by flow cytometry and depicted as histograms. FIG. 14E shows the viability of the cell preparations of wt^{xeno} and HIP^{xeno} before transplantation. The viability into the NHP recipients was above 90% (mean±s.d.). FIG. 14F shows representative BLI images and BLI signals over time of NSG mice subcutaneously injected with wt^{xeno} iPSCs. FIG. 14G shows representative BLI images and BLI signals over time of NSG mice subcutaneously injected with HIP^{xeno} iPSCs.

FIGS. 15A-15J show characterization of rhesus wt and HIP iPSCs before allogeneic transplantation into NHP recipients. FIGS. 15A-15C show the morphology of wt^{allo} (FIG. 15A) and HIP^{allo} (FIGS. 15B and 15C) cultures. Surface expression of HLA class I and class II and CD47 on wt^{allo} (FIG. 15D) and HIP^{allo} (FIGS. 15E and 15F) was assessed by flow cytometry and depicted as histograms. FIG. 15G shows the viability of the cell preparations of wt^{allo} and HIP^{allo} before transplantation. The viability into the NHP recipients was above 90% (mean±s.d.). FIG. 15H shows representative BLI images and BLI signals over time of NSG mice subcutaneously injected with wt^{allo} iPSCs. FIGS. 15I and 15J show representative BLI images and BLI signals over time of NSG mice subcutaneously injected with HIP^{allo} iPSCs.

FIG. 16 is a representative graph assessing CD47 expression in B2M^{indel/indel}, CIITA^{indel/indel} CD47tg iPSCs. In these iPSCs, the CD47 transgene was inserted into a safe harbor site (AAVS1, CYBL, or CCR5), and a CAG or EF1α promoter was used to control expression of the CD47 polynucleotide. As shown, the B2M^{indel/indel}, CIITA^{indel/indel} CD47tg iPSCs express CD47 at ˜30-200 fold over baseline.

FIG. 17 is a representative graph assessing CD47 expression in B2M^{indel/indel}, CIITA^{indel/indel} CD47tg iPSCs. In these iPSCs, the CD47 transgene was inserted into a CYBL safe harbor site, and an EF1α promoter was used to control expression of the CD47 polynucleotide. As shown, the B2M^{indel/indel}, CIITA^{indel/indel} CD47tg iPSCs overexpress CD47 at P23 and P27.

FIG. 18 is a representative graph assessing CD47 expression in B2M^{indel/indel}, CIITA^{indel/indel} CD47tg iPSCs at several timepoints (P20, P21, P23, and P27). In these iPSCs, the CD47 transgene was inserted into a CCR5 or CLYBL safe harbor site, and a CAG or EF1α promoter was used to control express of the CD47 polynucleotide. As shown, the B2M^{indel/indel}, CIITA^{indel/indel}, CD47t_{g} iPSCs overexpress CD47 at the various time points.

FIGS. 19A-C are representative graphs from a study to assess killing of B2M^{indel/indel}, CIITA^{indel/indel}, CD47tg iPSCs by innate immune cells (NK cells and macrophages). The CD47tg of the B2M^{indel/indel} and CIITA^{indel/indel} iPSCs was inserted into a safe harbor site (AAVS1 (FIG. 19A), CYBL (FIG. 19B), or CCR5 (FIG. 19C)). As shown, all cell clones were protected from NK and macrophage cell killing.

Other objects, advantages and embodiments of the technology will be apparent from the detailed description following.

### DETAILED DESCRIPTION

### I. Introduction

The present disclosure is related to methods and compositions for alleviating and/or avoiding the effects of immune system reactions to cell therapies. To overcome the problem of a subject's immune rejection of cell-derived and/or tissue transplants, the inventors have developed and disclose herein an immune-evasive cell (e.g., a hypoimmunogenic cell or a hypoimmunogenic pluripotent cell) that represents a viable source for any transplantable cell type. Advantageously, the cells disclosed herein are not rejected by the recipient subject's immune system, regardless of the subject's genetic make-up or any existing response within the subject to one or more previous allogeneic or autologous cell-derived and/or tissue transplants.

The technology disclosed herein utilize genetic modifications to modulate (e.g., reduce or eliminate) MHC I and/or MHC II expression. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of genes involved in an immune response (e.g., by deleting genomic DNA of genes involved in an immune response or by insertions of genomic DNA into such genes, such that gene expression is impacted) in human cells. In certain embodiments, genome editing technologies or other gene modulation technologies are used to insert tolerance-inducing (tolerogenic) factors in human cells, rendering them and the differentiated cells prepared therefrom cells that can evade immune recognition upon engrafting into a recipient subject. As such, the cells described herein exhibit modulated expression of one or more genes and/or factors that affect MHC I and/or MHC II expression.

The genome editing techniques described herein enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

Certain genome editing techniques described herein enable single-stranded DNA breaks at the desired locus site where base editing or prime editing can be used to change single nucleic acid bases to an alternate base in order to alter the genome sequence. In some embodiments, base editing is used to modulate MHC I and/or MHC II antigen, tolerogenic factor(s), and/or CAR expression. Descriptions of base editing can be found, for example, in Rothgangl et al., Nat Biotechnol., 2021, 39, 949-957; Porto et al., Nat Rev Drug Discov., 2020, 19, 839-859; and Rees and Lui, Nat Rev Genet., 2018, 19(12), 770-788. In some embodiments, prime editing is used to modulate MHC I and/or MHC II antigen, tolerogenic factor(s), and/or CAR expression. Descriptions of prime editing can be found, for example, in Anzalone et al., Nature, 2019, 576, 149-157; Kantor et al., Int J Mole Sci., 2020, 21(17), 6240; Schene et al., Nat. Commun., 2020, 11, 5232; and Scholefield and Harrison, Gene Therapy, 2021, doi.org/10.1038/s41434-021-00263-9.

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### II. Definitions

The term “autoimmune disease” refers to any disease or disorder in which the subject mounts a destructive immune response against its own tissues and/or cells. Autoimmune disorders can affect almost every organ system in the subject (e.g., human), including, but not limited to, diseases of the nervous, gastrointestinal, and endocrine systems, as well as skin and other connective tissues, eyes, blood and blood vessels. Examples of autoimmune diseases include, but are not limited to, Hashimoto's thyroiditis, Systemic lupus erythematosus, Sjogren's syndrome, Graves' disease, Scleroderma, Rheumatoid arthritis, Multiple sclerosis, Myasthenia gravis and Diabetes.

The term “cancer” as used herein is defined as a hyperproliferation of cells whose unique trait (e.g., loss of normal controls) results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues of the malignant type, unless otherwise specifically indicated, and does not include a benign type tissue.

The term “chronic infectious disease” refers to a disease caused by an infectious agent wherein the infection has persisted. Such a disease may include hepatitis (A, B, or C), herpes virus (e.g., VZV, HSV-1, HSV-6, HSV-II, CMV, and EBV), and HIV/AIDS. Non-viral examples may include chronic fungal diseases such Aspergillosis, Candidiasis, Coccidioidomycosis, and diseases associated with Cryptococcus and Histoplasmosis. None limiting examples of chronic bacterial infectious agents may be Chlamydia pneumoniae, Listeria monocytogenes, and Mycobacterium tuberculosis. In some embodiments, the disorder is human immunodeficiency virus (HIV) infection. In some embodiments, the disorder is acquired immunodeficiency syndrome (AIDS).

In some embodiments, an alteration or modification (including, for example, genetic alterations or modifications) described herein results in reduced expression of a target or selected polynucleotide sequence. In some embodiments, an alteration or modification described herein results in reduced expression of a target or selected polypeptide sequence. In some embodiments, an alteration or modification described herein results in increased expression of a target or selected polynucleotide sequence. In some embodiments, an alteration or modification described herein results in increased expression of a target or selected polypeptide sequence. The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level. In some embodiments, the cells are engineered to have reduced expression of one or more targets relative to an unaltered or unmodified wild-type cell. By “wild-type” or “wt” in the context of a cell means any cell found in nature. However, by way of example, in the context of an engineered cell or a hypoimmunogenic cell, as used herein, “wild-type” can also mean an engineered cell or a hypoimmunogenic cell that may contain nucleic acid changes resulting in reduced expression of MHC I and/or II and/or T-cell receptors, but did not undergo the gene editing procedures to result in overexpression of CD47 proteins, e.g., a cell can be “wild-type” for CD47 but altered with regard to MHC I and/or II and/or T-cell receptors. As used herein, “wild-type” can also mean an engineered cell or a hypoimmunogenic cell that may contain nucleic acid changes resulting in overexpression of CD47 proteins, but did not undergo the gene editing procedures to result in reduced expression of MHC I and/or II and/or T-cell receptors, e.g., a cell can be “wild-type” for MHC I and/or II and/or T-cell receptors but altered with regard to CD47. In the context of a PSC or a progeny thereof, “wild-type” also means a PSC or progeny thereof that may contain nucleic acid changes resulting in pluripotency but did not undergo the gene editing procedures of the present technology to achieve reduced expression of MHC I and/or II and/or T-cell receptors, and/or overexpression of CD47 proteins. Also in the context of a PSC or a progeny thereof, “wild-type” also means a PSC or progeny thereof that may contain nucleic acid changes resulting in overexpression of CD47 proteins, but did not undergo the gene editing procedures to result in reduced expression of MHC I and/or II and/or T-cell receptors. In the context of a primary cell or a progeny thereof, “wild-type” also means a primary cell or progeny thereof that may contain nucleic acid changes resulting in reduced expression of MHC I and/or II and/or T-cell receptors, but did not undergo the gene editing procedures to result in overexpression of CD47 proteins. Also in the context of a primary cell or a progeny thereof, “wild-type” also means a primary cell or progeny thereof that may contain nucleic acid changes resulting in overexpression of CD47 proteins, but did not undergo the gene editing procedures to result in reduced expression of MHC I and/or II and/or T-cell receptors. In some embodiments, the cells are engineered to have reduced or increased expression of one or more targets relative to a cell of the same cell type that does not comprise the modifications.

The term “endogenous” refers to a referenced molecule or polypeptide that is naturally present in the cell. Similarly, the term when used in reference to expression of an encoding nucleic acid refers to expression of an encoding nucleic acid naturally contained within the cell and not exogenously introduced.

As used herein, the term “exogenous” in intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. An “exogenous” molecule is a molecule, construct, factor and the like that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of neurons is an exogenous molecule with respect to an adult neuron cell. An exogenous molecule can comprise, for example, a functioning version of a malfunctioning endogenous molecule or a malfunctioning version of a normally-functioning endogenous molecule.

An exogenous molecule or factor can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and/or helicases.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and/or locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and/or glycosylation.

The term “genetic modification” and its grammatical equivalents as used herein can refer to one or more alterations of a nucleic acid, e.g., the nucleic acid within an organism's genome. For example, genetic modification can refer to alterations, additions, and/or deletion of genes or portions of genes or other nucleic acid sequences. A genetically modified cell can also refer to a cell with an added, deleted and/or altered gene or portion of a gene. A genetically modified cell can also refer to a cell with an added nucleic acid sequence that is not a gene or gene portion. Genetic modifications include, for example, both transient knock-in or knock-down mechanisms, and mechanisms that result in permanent knock-in, knock-down, or knock-out of target genes or portions of genes or nucleic acid sequences Genetic modifications include, for example, both transient knock-in and mechanisms that result in permanent knock-in of nucleic acids sequences.

As used herein, the terms “grafting”, “administering,” “introducing”, “implanting” and “transplanting” as well as grammatical variations thereof are used interchangeably in the context of the placement of cells (e.g., cells described herein) into a subject, by a method or route which results in localization or at least partial localization of the introduced cells at a desired site or systemic introduction (e.g., into circulation). The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e. g. twenty-four hours, to a few days, to as long as several years. In some embodiments, the cells can also be administered (e.g., injected) a location other than the desired site, such as in the brain or subcutaneously, for example, in a capsule to maintain the implanted cells at the implant location and avoid migration of the implanted cells.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

As used herein to characterize a cell, the term “hypoimmunogenic” generally means that such cell is less prone to immune rejection, e.g., innate or adaptive immune rejection by a subject into which such cells are transplanted, e.g., the cell is less prone to allorejection by a subject into which such cells are transplanted. For example, relative to an unaltered or unmodified wild-type or non-hypoimmune cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. In some embodiments, genome editing technologies are used to modulate the expression of MHC I and MHC II genes, and thus, contribute to generation of a hypoimmunogenic cell. In some embodiments, a hypoimmunogenic cell evades immune rejection in an MHC-mismatched allogeneic recipient. In some instance, differentiated cells produced from the hypoimmunogenic stem cells outlined herein evade immune rejection when administered (e.g., transplanted or grafted) to an MHC-mismatched allogeneic recipient. In some embodiments, a hypoimmunogenic cell is protected from T cell-mediated adaptive immune rejection and/or innate immune cell rejection. Detailed descriptions of hypoimmunogenic cells, methods of producing thereof, and methods of using thereof are found in WO2016183041 filed May 9, 2015; WO2018132783 filed Jan. 14, 2018; WO2018176390 filed Mar. 20, 2018; WO2020018615 filed Jul. 17, 2019; WO2020018620 filed Jul. 17, 2019; PCT/US2020/44635 filed Jul. 31, 2020; U.S. 62/881,840 filed Aug. 1, 2019; U.S. 62/891,180 filed Aug. 23, 2019; U.S. 63/016,190, filed Apr. 27, 2020; and U.S. 63/052,360 filed Jul. 15, 2020, the disclosures including the examples, sequence listings and figures are incorporated herein by reference in their entirety.

Hypoimmunogenicity of a cell can be determined by evaluating the immunogenicity of the cell such as the cell's ability to elicit adaptive and innate immune responses or to avoid eliciting such adaptive and innate immune responses. Such immune response can be measured using assays recognized by those skilled in the art. In some embodiments, an immune response assay measures the effect of a hypoimmunogenic cell on T cell proliferation, T cell activation, T cell killing, donor specific antibody generation, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

“Immune signaling factor” as used herein refers to, in some cases, a molecule, protein, peptide and the like that activates immune signaling pathways.

“Immunosuppressive factor” or “immune regulatory factor” or “tolerogenic factor” as used herein include hypoimmunity factors, complement inhibitors, and other factors that modulate or affect the ability of a cell to be recognized by the immune system of a host or recipient subject upon administration, transplantation, or engraftment. These maybe in combination with additional genetic modifications.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system of the present disclosure can be used to induce an indel of any length or a point mutation in a target polynucleotide sequence, e.g. using gene editing, base editing, or prime editing. The term “base editing” refers to a method for the programmable conversion of one base pair to another at a targeted gene locus, and in some instances, without making double-stranded DNA breaks and in other instances without making s single-stranded DNA breaks. In some embodiments, base editing utilize a catalytically impaired Cas9 to recognize the target DNA site, and with a range of PAM sequence recognition, a window of based editing within and/or outside the protospacer sequence. The term “prime editing” refers to a method for gene editing that utilize a programmable polymerase (such as but not limited to a napDNAbps as described in WO2020191242) and particular guide RNAs. In some embodiments, the guide RNAs include a DNA synthesis template for encoding genetic information (or for deleting genetic information) that is incorporated into a target DNA sequence. As is recognized by those skilled in the art, base editing and prime editing are useful for modulating (e.g., reducing, eliminating, increasing, and enhancing) expression of polynucleotides and polypeptides described.

As used herein, “knock out” and “knock down” refers to genetic modifications that result in no expression and reduced expression of the edited gene, respectively. As used herein, “knock down” refers to a reduction in expression of the target mRNA or the corresponding target protein. Knock down is commonly reported relative to levels present following administration or expression of a control molecule that does not mediate reduction in expression levels of RNA (e.g., a non-targeting control shRNA, siRNA, guide RNA, or miRNA). In some embodiments, knock down of a target gene is achieve by way of shRNAs, siRNAs, miRNAs, or CRISPR interference (CRISPRi). In some embodiments, knock down of a target gene is achieved by way of a protein-based method, such as a degron method. In some embodiments, knock down of a target gene is achieved by genetic modification, including shRNAs, siRNAs, miRNAs, or use of gene editing systems (e.g., CRISPR/Cas).

Knock down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock down include RNA solution hybridization, nuclease protection, northern hybridization, gene expression monitoring with a microarray, antibody binding, radioimmunoassay, and fluorescence activated cell analysis. Those skilled in the art will readily appreciate how to use the gene editing systems (e.g., CRISPR/Cas) of the present disclosure to knock out a target polynucleotide sequence or a portion thereof based upon the details described herein.

By “knock in” herein is meant a genetic modification resulting from the insertion of a DNA sequence into a chromosomal locus in a host cell. This causes increased levels of expression of the knocked in gene, portion of gene, or nucleic acid sequence inserted product, e.g., an increase in RNA transcript levels and/or encoded protein levels. As will be appreciated by those in the art, this can be accomplished in several ways, including inserting or adding one or more additional copies of the gene or portion thereof to the host cell or altering a regulatory component of the endogenous gene increasing expression of the protein is made or inserting a specific nucleic acid sequence whose expression is desired. This may be accomplished by modifying a promoter, adding a different promoter, adding an enhancer, adding other regulatory elements, or modifying other gene expression sequences. A CRISPR/Cas system of the present disclosure can be used to knock-in a sequence, whether by homologous DNA repair using a template with homology arms or prime editing or gene writing wherein a specific sequence is edited in. In some instances, the term “knock in” is meant as a process that adds a genetic function to a host cell. This causes increased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences

As used herein, “knock out” includes deleting all or a portion of the target polynucleotide sequence in a way that interferes with the translation or function of the target polynucleotide sequence. For example, a knock out can be achieved by altering a target polynucleotide sequence by inducing an insertion or a deletion (“indel”) in the target polynucleotide sequence, including in a functional domain of the target polynucleotide sequence (e.g., a DNA binding domain). Those skilled in the art will readily appreciate how to use the gene editing systems (e.g., CRISPR/Cas) of the present disclosure to knock out a target polynucleotide sequence or a portion thereof based upon the details described herein.

In some embodiments, a genetic modification or alteration results in a knock out or knock down of the target polynucleotide sequence or a portion thereof. Knocking out a target polynucleotide sequence or a portion thereof using a gene editing systems (e.g., CRISPR/Cas) of the present technology can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject) or for changing the genotype or phenotype of a cell. In some instances and as used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use a gene editing system (e.g., a CRISPR/Cas system) of the present disclosure to knock out a target polynucleotide sequence or a portion thereof based upon the details described herein. In some embodiments, the alteration results in a knock out of the target polynucleotide sequence or a portion thereof. Knocking out a target polynucleotide sequence or a portion thereof using a CRISPR/Cas system of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

“Modulation” of gene expression refers to a change in the expression level of a gene. Modulation of expression can include, but is not limited to, gene activation and gene repression. Modulation may also be complete, i.e., wherein gene expression is totally inactivated or is activated to wildtype levels or beyond; or it may be partial, wherein gene expression is partially reduced, or partially activated to some fraction of wildtype levels.

In additional or alternative aspects, the present technology contemplates altering target polynucleotide sequences in any manner which is available to the skilled artisan, e.g., utilizing a nuclease system such as a TAL effector nuclease (TALEN) or zinc finger nuclease (ZFN) system. It should be understood that although examples of methods utilizing CRISPR/Cas (e.g., Cas9 and Cpf1) and TALEN are described in detail herein, the technology is not limited to the use of these methods/systems. Other methods of targeting to reduce or ablate expression in target cells known to the skilled artisan can be utilized herein. The methods provided herein can be used to alter a target polynucleotide sequence in a cell. The present technology contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the gene editing systems (e.g., CRISPR/Cas) of the present disclosure. In other instances, a “mutant cell” exhibits a wild-type phenotype, for example when a gene editing system (e.g., CRISPR/Cas) of the present disclosure is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence but need not be directly adjacent to it. For example, an enhancer is a transcriptional regulatory sequence that is operatively linked to a coding sequence, even though they are not contiguous.

“Pluripotent stem cells” as used herein have the potential to differentiate into any of the three germ layers: endoderm (e.g., the stomach linking, gastrointestinal tract, lungs, etc.), mesoderm (e.g., muscle, bone, blood, urogenital tissue, etc.) or ectoderm (e.g., epidermal tissues and nervous system tissues). The term “pluripotent stem cells,” as used herein, also encompasses “induced pluripotent stem cells”, or “iPSCs”, or a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells.

“Safe harbor locus” as used herein refers to a gene locus that allows expression of a transgene or an exogenous gene in a manner that enables the newly inserted genetic elements to function predictably and that also may not cause alterations of the host genome in a manner that poses a risk to the host cell. Exemplary “safe harbor” loci include, but are not limited to, a CCR5 gene, a PPP1R12C (also known as AAVS1) gene, a CLYBL gene, and/or a Rosa gene (e.g., ROSA26). “Target locus” as used herein refers to a gene locus that allows expression of a transgene or an exogenous gene. Exemplary “target loci” include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, and/or a KDM5D gene (also known as HY). The exogenous gene can be inserted in the CDS region for B2M, CIITA, TRAC, TRBC, CCR5, F3 (i.e., CD142), MICA, MICB, LRP1, HMGB1, ABO, RHD, FUT1, KDM5D (i.e., HY), PDGFRa, OLIG2, and/or GFAP. The exogenous gene can be inserted in introns 1 or 2 for PPP1R12C (i.e., AAVS1) or CCR5. The exogenous gene can be inserted in exons 1 or 2 or 3 for CCR5. The exogenous gene can be inserted in intron 2 for CLYBL. The exogenous gene can be inserted in a 500 bp window in Ch-4:58,976,613 (i.e., SHS231). The exogenous gene can be insert in any suitable region of the aforementioned safe harbor or target loci that allows for expression of the exogenous, including, for example, an intron, an exon or a coding sequence region in a safe harbor or target locus.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states, which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and/or non-human primates. The term “subject” also encompasses any vertebrate including but not limited to mammals, reptiles, amphibians and/or fish. However, advantageously, the subject is a mammal such as a human, or other mammals such as a domesticated mammal, e.g., dog, cat, horse, and the like, or production mammal, e.g., cow, sheep, pig, and the like.

As used herein, the term “treating” and “treatment” includes administering to a subject an effective amount of cells described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition but may not be a complete cure for the disease. In some embodiments, one or more symptoms of a disease or disorder are alleviated by at least 5%, at least 10%, at least 20%, at least 30%, at least 40%, or at least 50% upon treatment of the disease.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and/or viral vector-mediated transfer.

It is noted that the claims may be drafted to exclude any optional element. As such, this statement is intended to serve as antecedent basis for use of such exclusive terminology as “solely,” “only,” and the like in connection with the recitation of claim elements or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the technology. Any recited method may be carried out in the order of events recited or in any other order that is logically possible. Although any methods and materials similar or equivalent to those described herein may also be used in the practice or testing of the technology, representative illustrative methods and materials are now described.

Before the technology is further described, it is to be understood that this technology is not limited to particular embodiments described, as such may, of course, vary. It is also to be understood that the terminology used herein is for the purpose of describing particular embodiments only, and is not intended to be limiting, since the scope of the present technology will be limited only by the appended claims.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the technology. The upper and lower limits of these smaller ranges may independently be included in the smaller ranges and are also encompassed within the technology, subject to any specifically excluded limit in the stated range. Where the stated range includes one or both of the limits, ranges excluding either or both of those included limits are also included in the technology. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the technology described herein is not entitled to antedate such publication by virtue of prior technology. Further, the dates of publication provided might be different from the actual publication dates, which may need to be independently confirmed.

Before the technology is further described, it is to be understood that this technology is not limited to particular embodiments described, as such may, of course, vary. It is also to be understood that the terminology used herein is for the purpose of describing particular embodiments only, and is not intended to be limiting, since the scope of the present technology will be limited only by the appended claims. It should also be understood that the headers used herein are not limiting and are merely intended to orient the reader, but the subject matter generally applies to the technology disclosed herein.

### III. Detailed Description of the Embodiments

A. Administering Hypoimmunogenic Cells to Patients

In one aspect provided herein is a method of treating a patient by administering a population of the hypoimmunogenic cells described herein. The subject hypoimmunogenic cells provided herein (e.g., cells differentiated from hypoimmunogenic stem cells as described herein) can be administered to any suitable patients including, for example, a candidate for a cellular therapy for the treatment of a disease or disorder. Candidates for cellular therapy include any patient having a disease or condition that may potentially benefit from the therapeutic effects of the subject hypoimmunogenic cells provided herein. In some embodiments, the patient has a cellular deficiency. A candidate who benefits from the therapeutic effects of the subject hypoimmunogenic cells provided herein exhibit an elimination, reduction or amelioration of to disease or condition. As used herein, a “cellular deficiency” refers to any disease or condition that causes a dysfunction or loss of a population of cells in the patient, wherein the patient is unable to naturally replace or regenerate the population of cells. Exemplary cellular deficiencies include, but are not limited to, autoimmune diseases (e.g., multiple sclerosis, myasthenia gravis, rheumatoid arthritis, diabetes, systemic lupus and erythematosus), neurodegenerative diseases (e.g., Huntington's disease and Parkinson's disease), cardiovascular conditions and diseases, vascular conditions and diseases, corneal conditions and diseases, liver conditions and diseases, thyroid conditions and diseases, and/or kidney conditions and diseases. In some embodiments, the patient administered the hypoimmunogenic cells has a cancer. Exemplary cancers that can be treated by the hypoimmunogenic cells provided herein include, but are not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and/or bladder cancer. In certain embodiments, the cancer patient is treated by administration of a hypoimmunogenic CAR-T-cell provided herein.

In some embodiments, the hypoimmunogenic cells provided herein are useful for the treatment of a patient sensitized from one or more antigens present in a previous transplant such as, for example, a cell transplant, a blood transfusion, a tissue transplant, and/or an organ transplant. In certain embodiments, the previous transplant is an allogeneic transplant and the patient is sensitized against one or more alloantigens from the allogeneic transplant. Allogeneic transplants include, but are not limited to, allogeneic cell transplants, allogeneic blood transfusions, allogeneic tissue transplants, and/or allogeneic organ transplants. In some embodiments, the patient is sensitized patient who is or has been pregnant (e.g., having or having had alloimmunization in pregnancy). In certain embodiments, the patient is sensitized from one or more antigens included in a previous transplant, wherein the previous transplant is a modified human cell, tissue, and/or organ. In some embodiments, the modified human cell, tissue, and/or organ is a modified autologous human cell, tissue, and/or organ. In some embodiments, the previous transplant is a non-human cell, tissue, and/or organ. In exemplary embodiments, the previous transplant is a modified non-human cell, tissue, and/or organ. In certain embodiments, the previous transplant is a chimera that includes a human component. In certain embodiments, the previous transplant is and/or comprises a CAR-T-cell. In certain embodiments, the previous transplant is an autologous transplant and the patient is sensitized against one or more autologous antigens from the autologous transplant. In certain embodiments, the previous transplant is an autologous cell, tissue, and/or organ. In some embodiments, the sensitized patient has previously received an allogeneic CAR-T cell based therapy or an autologous CAR-T cell based therapy. Non-limiting examples of an autologous CAR-T cell based therapy include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL110 from Novartis, P-BMCA-101 from Poseida Therapeutics, and AUTO4 from Autolus Limited. Non-limiting examples of an allogeneic CAR-T cell based therapy include UCARTCS from Cellectis, PBCAR19B and PBCAR269A from Precision Biosciences, FT819 from Fate Therapeutics, and CYAD-211 from Clyad Oncology. In some embodiments, after the patient has previously received a first therapy comprising an allogeneic CAR-T cell based therapy or an autologous CAR-T cell based therapy that does not include the cells of the present technology, the sensitized patient is administered a second therapy comprising the cells of the present technology. In some embodiments, after the patient has previously received a first and/or second therapy comprising either an allogeneic CAR-T cell based therapy or an autologous CAR-T cell based therapy that does not include the cells of the present technology, then the sensitized patient is administered a third therapy comprising the cells of the present technology. In some embodiments, after the patient has previously received a series of therapies comprising an allogeneic CAR-T cell based therapy or an autologous CAR-T cell based therapy that does not include the cells of the present technology, then the sensitized patient is administered a subsequent therapy comprising the cells of the present technology. In some embodiments, the methods provided herein is used as next in-line treatment for a particular condition or disease (i) after a failed treatment such as, but not limited to, an allogeneic or autologous CAR-T cell based therapy that does not comprise the cells provided herein, (ii) after a therapeutically ineffective treatment such as, but not limited to, an allogeneic or autologous CAR-T cell based therapy that does not comprise the cells provided herein, or (iii) after an effective treatment such as, but not limited to, an allogeneic or autologous CAR-T cell based therapy that does not comprise the cells provided herein, including in each case in some embodiments following a first-line, second-line, third-line, and additional lines of treatment.

In certain embodiments, the sensitized patient has an allergy and is sensitized to one or more allergens. In exemplary embodiments, the patient has a hay fever, a food allergy, an insect allergy, a drug allergy, and/or atopic dermatitis.

Any suitable method known in the art in view of the present disclosure can be used to determine whether a patient is a sensitized patient. Examples of methods for determining whether a patient is a sensitized patient include, but are not limited to, cell based assays, including complement-dependent cytotoxicity (CDC) and flow cytometry assays, and solid phase assays, including ELISAs and polystyrene bead-based array assays. Other examples of methods for determining whether a patient is a sensitized patient include, but are not limited to, antibody screening methods, percent panel-reactive antibody (PRA) testing, Luminex-based assays, e.g., using single-antigen beads (SABs) and Luminex IgG assays, evaluation of mean fluorescence intensity (MFI) values of HLA antibodies, calculated panel-reactive antibody (cPRA) assays, IgG titer testing, complement-binding assays, IgG subtyping assays, and/or those described in Colvin et al., Circulation. 2019 Mar. 19; 139(12):e553-e578,

In some embodiments, the patient undergoing a treatment using the subject hypoimmunogenic cells received a previous treatment. In some embodiments, the hypoimmunogenic cells are used to treat the same condition as the previous treatment. In some embodiments, the hypoimmunogenic cells are used to treat a different condition from the previous treatment. In some embodiments, the hypoimmunogenic cells administered to the patient exhibit an enhanced therapeutic effect for the treatment of the same condition or disease treated by the previous treatment. In some embodiments, the administered hypoimmunogenic cells exhibit a longer therapeutic effect for the treatment of the condition or disease in the patient as compared to the previous treatment. In exemplary embodiments, the administered cells exhibit an enhanced potency, efficacy, and/or specificity against the cancer cells as compared to the previous treatment. In particular embodiments, the hypoimmunogenic cells are CAR-T-cells for the treatment of a cancer.

In some embodiments, the methods provided herein can be used as a next in-line treatment for a particular condition or disease after a failed treatment, after a therapeutically ineffective treatment, or after an effective treatment, including in each case following a first-line, second-line, third-line, and additional lines of treatment. In some embodiments, the previous treatment (e.g., the first-line treatment) is a therapeutically ineffective treatment. As used herein, a “therapeutically ineffective” treatment refers to a treatment that produces a less than desired clinical outcome in a patient. For example, with respect to a treatment for a cellular deficiency, a therapeutically ineffective treatment may refer to a treatment that does not achieve a desired level of functional cells and/or cellular activity to replace the deficient cells in a patient, and/or lacks therapeutic durability. With respect to a cancer treatment, a therapeutically ineffective treatment refers to a treatment that does not achieve a desired level of potency, efficacy, and/or specificity. Therapeutic effectiveness can be measured using any suitable technique known in the art. In some embodiments, the patient produces an immune response to the previous treatment. In some embodiments, the previous treatment is a cell, tissue, and/or organ graft that is rejected by the patient. In some embodiments, the previous treatment included a mechanically assisted treatment. In some embodiments, the mechanically assisted treatment included a hemodialysis or a ventricle assist device. In some embodiments, the patient produced an immune response to the mechanically assisted treatment. In some embodiments, the previous treatment included a population of therapeutic cells that include a safety switch that can cause the death of the therapeutic cells, when the safety switch is activated, should they grow and divide in an undesired manner. In some embodiments, the patient produces an immune response as a result of the safety switch induced death of therapeutic cells. In some embodiments, the patient is sensitized from the previous treatment. In exemplary embodiments, the patient is not sensitized by the administered hypoimmunogenic cells.

In some embodiments, the subject hypoimmunogenic cells are administered prior to, concurrently with, and/or after, providing a tissue, organ, and/or partial organ transplant to a patient in need thereof. In some embodiments, the patient does not exhibit an immune response to the hypoimmunogenic cells. In some embodiments, the hypoimmunogenic cells are administered to the patient for the treatment of a cellular deficiency in a particular tissue and/or organ and the patient subsequently receives a tissue or organ transplant for the same particular tissue or organ. In some embodiments, the hypoimmunogenic cells are administered to the patient as in situ in a tissue or organ for transplantation. In some embodiments, the hypoimmunogenic cells are administered to the patient as in situ in a tissue or organ before or after a tissue or organ transplant. In such embodiments, the hypoimmunogenic cell treatment functions as a bridge therapy to the eventual tissue or organ replacement. For example, in some embodiments, the patient has a liver disorder and receives a hypoimmunogenic hepatocyte treatment as provided herein, prior to receiving a liver transplant. In some embodiments, the patient has a liver disorder and receives a hypoimmunogenic hepatocyte treatment as provided herein, after receiving a liver transplant. In some embodiments, the hypoimmunogenic cells are administered to the patient for the treatment of a cellular deficiency in a particular tissue and/or organ and the patient subsequently receives a tissue and/or organ transplant for a different tissue or organ. For example, in some embodiments, the patient is a diabetes patient who is treated with hypoimmunogenic pancreatic beta cells prior to receiving a kidney transplant. In some embodiments, the patient is a diabetes patient who is treated with hypoimmunogenic pancreatic beta cells after receiving a kidney transplant. In some embodiments, the hypoimmunogenic cell treatment is administered to the donor tissue and/or organ before and/or after the patient receives the tissue or organ transplant. In some embodiments, the method is for the treatment of a cellular deficiency. In exemplary embodiments, the tissue or organ transplant is a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, and/or a bone transplant.

The methods of treating a patient are generally through administrations of cells, particularly the hypoimmunogenic cells provided herein. As will be appreciated, for all the multiple embodiments described herein related to the cells and/or the timing of therapies, the administering of the cells is accomplished by a method or route that results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. In some embodiments, the cells are implanted in situ in the desired organ or the desired location of the organ, In some embodiments, the cells can be implanted into the donor tissue and/or organ before and/or after the patient receives the tissue or organ transplant. In some embodiments, the cells are administered to treat a disease or disorder, such as any disease, disorder, condition, and/or symptom thereof that can be alleviated by cell therapy.

In some embodiments, the population of cells is administered at least 1 day, at least 2 days, at least 3 days, at least 4 days, at least 5, days, at least 6 days, at least 1 week, or at least 1 month or more after the patient is sensitized. In some embodiments, the population of cells is administered at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the patient is sensitized or exhibits characteristics or features of sensitization. In some embodiments, the population of cells is administered at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the patient has received the transplant (e.g., an allogeneic transplant), has been pregnant (e.g., having or having had alloimmunization in pregnancy) and/or is sensitized and/or exhibits characteristics and/or features of sensitization.

In some embodiments, the patient who has received a transplant, who has been pregnant (e.g., having or having had alloimmunization in pregnancy), and/or who is sensitized against an antigen (e.g., alloantigens) is administered a dosing regimen comprising a first dose administration of a population of cells described herein, a recovery period after the first dose, and a second dose administration of a population of cells described. In some embodiments, the composite of cell types present in the first population of cells and the second population of cells are different. In certain embodiments, the composite of cell types present in the first population of cells and the second population of cells are the same or substantially equivalent. In many embodiments, the first population of cells and the second population of cells comprises the same cell types. In some embodiments, the first population of cells and the second population of cells comprises different cell types. In some embodiments, the first population of cells and the second population of cells comprises the same percentages of cell types. In other embodiments, the first population of cells and the second population of cells comprises different percentages of cell types.

In some embodiments, the population of cells is administered for treatment of a cellular deficiency and/or as a cellular therapy for the treatment of a condition or disease in a tissue and/or organ selected from the group consisting of heart, lung, kidney, liver, pancreas, intestine, stomach, cornea, bone marrow, blood vessel, heart valve, brain, spinal cord, and/or bone.

In some embodiments, the cellular deficiency is associated with a neurodegenerative disease and the cellular therapy is for the treatment of a neurodegenerative disease. In some embodiments, the neurodegenerative disease is selected from the group consisting of leukodystrophy, Huntington's disease, Parkinson's disease, multiple sclerosis, transverse myelitis, and/or Pelizaeus-Merzbacher disease (PMD). In some embodiments, the cells are selected from the group consisting of glial progenitor cells, oligodendrocytes, astrocytes, and dopaminergic neurons, optionally wherein the dopaminergic neurons are selected from the group consisting of neural stem cells, neural progenitor cells, immature dopaminergic neurons, and mature dopaminergic neurons. In some embodiments, the cellular deficiency is associated with a liver disease and the cellular therapy is for the treatment of liver disease. In some embodiments, the liver disease comprises cirrhosis of the liver. In some embodiments, the cells are hepatocytes or hepatic progenitor cells. In some embodiments, the cellular deficiency is associated with a corneal disease and the cellular therapy is for the treatment of corneal disease. In some embodiments, the corneal disease is Fuchs dystrophy or congenital hereditary endothelial dystrophy. In some embodiments, the cells are corneal endothelial progenitor cells or corneal endothelial cells. In some embodiments, the cellular deficiency is associated with a cardiovascular condition or disease and the cellular therapy is for the treatment of a cardiovascular condition or disease. In some embodiments, the cardiovascular disease is myocardial infarction and/or congestive heart failure. In some embodiments, the cells are cardiomyocytes or cardiac progenitor cells. In some embodiments, the cellular deficiency is associated with diabetes and the cellular therapy is for the treatment of diabetes. In some embodiments, the cells are pancreatic islet cells, including pancreatic beta islet cells, optionally wherein the pancreatic islet cells are selected from the group consisting of a pancreatic islet progenitor cell, an immature pancreatic islet cell, and a mature pancreatic islet cell. In some embodiments, the cellular deficiency is associated with a vascular condition or disease and the cellular therapy is for the treatment of a vascular condition or disease. In some embodiments, the cells are endothelial cells. In some embodiments, the cellular deficiency is associated with autoimmune thyroiditis and the cellular therapy is for the treatment of autoimmune thyroiditis. In some embodiments, the cells are thyroid progenitor cells. In some embodiments, the cellular deficiency is associated with a kidney disease and the cellular therapy is for the treatment of a kidney disease. In some embodiments, the cells are renal precursor cells or renal cells.

In some embodiments, the population of cells is administered for the treatment of cancer. In some embodiments, the population of cells is administered for the treatment of cancer and the population of cells is a population of CAR-T cells. In some embodiments, the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

In some embodiments, the patient is receiving a tissue or organ transplant, optionally wherein the tissue or organ transplant or partial organ transplant is selected from the group consisting of a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, a bone transplant, a partial lung transplant, a partial kidney transplant, a partial liver transplant, a partial pancreas transplant, a partial intestine transplant, and/or a partial cornea transplant.

In some embodiments, the tissue or organ transplant is an allograft transplant. In some embodiments, the tissue or organ transplant is an autograft transplant. In some embodiments, the population of cells is administered for the treatment of a cellular deficiency in a tissue or organ and the tissue or organ transplant is for the replacement of the same tissue or organ. In some embodiments, the population of cells is administered for the treatment of a cellular deficiency in a tissue and/or organ and the tissue and/or organ transplant is for the replacement of a different tissue or organ. In some embodiments, the organ transplant is a kidney transplant and the population of cells is a population of renal precursor cells or renal cells. In some embodiments, the patient has diabetes and the population of cells is a population of beta islet cells. In some embodiments, the organ transplant is a heart transplant and the population of cells is a population of cardiac progenitor cells or pacemaker cells. In some embodiments, the organ transplant is a pancreas transplant and the population of cells is a population of pancreatic beta islet cells. In some embodiments, the organ transplant is a partial liver transplant and the population of cells is a population of hepatocytes or hepatic progenitor cells.

In some embodiments, the recovery period begins following the first administration of the population of hypoimmunogenic cells and ends when such cells are no longer present or detectable in the patient. In some embodiments, the duration of the recovery period is at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the initial administration of the cells. In some embodiments, the duration of the recovery period is at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the initial administration of the cells.

In some embodiments, the administered population of hypoimmunogenic cells elicits a decreased or lower level of systemic TH1 activation in the patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, the administered population of hypoimmunogenic cells fails to elicit systemic TH1 activation in the patient.

In some embodiments, the administered population of hypoimmunogenic cells elicits a decreased or lower level of immune activation of peripheral blood mononuclear cells (PBMCs) in the patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, the administered population of hypoimmunogenic cells fails to elicit immune activation of PBMCs in the patient.

In some embodiments, the administered population of hypoimmunogenic cells elicits a decreased or lower level of donor-specific IgG antibodies in the patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells. In some embodiments, the administered population of hypoimmunogenic cells fails to elicit donor-specific IgG antibodies in the patient.

In some embodiments, the administered population of hypoimmunogenic cells elicits a decreased or lower level of IgM and IgG antibody production in the patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, the administered population of hypoimmunogenic cells fails to elicit IgM and IgG antibody production in the patient.

In some embodiments, the administered population of hypoimmunogenic cells elicits a decreased or lower level of cytotoxic T cell killing in the patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells. In some embodiments, the administered population of hypoimmunogenic cells fails to elicit cytotoxic T cell killing in the patient.

As discussed above, provided herein are cells that in certain embodiments can be administered to a patient sensitized against alloantigens such as human leukocyte antigens. In some embodiments, the patient is or has been pregnant, e.g., with alloimmunization in pregnancy (e.g., hemolytic disease of the fetus and newborn (HDFN), neonatal alloimmune neutropenia (NAN) or fetal and neonatal alloimmune thrombocytopenia (FNAIT)). In other words, the patient has or has had a disorder or condition associated with alloimmunization in pregnancy such as, but not limited to, hemolytic disease of the fetus and newborn (HDFN), neonatal alloimmune neutropenia (NAN), and fetal and neonatal alloimmune thrombocytopenia (FNAIT). In some embodiments, the patient has received an allogeneic transplant such as, but not limited to, an allogeneic cell transplant, an allogeneic blood transfusion, an allogeneic tissue transplant, or an allogeneic organ transplant. In some embodiments, the patient exhibits memory B cells against alloantigens. In some embodiments, the patient exhibits memory T cells against alloantigens. Such patients can exhibit both memory B and memory T cells against alloantigens.

Upon administration of the cells described, the patient exhibits no systemic immune response or a reduced level of systemic immune response compared to responses to cells that are not hypoimmunogenic. In some embodiments, the patient exhibits no adaptive immune response or a reduced level of adaptive immune response compared to responses to cells that are not hypoimmunogenic. In some embodiments, the patient exhibits no innate immune response or a reduced level of innate immune response compared to responses to cells that are not hypoimmunogenic. In some embodiments, the patient exhibits no T cell response or a reduced level of T cell response compared to responses to cells that are not hypoimmunogenic. In some embodiments, the patient exhibits no B cell response or a reduced level of B cell response compared to responses to cells that are not hypoimmunogenic.

As is described in further detail herein, provided herein is a population of hypoimmunogenic cells including exogenous CD47 polypeptides and reduced expression of MHC class I human leukocyte antigens, a population of hypoimmunogenic cells including exogenous CD47 polypeptides and reduced expression of MHC class II human leukocyte antigens, and a population of hypoimmunogenic cells including exogenous CD47 polypeptides and reduced expression of MHC class I and class II human leukocyte antigens.

B. Hypoimmunogenic Cells

Provided herein are cells comprising a modification of one or more target polynucleotide sequences that modulates the expression of MHC I molecules, MHC II molecules, or MHC I and MHC II molecules. In certain aspects, the modification comprising increasing expression of CD47. In some embodiments, the cells include one or more transient modifications or genomic modifications that reduce expression of MHC class I molecules and a modification that increases expression of CD47. In other words, the engineered cells comprise exogenous polynucleotides encoding CD47 proteins and exhibit reduced or silenced surface expression of one or more MHC class I molecules. In some embodiments, the cells include one or more genomic modifications that reduce expression of MHC class II molecules and a modification that increases expression of CD47. In some instances, the engineered cells comprise exogenous CD47 nucleic acids and proteins and exhibit reduced or silenced surface expression of one or more MHC class I molecules. In some embodiments, the cells include one or more genomic modifications that reduce or eliminate expression of MHC class II molecules, one or more genomic modifications that reduce or eliminate expression of MHC class II molecules, and a modification that increases expression of CD47. In some embodiments, the engineered cells comprise exogenous CD47 proteins, exhibit reduced or silenced surface expression of one or more MHC class I molecules and exhibit reduced or lack surface expression of one or more MHC class II molecules. In many embodiments, the cells are B2M^{indel/indel}, CIITA^{indel/indel}, CD47tg cells.

Reduction of MHC I and/or MHC II expression can be accomplished, for example, by one or more of the following: (1) targeting the polymorphic HLA alleles (HLA-A, HLA-B, HLA-C) and MHC-II genes directly; (2) removal of B2M, which will reduce surface trafficking of all MHC-I molecules; and/or (3) deletion of one or more components of the MHC enhanceosomes, such as LRC5, RFX-5, RFXANK, RFXAP, IRF1, NF-Y (including NFY-A, NFY-B, NFY-C), and CIITA that are important for HLA expression.

In certain embodiments, HLA expression is interfered with. In some embodiments, HLA expression is interfered with by targeting individual HLAs (e.g., knocking out expression of HLA-A, HLA-B and/or HLA-C), targeting transcriptional regulators of HLA expression (e.g., knocking out expression of NLRC5, CIITA, RFX5, RFXAP, RFXANK, NFY-A, NFY-B, NFY-C and/or IRF-1), blocking surface trafficking of MHC class I molecules (e.g., knocking out expression of B2M and/or TAP1), and/or targeting with HLA-Razor (see, e.g., WO2016183041).

In certain aspects, the cells, including stem cells or differentiated stem cells, disclosed herein do not express one or more human leukocyte antigens (e.g., HLA-A, HLA-B and/or HLA-C) corresponding to MHC-I and/or MHC-II and are thus characterized as being hypoimmunogenic. For example, in certain aspects, the cells, including stem cells or differentiated stem cells, disclosed herein have been modified such that the stem cell or a differentiated stem cell prepared therefrom do not express or exhibit reduced expression of one or more of the following MHC-I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In certain embodiments, guide RNAs that allow simultaneous deletion of all MHC class I alleles by targeting a conserved region in the HLA genes are identified as HLA Razors. In some embodiments, the guide RNAs are part of a CRISPR system, e.g., a CRISPR-Cas9 system. In alternative aspects, the gRNAs are part of a TALEN system. In one aspect, an HLA Razor targeting an identified conserved region in HLAs is described in WO2016183041. In other aspects, multiple HLA Razors targeting identified conserved regions are utilized. It is generally understood that any guide that targets a conserved region in HLAs can act as an HLA Razor.

In some embodiments, the cell includes a modification to increase expression of CD47 and one or more factors selected from the group consisting of DUX4, CD24, CD27, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, CD16, CD52, H2-M3, and Serpinb9.

In some embodiments, the cell comprises a genomic modification of one or more target polynucleotide sequences that regulate the expression of either MHC class I molecules, MHC class II molecules, or MHC class I and MHC class II molecules. In some embodiments, a genetic editing system is used to modify one or more target polynucleotide sequences. In some embodiments, the targeted polynucleotide sequence is one or more selected from the group including B2M, CIITA, and NLRC5. In some embodiments, the cell comprises a genetic editing modification to the B2M gene. In some embodiments, the cell comprises a genetic editing modification to the CIITA gene. In some embodiments, the cell comprises a genetic editing modification to the NLRC5 gene. In some embodiments, the cell comprises genetic editing modifications to the B2M and CIITA genes. In some embodiments, the cell comprises genetic editing modifications to the B2M and NLRC5 genes. In some embodiments, the cell comprises genetic editing modifications to the CIITA and NLRC5 genes. In particular embodiments, the cell comprises genetic editing modifications to the B2M, CIITA and NLRC5 genes. In some embodiments, the genome of the cell has been altered to reduce or delete important components of HLA expression.

In some embodiments, the present disclosure provides a cell (e.g., stem cell, induced pluripotent stem cell, differentiated cell, hematopoietic stem cell, primary cell or CAR-T cell) or population thereof comprising a genome in which a gene has been edited to delete a contiguous stretch of genomic DNA, thereby reducing or eliminating expression of MHC class I molecules in the cell or population thereof, e.g., surface expression of MHC class I molecules in the cell or population thereof. In certain aspects, the present disclosure provides a cell (e.g., stem cell, induced pluripotent stem cell, differentiated cell, hematopoietic stem cell, primary cell or CAR-T cell) or population thereof comprising a genome in which a gene has been edited to delete a contiguous stretch of genomic DNA, thereby reducing or eliminating surface expression of MHC class II molecules in the cell or population thereof. In particular aspects, the present disclosure provides a cell (e.g., stem cell, induced pluripotent stem cell, differentiated cell, hematopoietic stem cell, primary cell or CAR-T cell) or population thereof comprising a genome in which one or more genes has been edited to delete a contiguous stretch of genomic DNA, thereby reducing or eliminating surface expression of MHC class I and II molecules in the cell or population thereof.

In certain embodiments, the expression of MHC I molecules and/or MHC II molecules is modulated by targeting and deleting a contiguous stretch of genomic DNA, thereby reducing or eliminating expression of a target gene selected from the group consisting of B2M, CIITA, and NLRC5. In some embodiments, described herein are genetically edited cells (e.g., modified human cells) comprising exogenous CD47 proteins and inactivated or modified CIITA gene sequences, and in some instances, additional gene modifications that inactivate or modify B2M gene sequences. In some embodiments, described herein are genetically edited cells comprising exogenous CD47 proteins and inactivated or modified CIITA gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify CIITA gene sequences and NLRC5 gene sequences.

In some embodiments, the cells are B2M^{-/-}, CIITA^{-/-}, TRAC^{-/-}, TRB^{-/-}, CD47tg cells. In some embodiments, the B2M^{-/-}, CIITA^{-/-}, TRAC^{-/-}, TRB^{-/-}, CD47tg cell is a primary T cell or a T cell derived from a hypoimmunogenic pluripotent cell (e.g., a hypoimmunogenic iPSC).

In some embodiments, the cells are B2M^{-/-}, CIITA^{-/-}, TRAC^{-/-}, and CD47tg cells. In some embodiments, the B2M^{-/-}, CIITA^{-/-}, TRAC^{-/-}, and CD47tg cell is a primary T cell or a T cell derived from a hypoimmunogenic pluripotent cell (e.g., a hypoimmunogenic iPSC).

In some embodiments, the cells described herein include, but are not limited to, pluripotent stem cells, induced pluripotent stem cells, differentiated cells derived or produced from such stem cells, hematopoietic stem cells, primary T cells, chimeric antigen receptor (CAR) T cells, and any progeny thereof.

In some embodiments, the primary T cells are selected from a group that includes cytotoxic T-cells, helper T-cells, memory T-cells, regulatory T-cells, tumor infiltrating lymphocytes, and combinations thereof.

In some embodiments, hypoimmune T cells and primary T cells overexpress CD47 and a chimeric antigen receptor (CAR), and include a genomic modification of the B2M gene. In some embodiments, hypoimmune T cells and primary T cells overexpress CD47 and include a genomic modification of the CIITA gene. In some embodiments, hypoimmune T cells and primary T cells overexpress CD47 and a CAR, and include a genomic modification of the TRAC gene. In some embodiments, hypoimmune T cells and primary T cells overexpress CD47 and a CAR, and include a genomic modification of the TRB gene. In some embodiments, hypoimmune T cells and primary T cells overexpress CD47 and a CAR, and include one or more genomic modifications selected from the group consisting of the B2M, CIITA, TRAC, and TRB genes. In some embodiments, hypoimmune T cells and primary T cells overexpress CD47 and a CAR, and include genomic modifications of the B2M, CIITA, TRAC, and TRB genes. In some embodiments, the cells are B2M^{-/-}, CIITA^{-/-}, TRAC^{-/-}, and CD47tg cells that also express CARs.

In some embodiments, the cells are B2M^{-/-}, CIITA^{-/-}, TRB^{-/-}, and CD47tg cells that also express CARs. In some embodiments, the cells are B2M^{-/-}, CIITA^{-/-}, TRAC^{-/-}, TRB^{-/-}, and CD47tg cells that also express CARs. In many embodiments, the cells are B2M^{indel/indel}, CIITA^{indel/indel}, TRAC^{indel/indel}, and CD47tg cells that also express CARs. In many embodiments, the cells are B2M^{indel/indel}, CIITA^{indel/indel}, TRB^{indel/indel}, and CD47tg cells that also express CARs. In many embodiments, the cells are B2M^{indel/indel}, CIITA^{indel/indel}, TRAC^{indel/indel}, TRB^{indel/indel}, and CD47tg cells that also express CARs. In some embodiments, the modified cells described are pluripotent stem cells, induced pluripotent stem cells, cells differentiated from such pluripotent stem cells and induced pluripotent stem cells, or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the primary T cells are selected from a group that includes cytotoxic T-cells, helper T-cells, memory T-cells, regulatory T-cells, tumor infiltrating lymphocytes, and/or combinations thereof.

In some embodiments, the primary T cells are from a pool of primary T cells from one or more donor subjects that are different than the recipient subject (e.g., the patient administered the cells). The primary T cells can be obtained from 1, 2, 3, 4, 5, 6, 7, 8, 9, 10, 20, 50, 100 or more donor subjects and pooled together. The primary T cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary T cells are harvested from one or a plurality of individuals, and in some instances, the primary T cells or the pool of primary T cells are cultured in vitro. In some embodiments, the primary T cells or the pool of primary T cells are engineered to exogenously express CD47 and cultured in vitro.

In some embodiments, the primary T cells or the pool of primary T cells are engineered to express a chimeric antigen receptor (CAR). The CAR can be any known to those skilled in the art. Useful CARs include those that bind an antigen selected from a group that includes CD19, CD20, CD22, CD38, CD123, CD138, and BCMA. In some cases, the CAR is the same or equivalent to those used in FDA-approved CAR-T cell therapies such as, but not limited to, those used in brexucabtagene autoleucel, axicabtagene ciloleucel, idecabtagene vicleucel, lisocabtagene maraleucel, tisagenlecleucel, or others under investigation in clinical trials.

In some embodiments, the primary T cells or the pool of primary T cells are engineered to exhibit reduced expression of an endogenous T cell receptor compared to unmodified primary T cells. In some embodiments, the primary T cells or the pool of primary T cells are engineered to exhibit reduced expression of CTLA4, PD1, or both CTLA4 and PD1, as compared to unmodified primary T cells. Methods of genetically modifying a cell including a T cell are described in detail, for example, in WO2020018620 and WO2016183041, the disclosure are herein incorporated by reference in its entirety including the tables, appendices, sequence listing and figures.

In some embodiments, the CAR-T cells comprise a CAR selected from a group including: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the CAR-T cells comprise a CAR comprising an antigen binding domain, a transmembrane, and one or more signaling domains. In some embodiments, the CAR also comprises a linker. In some embodiments, the CAR comprises a CD19 antigen binding domain. In some embodiments, the CAR comprises a CD28 or a CD8α transmembrane domain. In some embodiments, the CAR comprises a CD8α signal peptide. In some embodiments, the CAR comprises a Whitlow linker GSTSGSGKPGSGEGSTKG (SEQ ID NO:14),In some embodiments, the antigen binding domain of the CAR is selected from a group including, but not limited to, (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the antigen binding domain is selected from a group that includes an antibody, an antigen-binding portion or fragment thereof, an scFv, and a Fab. In some embodiments, the antigen binding domain binds to CD19, CD20, CD22, CD38, CD123, CD138, or BCMA. In some embodiments, the antigen binding domain is an anti-CD19 scFv such as but not limited to FMC63.

In some embodiments, the transmembrane domain comprises one selected from a group that includes a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments, the signaling domain(s) of the CAR comprises a costimulatory domain(s). For instance, a signaling domain can contain a costimulatory domain. Or, a signaling domain can contain one or more costimulatory domains. In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR-T cell proliferation, and/or CAR-T cell persistence during T cell activation. In some embodiments, the costimulatory domains enhance cytokine production, CAR-T cell proliferation, and/or CAR-T cell persistence during T cell activation.

As described herein, a fourth generation CAR can contain an antigen binding domain, a transmembrane domain, three or four signaling domains, and a domain which upon successful signaling of the CAR induces expression of a cytokine gene. In some instances, the cytokine gene is an endogenous or exogenous cytokine gene of the hypoimmunogenic cells. In some cases, the cytokine gene encodes a pro-inflammatory cytokine. In some embodiments, the pro-inflammatory cytokine is selected from a group that includes IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta (CD3ζ) domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain or functional variant thereof.

Methods for introducing a CAR construct or producing a CAR-T cells are well known to those skilled in the art. Detailed descriptions are found, for example, in Vormittag et al., Curr Opin Biotechnol., 2018, 53, 162-181; and Eyquem et al., Nature, 2017, 543, 113-117.

In some embodiments, the cells derived from primary T cells comprise reduced expression of an endogenous T cell receptor, for example by disruption of an endogenous T cell receptor gene (e.g., T cell receptor alpha constant region (referred to as “TRAC”) and/or T cell receptor beta constant region (referred to as “TRBC” or “TRB”). In some embodiments, an exogenous nucleic acid encoding a polypeptide as disclosed herein (e.g., a chimeric antigen receptor, CD47, or another tolerogenic factor disclosed herein) is inserted at the disrupted T cell receptor gene. In some embodiments, an exogenous nucleic acid encoding a polypeptide is inserted at a TRAC or a TRB gene locus.

In some embodiments, the cells derived from primary T cells comprise reduced expression of cytotoxic T-lymphocyte-associated protein 4 (CTLA4) and/or programmed cell death (PD1). Methods of reducing or eliminating expression of CTLA4, PD1 and both CTLA4 and PD1 can include any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, TALEN, zinc finger nuclease, meganuclease, and/or homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA4 and/or PD1 gene locus.

In some embodiments, a CD47 transgene is inserted into a pre-selected locus of the cell. In some embodiments, a transgene encoding a CAR is inserted into a pre-selected locus of the cell. In many embodiments, a CD47 transgene and a transgene encoding a CAR are inserted into a pre-selected locus of the cell. The pre-selected locus can be a safe harbor locus or a target locus. Non-limiting examples of a safe harbor locus include, but are not limited to, a CCR5 gene locus, a PPP1R12C (also known as AAVS1) gene locus, a CLYBL gene locus, and/or a Rosa gene locus (e.g., ROSA26 gene locus). Non-limiting examples of a target locus include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, a KDM5D gene (also known as HY), a B2M gene, a CIITA gene, a TRAC gene, a TRBC gene, a CCR5 gene, a F3 (i.e., CD142) gene, a MICA gene, a MICB gene, a LRP1 gene, a HMGB1 gene, an ABO gene, a RHD gene, a FUT1 gene, a KDM5D (i.e., HY) gene, a PDGFRa gene, a OLIG2 gene, and/or a GFAP gene. In some embodiments, the pre-selected locus is selected from the group consisting of the B2M locus, the CIITA locus, the TRAC locus, and the TRB locus. In some embodiments, the pre-selected locus is the B2M locus. In some embodiments, the pre-selected locus is the CIITA locus. In some embodiments, the pre-selected locus is the TRAC locus. In some embodiments, the pre-selected locus is the TRB locus.

In some embodiments, a CD47 transgene and a transgene encoding a CAR are inserted into the same locus. In some embodiments, a CD47 transgene and a transgene encoding a CAR are inserted into different loci. In many instances, a CD47 transgene is inserted into a safe harbor or a target locus. In many instances, a transgene encoding a CAR is inserted into a safe harbor or a target locus. In some instances, a CD47 transgene is inserted into a B2M locus. In some instances, a transgene encoding a CAR is inserted into a B2M locus. In some embodiments, a CD47 transgene is inserted into a CIITA locus. In some embodiments, a transgene encoding a CAR is inserted into a CIITA locus. In some embodiments, a CD47 transgene is inserted into a TRAC locus. In some embodiments, a transgene encoding a CAR is inserted into a TRAC locus. In other embodiments, a CD47 transgene is inserted into a TRB locus. In other embodiments, a transgene encoding a CAR is inserted into a TRB locus. In some embodiments, a CD47 transgene and a transgene encoding a CAR are inserted into a safe harbor locus (e.g., a CCR5 gene locus, a PPP1R12C gene locus, a CLYBL gene locus, and/or a Rosa gene locus. In some embodiments, a CD47 transgene and a transgene encoding a CAR are inserted into a target locus (e.g., a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, a KDM5D gene (also known as HY), a B2M gene, a CIITA gene, a TRAC gene, a TRBC gene, a CCR5 gene, a F3 (i.e., CD142) gene, a MICA gene, a MICB gene, a LRP1 gene, a HMGB1 gene, an ABO gene, a RHD gene, a FUT1 gene, a KDM5D (i.e., HY) gene, a PDGFRa gene, a OLIG2 gene, and/or a GFAP gene.

In many embodiments, a CD47 transgene and a transgene encoding a CAR are inserted into a safe harbor or a target locus. In many embodiments, a CD47 transgene and a transgene encoding a CAR are controlled by a single promoter and are inserted into a safe harbor or a target locus. In many embodiments, a CD47 transgene and a transgene encoding a CAR are controlled by their own promoters and are inserted into a safe harbor or a target locus. In many embodiments, a CD47 transgene and a transgene encoding a CAR are inserted into a TRAC locus. In many embodiments, a CD47 transgene and a transgene encoding a CAR are controlled by a single promoter and are inserted into a TRAC locus. In many embodiments, a CD47 transgene and a transgene encoding a CAR are controlled by their own promoters and are inserted into a TRAC locus. In some embodiments, a CD47 transgene and a transgene encoding a CAR are inserted into a TRB locus. In some embodiments, a CD47 transgene and a transgene encoding a CAR are controlled by a single promoter and are inserted into a TRB locus. In some embodiments, a CD47 transgene and a transgene encoding a CAR are controlled by their own promoters and are inserted into a TRB locus. In other embodiments, a CD47 transgene and a transgene encoding a CAR are inserted into a B2M locus. In other embodiments, a CD47 transgene and a transgene encoding a CAR are controlled by a single promoter and are inserted into a B2M locus. In other embodiments, a CD47 transgene and a transgene encoding a CAR are controlled by their own promoters and are inserted into a B2M locus. In various embodiments, a CD47 transgene and a transgene encoding a CAR are inserted into a CIITA locus. In various embodiments, a CD47 transgene and a transgene encoding a CAR are controlled by a single promoter and are inserted into a CIITA locus. In various embodiments, a CD47 transgene and a transgene encoding a CAR are controlled by their own promoters and are inserted into a CIITA locus. In some instances, the promoter controlling expression of any transgene described is a constitutive promoter. In other instances, the promoter for any transgene described is an inducible promoter. In some embodiments, the promoter is an EF1 alpha (EF1α) promoter. In some embodiments, the promoter is a CAG promoter. In some embodiments, a CD47 transgene and a transgene encoding a CAR are both controlled by a constitutive promoter. In some embodiments, a CD47 transgene and a transgene encoding a CAR are both controlled by an inducible promoter. In some embodiments, a CD47 transgene is controlled by a constitutive promoter and a transgene encoding a CAR is controlled by an inducible promoter. In some embodiments, a CD47 transgene is controlled by an inducible promoter and a transgene encoding a CAR is controlled by a constitutive promoter. In various embodiments, a CD47 transgene is controlled by an EF1 alpha promoter and a transgene encoding a CAR is controlled by an EF1 alpha promoter. In other embodiments, expression of both a CD47 transgene and a transgene encoding a CAR is controlled by a single EF1 alpha promoter. In various embodiments, a CD47 transgene is controlled by a CAG promoter and a transgene encoding a CAR is controlled by a CAG promoter. In other embodiments, expression of both a CD47 transgene and a transgene encoding a CAR is controlled by a single CAG promoter. In some embodiments, a CD47 transgene is controlled by a CAG promoter and a transgene encoding a CAR is controlled by an EF1 alpha promoter. In some embodiments, a CD47 transgene is controlled by an EF1 alpha promoter and a transgene encoding a CAR is controlled by a CAG promoter.

In some embodiments, the cells described herein comprise a safety switch. The term “safety switch” used herein refers to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch includes a protein or molecule that allows for the control of cellular activity in response to an adverse event. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis. In some embodiments, the cells described herein, e.g., stem cells, induced pluripotent stem cells, hematopoietic stem cells, primary cells, or differentiated cell, including, but not limited to, cardiac cells, cardiac progenitor cells, neural cells, glial progenitor cells, endothelial cells, T cells, B cells, pancreatic islet cells, retinal pigmented epithelium cells, hepatocytes, thyroid cells, skin cells, blood cells, plasma cells, platelets, renal cells, epithelial cells, CART cells, NK cells, and/or CAR-NK cells, comprise a safety switch.

In some embodiments, the cells described herein comprise a “suicide gene” (or “suicide switch”). The suicide gene can cause the death of the hypoimmunogenic cells should they grow and divide in an undesired manner. The suicide gene ablation approach includes a suicide gene in a gene transfer vector encoding a protein that results in cell killing only when activated by a specific compound. A suicide gene can encode an enzyme that selectively converts a nontoxic compound into highly toxic metabolites. In some embodiments, the cells described herein, e.g., stem cells, induced pluripotent stem cells, hematopoietic stem cells, primary cells, or differentiated cell, including, but not limited to, cardiac cells, cardiac progenitor cells, neural cells, glial progenitor cells, endothelial cells, T cells, B cells, pancreatic islet cells, retinal pigmented epithelium cells, hepatocytes, thyroid cells, skin cells, blood cells, plasma cells, platelets, renal cells, epithelial cells, CART cells, NK cells, and/or CAR-NK cells, comprise a suicide gene.

In some embodiments, the population of engineered cells described elicits a reduced level of immune activation or no immune activation upon administration to a recipient subject. In some embodiments, the reduced immune response is compared to the immune response in a patient or control subject administered a “wild-type” population of cells. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

1. Therapeutic Cells Derived from T Cells and from iPSCs

Provided herein are hypoimmunogenic cells including, but not limited to, T cells that evade immune recognition. In some embodiments, the hypoimmunogenic cells are produced (e.g., generated, cultured, or derived) from pluripotent stem cells, such as iPSCs, MSCs, and/or ESCs. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient.

In some embodiments, the hypoimmunogenic cells do not activate an immune response in the patient (e.g., recipient upon administration). Provided are methods of treating a disorder comprising repeat dosing of a population of hypoimmunogenic cells to a subject (e.g., recipient) or patient in need thereof. In some embodiments, a population of hypoimmunogenic cells (e.g., hypoimmunogenic primary T cells) is administered at least twice (e.g., 2, 3, 4, 5, or more) to a human patient.

In some embodiments, the hypoimmunogenic cells do not activate an immune response in the patient (e.g., recipient upon administration). Provided are methods of treating a disease by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the present technology is directed to hypoimmunogenic primary T cells that overexpress CD47 and CARs, and have reduced expression or lack expression of MHC class I and/or MHC class II human leukocyte antigens and have reduced expression or lack expression of TCR complex molecules. The cells outlined herein overexpress CD47 and CARs and evade immune recognition. In some embodiments, the primary T cells display reduced levels or activity of MHC class I antigens, MHC class II antigens, and/or TCR complex molecules. In certain embodiments, primary T cells overexpress CD47 and CARs and harbor a genomic modification in the B2M gene. In some embodiments, T cells overexpress CD47 and CARs and harbor a genomic modification in the CIITA gene. In some embodiments, primary T cells overexpress CD47 and CARs and harbor a genomic modification in the TRAC gene. In some embodiments, primary T cells overexpress CD47 and CARs and harbor a genomic modification in the TRB gene. In some embodiments, T cells overexpress CD47 and CARs and harbor genomic modifications in one or more of the following genes: the B2M, CIITA, TRAC and TRB genes.

Exemplary T cells of the present disclosure are selected from the group consisting of cytotoxic T cells, helper T cells, memory T cells, central memory T cells, effector memory T cells, effector memory RA T cells, regulatory T cells, tissue infiltrating lymphocytes, and combinations thereof. In many embodiments, the T cells express CCR7, CD27, CD28, and CD45RA. In some embodiments, the central T cells express CCR7, CD27, CD28, and CD45RO. In other embodiments, the effector memory T cells express PD1, CD27, CD28, and CD45RO. In other embodiments, the effector memory RA T cells express PD1, CD57, and CD45RA.

In some embodiments, the T cell is a modified T cell. In some cases, the modified T cell comprise a modification causing the cell to express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof. In other cases, the modified T cell comprise a modification causing the cell to express at least one protein that modulates a biological effect of interest in an adjacent cell, tissue, or organ when the cell is in proximity to the adjacent cell, tissue, or organ. Useful modifications to primary T cells are described in detail in US2016/0348073 and WO2020/018620, the disclosures of which are incorporated herein in their entireties.

In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells include reduced expression of an endogenous T cell receptor. In some embodiments, the T cells described herein such as the engineered or modified T cells include reduced expression of cytotoxic T-lymphocyte-associated protein 4 (CTLA4). In other embodiments, the T cells described herein such as the engineered or modified T cells include reduced expression of programmed cell death (PD1). In certain embodiments, the T cells described herein such as the engineered or modified T cells include reduced expression of CTLA4 and PD1. In certain embodiments, the T cells described herein such as the engineered or modified T cells include enhanced expression of PD-L1.

In some embodiments, the hypoimmunogenic T cell includes a polynucleotide encoding a CAR, wherein the polynucleotide is inserted in a genomic locus. In some embodiments, the polynucleotide is inserted into a safe harbor or a target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, PDGFRa, OLIG2, GFAP, or KDM5D gene locus. In some embodiments, the polynucleotide is inserted in a B2M, CIITA, TRAC, TRB, PD1 or CTLA4 gene.

2. Chimeric Antigen Receptors

Provided herein are hypoimmunogenic cells comprising a chimeric antigen receptor (CAR). In some embodiments, the hypoimmunogenic cell is a primary T cell or a T cell derived from a hypoimmunogenic pluripotent cell (HIP) provided herein (e.g., a pluripotent stem cell). In some embodiments, the CAR is selected from the group consisting of a first generation CAR, a second generation CAR, a third generation CAR, and a fourth generation CAR.

In some embodiments, a hypoimmunogenic cell described herein comprises a polynucleotide encoding a chimeric antigen receptor (CAR) comprising an antigen binding domain. In some embodiments, a hypoimmunogenic cell described herein comprises a chimeric antigen receptor (CAR) comprising an antigen binding domain. In some embodiments, the polynucleotide is or comprises a chimeric antigen receptor (CAR) comprising an antigen binding domain. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

In some embodiments, a hypoimmunogenic cell described herein (e.g., hypoimmunogenic primary T cell or HIP-derived T cell) includes a polynucleotide encoding a CAR, wherein the polynucleotide is inserted in a genomic locus. In some embodiments, the polynucleotide is inserted into a safe harbor or a target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, PDGFRa, OLIG2, GFAP, and/or KDM5D gene locus. In some embodiments, the polynucleotide is inserted in a B2M, CIITA, TRAC, TRB, PD1 or CTLA4 gene. Any suitable method can be used to insert the CAR into the genomic locus of the hypoimmunogenic cell including the gene editing methods described herein (e.g., a CRISPR/Cas system).

a) Antigen Binding Domain (ABD) Targets an Antigen Characteristic of a Neoplastic or Cancer Cell

In some embodiments, the antigen binding domain (ABD) targets an antigen characteristic of a neoplastic cell. In other words, the antigen binding domain targets an antigen expressed by a neoplastic or cancer cell. In some embodiments, the ABD binds a tumor associated antigen. In some embodiments, the antigen characteristic of a neoplastic cell (e.g., antigen associated with a neoplastic or cancer cell) or a tumor associated antigen is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs; T-cell alpha chains; T-cell β chains; T-cell γ chains; T-cell δ chains, CCR7, CD3, CD4, CD5, CD7, CD8, CD11b, CD11c, CD16, CD19, CD20, CD21, CD22, CD25, CD28, CD34, CD35, CD40, CD45RA, CD45RO, CD52, CD56, CD62L, CD68, CD80, CD95, CD117, CD127, CD133, CD137 (4-1 BB), CD163, F4/80, IL-4Ra, Sca-1, CTLA-4, GITR, GARP, LAP, granzyme B, LFA-1, transferrin receptor, NKp46, perforin, CD4+, Th1, Th2, Th17, Th40, Th22, Th9, Tfh, Canonical Treg, FoxP3+, Tr1, Th3, Treg17, TREG, CDCP, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), L1CAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, ETV6-AML, sperm protein 17, XAGE1, Tie 2, MAD-CT-1, MAD-CT-2, Major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, and/or an antigenic fragment or antigenic portion thereof.

b) ABD Targets an Antigen Characteristic of a T Cell

In some embodiments, the antigen binding domain targets an antigen characteristic of a T cell. In some embodiments, the ABD binds an antigen associated with a T cell. In some instances, such an antigen is expressed by a T cell or is located on the surface of a T cell. In some embodiments, the antigen characteristic of a T cell or the T cell associated antigen is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; and/or ZAP70.

c) ABD Targets an Antigen Characteristic of an Autoimmune or Inflammatory Disorder

In some embodiments, the antigen binding domain targets an antigen characteristic of an autoimmune or inflammatory disorder. In some embodiments, the ABD binds an antigen associated with an autoimmune or inflammatory disorder. In some instances, the antigen is expressed by a cell associated with an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, hemophilia A, Primary Sjogren's Syndrome, thrombotic thrombocytopenia purrpura, neuromyelits optica, Evan's syndrome, IgM mediated neuropathy, cryoglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) and/or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and/or gene therapy. Allosensitization, in some instances, refers to the development of an immune response (such as circulating antibodies) against human leukocyte antigens that the immune system of the recipient subject or pregnant subject considers to be non-self antigens. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, and/or histidine kinase associated receptor.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See, US 2003/0077249; WO 2017/058753; WO 2017/058850, the contents of which are herein incorporated by reference.

d) ABD Targets an Antigen Characteristic of Senescent Cells

In some embodiments, the antigen binding domain targets an antigen characteristic of senescent cells, e.g., urokinase-type plasminogen activator receptor (uPAR). In some embodiments, the ABD binds an antigen associated with a senescent cell. In some instances, the antigen is expressed by a senescent cell. In some embodiments, the CAR may be used for treatment or prophylaxis of disorders characterized by the aberrant accumulation of senescent cells, e.g., liver and lung fibrosis, atherosclerosis, diabetes and osteoarthritis.

e) ABD Targets an Antigen Characteristic of an Infectious Disease

In some embodiments, the antigen binding domain targets an antigen characteristic of an infectious disease. In some embodiments, the ABD binds an antigen associated with an infectious disease. In some instances, the antigen is expressed by a cell affected by an infectious disease. In some embodiments, wherein the infectious disease is selected from HIV, hepatitis B virus, hepatitis C virus, human herpes virus, human herpes virus 8 (HHV-8, Kaposi sarcoma-associated herpes virus (KSHV)), human T-lymphotrophic virus-1 (HTLV-1), Merkel cell polyomavirus (MCV), simian virus 40 (SV40), Epstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gp120, or CD4-induced epitope on HIV-1 Env.

f) ABD Binds to a Cell Surface Antigen of a Cell

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell.

In some embodiments, a CAR antigen binding domain binds a cell surface antigen characteristic of a T cell, such as a cell surface antigen on a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, and/or histidine kinase associated receptor.

In some embodiments, an antigen binding domain of a CAR binds a T cell receptor. In some embodiments, a T cell receptor may be AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

g) Transmembrane Domain

In some embodiments, the CAR-Transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and/or FGFR2B, and/or functional variant thereof.

h) Signaling Domain or Plurality of Signaling Domains

In some embodiments, a CAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD1, ICOS, lymphocyte function-associated antigen-1 (LFA-1), CD2, CD7, LIGHT, NKG2C, B7-H3, a ligand that specifically binds with CD83, and/or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof, (ii) a CD28 domain or functional variant thereof; (iii) a 4-1BB domain, or a CD134 domain, or functional variant thereof, and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof, (ii) a CD28 domain or functional variant thereof, and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof, (ii) a CD28 domain or functional variant thereof, (iii) a 4-1BB domain, or a CD134 domain, or functional variant thereof, and (iv) a cytokine or costimulatory ligand transgene.

In some embodiments, the at least three signaling domains comprise a CD8α or functional variant thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof, and (ii) a CD28 domain, or a 4-1BB domain, or functional variant thereof.

In some embodiments, the CAR comprises a (i) a CD3 zeta domain, or an immunoreceptor tyrosine-based activation motif (ITAM), or functional variant thereof, (ii) a CD28 domain or functional variant thereof, and (iii) a 4-1BB domain, or a CD134 domain, or functional variant thereof.

In some embodiments, the CAR comprises (i) a CD3 zeta domain, or an immunoreceptor tyrosine-based activation motif (ITAM), or functional variant thereof, (ii) a CD28 domain, or a 4-1BB domain, or functional variant thereof, and/or (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

i) Domain which Upon Successful Signaling of the CAR Induces Expression of a Cytokine Gene

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments, a cytokine gene encodes a pro-inflammatory cytokine. In some embodiments, a cytokine gene encodes IL-1, IL-2, IL-9, IL-12, IL-18, TNF, or IFN-gamma, or functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments, a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, the CAR further comprises one or more spacers, or hinges, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine and serine residues such as but not limited to glycine-serine doublets. In some embodiments, the CAR comprises two or more spacers, e.g., a spacer between the antigen binding domain and the transmembrane domain and a spacer between the transmembrane domain and a signaling domain. In some embodiments, the spacer is a CD28 hinge, a CD8a hinge, or a IgG4 hinge.

In some embodiments, the CAR further comprises one or more linkers. The format of an scFv is generally two variable domains linked by a flexible peptide sequence, or a “linker,” either in the orientation VH-linker-VL or VL-linker-VH. Any suitable linker known to those in the art in view of the specification can be used in the CARs. Examples of suitable linkers include, but are not limited to, a GS based linker sequence, and a Whitlow linker GSTSGSGKPGSGEGSTKG (SEQ ID NO:14). In some embodiments, the linker is a GS or a gly-ser linker. Exemplary gly-ser polypeptide linkers comprise the amino acid sequence Ser(Gly₄Ser)ₙ, as well as (Gly₄Ser)ₙ and/or (Gly₄Ser₃)ₙ. In some embodiments, n=1. In some embodiments, n=2. In some embodiments, n=3, i.e., Ser(Gly₄Ser)₃. In some embodiments, n=4, i.e., Ser(Gly₄Ser)₄. In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser(Gly₄Ser)ₙ. In some embodiments, n=1. In some embodiments, n=2. In some embodiments, n=3. In another embodiment, n=4. In some embodiments, n=5. In some embodiments, n=6. Another exemplary gly-ser polypeptide linker comprises (Gly₄Ser)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6. Another exemplary gly-ser polypeptide linker comprises (Gly₄Ser₃)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6.

In some embodiments, any one of the cells described herein comprises a nucleic acid encoding a CAR or a first generation CAR. In some embodiments, a first generation CAR comprises an antigen binding domain, a transmembrane domain, and signaling domain. In some embodiments, a signaling domain mediates downstream signaling during T cell activation.

In some embodiments, any one of the cells described herein comprises a nucleic acid encoding a CAR or a second generation CAR. In some embodiments, a second generation CAR comprises an antigen binding domain, a transmembrane domain, and two signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and/or CAR-T cell persistence during T cell activation.

In some embodiments, any one of the cells described herein comprises a nucleic acid encoding a CAR or a third generation CAR. In some embodiments, a third generation CAR comprises an antigen binding domain, a transmembrane domain, and at least three signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and or CAR-T cell persistence during T cell activation. In some embodiments, a third generation CAR comprises at least two costimulatory domains. In some embodiments, the at least two costimulatory domains are not the same.

In some embodiments, any one of the cells described herein comprises a nucleic acid encoding a CAR or a fourth generation CAR. In some embodiments, a fourth generation CAR comprises an antigen binding domain, a transmembrane domain, and at least two, three, or four signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and or CAR-T cell persistence during T cell activation.

j) ABD Comprising an Antibody or Antigen-Binding Portion Thereof.

In some embodiments, a CAR antigen binding domain is or comprises an antibody or antigen-binding portion thereof. In some embodiments, a CAR antigen binding domain is or comprises an scFv or Fab. In some embodiments, a CAR antigen binding domain comprises an scFv or Fab fragment of a T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD19 antibody; CD20 antibody; CD21 antibody; CD22 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-1BB, CD134/OX40, CD30, CD40, PD1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

In addition to the CARs described herein, various CARs and nucleotide sequences encoding the same are known in the art and would be suitable for fusosomal delivery and reprogramming of target cells in vivo and in vitro as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

3. Therapeutic Cells Derived from Pluripotent Stem Cells

Provided herein are hypoimmunogenic cells including, cells derived from pluripotent stem cells, that evade immune recognition. In some embodiments, the cells do not activate an immune response in the patient or subject (e.g., recipient upon administration). Provided are methods of treating a disorder comprising repeat dosing of a population of hypoimmunogenic cells to a recipient subject in need thereof.

In some embodiments, the pluripotent stem cell and any cell differentiated from such a pluripotent stem cell is modified to exhibit reduced expression of MHC class I human leukocyte antigens. In other embodiments, the pluripotent stem cell and any cell differentiated from such a pluripotent stem cell is modified to exhibit reduced expression of MHC class II human leukocyte antigens. In some embodiments, the pluripotent stem cell and any cell differentiated from such a pluripotent stem cell is modified to exhibit reduced expression of MHC class I and II human leukocyte antigens. In some embodiments, the pluripotent stem cell and any cell differentiated from such a pluripotent stem cell is modified to exhibit reduced expression of MHC class I and/or II human leukocyte antigens and exhibit increased CD47 expression. In some instances, the cell overexpresses CD47 by harboring one or more transgenes encoding tolerogenic factors. In some embodiments, the pluripotent stem cell and any cell differentiated from such a pluripotent stem cell is modified to exhibit reduced expression of MHC class I and/or II human leukocyte antigens and exhibit increased tolerogenic factor expression. In some instances, the cell overexpresses CD24 by harboring one or more CD24 transgenes. In some instances, the cell overexpresses DUX4 by harboring one or more DUX4 transgenes. Such pluripotent stem cells are hypoimmunogenic pluripotent cells. Such differentiated cells are hypoimmunogenic cells. Examples of differentiated cells include, but are not limited to, cardiac cells, cardiac progenitor cells, neural cells, glial progenitor cells, endothelial cells, T cells, B cells, pancreatic islet cells, retinal pigmented epithelium cells, hepatocytes, thyroid cells, skin cells, blood cells, plasma cells, platelets, renal cells, epithelial cells, chimeric antigen receptor (CAR) T cells, NK cells, and/or CAR-NK cells.

Any of the pluripotent stem cells described herein can be differentiated into any cells of an organism and tissue. In some embodiments, the cells exhibit reduced expression of MHC class I and/or II human leukocyte antigens. In some instances, expression of MHC class I and/or II human leukocyte antigens is reduced compared to unmodified or wildtype cell of the same cell type. In some embodiments, the cells exhibit increased CD47 expression. In some instances, expression of CD47 is increased in in the cells described herein as compared to unmodified or wildtype cells of the same cell type. Methods for reducing levels of MHC class I and/or II human leukocyte antigens and increasing the expression of CD47 and one or more tolerogenic factors are described herein.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a pluripotent stem cell and any cell differentiated from such a pluripotent stem cell evades immune recognition include, but are not limited to, IFN-γ Elispot assays, microglia killing assays, cell engraftment animal models, cytokine release assays, ELISAs, killing assays using bioluminescence imaging or chromium release assay or Xcelligence analysis, mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, a neurological disorder, and the like.

4. Exemplary Embodiments of Modified Cells

In some embodiments, the cells and populations thereof exhibit increased expression of CD47 and reduced expression of one or more molecules of the MHC class I complex. In some embodiments, the cells and populations thereof exhibit increased expression of CD47 and reduced expression of one or more molecules of the MHC class II complex. In some embodiments, the cells and populations thereof exhibit increased expression of CD47 and reduced expression of one or more molecules of the MHC class II and MHC class II complexes.

In some embodiments, the cells and populations thereof exhibit increased expression of CD47 and reduced expression of B2M. In some embodiments, the cells and populations thereof exhibit increased expression of CD47 and reduced expression of CIITA. In some embodiments, the cells and populations thereof exhibit increased expression of CD47 and reduced expression of NLRC5. In some embodiments, the cells and populations thereof exhibit increased expression of CD47 and reduced expression of one or more molecules of B2M and CIITA. In some embodiments, the cells and populations thereof exhibit increased expression of CD47 and reduced expression of one or more molecules of B2M and NLRC5. In some embodiments, the cells and populations thereof exhibit increased expression of CD47 and reduced expression of one or more molecules of CIITA and NLRC5. In some embodiments, the cells and populations thereof exhibit increased expression of CD47 and reduced expression of one or more molecules of B2M, CIITA and NLRC5. Any of the cells described herein can also exhibit increased expression of one or more factors selected from the group including, but not limited to, DUX4, CD24, CD27, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, and Serpinb9.

In some embodiments, the cells and populations thereof exhibit increased expression of CD47 and at least one other tolerogenic factor, and reduced expression of one or more molecules of the MHC class I complex. In some embodiments, the cells and populations thereof exhibit increased expression of CD47 and at least one other tolerogenic factor, and reduced expression of one or more molecules of the MHC class II complex. In some embodiments, the cells and populations thereof exhibit increased expression of CD47 and at least one other tolerogenic factor, and reduced expression of one or more molecules of the MHC class II and MHC class II complexes. In some embodiments, the cells and populations thereof exhibit increased expression of CD47 and at least one other tolerogenic factor, and reduced expression of B2M. In some embodiments, the cells and populations thereof exhibit increased expression of CD47 and at least one other tolerogenic factor, and reduced expression of CIITA. In some embodiments, the cells and populations thereof exhibit increased expression of CD47 and at least one other tolerogenic factor, and reduced expression of NLRC5. In some embodiments, the cells and populations thereof exhibit increased expression of CD47 and at least one other tolerogenic factor, and reduced expression of one or more molecules of B2M and CIITA. In some embodiments, the cells and populations thereof exhibit increased expression of CD47 and at least one other tolerogenic factor, and reduced expression of one or more molecules of B2M and NLRC5. In some embodiments, the cells and populations thereof exhibit increased expression of CD47 and at least one other tolerogenic factor, and reduced expression of one or more molecules of CIITA and NLRC5. In some embodiments, the cells and populations thereof exhibit increased expression of CD47 and at least one other tolerogenic factor, and reduced expression of one or more molecules of B2M, CIITA and NLRC5. In some embodiments, a tolerogenic factor includes any from the group including, but not limited to, DUX4, CD24, CD27, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, and Serpinb9.

One skilled in the art will appreciate that levels of expression such as increased or reduced expression of a gene, protein or molecule can be referenced or compared to a comparable cell. In some embodiments, an engineered stem cell having increased expression of CD47 refers to a modified stem cell having a higher level of CD47 protein compared to an unmodified stem cell.

In one embodiment, provided herein are cells (e.g., stem cell, induced pluripotent stem cell, differentiated cell, hematopoietic stem cell, primary cell, CAR-T cell, and/or CAR-NK cell) expressing exogenous CD47 polypeptides and having reduced expression of either one or more MHC class I complex proteins, one or more MHC class II complex proteins, or any combination of MHC class I and class II complex proteins. In another embodiment, the cells express exogenous CD47 polypeptides and express reduced levels of B2M and CIITA polypeptides. In some embodiments, the cells express exogenous CD47 polypeptides and possess genetic modifications of the B2M and CIITA genes. In some instances, the genetic modifications inactivate the B2M and CIITA genes.

In some embodiments, the cells (e.g., stem cell, induced pluripotent stem cell, differentiated cell, hematopoietic stem cell, primary cell, CAR-T cell and/or CAR-NK cell) possess genetic modifications that inactivate the B2M and CIITA genes and express a plurality of exogenous polypeptides selected from the group including CD47 and DUX4, CD47 and CD24, CD47 and CD27, CD47 and CD46, CD47 and CD55, CD47 and CD59, CD47 and CD200, CD47 and HLA-C, CD47 and HLA-E, CD47 and HLA-E heavy chain, CD47 and HLA-G, CD47 and PD-L1, CD47 and IDO1, CD47 and CTLA4-Ig, CD47 and C1-Inhibitor, CD47 and IL-10, CD47 and IL-35, CD47 and IL-39, CD47 and FasL, CD47 and CCL21, CD47 and CCL22, CD47 and Mfge8, and CD47 and Serpinb9, and any combination thereof. In some instances, such cells also possess a genetic modification that inactivates the CD142 gene.

C. CD47

In some embodiments, the present disclosure provides a cell or population thereof that has been modified to express the tolerogenic factor (e.g., immunomodulatory polypeptide) CD47. In some embodiments, the present disclosure provides a method for altering a cell genome to express CD47. In some embodiments, the stem cell expresses exogenous CD47. In some instances, the cell expresses an expression vector comprising a nucleotide sequence encoding a human CD47 polypeptide. In some embodiments, the cell is genetically modified to comprise an integrated exogenous polynucleotide encoding CD47 using homology-directed repair.

CD47 is a leukocyte surface antigen and has a role in cell adhesion and modulation of integrins. It is expressed on the surface of a cell and signals to circulating macrophages not to eat the cell.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2. In some embodiments, the nucleotide sequence encoding a CD47 polynucleotide is a codon optimized sequence. In some embodiments, the nucleotide sequence encoding a CD47 polynucleotide is a human codon optimized sequence.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

Exemplary amino acid sequences of human CD47 with a signal sequence and without a signal sequence are provided in Table 1.

TABLE 1
Amino acid sequences of human CD47

| | SEQ | | Amino |
|---|---|---|---|
| | ID | | acid |
| Protein | NO: | Sequence | residues |
| Human | 12 | QLLFNKTKSVEFTFCNDTVVIPCFVTNMEAQNTTEV | aa 19-323 |
| CD47 | | YVKWKFKGRDIYTFDGALNKSTVPTDFSSAKIEVS | |
| (without | | QLLKGDASLKMDKSDAVSHTGNYTCEVTELTREG | |
| signal | | ETIIELKYRVVSWFSPNENILIVIFPIFAILLFWGQFGI | |
| sequence) | | KTLKYRSGGMDEKTIALLVAGLVITVIVIVGAILFVP | |
| | | GEYSLKNATGLGLIVTSTGILILLHYYVFSTAIGLTSF | |
| | | VIAILVIQVIAYILAVVGLSLCIAACIPMHGPLLISGL | |
| | | SILALAQLLGLVYMKFVASNQKTIQPPRKAVEEPLN | |
| | | AFKESKGMMNDE | |
| Human | 13 | MWPLVAALLLGSACCGSAQLLFNKTKSVEFTFCND | aa 1-323 |
| CD47 (with | | TVVIPCFVTNMEAQNTTEVYVKWKFKGRDIYTFDG | |
| signal | | ALNKSTVPTDFSSAKIEVSQLLKGDASLKMDKSDA | |
| sequence) | | VSHTGNYTCEVTELTREGETIIELKYRVVSWFSPNE | |
| | | NILIVIFPIFAILLFWGQFGIKTLKYRSGGMDEKTIAL | |
| | | LVAGLVITVIVIVGAILFVPGEYSLKNATGLGLIVTS | |
| | | TGILILLHYYVFSTAIGLTSFVIAILVIQVIAYILAVVG | |
| | | LSLCIAACIPMHGPLLISGLSILALAQLLGLVYMKFV | |
| | | ASNQKTIQPPRKAVEEPLNAFKESKGMMNDE | |

In some embodiments, the cell comprises a CD47 polypeptide having at least 95% sequence identity (e.g., 95%, 96%, 97%, 98%, 99%, or more) to the amino acid sequence of SEQ ID NO:12. In some embodiments, the cell comprises a CD47 polypeptide having the amino acid sequence of SEQ ID NO:12. In some embodiments, the cell comprises a CD47 polypeptide having at least 95% sequence identity (e.g., 95%, 96%, 97%, 98%, 99%, or more) to the amino acid sequence of SEQ ID NO:12. In some embodiments, the cell comprises a CD47 polypeptide having the amino acid sequence of SEQ ID NO:12.

In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO:13. In some embodiments, the cell comprises a nucleotide sequence encoding a CD47 polypeptide having the amino acid sequence of SEQ ID NO:13. In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO:13. In some embodiments, the cell comprises a nucleotide sequence encoding a CD47 polypeptide having the amino acid sequence of SEQ ID NO:13. In some embodiments, the nucleotide sequence is codon optimized for expression in a particular cell.

In some embodiments, a suitable gene editing system (e.g., CRISPR/Cas system or any of the gene editing systems described herein) is used to facilitate the insertion of a polynucleotide encoding CD47, into a genomic locus of the hypoimmunogenic cell. In some cases, the polynucleotide encoding CD47 is inserted into a safe harbor or a target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, PDGFRa, OLIG2, GFAP, or KDM5D gene locus. In some embodiments, the polynucleotide encoding CD47 is inserted into a B2M gene locus, a CIITA gene locus, a TRAC gene locus, or a TRB gene locus. In some embodiments, the polynucleotide encoding CD47 is inserted into any one of the gene loci depicted in Table 4 provided herein. In certain embodiments, the polynucleotide encoding CD47 is operably linked to a promoter.

In another embodiment, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

D. CD24

In some embodiments, the present disclosure provides a cell or population thereof that has been modified to express the tolerogenic factor (e.g., immunomodulatory polypeptide) CD24. In some embodiments, the present disclosure provides a method for altering a cell genome to express CD24. In some embodiments, the stem cell expresses exogenous CD24. In some instances, the cell expresses an expression vector comprising a nucleotide sequence encoding a human CD24 polypeptide. In some embodiments, the cell is genetically modified to comprise an integrated exogenous polynucleotide encoding CD24 using homology-directed repair.

CD24 which is also referred to as a heat stable antigen or small-cell lung cancer cluster 4 antigen is a glycosylated glycosylphosphatidylinositol-anchored surface protein (Pirruccello et al., J Immunol., 1986, 136, 3779-3784; Chen et al., Glycobiology, 2017, 57, 800-806). It binds to Siglec-10 on innate immune cells. Recently it has been shown that CD24 via Siglec-10 acts as an innate immune checkpoint (Barkal et al., Nature, 2019, 572, 392-396).

In some embodiments, the cell outlined herein comprises a nucleotide sequence encoding a CD24 polypeptide has at least 95% sequence identity (e.g., 95%, 96%, 97%, 98%, 99%, or more) to an amino acid sequence set forth in NCBI Ref. Nos. NP_001278666.1, NP_001278667.1, NP_001278668.1, and NP_037362.1. In some embodiments, the cell outlined herein comprises a nucleotide sequence encoding a CD24 polypeptide having an amino acid sequence set forth in NCBI Ref. Nos. NP_001278666.1, NP_001278667.1, NP_001278668.1, and NP_037362.1.

In some embodiments, the cell comprises a nucleotide sequence having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_00129737.1, NM_00129738.1, NM_001291739.1, and NM_013230.3. In some embodiments, the cell comprises a nucleotide sequence as set forth in NCBI Ref. Nos. NM_00129737.1, NM_00129738.1, NM_001291739.1, and NM_013230.3.

In another embodiment, CD24 protein expression is detected using a Western blot of cells lysates probed with antibodies against the CD24 protein. In another embodiment, reverse transcriptase polymerase chain reactions (RT-PCR) are used to confirm the presence of the exogenous CD24 mRNA.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some cases, the polynucleotide encoding CD24 is inserted into a safe harbor or a target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, PDGFRa, OLIG2, GFAP, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is inserted into any one of the gene loci depicted in Table 4 provided herein. In some embodiments, the polynucleotide encoding CD24 is operably linked to a promoter.

E. DUX4

In some embodiments, the present disclosure provides a cell (e.g., stem cell, induced pluripotent stem cell, differentiated cell, hematopoietic stem cell, primary cell or CAR-T cell) or population thereof comprising a genome modified to increase expression of a tolerogenic or immunosuppressive factor such as DUX4. In some embodiments, the present disclosure provides a method for altering a cell's genome to provide increased expression of DUX4. In one aspect, the disclosure provides a cell or population thereof comprising exogenously expressed DUX4 proteins. In some embodiments, the cell is genetically modified to comprise an integrated exogenous polynucleotide encoding DUX4 using homology-directed repair. In some embodiments, increased expression of DUX4 suppresses, reduces or eliminates expression of one or more of the following MHC I molecules—HLA-A, HLA-B, and HLA-C.

DUX4 is a transcription factor that is active in embryonic tissues and induced pluripotent stem cells, and is silent in normal, healthy somatic tissues (Feng et al., 2015, ELife4; De Iaco et al., 2017, Nat Genet., 49, 941-945; Hendrickson et al., 2017, Nat Genet., 49, 925-934; Snider et al., 2010, PLoS Genet., e1001181; Whiddon et al., 2017, Nat Genet.). DUX4 expression acts to block IFN-gamma mediated induction of major histocompatibility complex (MHC) class I gene expression (e.g., expression of B2M, HLA-A, HLA-B, and HLA-C). DUX4 expression has been implicated in suppressed antigen presentation by MHC class I (Chew et al., Developmental Cell, 2019, 50:1-14). DUX4 functions as a transcription factor in the cleavage-stage gene expression (transcriptional) program. Its target genes include, but are not limited to, coding genes, noncoding genes, and repetitive elements.

There are at least two isoforms of DUX4, with the longest isoform comprising the DUX4 C-terminal transcription activation domain. The isoforms are produced by alternative splicing. See, e.g., Geng et al., 2012, Developmental Cell, 22, 38-51; Snider et al., 2010, PLoS Genet., e1001181. Active isoforms for DUX4 comprise its N-terminal DNA-binding domains and its C-terminal activation domain. See, e.g., Choi et al., 2016, Nucleic Acid Res., 44, 5161-5173.

It has been shown that reducing the number of CpG motifs of DUX4 decreases silencing of a DUX4 transgene (Jagannathan et al., Human Molecular Genetics, 2016, 25(20):4419-4431). The nucleic acid sequence provided in Jagannathan et al., supra represents a codon altered sequence of DUX4 comprising one or more base substitutions to reduce the total number of CpG sites while preserving the DUX4 protein sequence. The nucleic acid sequence is commercially available from Addgene, Catalog No. 99281.

In certain aspects, at least one or more polynucleotides may be utilized to facilitate the exogenous expression of DUX4 by a cell, e.g., a stem cell, induced pluripotent stem cell, differentiated cell, hematopoietic stem cell, primary cell or CAR-T cell.

In some embodiments, a suitable gene editing system (e.g., CRISPR/Cas system or any of the gene editing systems described herein) is used to facilitate the insertion of a polynucleotide encoding DUX4, into a genomic locus of the hypoimmunogenic cell. In some cases, the polynucleotide encoding DUX4 is inserted into a safe harbor or a target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, PDGFRa, OLIG2, GFAP, or KDM5D gene locus. In some embodiments, the polynucleotide encoding DUX4 is inserted into a B2M gene locus, a CIITA gene locus, a TRAC gene locus, or a TRB gene locus. In some embodiments, the polynucleotide encoding DUX4 is inserted into any one of the gene loci depicted in Table 4 provided herein. In certain embodiments, the polynucleotide encoding DUX4 is operably linked to a promoter.

In some embodiments, the polynucleotide sequence encoding DUX4 comprises a polynucleotide sequence comprising a codon altered nucleotide sequence of DUX4 comprising one or more base substitutions to reduce the total number of CpG sites while preserving the DUX4 protein sequence. In some embodiments, the polynucleotide sequence encoding DUX4 comprising one or more base substitutions to reduce the total number of CpG sites has at least 85% (e.g., 85%, 86%, 87%, 88%, 89%, 90%, 91%, 92%, 93%, 94%, 95%, 96%, 97%, 98%, 99% or 100%) sequence identity to SEQ ID NO:1 of PCT/US2020/44635, filed Jul. 31, 2020. In some embodiments, the polynucleotide sequence encoding DUX4 is SEQ ID NO:1 of PCT/US2020/44635.

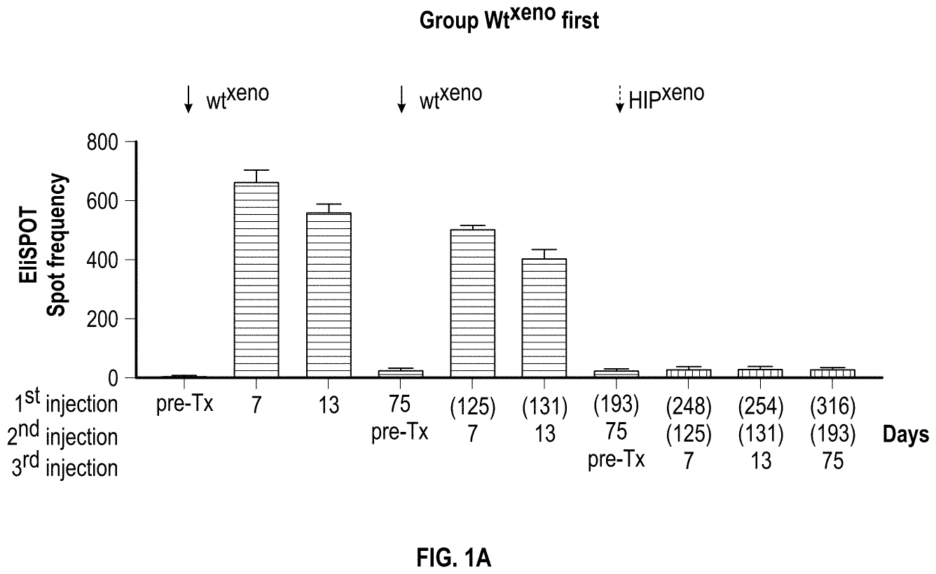

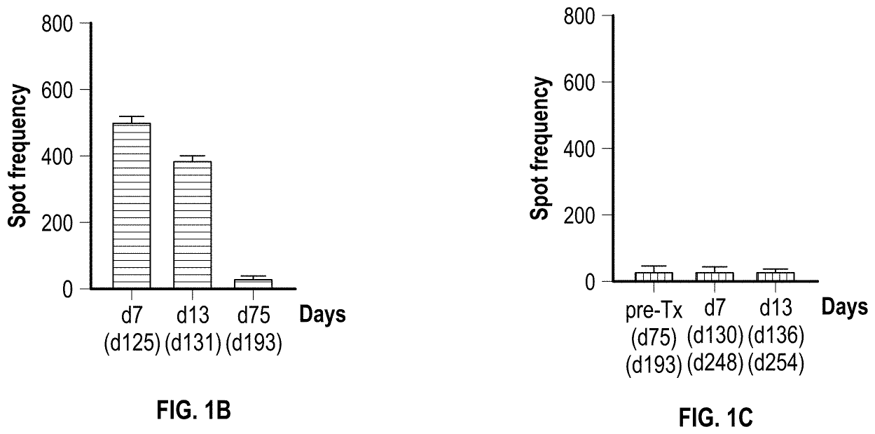

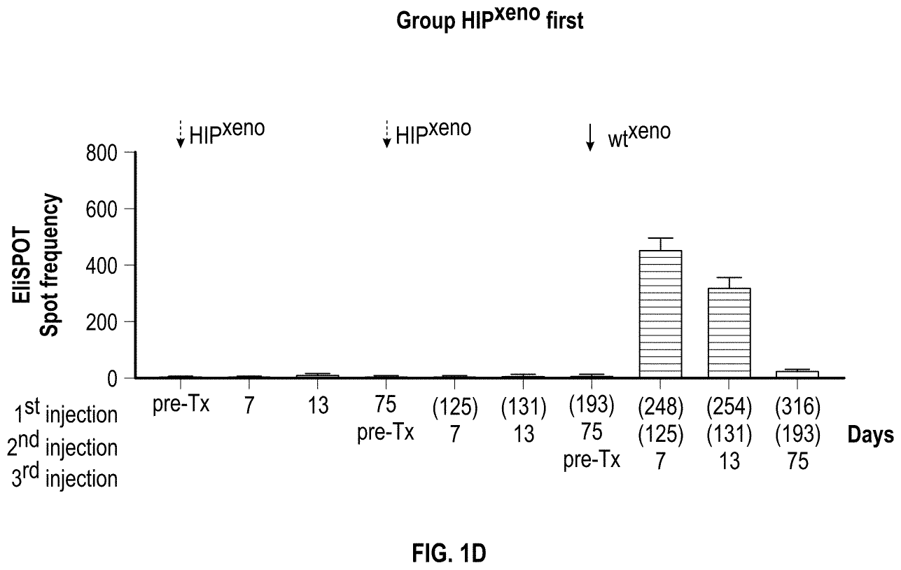

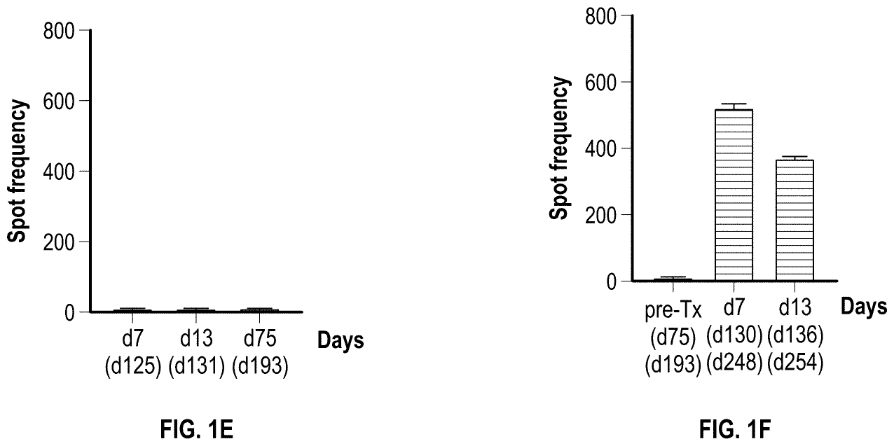

In some embodiments, the polynucleotide sequence encoding DUX4 is a nucleotide sequence encoding a polypeptide sequence having at least 95% (e.g., 95%, 96%, 97%, 98%, 99% or 100%) sequence identity to a sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29, as provided in PCT/US2020/44635. In some embodiments, the polynucleotide sequence encoding DUX4 is a nucleotide sequence encoding a polypeptide sequence is selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29. Amino acid sequences set forth as SEQ ID NOS:2-29 are shown in FIG. 1A-1G of PCT/US2020/44635.

In some instances, the DUX4 polypeptide comprises an amino acid sequence having at least 95% sequence identity to the sequence set forth in GenBank Accession No. ACN62209.1 or an amino acid sequence set forth in GenBank Accession No. ACN62209.1. In some instances, the DUX4 polypeptide comprises an amino acid sequence having at least 95% sequence identity to the sequence set forth in NCBI RefSeq No. NP_001280727.1 or an amino acid sequence set forth in NCBI RefSeq No. NP_001280727.1. In some instances, the DUX4 polypeptide comprises an amino acid sequence having at least 95% sequence identity to the sequence set forth in GenBank Accession No. ACP30489.1 or an amino acid sequence set forth in GenBank Accession No. ACP30489.1. In some instances, the DUX4 polypeptide comprises an amino acid sequence having at least 95% sequence identity to the sequence set forth in UniProt No. P0CJ85.1 or an amino acid sequence set forth in UniProt No. P0CJ85.1. In some instances, the DUX4 polypeptide comprises an amino acid sequence having at least 95% sequence identity to the sequence set forth in GenBank Accession No. AUA60622.1 or an amino acid sequence set forth in GenBank Accession No. AUA60622.1. In some instances, the DUX4 polypeptide comprises an amino acid sequence having at least 95% sequence identity to the sequence set forth in GenBank Accession No. ADK24683.1 or an amino acid sequence set forth in GenBank Accession No. ADK24683.1. In some instances, the DUX4 polypeptide comprises an amino acid sequence having at least 95% sequence identity to the sequence set forth in GenBank Accession No. ACN62210.1 or an amino acid sequence set forth in GenBank Accession No. ACN62210.1. In some instances, the DUX4 polypeptide comprises an amino acid sequence having at least 95% sequence identity to the sequence set forth in GenBank Accession No. ADK24706.1 or an amino acid sequence set forth in GenBank Accession No. ADK24706.1. In some instances, the DUX4 polypeptide comprises an amino acid sequence having at least 95% sequence identity to the sequence set forth in GenBank Accession No. ADK24685.1 or an amino acid sequence set forth in GenBank Accession No. ADK24685.1. In some instances, the DUX4 polypeptide comprises an amino acid sequence having at least 95% sequence identity to the sequence set forth in GenBank Accession No. ACP30488.1 or an amino acid sequence set forth in GenBank Accession No. ACP30488.1. In some instances, the DUX4 polypeptide comprises an amino acid sequence having at least 95% sequence identity to the sequence set forth in GenBank Accession No. ADK24687.1 or an amino acid sequence set forth in GenBank Accession No. ADK24687.1. In some instances, the DUX4 polypeptide comprises an amino acid sequence having at least 95% sequence identity to the sequence set forth in GenBank Accession No. ACP30487.1 or an amino acid sequence set forth in GenBank Accession No. ACP30487.1. In some instances, the DUX4 polypeptide comprises an amino acid sequence having at least 95% sequence identity to the sequence set forth in GenBank Accession No. ADK24717.1 or an amino acid sequence set forth in GenBank Accession No. ADK24717.1. In some instances, the DUX4 polypeptide comprises an amino acid sequence having at least 95% sequence identity to the sequence set forth in GenBank Accession No. ADK24690.1 or an amino acid sequence set forth in GenBank Accession No. ADK24690.1. In some instances, the DUX4 polypeptide comprises an amino acid sequence having at least 95% sequence identity to the sequence set forth in GenBank Accession No. ADK24689.1 or an amino acid sequence set forth in GenBank Accession No. ADK24689.1. In some instances, the DUX4 polypeptide comprises an amino acid sequence having at least 95% sequence identity to the sequence set forth in GenBank Accession No. ADK24692.1 or an amino acid sequence set forth in GenBank Accession No. ADK24692.1. In some instances, the DUX4 polypeptide comprises an amino acid sequence having at least 95% sequence identity to the sequence set forth in GenBank Accession No. ADK24693.1 or an amino acid sequence of set forth in GenBank Accession No. ADK24693.1. In some instances, the DUX4 polypeptide comprises an amino acid sequence having at least 95% sequence identity to the sequence set forth in GenBank Accession No. ADK24712.1 or an amino acid sequence set forth in GenBank Accession No. ADK24712.1. In some instances, the DUX4 polypeptide comprises an amino acid sequence having at least 95% sequence identity to the sequence set forth in GenBank Accession No. ADK24691.1 or an amino acid sequence set forth in GenBank Accession No. ADK24691.1. In some instances, the DUX4 polypeptide comprises an amino acid sequence having at least 95% sequence identity to the sequence set forth in UniProt No. P0CJ87.1 or an amino acid sequence of set forth in UniProt No. P0CJ87.1. In some instances, the DUX4 polypeptide comprises an amino acid sequence having at least 95% sequence identity to the sequence set forth in GenBank Accession No. ADK24714.1 or an amino acid sequence set forth in GenBank Accession No. ADK24714.1. In some instances, the DUX4 polypeptide comprises an amino acid sequence having at least 95% sequence identity to the sequence set forth in GenBank Accession No. ADK24684.1 or an amino acid sequence of set forth in GenBank Accession No. ADK24684.1. In some instances, the DUX4 polypeptide comprises an amino acid sequence having at least 95% sequence identity to the sequence set forth in GenBank Accession No. ADK24695.1 or an amino acid sequence set forth in GenBank Accession No. ADK24695.1. In some instances, the DUX4 polypeptide comprises an amino acid sequence having at least 95% sequence identity to the sequence set forth in GenBank Accession No. ADK24699.1 or an amino acid sequence set forth in GenBank Accession No. ADK24699.1. In some instances, the DUX4 polypeptide comprises an amino acid sequence having at least 95% sequence identity to the sequence set forth in NCBI RefSeq No. NP_001768.1 or an amino acid sequence set forth in NCBI RefSeq No. NP_001768. In some instances, the DUX4 polypeptide comprises an amino acid sequence having at least 95% sequence identity to the sequence set forth in NCBI RefSeq No. NP_942088.1 or an amino acid sequence set forth in NCBI RefSeq No. NP_942088.1. In some instances, the DUX4 polypeptide comprises an amino acid sequence having at least 95% sequence identity to SEQ ID NO:28 provided in PCT/US2020/44635 or an amino acid sequence of SEQ ID NO:28 provided in PCT/US2020/44635. In some instances, the DUX4 polypeptide comprises an amino acid sequence having at least 95% sequence identity to SEQ ID NO:29 provided in PCT/US2020/44635 or an amino acid sequence of SEQ ID NO:29 provided in PCT/US2020/44635.

In other embodiments, expression of tolerogenic factors is facilitated using an expression vector. In some embodiments, the expression vector comprises a polynucleotide sequence encoding DUX4 is a codon altered sequence comprising one or more base substitutions to reduce the total number of CpG sites while preserving the DUX4 protein sequence. In some cases, the codon altered sequence of DUX4 comprises SEQ ID NO:1 of PCT/US2020/44635. In some cases, the codon altered sequence of DUX4 is SEQ ID NO:1 of PCT/US2020/44635. In other embodiments, the expression vector comprises a polynucleotide sequence encoding DUX4 comprising SEQ ID NO:1 of PCT/US2020/44635. In some embodiments, the expression vector comprises a polynucleotide sequence encoding a DUX4 polypeptide sequence having at least 95% sequence identity to a sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 of PCT/US2020/44635. In some embodiments, the expression vector comprises a polynucleotide sequence encoding a DUX4 polypeptide sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 of PCT/US2020/44635.

An increase of DUX4 expression can be assayed using known techniques, such as Western blots, ELISA assays, FACS assays, immunoassays, and the like.

F. CIITA

In certain aspects, the technology disclosed herein modulate (e.g., reduce or eliminate) the expression of MHC II genes by targeting and modulating (e.g., reducing or eliminating) Class II transactivator (CIITA) expression. In some embodiments, the modulation occurs using a gene editing (e.g., CRISPR/Cas) system. In some embodiments, the modification is transient (including, for example, by employing siRNA methods). In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of CIITA expression includes, but is not limited, to reduced transcription, decreased mRNA stability (such as by way of RNAi mechanisms), and reduced protein levels.

CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Appendix 1 or Table 12 of WO2016183041, the disclosure is incorporated by reference in its entirety. In some embodiments, an exogenous nucleic acid encoding a polypeptide as disclosed herein (e.g., a chimeric antigen receptor, CD47, or another tolerogenic factor disclosed herein) is inserted at the CIITA gene.

Assays to test whether the CIITA gene has been inactivated are known and described herein. In one embodiment, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

G. B2M

In certain embodiments, the technology disclosed herein modulate (e.g., reduce or eliminate) the expression of MHC-I genes by targeting and modulating (e.g., reducing or eliminating) expression of the accessory chain B2M. In some embodiments, the modulation occurs using a gene editing (e.g., CRISPR/Cas) system. In some embodiments, the modification is transient (including, for example, by employing siRNA methods). In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of B2M expression includes, but is not limited, to reduced transcription, decreased mRNA stability (such as by way of RNAi mechanisms), and reduced protein levels.

By modulating (e.g., reducing or deleting) expression of B2M, surface trafficking of MHC-I molecules is blocked and such cells exhibit immune tolerance when engrafted into a recipient subject. In some embodiments, the cell is considered hypoimmunogenic, e.g., in a recipient subject or patient upon administration.

In some embodiments, the target polynucleotide sequence provided herein is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules—HLA-A, HLA-B, and HLA-C.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Appendix 2 or Table 15 of WO2016/183041, the disclosure is incorporated by reference in its entirety. In some embodiments, an exogenous nucleic acid encoding a polypeptide as disclosed herein (e.g., a chimeric antigen receptor, CD47, or another tolerogenic factor disclosed herein) is inserted at the B2M gene.

Assays to test whether the B2M gene has been inactivated are known and described herein. In one embodiment, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

H. NLRC5

In certain aspects, the technology disclosed herein modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some embodiments, the modulation occurs using a gene editing (e.g., CRISPR/Cas) system. In some embodiments, the modification is transient (including, for example, by employing siRNA methods). In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of NLRC5 expression includes, but is not limited, to reduced transcription, decreased mRNA stability (such as by way of RNAi mechanisms), and reduced protein levels.

NLRC5 is a regulator of MHC-I-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, the target polynucleotide sequence is a variant of NLRC5. In some embodiments, the target polynucleotide sequence is a homolog of NLRC5. In some embodiments, the target polynucleotide sequence is an ortholog of NLRC5.

In some embodiments, decreased or eliminated expression of NLRC5 reduces or eliminates expression of one or more of the following MHC I molecules—HLA-A, HLA-B, and HLA-C.

In some embodiments, the cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOS:36353-81239 of Appendix 3 or Table 14 of WO2016183041, the disclosure is incorporated by reference in its entirety.

Assays to test whether the NLRC5 gene has been inactivated are known and described herein. In one embodiment, the resulting genetic modification of the NLRC5 gene by PCR and the reduction of HLA-I expression can be assays by FACS analysis. In another embodiment, NLRC5 protein expression is detected using a Western blot of cells lysates probed with antibodies to the NLRC5 protein. In another embodiment, reverse transcriptase polymerase chain reactions (RT-PCR) are used to confirm the presence of the inactivating genetic modification.

I. TRAC

In many embodiments, the technologies disclosed herein regulatably modulate (e.g., reduce or eliminate) the expression of TCR genes including the TRAC gene by regulatably targeting and modulating (e.g., reducing or eliminating) expression of the constant region of the T cell receptor alpha chain. In some embodiments, the modulation occurs using a gene editing (e.g., CRISPR/Cas) system. In some embodiments, the modification is transient (including, for example, by employing siRNA methods). In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of TRAC expression includes, but is not limited, to reduced transcription, decreased mRNA stability (such as by way of RNAi mechanisms), and reduced protein levels.

By modulating (e.g., reducing or deleting) expression of TRAC, surface trafficking of TCR molecules is blocked. In some embodiments, the cell also has a reduced ability to induce an immune response in a recipient subject.

In some embodiments, the target polynucleotide sequence of the present technology is a variant of TRAC. In some embodiments, the target polynucleotide sequence is a homolog of TRAC. In some embodiments, the target polynucleotide sequence is an ortholog of TRAC.

In some embodiments, decreased or eliminated expression of TRAC reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise regulatable gene modifications at the gene locus encoding the TRAC protein. In other words, the cells comprise a regulatable genetic modification at the TRAC locus. In some instances, the nucleotide sequence encoding the TRAC protein is set forth in Genbank No. X02592.1. In some instances, the TRAC gene locus is described in RefSeq. No. NG_001332.3 and NCBI Gene ID No. 28755. In certain cases, the amino acid sequence of TRAC is depicted as Uniprot No. P01848. Additional descriptions of the TRAC protein and gene locus can be found in Uniprot No. P01848, HGNC Ref. No. 12029, and OMIM Ref. No. 186880.

In some embodiments, the hypoimmunogenic cells outlined herein comprise a regulatable genetic modification targeting the TRAC gene. In some embodiments, the regulatable genetic modification targeting the TRAC gene is by way of a regulatable rare-cutting endonuclease comprising a regulatable Cas protein or a regulatable polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOS:532-609 and 9102-9797 of US20160348073, which is herein incorporated by reference.

Assays to test whether the TRAC gene has been inactivated are known and described herein. In some embodiments, the resulting genetic modification of the TRAC gene by PCR and the reduction of TCR expression can be assays by FACS analysis. In another embodiment, TRAC protein expression is detected using a Western blot of cells lysates probed with antibodies to the TRAC protein. In another embodiment, reverse transcriptase polymerase chain reactions (RT-PCR) are used to confirm the presence of the inactivating genetic modification.

In some embodiments, the hypoimmunogenic cells outlined herein comprise regulatable knock out of TRAC expression, such that the cells are regulatably TRAC^{-/-}. In some embodiments, the hypoimmunogenic cells outlined herein regulatably introduce an indel into the TRAC gene locus, such that the cells are regulatably TRAC^{indel/indel}. In some embodiments, the hypoimmunogenic cells outlined herein comprise regulatable knock down of TRAC expression, such that the cells are regulatably TRAC^{knock down}.

J. TRB

In many embodiments, the technologies disclosed herein regulatably modulate (e.g., reduce or eliminate) the expression of TCR genes including the gene encoding T cell antigen receptor, beta chain (e.g., the TRB, TRBC, or TCRB gene) by regulatably targeting and modulating (e.g., reducing or eliminating) expression of the constant region of the T cell receptor beta chain. In some embodiments, the modulation occurs using a gene editing (e.g., CRISPR/Cas) system. In some embodiments, the modification is transient (including, for example, by employing siRNA methods). In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of TRB expression includes, but is not limited, to reduced transcription, decreased mRNA stability (such as by way of RNAi mechanisms), and reduced protein levels.

By modulating (e.g., reducing or deleting) expression of TRB, surface trafficking of TCR molecules is blocked. In some embodiments, the cell also has a reduced ability to induce an immune response in a recipient subject.

In some embodiments, the target polynucleotide sequence of the present technology is a variant of TRB. In some embodiments, the target polynucleotide sequence is a homolog of TRB. In some embodiments, the target polynucleotide sequence is an ortholog of TRB.

In some embodiments, decreased or eliminated expression of TRB reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise regulatable gene modifications at the gene locus encoding the TRB protein. In other words, the cells comprise a regulatable genetic modification at the TRB gene locus. In some instances, the nucleotide sequence encoding the TRB protein is set forth in UniProt No. P0DSE2. In some instances, the TRB gene locus is described in RefSeq. No. NG_001333.2 and NCBI Gene ID No. 6957. In certain cases, the amino acid sequence of TRB is depicted as Uniprot No. P01848. Additional descriptions of the TRB protein and gene locus can be found in GenBank No. L36092.2, Uniprot No. P0DSE2, and HGNC Ref. No. 12155.

In some embodiments, the hypoimmunogenic cells outlined herein comprise a regulatable genetic modification targeting the TRB gene. In some embodiments, the regulatable genetic modification targeting the TRB gene is by way of a regulatable rare-cutting endonuclease comprising a regulatable Cas protein or a regulatable polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOS:610-765 and 9798-10532 of US20160348073, which is herein incorporated by reference.

Assays to test whether the TRB gene has been inactivated are known and described herein. In some embodiments, the resulting genetic modification of the TRB gene by PCR and the reduction of TCR expression can be assays by FACS analysis. In another embodiment, TRB protein expression is detected using a Western blot of cells lysates probed with antibodies to the TRB protein. In another embodiment, reverse transcriptase polymerase chain reactions (RT-PCR) are used to confirm the presence of the inactivating genetic modification.

In some embodiments, the hypoimmunogenic cells outlined herein comprise regulatable knock out of TRB expression, such that the cells are regulatably TRB^{-/-}. In some embodiments, the hypoimmunogenic cells outlined herein regulatably introduce an indel into the TRB gene locus, such that the cells are regulatably TRB^{indel/indel}. In some embodiments, the hypoimmunogenic cells outlined herein comprise regulatable knock down of TRB expression, such that the cells are regulatably TRB^{knock down}.

K. CD142

In certain aspects, the technology disclosed herein modulate (e.g., reduce or eliminate) the expression of CD142, which is also known as tissue factor, factor III, and F3. In some embodiments, the modulation occurs using a gene editing (e.g., CRISPR/Cas) system. In some embodiments, the modification is transient (including, for example, by employing siRNA methods). In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of CD142 expression includes, but is not limited, to reduced transcription, decreased mRNA stability (such as by way of RNAi mechanisms), and reduced protein levels.

In some embodiments, the target polynucleotide sequence is CD142 or a variant of CD142. In some embodiments, the target polynucleotide sequence is a homolog of CD142. In some embodiments, the target polynucleotide sequence is an ortholog of CD142.

In some embodiments, the cells outlined herein comprise a genetic modification targeting the CD142 gene. In some embodiments, the genetic modification targeting the CD142 gene by the rare-cutting endonuclease comprises a Cas protein or a polynucleotide encoding a Cas protein, and at least one guide ribonucleic acid (gRNA) sequence for specifically targeting the CD142 gene. Useful methods for identifying gRNA sequences to target CD142 are described below.

Assays to test whether the CD142 gene has been inactivated are known and described herein. In one embodiment, the resulting genetic modification of the CD142 gene by PCR and the reduction of CD142 expression can be assays by FACS analysis. In another embodiment, CD142 protein expression is detected using a Western blot of cells lysates probed with antibodies to the CD142 protein. In another embodiment, reverse transcriptase polymerase chain reactions (RT-PCR) are used to confirm the presence of the inactivating genetic modification.

Useful genomic, polynucleotide and polypeptide information about the human CD142 are provided in, for example, the GeneCard Identifier GC01M094530, HGNC No. 3541, NCBI Gene ID 2152, NCBI RefSeq Nos. NM_001178096.1, NM_001993.4, NP_001171567.1, and NP_001984.1, UniProt No. P13726, and the like.

L. CTLA4

In certain aspects, the technology disclosed herein modulate (e.g., reduce or eliminate) the expression of CTLA4. In some embodiments, the modulation occurs using a gene editing (e.g., CRISPR/Cas) system. In some embodiments, the modification is transient (including, for example, by employing siRNA methods). In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of CTLA4 expression includes, but is not limited, to reduced transcription, decreased mRNA stability (such as by way of RNAi mechanisms), and reduced protein levels.

In some embodiments, the target polynucleotide sequence is CTLA4 or a variant of CTLA4. In some embodiments, the target polynucleotide sequence is a homolog of CTLA4. In some embodiments, the target polynucleotide sequence is an ortholog of CTLA4.

In some embodiments, the cells outlined herein comprise a genetic modification targeting the CTLA4 gene. In certain embodiments, primary T cells comprise a genetic modification targeting the CTLA4 gene. The genetic modification can reduce expression of CTLA4 polynucleotides and CTLA4 polypeptides in T cells includes primary T cells and CAR-T cells. In some embodiments, the genetic modification targeting the CTLA4 gene by the rare-cutting endonuclease comprises a Cas protein or a polynucleotide encoding a Cas protein, and at least one guide ribonucleic acid (gRNA) sequence for specifically targeting the CTLA4 gene. Useful methods for identifying gRNA sequences to target CTLA4 are described below.

Assays to test whether the CTLA4 gene has been inactivated are known and described herein. In one embodiment, the resulting genetic modification of the CTLA4 gene by PCR and the reduction of CTLA4 expression can be assays by FACS analysis. In another embodiment, CTLA4 protein expression is detected using a Western blot of cells lysates probed with antibodies to the CTLA4 protein. In another embodiment, reverse transcriptase polymerase chain reactions (RT-PCR) are used to confirm the presence of the inactivating genetic modification.

Useful genomic, polynucleotide and polypeptide information about the human CTLA4 are provided in, for example, the GeneCard Identifier GC02P203867, HGNC No. 2505, NCBI Gene ID 1493, NCBI RefSeq Nos. NM_005214.4, NM_001037631.2, NP_001032720.1 and NP_005205.2, UniProt No. P16410, and the like.

M. PD1

In certain aspects, the technology disclosed herein modulate (e.g., reduce or eliminate) the expression of PD1. In some embodiments, the modulation occurs using a gene editing (e.g., CRISPR/Cas) system. In some embodiments, the modification is transient (including, for example, by employing siRNA methods). In some embodiments, the modulation occurs using a DNA-based method selected from the group consisting of a knock out or knock down using a method selected from the group consisting of CRISPRs, TALENs, zinc finger nucleases, homing endonucleases, and meganucleases. In some embodiments, the modification is transient (including, for example, by employing siRNA methods). In some embodiments, the modulation occurs using an RNA-based method selected from the group consisting of shRNAs, siRNAs, miRNAs, and CRISPR interference (CRISPRi). In some embodiments, modulation of PD1 expression includes, but is not limited, to reduced transcription, decreased mRNA stability (such as by way of RNAi mechanisms), and reduced protein levels.

In some embodiments, the target polynucleotide sequence is PD1 or a variant of PD1. In some embodiments, the target polynucleotide sequence is a homolog of PD1. In some embodiments, the target polynucleotide sequence is an ortholog of PD1.

In some embodiments, the cells outlined herein comprise a genetic modification targeting the gene encoding the programmed cell death protein 1 (PD1) protein or the PDCD1 gene. In certain embodiments, primary T cells comprise a genetic modification targeting the PDCD1 gene. The genetic modification can reduce expression of PD1 polynucleotides and PD1 polypeptides in T cells includes primary T cells and CAR-T cells. In some embodiments, the genetic modification targeting the PDCD1 gene by the rare-cutting endonuclease comprises a Cas protein or a polynucleotide encoding a Cas protein, and at least one guide ribonucleic acid (gRNA) sequence for specifically targeting the PDCD1 gene. Useful methods for identifying gRNA sequences to target PD1 are described below.

Assays to test whether the PDCD1 gene has been inactivated are known and described herein. In one embodiment, the resulting genetic modification of the PDCD1 gene by PCR and the reduction of PD1 expression can be assays by FACS analysis. In another embodiment, PD1 protein expression is detected using a Western blot of cells lysates probed with antibodies to the PD1 protein. In another embodiment, reverse transcriptase polymerase chain reactions (RT-PCR) are used to confirm the presence of the inactivating genetic modification.

Useful genomic, polynucleotide and polypeptide information about human PD1 including the PDCD1 gene are provided in, for example, the GeneCard Identifier GC02M241849, HGNC No. 8760, NCBI Gene ID 5133, Uniprot No. Q15116, and NCBI RefSeq Nos. NM_005018.2 and NP_005009.2.

N. Additional Tolerogenic Factors

In certain embodiments, one or more tolerogenic factors can be inserted or reinserted into genome-edited cells to create immune-privileged universal donor cells, such as universal donor stem cells, universal donor T cells, or universal donor cells. In certain embodiments, the hypoimmunogenic cells disclosed herein have been further modified to express one or more tolerogenic factors.

Exemplary tolerogenic factors include, without limitation, CD47, DUX4, CD24, CD27, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, Serpinb9, CD16 Fc receptor, IL15-RF, CD16, CD52, H2-M3, and CD35. In some embodiments, the tolerogenic factors are selected from the group consisting of CD200, HLA-G, HLA-E, HLA-C, HLA-E heavy chain, PD-L1, IDO1, CTLA4-Ig, IL-10, IL-35, FasL, Serpinb9, CCL21, CCL22, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35.

In some instances, a gene editing system such as the CRISPR/Cas system is used to facilitate the insertion of tolerogenic factors, such as the tolerogenic factors into a safe harbor or a target locus, such as the AAVS1 locus, to actively inhibit immune rejection. In some instances, the tolerogenic factors are inserted into a safe harbor or a target locus using an expression vector. In some embodiments, the safe harbor or target locus is an AAVS1, CCR5, CLYBL, ROSA26, SHS231, F3 (also known as CD142), MICA, MICB, LRP1 (also known as CD91), HMGB1, ABO, RHD, FUT1, PDGFRa, OLIG2, GFAP, or KDM5D gene locus.

In some embodiments, the present disclosure provides a cell (e.g., a primary cell and/or a hypoimmunogenic stem cell and derivative thereof) or population thereof comprising a genome in which the cell genome has been modified to express CD47. In some embodiments, the present disclosure provides a method for altering a cell genome to express CD47. In certain aspects at least one ribonucleic acid or at least one pair of ribonucleic acids may be utilized to facilitate the insertion of CD47 into a cell line. In certain embodiments, the at least one ribonucleic acid or the at least one pair of ribonucleic acids is selected from the group consisting of SEQ ID NOS:200784-231885 of Table 29 of WO2016183041, which is herein incorporated by reference. In some embodiments, the primary cell includes, but are not limited to, a cardiac cell, cardiac progenitor cell, neural cell, glial progenitor cell, endothelial cell, pancreatic islet cell, retinal pigmented epithelium cell, hepatocyte, thyroid cell, skin cell, blood cell, plasma cell, platelet, renal cell, epithelial cell, T cell, B cell, or NK cell. In some embodiments, the stem cell includes, but are not limited to, an embryonic stem cell, induced stem cell, mesenchymal stem cell, and hematopoietic stem cell.

In some embodiments, the present disclosure provides a cell (e.g., a primary cell and/or a hypoimmunogenic stem cell and derivative thereof) or population thereof comprising a genome in which the cell genome has been modified to express HLA-C. In some embodiments, the present disclosure provides a method for altering a cell genome to express HLA-C. In certain aspects at least one ribonucleic acid or at least one pair of ribonucleic acids may be utilized to facilitate the insertion of HLA-C into a cell line. In certain embodiments, the at least one ribonucleic acid or the at least one pair of ribonucleic acids is selected from the group consisting of SEQ ID NOS:3278-5183 of Table 10 of WO2016183041, which is herein incorporated by reference. In some embodiments, the primary cell includes, but are not limited to, a cardiac cell, cardiac progenitor cell, neural cell, glial progenitor cell, endothelial cell, pancreatic islet cell, retinal pigmented epithelium cell, hepatocyte, thyroid cell, skin cell, blood cell, plasma cell, platelet, renal cell, epithelial cell, T cell, B cell, or NK cell. In some embodiments, the stem cell includes, but are not limited to, an embryonic stem cell, induced stem cell, mesenchymal stem cell, and hematopoietic stem cell.

In some embodiments, the present disclosure provides a cell (e.g., a primary cell and/or a hypoimmunogenic stem cell and derivative thereof) or population thereof comprising a genome in which the cell genome has been modified to express HLA-E. In some embodiments, the present disclosure provides a method for altering a cell genome to express HLA-E. In certain aspects at least one ribonucleic acid or at least one pair of ribonucleic acids may be utilized to facilitate the insertion of HLA-E into a cell line. In certain embodiments, the at least one ribonucleic acid or the at least one pair of ribonucleic acids is selected from the group consisting of SEQ ID NOS:189859-193183 of Table 19 of WO2016183041, which is herein incorporated by reference. In some embodiments, the primary cell includes, but are not limited to, a cardiac cell, cardiac progenitor cell, neural cell, glial progenitor cell, endothelial cell, pancreatic islet cell, retinal pigmented epithelium cell, hepatocyte, thyroid cell, skin cell, blood cell, plasma cell, platelet, renal cell, epithelial cell, T cell, B cell, or NK cell. In some embodiments, the stem cell includes, but are not limited to, an embryonic stem cell, induced stem cell, mesenchymal stem cell, and hematopoietic stem cell.

In some embodiments, the present disclosure provides a cell (e.g., a primary cell and/or a hypoimmunogenic stem cell and derivative thereof) or population thereof comprising a genome in which the cell genome has been modified to express HLA-F. In some embodiments, the present disclosure provides a method for altering a cell genome to express HLA-F. In certain aspects at least one ribonucleic acid or at least one pair of ribonucleic acids may be utilized to facilitate the insertion of HLA-F into a cell line. In certain embodiments, the at least one ribonucleic acid or the at least one pair of ribonucleic acids is selected from the group consisting of SEQ ID NOS: 688808-399754 of Table 45 of WO2016183041, which is herein incorporated by reference. In some embodiments, the primary cell includes, but are not limited to, a cardiac cell, cardiac progenitor cell, neural cell, glial progenitor cell, endothelial cell, pancreatic islet cell, retinal pigmented epithelium cell, hepatocyte, thyroid cell, skin cell, blood cell, plasma cell, platelet, renal cell, epithelial cell, T cell, B cell, or NK cell. In some embodiments, the stem cell includes, but are not limited to, an embryonic stem cell, induced stem cell, mesenchymal stem cell, and hematopoietic stem cell.

In some embodiments, the present disclosure provides a cell (e.g., a primary cell and/or a hypoimmunogenic stem cell and derivative thereof) or population thereof comprising a genome in which the cell genome has been modified to express HLA-G. In some embodiments, the present disclosure provides a method for altering a cell genome to express HLA-G. In certain aspects at least one ribonucleic acid or at least one pair of ribonucleic acids may be utilized to facilitate the insertion of HLA-G into a stem cell line. In certain embodiments, the at least one ribonucleic acid or the at least one pair of ribonucleic acids is selected from the group consisting of SEQ ID NOS:188372-189858 of Table 18 of WO2016183041, which is herein incorporated by reference. In some embodiments, the primary cell includes, but are not limited to, a cardiac cell, cardiac progenitor cell, neural cell, glial progenitor cell, endothelial cell, pancreatic islet cell, retinal pigmented epithelium cell, hepatocyte, thyroid cell, skin cell, blood cell, plasma cell, platelet, renal cell, epithelial cell, T cell, B cell, or NK cell. In some embodiments, the stem cell includes, but are not limited to, an embryonic stem cell, induced stem cell, mesenchymal stem cell, and hematopoietic stem cell.

In some embodiments, the present disclosure provides a cell (e.g., a primary cell and/or a hypoimmunogenic stem cell and derivative thereof) or population thereof comprising a genome in which the cell genome has been modified to express PD-L1. In some embodiments, the present disclosure provides a method for altering a cell genome to express PD-L1. In certain aspects at least one ribonucleic acid or at least one pair of ribonucleic acids may be utilized to facilitate the insertion of PD-L1 into a stem cell line. In certain embodiments, the at least one ribonucleic acid or the at least one pair of ribonucleic acids is selected from the group consisting of SEQ ID NOS:193184-200783 of Table 21 of WO2016183041, which is herein incorporated by reference. In some embodiments, the primary cell includes, but are not limited to, a cardiac cell, cardiac progenitor cell, neural cell, glial progenitor cell, endothelial cell, pancreatic islet cell, retinal pigmented epithelium cell, hepatocyte, thyroid cell, skin cell, blood cell, plasma cell, platelet, renal cell, epithelial cell, T cell, B cell, or NK cell. In some embodiments, the stem cell includes, but are not limited to, an embryonic stem cell, induced stem cell, mesenchymal stem cell, and hematopoietic stem cell.

In some embodiments, the present disclosure provides a cell (e.g., a primary cell and/or a hypoimmunogenic stem cell and derivative thereof) or population thereof comprising a genome in which the cell genome has been modified to express CTLA4-Ig. In some embodiments, the present disclosure provides a method for altering a cell genome to express CTLA4-Ig. In certain aspects at least one ribonucleic acid or at least one pair of ribonucleic acids may be utilized to facilitate the insertion of CTLA4-Ig into a stem cell line. In certain embodiments, the at least one ribonucleic acid or the at least one pair of ribonucleic acids is selected from any one disclosed in WO2016183041, including the sequence listing.

In some embodiments, the present disclosure provides a cell (e.g., a primary cell and/or a hypoimmunogenic stem cell and derivative thereof) or population thereof comprising a genome in which the cell genome has been modified to express CI-inhibitor. In some embodiments, the present disclosure provides a method for altering a cell genome to express CI-inhibitor. In certain aspects at least one ribonucleic acid or at least one pair of ribonucleic acids may be utilized to facilitate the insertion of CI-inhibitor into a stem cell line. In certain embodiments, the at least one ribonucleic acid or the at least one pair of ribonucleic acids is selected from any one disclosed in WO2016183041, including the sequence listing. In some embodiments, the primary cell includes, but are not limited to, a cardiac cell, cardiac progenitor cell, neural cell, glial progenitor cell, endothelial cell, pancreatic islet cell, retinal pigmented epithelium cell, hepatocyte, thyroid cell, skin cell, blood cell, plasma cell, platelet, renal cell, epithelial cell, T cell, B cell, or NK cell. In some embodiments, the stem cell includes, but are not limited to, an embryonic stem cell, induced stem cell, mesenchymal stem cell, and hematopoietic stem cell.

In some embodiments, the present disclosure provides a cell (e.g., a primary cell and/or a hypoimmunogenic stem cell and derivative thereof) or population thereof comprising a genome in which the cell genome has been modified to express IL-35. In some embodiments, the present disclosure provides a method for altering a cell genome to express IL-35. In certain aspects at least one ribonucleic acid or at least one pair of ribonucleic acids may be utilized to facilitate the insertion of IL-35 into a stem cell line. In certain embodiments, the at least one ribonucleic acid or the at least one pair of ribonucleic acids is selected from any one disclosed in WO2016183041, including the sequence listing. In some embodiments, the primary cell includes, but are not limited to, a cardiac cell, cardiac progenitor cell, neural cell, glial progenitor cell, endothelial cell, pancreatic islet cell, retinal pigmented epithelium cell, hepatocyte, thyroid cell, skin cell, blood cell, plasma cell, platelet, renal cell, epithelial cell, T cell, B cell, or NK cell. In some embodiments, the stem cell includes, but are not limited to, an embryonic stem cell, induced stem cell, mesenchymal stem cell, and hematopoietic stem cell.

In some embodiments, the tolerogenic factors are expressed in a cell using an expression vector. For example, the expression vector for expressing CD47 in a cell comprises a polynucleotide sequence encoding CD47. The expression vector can be an inducible expression vector. The expression vector can be a viral vector, such as but not limited to, a lentiviral vector.

In some embodiments, the present disclosure provides a cell (e.g., a primary cell and/or a hypoimmunogenic stem cell and derivative thereof) or population thereof comprising a genome in which the cell genome has been modified to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In some embodiments, the present disclosure provides a method for altering a cell genome to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In certain aspects at least one ribonucleic acid or at least one pair of ribonucleic acids may be utilized to facilitate the insertion of the selected polypeptide into a stem cell line. In certain embodiments, the at least one ribonucleic acid or the at least one pair of ribonucleic acids is selected from any one disclosed in Appendices 1-47 and the sequence listing of WO2016183041, the disclosures of which are incorporated herein by reference.

In some embodiments, a suitable gene editing system (e.g., CRISPR/Cas system or any of the gene editing systems described herein) is used to facilitate the insertion of a polynucleotide encoding a tolerogenic factor, into a genomic locus of the hypoimmunogenic cell. In some cases, the polynucleotide encoding the tolerogenic factor is inserted into a safe harbor or a target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, PDGFRa, OLIG2, GFAP, or KDM5D gene locus. In some embodiments, the polynucleotide encoding the tolerogenic factor is inserted into a B2M gene locus, a CIITA gene locus, a TRAC gene locus, or a TRB gene locus. In some embodiments, the polynucleotide encoding the tolerogenic factor is inserted into any one of the gene loci depicted in Table 4 provided herein. In certain embodiments, the polynucleotide encoding the tolerogenic factor is operably linked to a promoter.

O. Methods of Genetic Modifications

In some embodiments, the rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

Target polynucleotide sequences described herein may be altered in any manner which is available to the skilled artisan utilizing a gene editing system (e.g., CRISPR/Cas) of the present disclosure. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The gene editing (e.g., CRISPR/Cas) systems disclosed herein can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems disclosed herein can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system provided herein includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein, isoform thereof, or any Cas-like protein with similar function or activity of any Cas protein or isoform thereof. Exemplary Cas core proteins include, but are not limited to, Cas1, Cas2, Cas3, Cas4, Cas5, Cas6, Cas7, Cas8 and Cas9. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to, Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019). In some embodiments, a Cas protein comprises a Cas protein of the Type I subtype. Type I CRISPR/Cas effector proteins are a subtype of Class 1 CRISPR/Cas effector proteins. Examples include, but are not limited to: Cas3, Cas8a, Cas5, Cas8b, Cas8c, Cas10d, Cse1, Cse2, Csy1, Csy2, Csy3, and/or GSU0054. In some embodiments, a Cas protein comprises Cas3, Cas8a, Cas5, Cas8b, Cas8c, Cas10d, Cse1, Cse2, Csy1, Csy2, Csy3, and/or GSU0054. In some embodiments, a Cas protein comprises a Cas protein of the Type II subtype. Type II CRISPR/Cas effector proteins are a subtype of Class 2 CRISPR/Cas effector proteins. Examples include, but are not limited to: Cas9, Csn2, and/or Cas4. In some embodiments, a Cas protein comprises Cas9, Csn2, and/or Cas4. In some embodiments, a Cas protein comprises a Cas protein of the Type III subtype. Type III CRISPR/Cas effector proteins are a subtype of Class 1 CRISPR/Cas effector proteins. Examples include, but are not limited to: Cas10, Csm2, Cmr5, Cas10, Csx11, and/or Csx10. In some embodiments, a Cas protein comprises a Cas10, Csm2, Cmr5, Cas10, Csx11, and/or Csx10. In some embodiments, a Cas protein comprises a Cas protein of the Type IV subtype. Type IV CRISPR/Cas effector proteins are a subtype of Class 1 CRISPR/Cas effector proteins. Examples include, but are not limited to: Csf1. In some embodiments, a Cas protein comprises Csf1. In some embodiments, a Cas protein comprises a Cas protein of the Type V subtype. Type V CRISPR/Cas effector proteins are a subtype of Class 2 CRISPR/Cas effector proteins. For examples of type V CRISPR/Cas systems and their effector proteins (e.g., Cas12 family proteins such as Cas12a), see, e.g., Shmakov et al., Nat Rev Microbiol. 2017; 15(3):169-182: “Diversity and evolution of class 2 CRISPR-Cas systems.” Examples include, but are not limited to: Cas12 family (Cas12a, Cas12b, Cas12c), C2c4, C2c8, C2c5, C2c10, and C2c9; as well as CasX (Cas12e) and CasY (Cas12d). Also see, e.g., Koonin et al., Curr Opin Microbiol. 2017; 37:67-78: “Diversity, classification and evolution of CRISPR-Cas systems.” In some embodiments, a Cas protein comprises a Cas12 protein such as Cas12a, Cas12b, Cas12c, Cas12d, and/or Cas12e.

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods disclosed herein contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids provided herein can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA sequences useful for CRISPR/Cas-based targeting of genes described herein are provided in Table 2. The sequences can be found in WO2016183041 filed May 9, 2016, the disclosure including the Tables, Appendices, and Sequence Listing is incorporated herein by reference in its entirety.

TABLE 2
Exemplary gRNA sequences useful for targeting genes

| Gene Name | SEQ ID NO: | WO2016183041 |
|---|---|---|
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |
| CD47 | SEQ ID NOS: 200784-231885 | Table 29, Appendix 22 |
| HLA-E | SEQ ID NOS: 189859-193183 | Table 19, Appendix 12 |
| HLA-F | SEQ ID NOS: 688808-699754 | Table 45, Appendix 38 |
| HLA-G | SEQ ID NOS: 188372-189858 | Table 18, Appendix 11 |
| PD-Ll | SEQ ID NOS: 193184-200783 | Table 21, Appendix 14 |
| Gene Name | SEQ ID NO: | US20160348073 |
| TRAC | SEQ ID NOS: 532-609 | |
| | and 9102-9797 | |
| TRB (also | SEQ ID NOS: 610-765 | |
| TCRB, and | and 9798-10532 | |
| TRBC) | | |

Other exemplary gRNA sequences useful for CRISPR/Cas-based targeting of genes described herein are provided in U.S. Provisional Patent Application No. 63/190,685, filed May 19, 2021, and in U.S. Provisional Patent Application No. 63/221,887, filed Jul. 14, 2021, the disclosures of which, including the Tables, Appendices, and Sequence Listings, are incorporated herein by reference in their entireties.

In some embodiments, the cells described herein are made using Transcription Activator-Like Effector Nucleases (TALEN) methodologies. By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

In some embodiments, the cells described herein are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease may for example correspond to a LAGLIDADG endonuclease, to an HNH endonuclease, or to a GIY-YIG endonuclease. In some embodiments, the homing endonuclease can be an I-CreI variant.

In some embodiments, the cells described herein are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol., 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells provided herein are made using RNA silencing or RNA interference (RNAi, also referred to as siRNA) to knockdown (e.g., decrease, eliminate, or inhibit) the expression of a polypeptide such as a tolerogenic factor. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, CIITA can be knocked down in a pluripotent stem cell by introducing a CIITA siRNA or transducing a CIITA shRNA-expressing virus into the cell. In some embodiments, RNA interference is employed to reduce or inhibit the expression of at least one selected from the group consisting of CIITA, B2M, and NLRC5.

1. Gene Editing Systems

In some embodiments, the methods for genetically modifying cells to knock out, knock down, or otherwise modify one or more genes comprise using a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems, as well as nickase systems, base editing systems, prime editing systems, and gene writing systems known in the art.

a) ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

b) TALENs

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

c) Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

d) Transposases

Transposases are enzymes that bind to the end of a transposon and catalyze its movement to another part of the genome by a cut and paste mechanism or a replicative transposition mechanism. By linking transposases to other systems such as the CRISPER/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

e) CRISPR/Cas Systems

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complex. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 3 below.

TABLE 3
Exemplary Cas nuclease variants and their PAM sequences

| CRISPR Nuclease | Source Organism | PAM Sequence (5′→3′) |
|---|---|---|
| SpCas9 | Streptococcus pyogenes | NGG or NAG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| TdCas9 | Treponema denticola | NAAAAC |
| LbCas12a (Cpf1) | Lachnospiraceae bacterium | TTTV |
| AsCas12a (Cpf1) | Acidaminococcus sp. | TTTV |
| AacCas12b | Alicyclobacillus acidiphilus | TTN |
| BhCas12b v4 | Bacillus hisashii | ATTN, TTTN, or GTTN |

R = A or G; Y = C or T; W = A or T; V = A or C or G; N = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example the Cas nuclease may have one or more mutations that alter its PAM specificity.

In some embodiments, the cells provided herein are genetically modified to reduce expression of one or more immune factors (including target polypeptides) to create immune-privileged or hypoimmunogenic cells. In certain embodiments, the cells (e.g., stem cells, induced pluripotent stem cells, differentiated cells, hematopoietic stem cells, primary T cells and CAR-T cells) disclosed herein comprise one or more genetic modifications to reduce expression of one or more target polynucleotides. Non-limiting examples of such target polynucleotides and polypeptides include CIITA, B2M, NLRC5, CTLA4, PD1, HLA-A, HLA-BM, HLA-C, RFX-ANK, NFY-A, RFX5, RFX-AP, NFY-B, NFY-C, IRF1, and/or TAP1.

In some embodiments, the genetic modification occurs using a CRISPR/Cas system. By modulating (e.g., reducing or deleting) expression of one or a plurality of the target polynucleotides, such cells exhibit decreased immune activation when engrafted into a recipient subject. In some embodiments, the cell is considered hypoimmunogenic, e.g., in a recipient subject or patient upon administration.

f) Nickases

Nuclease domains of the Cas, in particular the Cas9, nuclease can be mutated independently to generate enzymes referred to as DNA “nickases”. Nickases are capable of introducing a single-strand cut with the same specificity as a regular CRISPR/Cas nuclease system, including for example CRISPR/Cas9. Nickases can be employed to generate double-strand breaks which can find use in gene editing systems (Mali et al., Nat Biotech, 31(9):833-838 (2013); Mali et al. Nature Methods, 10:957-963 (2013); Mali et al., Science, 339(6121):823-826 (2013)). In some instances, when two Cas nickases are used, long overhangs are produced on each of the cleaved ends instead of blunt ends which allows for additional control over precise gene integration and insertion (Mali et al., Nat Biotech, 31(9):833-838 (2013); Mali et al. Nature Methods, 10:957-963 (2013); Mali et al., Science, 339(6121):823-826 (2013)). As both nicking Cas enzymes must effectively nick their target DNA, paired nickases can have lower off-target effects compared to the double-strand-cleaving Cas-based systems (Ran et al., Cell, 155(2):479-480 (2013); Mali et al., Nat Biotech, 31(9):833-838 (2013); Mali et al. Nature Methods, 10:957-963 (2013); Mali et al., Science, 339(6121):823-826 (2013)).

P. Methods of Recombinant Expression of Tolerogenic Factors and/or Chimeric Antigen Receptors

For all of these technologies, well-known recombinant techniques are used, to generate recombinant nucleic acids as outlined herein. In certain embodiments, the recombinant nucleic acids encoding a tolerogenic factor or a chimeric antigen receptor may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

Examples of suitable mammalian promoters include, for example, promoters from the following genes: elongation factor 1 alpha (EF1α) promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al., Nature 273: 113-120 (1978)). The immediate early promoter of the human cytomegalovirus is conveniently obtained as a HindIII restriction enzyme fragment (Greenaway et al., Gene 18: 355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

The process of introducing the polynucleotides described herein into cells can be achieved by any suitable technique. Suitable techniques include calcium phosphate or lipid-mediated transfection, electroporation, fusogens, and transduction or infection using a viral vector. In some embodiments, the polynucleotides are introduced into a cell via viral transduction (e.g., lentiviral transduction) or otherwise delivered on a viral vector (e.g., fusogen-mediated delivery).

Provided herein are cells that do not trigger or activate an immune response upon administration to a recipient subject. As described above, in some embodiments, the cells are modified to increase expression of genes and tolerogenic (e.g., immune) factors that affect immune recognition and tolerance in a recipient.

In certain embodiments, any of the cells (e.g., stem cells, induced pluripotent stem cells, differentiated cells, hematopoietic stem cells, primary T cells CAR-T cells, and CAR-NK cells) disclosed herein that harbor a genomic modification that modulates expression of one or more target proteins listed herein are also modified to express one or more tolerogenic factors. Exemplary tolerogenic factors include, without limitation, one or more of CD47, DUX4, CD24, CD27, CD35, CD46, CD55, CD59, CD200, HLA-C, HLA-E, HLA-E heavy chain, HLA-G, PD-L1, IDOL CTLA4-Ig, C1-Inhibitor, IL-10, IL-35, FasL, CCL21, CCL22, Mfge8, and Serpinb9. In some embodiments, the tolerogenic factors are selected from a group including DUX4, CD47, CD24, CD27, CD35, CD46, CD55, CD59, CD200, HLA-C, HLA-E, HLA-E heavy chain, HLA-G, PD-L1, IDOL CTLA4-Ig, C1-Inhibitor, IL-10, IL-35, FasL, CCL21, CCL22, Mfge8, and Serpinb9.

Useful genomic, polynucleotide and polypeptide information about human CD27 (which is also known as CD27L receptor, Tumor Necrosis Factor Receptor Superfamily Member 7 (TNFSF7), T Cell Activation Antigen S152, Tp55, and T14) are provided in, for example, the GeneCard Identifier GC12P008144, HGNC No. 11922, NCBI Gene ID 939, Uniprot No. P26842, and NCBI RefSeq Nos. NM_001242.4 and NP_001233.1.

Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI RefSeq Nos. NM_002389.4, NM_153826.3, NM_172350.2, NM_172351.2, NM_172352.2 NP_758860.1, NM_172353.2, NM_172359.2, NM_172361.2, NP_002380.3, NP_722548.1, NP_758860.1, NP_758861.1, NP_758862.1, NP_758863.1, NP_758869.1, and NP_758871.1.

Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM_001300904.1, NP_000565.1, NP_001108224.1, NP_001287832.1, and NP_001287833.1.

Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP_001120697.1, NM_001127225.1, NP_001120698.1, NM_001127226.1, NP_001120699.1, NM_001127227.1, NP_976074.1, NM_203329.2, NP_976075.1, NM_203330.2, NP_976076.1, and NM_203331.2.

Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP 005247539.1, and XM_005247482.2.

Useful genomic, polynucleotide and polypeptide information about human HLA-C are provided in, for example, the GeneCard Identifier GC06M031272, HGNC No. 4933, NCBI Gene ID 3107, Uniprot No. P10321, and NCBI RefSeq Nos. NP_002108.4 and NM_002117.5.

Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5.

Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5.

Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3.

Useful genomic, polynucleotide and polypeptide information about human IDO1 are provided in, for example, the GeneCard Identifier GC08P039891, HGNC No. 6059, NCBI Gene ID 3620, Uniprot No. P14902, and NCBI RefSeq Nos. NP_002155.1 and NM_002164.5.

Useful genomic, polynucleotide and polypeptide information about human IL-10 are provided in, for example, the GeneCard Identifier GC01M206767, HGNC No. 5962, NCBI Gene ID 3586, Uniprot No. P22301, and NCBI RefSeq Nos. NP_000563.1 and NM_000572.2.

Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1.

Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3.

Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP 016879020.1, and XM_017023531.1.

Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3.

Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP 005249241.1, and XM_005249184.4.

Methods for modulating expression of genes and factors (proteins) include genome editing technologies, and, RNA or protein expression technologies and the like. For all of these technologies, well known recombinant techniques are used, to generate recombinant nucleic acids as outlined herein.

In some embodiments, expression of a target gene (e.g., DUX4, CD47, or another tolerogenic factor) is increased by expression of fusion protein or a protein complex containing (1) a site-specific binding domain specific for the endogenous DUX4, CD47, or other gene and (2) a transcriptional activator.

In some embodiments, the method is achieved by genetic modification methods that comprise homology-directed repair/recombination.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perler et al., (1994) Nucleic Acids Res. 22, 1125-1127; Jasin (1996) Trends Genet. 12:224-228; Gimble et al., (1996) J. Mol. Biol. 263:163-180; Argast et al., (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al., (2002) Molec. Cell 10:895-905; Epinat et al., (2003) Nucleic Acids Res. 31:2952-2962; Ashworth et al., (2006) Nature 441:656-659; Paques et al., (2007) Current Gene Therapy 7:49-66; U.S. Patent Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, see, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see, e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a sequence with minimal off-target binding to a non-target gene.

In some embodiments, the regulatory factor further comprises a functional domain, e.g., a transcriptional activator.

A In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of a heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members, etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al., J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Sp1, AP-2, and CTF1 (Seipel et al, EMBO J. 11, 4961-4968 (1992) as well as p300, CBP, PCAF, SRC1 PvALF, AtHD2A and ERF-2. See, for example, Robyr et al., (2000) Mol. Endocrinol. 14:329-347; Collingwood et al., (1999) J. Mol. Endocrinol 23:255-275; Leo et al., (2000) Gene 245:1-11; Manteuffel-Cymborowska (1999) Acta Biochim. Pol. 46:77-89; McKenna et al., (1999) J. Steroid Biochem. Mol. Biol. 69:3-12; Malik et al., (2000) Trends Biochem. Sci. 25:277-283; and Lemon et al., (1999) Curr. Opin. Genet. Dev. 9:499-504. Additional exemplary activation domains include, but are not limited to, OsGAI, HALF-1, C1, API, ARF-5, -6, -1, and -8, CPRF1, CPRF4, MYC-RP/GP, and TRAB1, see, for example, Ogawa et al., (2000) Gene 245:21-29; Okanami et al., (1996) Genes Cells 1:87-99; Goff et al., (1991) Genes Dev. 5:298-309; Cho et al., (1999) Plant Mol Biol 40:419-429; Ulmason et al., (1999) Proc. Natl. Acad. Sci. USA 96:5844-5849; Sprenger-Haussels et al., (2000) Plant J. 22:1-8; Gong et al., (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al., (1999) Cell 99:451-454; Tyler et al., (1999) Cell 99:443-446; Knoepfler et al., (1999) Cell 99:447-450; and Robertson et al., (2000) Nature Genet. 25:338-342. Additional exemplary repression domains include, but are not limited to, ROM2 and AtHD2A. See, for example, Chem et al., (1996) Plant Cell 8:305-321; and Wu et al., (2000) Plant J. 22:19-27.

In some instances, the domain is involved in epigenetic regulation of a chromosome. In some embodiments, the domain is a histone acetyltransferase (HAT), e.g., type-A, nuclear localized such as MYST family members MOZ, Ybf2/Sas3, MOF, and Tip60, GNAT family members Gcn5 or pCAF, the p300 family members CBP, p300 or RttI09 (Bemdsen and Denu (2008) Curr Opin Struct Biol 18(6):682-689). In other instances the domain is a histone deacetylase (HD AC) such as the class I (HDAC-1, 2, 3, and 8), class II (HDAC IIA (HDAC-4, 5, 7 and 9), HD AC IIB (HDAC 6 and 10)), class IV (HDAC-1 1), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dot1, PRMT 1/6, PRMT 5/7, PR-Set7 and Suv4-20h, Domains involved in sumoylation and biotinylation (Lys9, 13, 4, 18 and 12) may also be used in some embodiments (for a review, see, Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, Ill.) Catalogue. Methods and compositions for making fusions between a minor groove binder and a polypeptide have been described. Mapp et al., (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

Provided herein are non-activated T cells comprising reduced expression of HLA-A, HLA-B, HLA-C, CIITA, TCR-alpha, and/or TCR-beta relative to a wild-type T cell, wherein the activated T cell further comprises a first gene encoding a chimeric antigen receptor (CAR).

In some embodiments, the non-activated T cell has not been treated with an anti-CD3 antibody, an anti-CD28 antibody, a T cell activating cytokine, or a soluble T cell costimulatory molecule. In some embodiments, the non-activated T cell does not express activation markers. In some embodiments, the non-activated T cell expresses CD3 and CD28, and wherein the CD3 and/or CD28 are inactive.

In some embodiments, the anti-CD3 antibody is OKT3. In some embodiments, the anti-CD28 antibody is CD28.2. In some embodiments, the T cell activating cytokine is selected from the group of T cell activating cytokines consisting of IL-2, IL-7, IL-15, and IL-21. In some embodiments, the soluble T cell costimulatory molecule is selected from the group of soluble T cell costimulatory molecules consisting of an anti-CD28 antibody, an anti-CD80 antibody, an anti-CD86 antibody, an anti-CD137L antibody, and an anti-ICOS-L antibody.

In some embodiments, the non-activated T cell is a primary T cell. In other embodiments, the non-activated T cell is differentiated from the hypoimmunogenic cells of the present technology. In some embodiments, the T cell is a CD8⁺ T cell.

In some embodiments, the first gene is carried by a lentiviral vector that comprises a CD8 binding agent. In some embodiments, the first gene is a CAR is selected from the group consisting of a CD19-specific CAR and a CD22-specific CAR. In some embodiments, the CAR is a bispecific CAR. In some embodiments, the bispecific CAR is a CD19/CD22 bispecific CAR.

In some embodiments, the first and/or second gene is carried by a lentiviral vector that comprises a CD8 binding agent. In some embodiments, the first and/or second gene is introduced into the cells using fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the non-activated T cell further comprises a second gene CD47. In some embodiments, the first and/or second genes are inserted into a specific locus of at least one allele of the T cell. In some embodiments, the specific locus is selected from the group consisting of a safe harbor locus, a target locus, a B2M locus, a CIITA locus, a TRAC locus, and a TRB locus. In some embodiments, the second gene encoding CD47 is inserted into the specific locus selected from the group consisting of a safe harbor locus, a target locus, a B2M locus, a CIITA locus, a TRAC locus and a TRB locus. In some embodiments, the first gene encoding the CAR is inserted into the specific locus selected from the group consisting of a safe harbor locus, a target locus, a B2M locus, a CIITA locus, a TRAC locus and a TRB locus. In some embodiments, the second gene encoding CD47 and the first gene encoding the CAR are inserted into different loci. In some embodiments, the second gene encoding CD47 and the first gene encoding the CAR are inserted into the same locus. In some embodiments the second gene encoding CD47 and the first gene encoding the CAR are inserted into the B2M locus. In some embodiments, the second gene encoding CD47 and the first gene encoding the CAR are inserted into the CIITA locus. In some embodiments, the second gene encoding CD47 and the first gene encoding the CAR are inserted into the TRAC locus. In some embodiments, the second gene encoding CD47 and the first gene encoding the CAR are inserted into the TRB locus. In some embodiments, the second gene encoding CD47 and the first gene encoding the CAR are inserted into the safe harbor or target locus. In some embodiments, the safe harbor or target locus is selected from the group consisting of a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene, locus a MICB gene, locus a LRP1 (CD91) gene locus, a HMGB1 gene locus, an ABO gene locus, ad RHD gene locus, a FUT1 locus, a PDGFRa gene locus, an OLIG2 gene locus, a GFAP gene locus, and a KDM5D gene locus).

In some embodiments, the non-activated T cell does not express HLA-A, HLA-B, and/or HLA-C antigens. In some embodiments, the non-activated T cell does not express B2M. In some embodiments the non-activated T cell does not express HLA-DP, HLA-DQ, and/or HLA-DR antigens. In some embodiments, the non-activated T cell does not express CIITA. In some embodiments, the non-activated T cell does not express TCR-alpha and TCR-beta.

In some embodiments, the non-activated T cell is an B2M^{indel/indel}, CIITA^{indel/indel}, TRAC^{indel/indel} cell comprising second gene encoding CD47 and/or the first gene encoding CAR inserted into the TRAC locus. In some embodiments, the non-activated T cell is an B2M^{indel/indel}, CIITA^{indel/indel}, TRAC^{indel/indel} cell comprising the second gene encoding CD47 and the first gene encoding CAR inserted into the TRAC locus. In some embodiments, the non-activated T cell is an B2M^{indel/indel}, CIITA^{indel/indel}, TRAC^{indel/indel} cell comprising second gene encoding CD47 and/or the first gene encoding CAR inserted into the TRB locus. In some embodiments, the non-activated T cell is an B2M^{indel/indel}, CIITA^{indel/indel}, TRAC^{indel/indel} cell comprising the second gene encoding CD47 and the first gene encoding CAR inserted into the TRB locus. In some embodiments, the non-activated T cell is an B2M^{indel/indel}, CIITA^{indel/indel}, TRAC^{indel/indel} cell comprising second gene encoding CD47 and/or the first gene encoding CAR inserted into the B2M locus. In some embodiments, the non-activated T cell is an B2M^{indel/indel}, CIITA^{indel/indel}, TRAC^{indel/indel} cell comprising the second gene encoding CD47 and the first gene encoding CAR inserted into a B2M locus. In some embodiments, the non-activated T cell is an B2M^{indel/indel}, CIITA^{indel/indel}, TRAC^{indel/indel} cell comprising second gene encoding CD47 and/or the first gene encoding CAR inserted into the CIITA locus. In some embodiments, the non-activated T cell is an B2M^{indel/indel}, CIITA^{indel/indel}, TRAC^{indel/indel} cell comprising the second gene encoding CD47 and the first gene encoding CAR inserted into a CIITA locus.

Provided herein are engineered T cells comprising reduced expression of HLA-A, HLA-B, HLA-C, CIITA, TCR-alpha, and/or TCR-beta relative to a wild-type T cell, wherein the engineered T cell further comprises a first gene encoding a chimeric antigen receptor (CAR) carried by a lentiviral vector that comprises a CD8 binding agent.

In some embodiments, the engineered T cell is a primary T cell. In other embodiments, the engineered T cell is differentiated from the hypoimmunogenic cell of the present technology. In some embodiments, the T cell is a CD8⁺ T cell. In some embodiments, the T cell is a CD4⁺ T cell.

In some embodiments, the engineered T cell does not express activation markers. In some embodiments, the engineered T cell expresses CD3 and CD28, and wherein the CD3 and/or CD28 are inactive.

In some embodiments, the engineered T cell has not been treated with an anti-CD3 antibody, an anti-CD28 antibody, a T cell activating cytokine, or a soluble T cell costimulatory molecule. In some embodiments, the anti-CD3 antibody is OKT3, wherein the anti-CD28 antibody is CD28.2, wherein the T cell activating cytokine is selected from the group of T cell activating cytokines consisting of IL-2, IL-7, IL-15, and IL-21, and wherein soluble T cell costimulatory molecule is selected from the group of soluble T cell costimulatory molecules consisting of an anti-CD28 antibody, an anti-CD80 antibody, an anti-CD86 antibody, an anti-CD137L antibody, and an anti-ICOS-L antibody. In some embodiments, the engineered T cell has not been treated with one or more T cell activating cytokines selected from the group consisting of IL-2, IL-7, IL-15, and IL-21. In some instances, the cytokine is IL-2. In some embodiments, the one or more cytokines is IL-2 and another selected from the group consisting of IL-7, IL-15, and IL-21.

In some embodiments, the engineered T cell further comprises a second gene CD47. In some embodiments, the first and/or second genes are inserted into a specific locus of at least one allele of the T cell. In some embodiments, the specific locus is selected from the group consisting of a safe harbor locus, a target locus, a B2M locus, a CIITA locus, a TRAC locus, and a TRB locus. In some embodiments, the second gene encoding CD47 is inserted into the specific locus selected from the group consisting of a safe harbor locus, a target locus, a B2M locus, a CIITA locus, a TRAC locus and a TRB locus. In some embodiments, the first gene encoding the CAR is inserted into the specific locus selected from the group consisting of a safe harbor locus, a target locus, a B2M locus, a CIITA locus, a TRAC locus and a TRB locus. In some embodiments, the second gene encoding CD47 and the first gene encoding the CAR are inserted into different loci. In some embodiments, the second gene encoding CD47 and the first gene encoding the CAR are inserted into the same locus. In some embodiments, the second gene encoding CD47 and the first gene encoding the CAR are inserted into the B2M locus, the CIITA locus, the TRAC locus, the TRB locus, or the safe harbor or target locus. In some embodiments, the safe harbor or target locus is selected from the group consisting of a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene, locus a MICB gene, locus a LRP1 (CD91) gene locus, a HMGB1 gene locus, an ABO gene locus, ad RHD gene locus, a FUT1 locus, a PDGFRa gene locus, an OLIG2 gene locus, a GFAP gene locus, and a KDM5D gene locus).

In some embodiments, the CAR is selected from the group consisting of a CD19-specific CAR and a CD22-specific CAR.

In some embodiments, the engineered T cell does not express HLA-A, HLA-B, and/or HLA-C antigens, wherein the engineered T cell does not express B2M, wherein the engineered T cell does not express HLA-DP, HLA-DQ, and/or HLA-DR antigens, wherein the engineered T cell does not express CIITA, and/or wherein the engineered T cell does not express TCR-alpha and TCR-beta.

In some embodiments, the engineered T cell is an B2M^{indel/indel}, CIITA^{indel/indel}, TRAC^{indel/indel} cell comprising the second gene encoding CD47 and/or the first gene encoding CAR inserted into the TRAC locus, into the TRB locus, into the B2M locus, or into the CIITA locus.

In some embodiments, the non-activated T cell and/or the engineered T cell of the present technology are in a subject. In other embodiments, the non-activated T cell and/or the engineered T cell of the present technology are in vitro.

In some embodiments, the non-activated T cell and/or the engineered T cell of the present technology express a CD8 binding agent. In some embodiments, the CD8 binding agent is an anti-CD8 antibody. In some embodiments, the anti-CD8 antibody is selected from the group consisting of a mouse anti-CD8 antibody, a rabbit anti-CD8 antibody, a human anti-CD8 antibody, a humanized anti-CD8 antibody, a camelid (e.g., llama, alpaca, camel) anti-CD8 antibody, and a fragment thereof. In some embodiments, the fragment thereof is an scFV or a VHH. In some embodiments, the CD8 binding agent binds to a CD8 alpha chain and/or a CD8 beta chain.

In some embodiments, the CD8 binding agent is fused to a transmembrane domain incorporated in the viral envelope. In some embodiments, the lentivirus vector is pseudotyped with a viral fusion protein. In some embodiments, the viral fusion protein comprises one or more modifications to reduce binding to its native receptor.

In some embodiments, the viral fusion protein is fused to the CD8 binding agent. In some embodiments, the viral fusion protein comprises Nipah virus F glycoprotein and Nipah virus G glycoprotein fused to the CD8 binding agent. In some embodiments, the lentivirus vector does not comprise a T cell activating molecule or a T cell costimulatory molecule. In some embodiments, the lentivirus vector encodes the first gene and/or the second gene.

In some embodiments, following transfer into a first subject, the non-activated T cell or the engineered T cell exhibits one or more responses selected from the group consisting of (a) a T cell response, (b) an NK cell response, and (c) a macrophage response, that are reduced as compared to a wild-type cell following transfer into a second subject. In some embodiments, the first subject and the second subject are different subjects. In some embodiments, the macrophage response is engulfment.

In some embodiments, following transfer into a subject, the non-activated T cell or the engineered T cell exhibits one or more selected from the group consisting of (a) reduced TH1 activation in the subject, (b) reduced NK cell killing in the subject, and (c) reduced killing by whole PBMCs in the subject, as compared to a wild-type cell following transfer into the subject.

In some embodiments, following transfer into a subject, the non-activated T cell or the engineered T cell elicits one or more selected from the group consisting of (a) reduced donor specific antibodies in the subject, (b) reduced IgM or IgG antibodies in the subject, and (c) reduced complement-dependent cytotoxicity (CDC) in a subject, as compared to a wild-type cell following transfer into the subject.

In some embodiments, the non-activated T cell or the engineered T cell is transduced with a lentivirus vector comprising a CD8 binding agent within the subject. In some embodiments, the lentivirus vector carries a gene encoding the CAR and/or CD47.

Provided herein are pharmaceutical compositions comprising a population of the non-activated T cells and/or the engineered T cells of the present technology and a pharmaceutically acceptable additive, carrier, diluent or excipient.

Provided herein are methods comprising administering to a subject a composition comprising a population of the non-activated T cells and/or the engineered T cells of the present technology, or one or more the pharmaceutical compositions of the present technology.

In some embodiments, the subject is not administered a T cell activating treatment before, after, and/or concurrently with administration of the composition. In some embodiments, the T cell activating treatment comprises lymphodepletion.

Provided herein are methods of treating a subject suffering from cancer, comprising administering to a subject a composition comprising a population of the non-activated T cells and/or the engineered T cells of the present technology, or one or more the pharmaceutical compositions of the present technology, wherein the subject is not administered a T cell activating treatment before, after, and/or concurrently with administration of the composition. In some embodiments, the T cell activating treatment comprises lymphodepletion.

Provided herein are methods for expanding T cells capable of recognizing and killing tumor cells in a subject in need thereof within the subject, comprising administering to a subject a composition comprising a population of the non-activated T cells and/or the engineered T cells of the present technology, or one or more the pharmaceutical compositions of the present technology, wherein the subject is not administered a T cell activating treatment before, after, and/or concurrently with administration of the composition. In some embodiments, the T cell activating treatment comprises lymphodepletion.

Provided herein are dosage regimens for treating a disease or disorder in a subject comprising administration of a pharmaceutical composition comprising a population of the non-activated T cells and/or the engineered T cells of the present technology, or one or more the pharmaceutical compositions of the present technology, and a pharmaceutically acceptable additive, carrier, diluent or excipient, wherein the pharmaceutical composition is administered in about 1-3 doses.

Once altered, the presence of expression of any of the molecule described herein can be assayed using known techniques, such as Western blots, ELISA assays, FACS assays, and the like.

Q. Generation of Induced Pluripotent Stem Cells

In one aspect, provided herein are methods of producing hypoimmunogenic pluripotent cells. In some embodiments, the method comprises generating pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see, Takahashi and Yamanaka Cell 126:663-676 (2006), hereby incorporated by reference in its entirety and specifically for the techniques outlined therein. Since then, a number of methods have been developed; see, Seki et al., World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In general, as is known in the art, iPSCs are made from non-pluripotent cells such as, but not limited to, blood cells, fibroblasts, etc., by transiently expressing the reprogramming factors as described herein.

R. Assays for Hypoimmunogenicity Phenotypes and Retention of Pluripotency

Once the hypoimmunogenic cells have been generated, they may be assayed for their hypoimmunogenicity and/or retention of pluripotency as is described in WO2016183041 and WO2018132783.

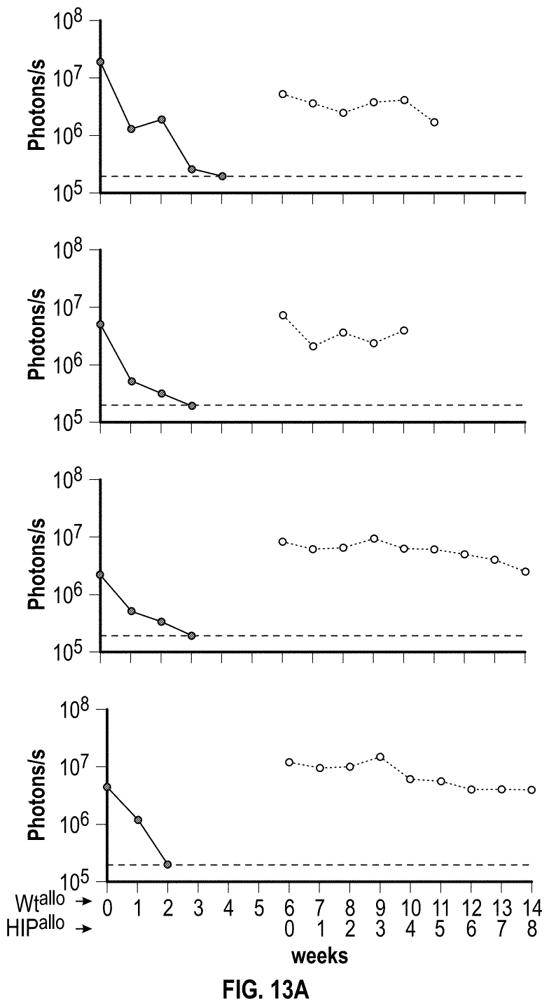

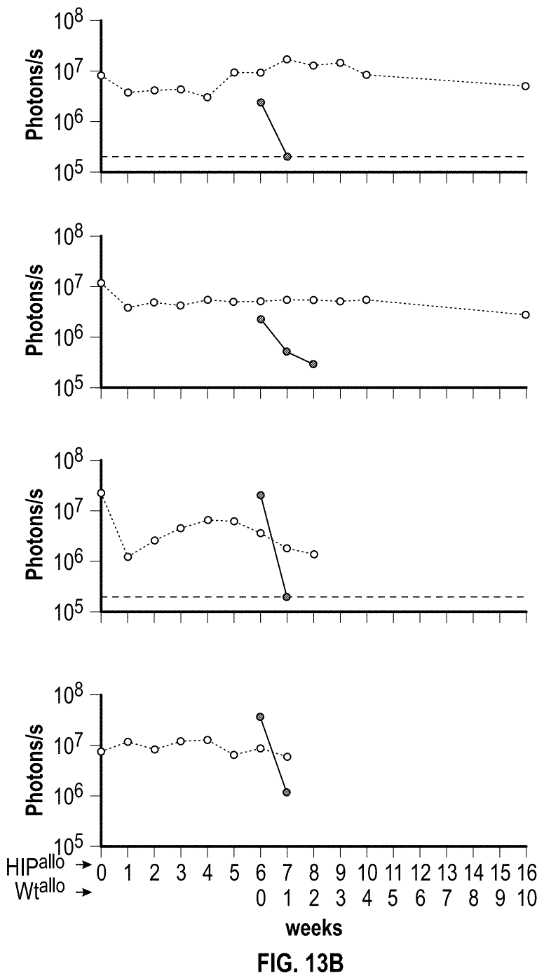

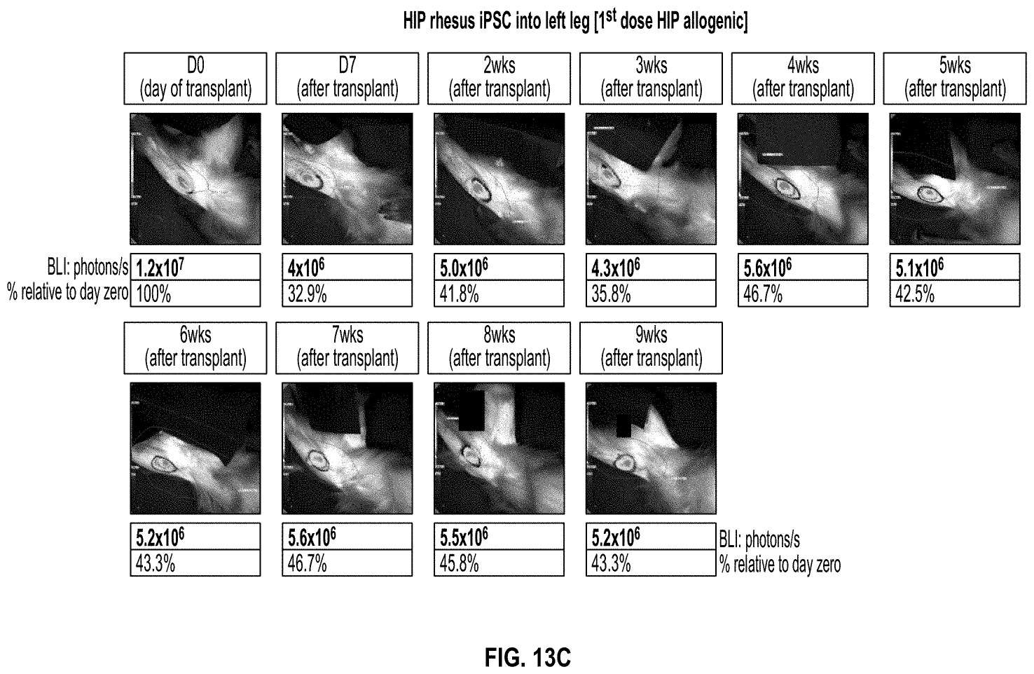

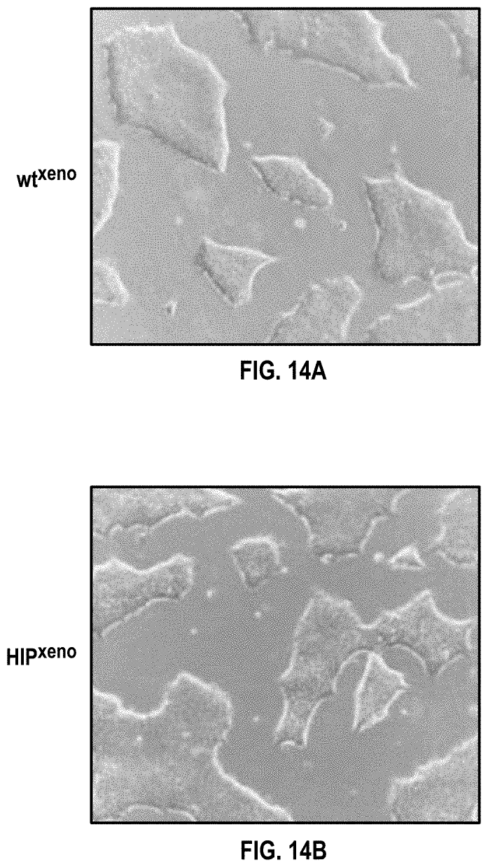

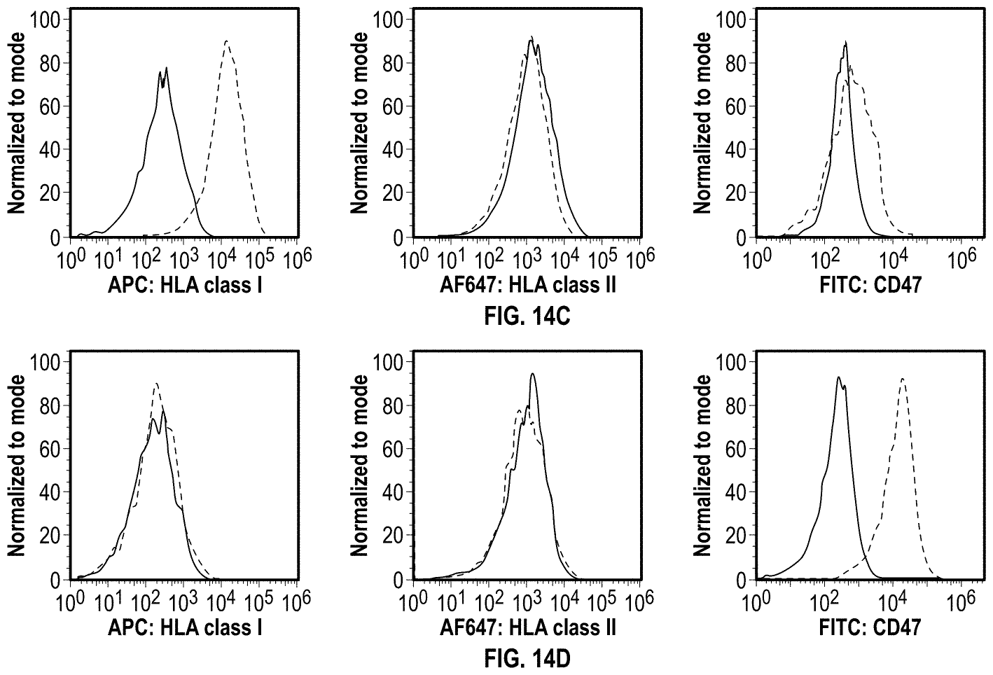

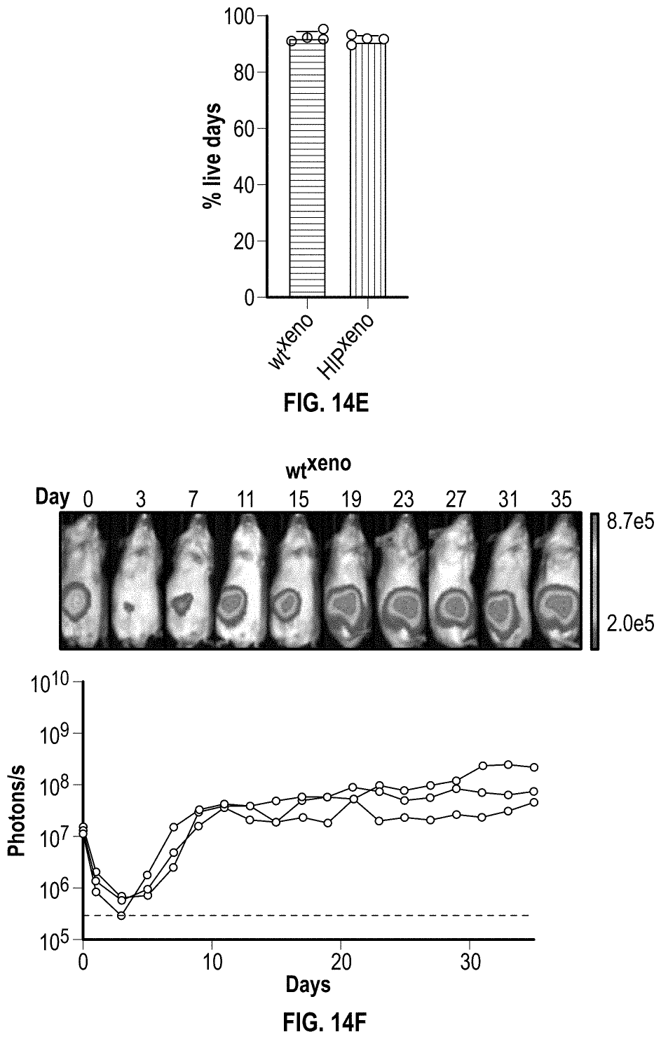

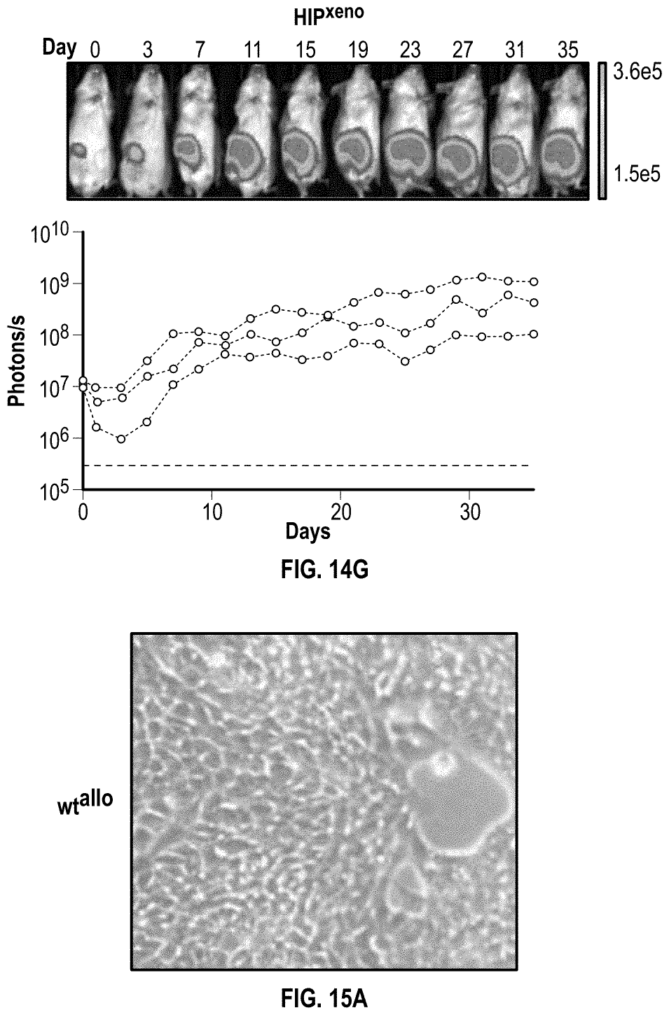

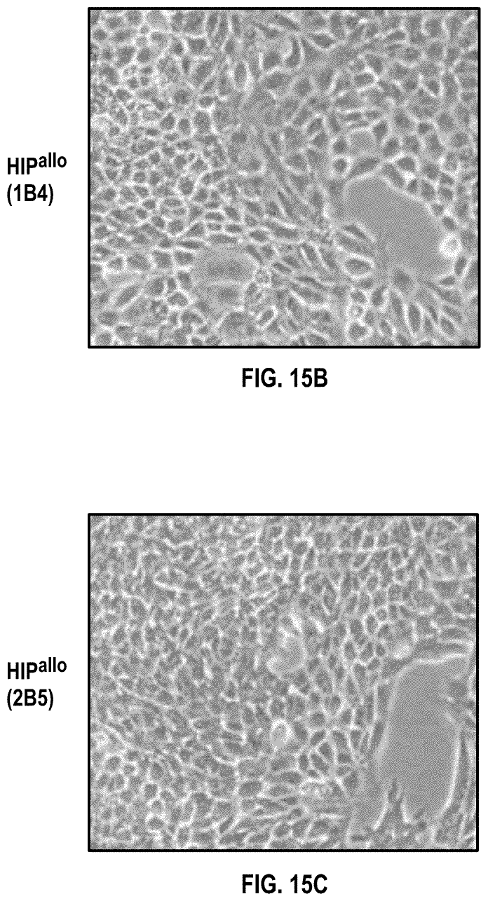

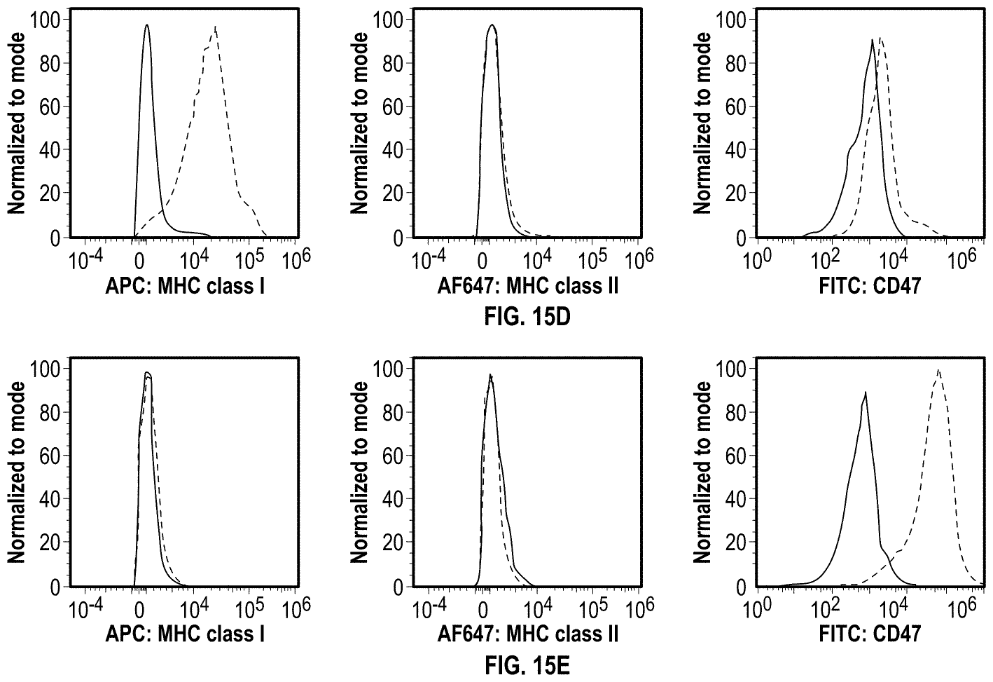

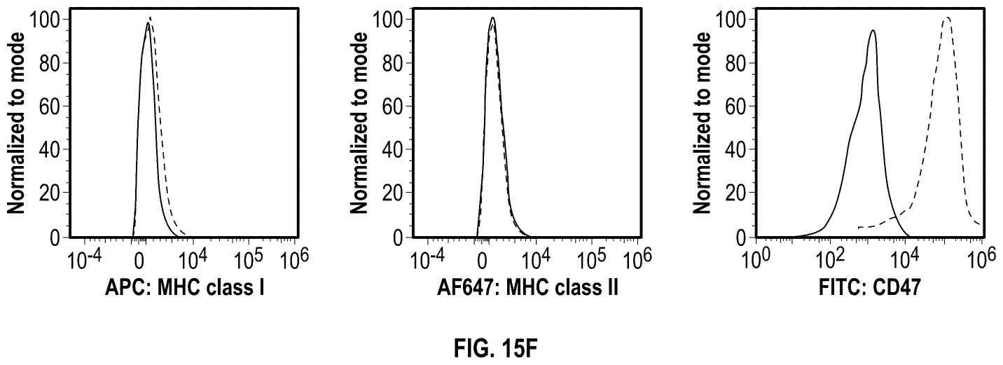

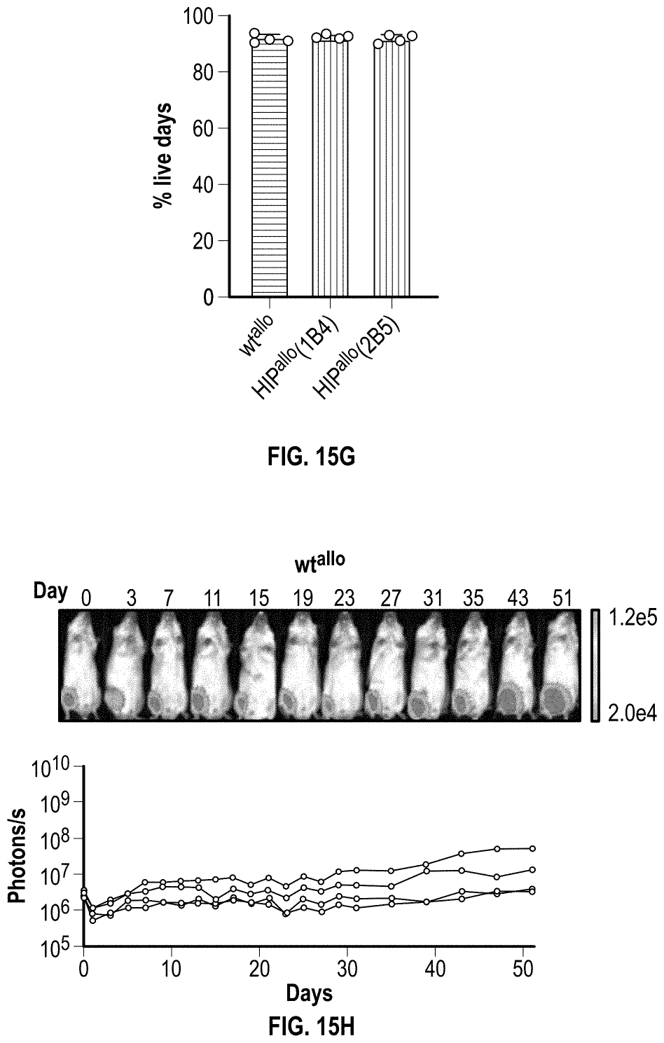

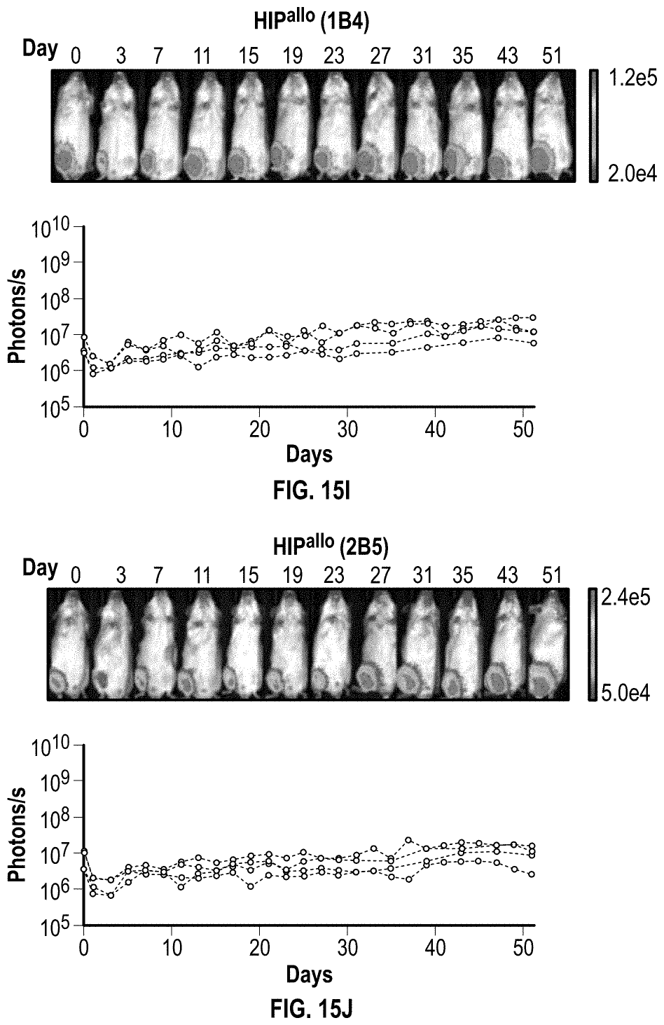

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogeneic humanized immunodeficient mouse model. In some instances, the hypoimmunogenic pluripotent stem cells are transplanted into an allogeneic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In one embodiment, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the hypoimmunogenic cells provided herein have a reduced susceptibility to macrophage phagocytosis and NK cell killing. The resulting hypoimmunogenic cells “escape” the immune macrophage and innate pathways due to the expression of one or more CD24 transgenes.

S. Maintenance of Pluripotent Stem Cells

Once the hypoimmunogenic pluripotent stem cells have been generated, they can be maintained an undifferentiated state as is known for maintaining iPSCs. For example, the cells can be cultured on Matrigel using culture media that prevents differentiation and maintains pluripotency. In addition, they can be in culture medium under conditions to maintain pluripotency.

T. Differentiated Cells from Hypoimmunogenic Induced Pluripotent (HIP) Stem Cells

In an aspect, provided herein are HIP cells that are differentiated into different cell types for subsequent transplantation into recipient subjects. Differentiation can be assayed as is known in the art, generally by evaluating the presence of cell-specific markers. As will be appreciated by those in the art, the differentiated hypoimmunogenic pluripotent cell derivatives can be transplanted using techniques known in the art that depends on both the cell type and the ultimate use of these cells.

1. Cardiac Cells Differentiated from Hypoimmunogenic Pluripotent Cells

Provided herein are cardiac cell types differentiated from HIP cells for subsequent transplantation or engraftment into subjects (e.g., recipients). As will be appreciated by those in the art, the methods for differentiation depend on the desired cell type using known techniques. Exemplary cardiac cell types include, but are not limited to, a cardiomyocyte, nodal cardiomyocyte, conducting cardiomyocyte, working cardiomyocyte, cardiomyocyte precursor cell, cardiomyocyte progenitor cell, cardiac stem cell, cardiac muscle cell, atrial cardiac stem cell, ventricular cardiac stem cell, epicardial cell, hematopoietic cell, vascular endothelial cell, endocardial endothelial cell, cardiac valve interstitial cell, cardiac pacemaker cell, and the like.

In some embodiments, cardiac cells described herein are administered to a recipient subject to treat a cardiac disorder selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, and autoimmune endocarditis.

Accordingly, provided herein are methods for the treatment and prevention of a cardiac injury or a cardiac disease or disorder in a subject in need thereof. The methods described herein can be used to treat, ameliorate, prevent or slow the progression of a number of cardiac diseases or their symptoms, such as those resulting in pathological damage to the structure and/or function of the heart. The terms “cardiac disease,” “cardiac disorder,” and “cardiac injury,” are used interchangeably herein and refer to a condition and/or disorder relating to the heart, including the valves, endothelium, infarcted zones, or other components or structures of the heart. Such cardiac diseases or cardiac-related disease include, but are not limited to, myocardial infarction, heart failure, cardiomyopathy, congenital heart defect, heart valve disease or dysfunction, endocarditis, rheumatic fever, mitral valve prolapse, infective endocarditis, hypertrophic cardiomyopathy, dilated cardiomyopathy, myocarditis, cardiomegaly, and/or mitral insufficiency, among others.

In some embodiments, the cardiomyocyte precursor includes a cell that is capable giving rise to progeny that include mature (end-stage) cardiomyocytes. Cardiomyocyte precursor cells can often be identified using one or more markers selected from GATA-4, Nkx2.5, and the MEF-2 family of transcription factors. In some instances, cardiomyocytes refer to immature cardiomyocytes or mature cardiomyocytes that express one or more markers (sometimes at least 2, 3, 4 or 5 markers) from the following list: cardiac troponin I (cTn1), cardiac troponin T (cTnT), sarcomeric myosin heavy chain (MHC), GATA-4, Nkx2.5, N-cadherin, β2-adrenoceptor, ANF, the MEF-2 family of transcription factors, creatine kinase MB (CK-MB), myoglobin, and atrial natriuretic factor (ANF). In some embodiments, the cardiac cells demonstrate spontaneous periodic contractile activity. In some cases, when that cardiac cells are cultured in a suitable tissue culture environment with an appropriate Ca²⁺ concentration and electrolyte balance, the cells can be observed to contract in a periodic fashion across one axis of the cell, and then release from contraction, without having to add any additional components to the culture medium. In some embodiments, the cardiac cells are hypoimmunogenic cardiac cells.

In some embodiments, the method of producing a population of hypoimmunogenic cardiac cells from a population of hypoimmunogenic pluripotent (HIP) cells by in vitro differentiation comprises: (a) culturing a population of HIP cells in a culture medium comprising a GSK inhibitor; (b) culturing the population of HIP cells in a culture medium comprising a WNT antagonist to produce a population of pre-cardiac cells; and (c) culturing the population of pre-cardiac cells in a culture medium comprising insulin to produce a population of hypoimmune cardiac cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the WNT antagonist is IWR1, a derivative thereof, or a variant thereof. In some instances, the WNT antagonist is at a concentration ranging from about 2 mM to about 10 mM.

In some embodiments, the population of hypoimmunogenic cardiac cells is isolated from non-cardiac cells. In some embodiments, the isolated population of hypoimmunogenic cardiac cells are expanded prior to administration. In certain embodiments, the isolated population of hypoimmunogenic cardiac cells are expanded and cryopreserved prior to administration.

In some embodiments, the pluripotent cells are differentiated into cardiomyocytes to address cardiovascular diseases. Techniques are known in the art for the differentiation of hiPSCs to cardiomyocytes and discussed in the Examples. Differentiation can be assayed as is known in the art, generally by evaluating the presence of cardiomyocyte associated or specific markers or by measuring functionally; see, for example Loh et al., Cell, 2016, 166, 451-467, hereby incorporated by reference in its entirety and specifically for the methods of differentiating stem cells including cardiomyocytes.

Other useful methods for differentiating induced pluripotent stem cells or pluripotent stem cells into cardiac cells are described, for example, in US2017/0152485; US2017/0058263; US2017/0002325; US2016/0362661; US2016/0068814; U.S. Pat. Nos. 9,062,289; 7,897,389; and 7,452,718. Additional methods for producing cardiac cells from induced pluripotent stem cells or pluripotent stem cells are described in, for example, Xu et al., Stem Cells and Development, 2006, 15(5): 631-9, Burridge et al., Cell Stem Cell, 2012, 10: 16-28, and Chen et al., Stem Cell Res, 2015, 15(2):365-375.

In various embodiments, hypoimmunogenic cardiac cells can be cultured in culture medium comprising a BMP pathway inhibitor, a WNT signaling activator, a WNT signaling inhibitor, a WNT agonist, a WNT antagonist, a Src inhibitor, a EGFR inhibitor, a PCK activator, a cytokine, a growth factor, a cardiotropic agent, a compound, and the like.

The WNT signaling activator includes, but is not limited to, CHIR99021. The PCK activator includes, but is not limited to, PMA. The WNT signaling inhibitor includes, but is not limited to, a compound selected from KY02111, SO3031 (KY01-I), SO2031 (KY02-I), and SO3042 (KY03-I), and XAV939. The Src inhibitor includes, but is not limited to, A419259. The EGFR inhibitor includes, but is not limited to, AG1478.

Non-limiting examples of an agent for generating a cardiac cell from an iPSC include activin A, BMP4, Wnt3a, VEGF, soluble frizzled protein, cyclosporin A, angiotensin II, phenylephrine, ascorbic acid, dimethylsulfoxide, 5-aza-2′-deoxycytidine, and the like.

The cells provided herein can be cultured on a surface, such as a synthetic surface to support and/or promote differentiation of hypoimmunogenic pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethylene glycol) dimethacrylate, 1,6-hexanediol propoxylate diacrylate, neopentyl glycol diacrylate, trimethylolpropane benzoate diacrylate, trimethylolpropane ethoxylate (1 EO/QH) methyl, tricyclo[5.2.1.0^{2,6}] decane dimethanol diacrylate, neopentyl glycol ethoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

The efficacy of cardiac cells prepared as described herein can be assessed in animal models for cardiac cryoinjury, which causes 55% of the left ventricular wall tissue to become sCAR-Tissue without treatment (Li et al., Ann. Thorac. Surg. 62:654, 1996; Sakai et al., Ann. Thorac. Surg. 8:2074, 1999, Sakai et al., Thorac. Cardiovasc. Surg. 118:715, 1999). Successful treatment can reduce the area of the scar, limit scar expansion, and improve heart function as determined by systolic, diastolic, and developed pressure. Cardiac injury can also be modeled using an embolization coil in the distal portion of the left anterior descending artery (Watanabe et al., Cell Transplant. 7:239, 1998), and efficacy of treatment can be evaluated by histology and cardiac function.

In some embodiments, the administration comprises implantation into the subject's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the patient administered the engineered cardiac cells is also administered a cardiac drug. Illustrative examples of cardiac drugs that are suitable for use in combination therapy include, but are not limited to, growth factors, polynucleotides encoding growth factors, angiogenic agents, calcium channel blockers, antihypertensive agents, antimitotic agents, inotropic agents, anti-atherogenic agents, anti-coagulants, beta-blockers, anti-arrhythmic agents, anti-inflammatory agents, vasodilators, thrombolytic agents, cardiac glycosides, antibiotics, antiviral agents, antifungal agents, agents that inhibit protozoans, nitrates, angiotensin converting enzyme (ACE) inhibitors, angiotensin II receptor antagonist, brain natriuretic peptide (BNP); antineoplastic agents, steroids, and the like.

The effects of therapy according to the methods provided herein can be monitored in a variety of ways. For instance, an electrocardiogram (ECG) or holier monitor can be utilized to determine the efficacy of treatment. An ECG is a measure of the heart rhythms and electrical impulses, and is a very effective and non-invasive way to determine if therapy has improved or maintained, prevented, or slowed degradation of the electrical conduction in a subject's heart. The use of a holier monitor, a portable ECG that can be worn for long periods of time to monitor heart abnormalities, arrhythmia disorders, and the like, is also a reliable method to assess the effectiveness of therapy. An ECG or nuclear study can be used to determine improvement in ventricular function.

2. Neural Cells Differentiated from Hypoimmunogenic Pluripotent Cells

Provided herein are different neural cell types differentiated from HIP cells that are useful for subsequent transplantation or engraftment into recipient subjects. As will be appreciated by those in the art, the methods for differentiation depend on the desired cell type using known techniques. Exemplary neural cell types include, but are not limited to, cerebral endothelial cells, neurons (e.g., dopaminergic neurons), glial cells, and the like.

In some embodiments, differentiation of induced pluripotent stem cells is performed by exposing or contacting cells to specific factors which are known to produce a specific cell lineage(s), so as to target their differentiation to a specific, desired lineage and/or cell type of interest. In some embodiments, terminally differentiated cells display specialized phenotypic characteristics or features. In certain embodiments, the stem cells described herein are differentiated into a neuroectodermal, neuronal, neuroendocrine, dopaminergic, cholinergic, serotonergic (5-HT), glutamatergic, GABAergic, adrenergic, noradrenergic, sympathetic neuronal, parasympathetic neuronal, sympathetic peripheral neuronal, or glial cell population. In some instances, the glial cell population includes a microglial (e.g., amoeboid, ramified, activated phagocytic, and activated non-phagocytic) cell population or a macroglial (central nervous system cell: astrocyte, oligodendrocyte, ependymal cell, and radial glia; and peripheral nervous system cell: Schwann cell and satellite cell) cell population, or the precursors and progenitors of any of the preceding cells.

Protocols for generating different types of neural cells are described in PCT Application No. WO2010144696, U.S. Pat. Nos. 9,057,053; 9,376,664; and 10,233,422. Additional descriptions of methods for differentiating hypoimmunogenic pluripotent cells can be found, for example, in Deuse et al., Nature Biotechnology, 2019, 37, 252-258 and Han et al., Proc Natl Acad Sci USA, 2019, 116(21), 10441-10446. Methods for determining the effect of neural cell transplantation in an animal model of a neurological disorder or condition are described in the following references: for spinal cord injury—Curtis et al., Cell Stem Cell, 2018, 22, 941-950; for Parkinson's disease—Kikuchi et al., Nature, 2017, 548:592-596; for ALS—Izrael et al., Stem Cell Research, 2018, 9(1):152 and Izrael et al., IntechOpen, DOI: 10.5772/intechopen.72862; for epilepsy—Upadhya et al., PNAS, 2019, 116(1):287-296.

a. Cerebral Endothelial Cells

In some embodiments, neural cells are administered to a subject to treat Parkinson's disease, Huntington disease, multiple sclerosis, other neurodegenerative disease or condition, attention deficit hyperactivity disorder (ADHD), Tourette Syndrome (TS), schizophrenia, psychosis, depression, other neuropsychiatric disorder. In some embodiments, neural cells described herein are administered to a subject to treat or ameliorate stroke. In some embodiments, the neurons and glial cells are administered to a subject with amyotrophic lateral sclerosis (ALS). In some embodiments, cerebral endothelial cells are administered to alleviate the symptoms or effects of cerebral hemorrhage. In some embodiments, dopaminergic neurons are administered to a patient with Parkinson's disease. In some embodiments, noradrenergic neurons, GABAergic interneurons are administered to a patient who has experienced an epileptic seizure. In some embodiments, motor neurons, interneurons, Schwann cells, oligodendrocytes, and microglia are administered to a patient who has experienced a spinal cord injury.

In some embodiments, cerebral endothelial cells (ECs), precursors, and progenitors thereof are differentiated from pluripotent stem cells (e.g., induced pluripotent stem cells) on a surface by culturing the cells in a medium comprising one or more factors that promote the generation of cerebral ECs or neural cell. In some instances, the medium includes one or more of the following: CHIR-99021, VEGF, basic FGF (bFGF), and Y-27632. In some embodiments, the medium includes a supplement designed to promote survival and functionality for neural cells.

In some embodiments, cerebral endothelial cells (ECs), precursors, and progenitors thereof are differentiated from pluripotent stem cells on a surface by culturing the cells in an unconditioned or conditioned medium. In some instances, the medium comprises factors or small molecules that promote or facilitate differentiation. In some embodiments, the medium comprises one or more factors or small molecules selected from the group consisting of VEGR, FGF, SDF-1, CHIR-99021, Y-27632, SB 431542, and any combination thereof. In some embodiments, the surface for differentiation comprises one or more extracellular matrix proteins. The surface can be coated with the one or more extracellular matrix proteins. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers.

In some embodiments, the cerebral endothelial cells express or secrete a factor selected from the group consisting of CD31, VE cadherin, and a combination thereof. In certain embodiments, the cerebral endothelial cells express or secrete one or more of the factors selected from the group consisting of CD31, CD34, CD45, CD117 (c-kit), CD146, CXCR4, VEGF, SDF-1, PDGF, GLUT-1, PECAM-1, eNOS, claudin-5, occludin, ZO-1, p-glycoprotein, von Willebrand factor, VE-cadherin, low density lipoprotein receptor LDLR, low density lipoprotein receptor-related protein 1 LRP1, insulin receptor INSR, leptin receptor LEPR, basal cell adhesion molecule BCAM, transferrin receptor TFRC, advanced glycation endproduct-specific receptor AGER, receptor for retinol uptake STRA6, large neutral amino acids transporter small subunit 1 SLC7A5, excitatory amino acid transporter 3 SLC1A1, sodium-coupled neutral amino acid transporter 5 SLC38A5, solute carrier family 16 member 1 SLC16A1, ATP-dependent translocase ABCB1, ATP-ABCC2-binding cassette transporter ABCG2, multidrug resistance-associated protein 1 ABCC1, canalicular multispecific organic anion transporter 1 ABCC2, multidrug resistance-associated protein 4 ABCC4, and multidrug resistance-associated protein 5 ABCC5.

In some embodiments, the cerebral ECs are characterized with one or more of the features selected from the group consisting of high expression of tight junctions, high electrical resistance, low fenestration, small perivascular space, high prevalence of insulin and transferrin receptors, and high number of mitochondria.

In some embodiments, cerebral ECs are selected or purified using a positive selection strategy. In some instances, the cerebral ECs are sorted against an endothelial cell marker such as, but not limited to, CD31. In other words, CD31 positive cerebral ECs are isolated. In some embodiments, cerebral ECs are selected or purified using a negative selection strategy. In some embodiments, undifferentiated or pluripotent stem cells are removed by selecting for cells that express a pluripotency marker including, but not limited to, TRA-1-60 and SSEA-1.

b. Dopaminergic Neurons

In some embodiments, HIP cells described herein are differentiated into dopaminergic neurons include neuronal stem cells, neuronal progenitor cells, immature dopaminergic neurons, and mature dopaminergic neurons.

In some cases, the term “dopaminergic neurons” includes neuronal cells which express tyrosine hydroxylase (TH), the rate-limiting enzyme for dopamine synthesis. In some embodiments, dopaminergic neurons secrete the neurotransmitter dopamine, and have little or no expression of dopamine hydroxylase. A dopaminergic (DA) neuron can express one or more of the following markers: neuron-specific enolase (NSE), 1-aromatic amino acid decarboxylase, vesicular monoamine transporter 2, dopamine transporter, Nurr-1, and dopamine-2 receptor (D2 receptor). In certain cases, the term “neural stem cells” includes a population of pluripotent cells that have partially differentiated along a neural cell pathway and express one or more neural markers including, for example, nestin. Neural stem cells may differentiate into neurons or glial cells (e.g., astrocytes and oligodendrocytes). The term “neural progenitor cells” includes cultured cells which express FOXA2 and low levels of b-tubulin, but not tyrosine hydroxylase. Such neural progenitor cells have the capacity to differentiate into a variety of neuronal subtypes; particularly a variety of dopaminergic neuronal subtypes, upon culturing the appropriate factors, such as those described herein.

In some embodiments, the DA neurons derived from HIP cells are administered to a patient, e.g., human patient to treat a neurodegenerative disease or condition. In some cases, the neurodegenerative disease or condition is selected from the group consisting of Parkinson's disease, Huntington disease, and multiple sclerosis. In other embodiments, the DA neurons are used to treat or ameliorate one or more symptoms of a neuropsychiatric disorder, such as attention deficit hyperactivity disorder (ADHD), Tourette Syndrome (TS), schizophrenia, psychosis, and depression. In yet other embodiments, the DA neurons are used to treat a patient with impaired DA neurons.

In some embodiments, DA neurons, precursors, and progenitors thereof are differentiated from pluripotent stem cells by culturing the stem cells in medium comprising one or more factors or additives. Useful factors and additives that promote differentiation, growth, expansion, maintenance, and/or maturation of DA neurons include, but are not limited to, Wnt1, FGF2, FGF8, FGF8a, sonic hedgehog (SHH), brain derived neurotrophic factor (BDNF), transforming growth factor a (TGF-a), TGF-b, interleukin 1 beta, glial cell line-derived neurotrophic factor (GDNF), a GSK-3 inhibitor (e.g., CHIR-99021), a TGF-b inhibitor (e.g., SB-431542), B-27 supplement, dorsomorphin, purmorphamine, noggin, retinoic acid, cAMP, ascorbic acid, neurturin, knockout serum replacement, N-acetyl cysteine, c-kit ligand, modified forms thereof, mimics thereof, analogs thereof, and variants thereof. In some embodiments, the DA neurons are differentiated in the presence of one or more factors that activate or inhibit the WNT pathway, NOTCH pathway, SHH pathway, BMP pathway, FGF pathway, and the like. Differentiation protocols and detailed descriptions thereof are provided in, e.g., U.S. Pat. Nos. 9,968,637, 7,674,620, Kim et al., Nature, 2002, 418, 50-56; Bjorklund et al., PNAS, 2002, 99(4), 2344-2349; Grow et al., Stem Cells Transl Med. 2016, 5(9): 1133-44, and Cho et al., PNAS, 2008, 105:3392-3397, the disclosures in their entirety including the detailed description of the examples, methods, figures, and results are herein incorporated by reference.

In some embodiments, the population of hypoimmunogenic dopaminergic neurons is isolated from non-neuronal cells. In some embodiments, the isolated population of hypoimmunogenic dopaminergic neurons are expanded prior to administration. In certain embodiments, the isolated population of hypoimmunogenic dopaminergic neurons are expanded and cryopreserved prior to administration.

To characterize and monitor DA differentiation and assess the DA phenotype, expression of any number of molecular and genetic markers can be evaluated. For example, the presence of genetic markers can be determined by various methods known to those skilled in the art. Expression of molecular markers can be determined by quantifying methods such as, but not limited to, qPCR-based assays, immunoassays, immunocytochemistry assays, immunoblotting assays, and the like. Exemplary markers for DA neurons include, but are not limited to, TH, b-tubulin, paired box protein (Pax6), insulin gene enhancer protein (Isl1), nestin, diaminobenzidine (DAB), G protein-activated inward rectifier potassium channel 2 (GIRK2), microtubule-associated protein 2 (MAP-2), NURR1, dopamine transporter (DAT), forkhead box protein A2 (FOXA2), FOX3, doublecortin, and LIM homeobox transcription factor 1-beta (LMX1B), and the like. In some embodiments, the DA neurons express one or more of the markers selected from corin, FOXA2, TuJ1, NURR1, and any combination thereof.

In some embodiments, DA neurons are assessed according to cell electrophysiological activity. The electrophysiology of the cells can be evaluated by using assays knowns to those skilled in the art. For instance, whole-cell and perforated patch clamp, assays for detecting electrophysiological activity of cells, assays for measuring the magnitude and duration of action potential of cells, and functional assays for detecting dopamine production of DA cells.

In some embodiments, DA neuron differentiation is characterized by spontaneous rhythmic action potentials, and high-frequency action potentials with spike frequency adaption upon injection of depolarizing current. In other embodiments, DA differentiation is characterized by the production of dopamine. The level of dopamine produced is calculated by measuring the width of an action potential at the point at which it has reached half of its maximum amplitude (spike half-maximal width).

In some embodiments, the differentiated DA neurons are transplanted either intravenously or by injection at particular locations in the patient. In some embodiments, the differentiated DA cells are transplanted into the substantia nigra (particularly in or adjacent of the compact region), the ventral tegmental area (VTA), the caudate, the putamen, the nucleus accumbens, the subthalamic nucleus, or any combination thereof, of the brain to replace the DA neurons whose degeneration resulted in Parkinson's disease. The differentiated DA cells can be injected into the target area as a cell suspension. Alternatively, the differentiated DA cells can be embedded in a support matrix or scaffold when contained in such a delivery device. In some embodiments, the scaffold is biodegradable. In other embodiments, the scaffold is not biodegradable. The scaffold can comprise natural or synthetic (artificial) materials.

The delivery of the DA neurons can be achieved by using a suitable vehicle such as, but not limited to, liposomes, microparticles, or microcapsules. In other embodiments, the differentiated DA neurons are administered in a pharmaceutical composition comprising an isotonic excipient. The pharmaceutical composition is prepared under conditions that are sufficiently sterile for human administration. In some embodiments, the DA neurons differentiated from HIP cells are supplied in the form of a pharmaceutical composition. General principles of therapeutic formulations of cell compositions are found in Cell Therapy: Stem Cell Transplantation, Gene Therapy, and Cellular Immunotherapy, G. Morstyn & W. Sheridan eds, Cambridge University Press, 1996, and Hematopoietic Stem Cell Therapy, E. Ball, J. Lister & P. Law, Churchill Livingstone, 2000, the disclosures are incorporated herein by reference.

Useful descriptions of neurons derived from stem cells and methods of making thereof can be found, for example, in Kirkeby et al., Cell Rep, 2012, 1:703-714; Kriks et al., Nature, 2011, 480:547-551; Wang et al., Stem Cell Reports, 2018, 11(1):171-182; Lorenz Studer, “Chapter 8—Strategies for Bringing Stem Cell-Derived Dopamine Neurons to the clinic—The NYSTEM Trial” in Progress in Brain Research, 2017, volume 230, pg. 191-212; Liu et al., Nat Protoc, 2013, 8:1670-1679; Upadhya et al., Curr Protoc Stem Cell Biol, 38, 2D.7.1-2D.7.47; US Publication Appl. No. 20160115448, and U.S. Pat. Nos. 8,252,586; 8,273,570; 9,487,752 and 10,093,897, the contents are incorporated herein by reference in their entirety.

In addition to DA neurons, other neuronal cells, precursors, and progenitors thereof can be differentiated from the HIP cells outlined herein by culturing the cells in medium comprising one or more factors or additive. Non-limiting examples of factors and additives include GDNF, BDNF, GM-CSF, B27, basic FGF, basic EGF, NGF, CNTF, SMAD inhibitor, Wnt antagonist, SHH signaling activator, and any combination thereof. In some embodiments, the SMAD inhibitor is selected from the group consisting of SB431542, LDN-193189, Noggin PD169316, SB203580, LY364947, A77-01, A-83-01, BMP4, GW788388, GW6604, SB-505124, lerdelimumab, metelimumab, GC-I008, AP-12009, AP-11014, LY550410, LY580276, LY364947, LY2109761, SB-505124, E-616452 (RepSox ALK inhibitor), SD-208, SMI6, NPC-30345, K 26894, SB-203580, SD-093, activin-M108A, P144, soluble TBR2-Fc, DMH-1, dorsomorphin dihydrochloride and derivatives thereof. In some embodiments, the Wnt antagonist is selected from the group consisting of XAV939, DKK1, DKK-2, DKK-3, DKK-4, SFRP-1, SFRP-2, SFRP-3, SFRP-4, SFRP-5, WIF-1, Soggy, IWP-2, IWR1, ICG-001, KY0211, Wnt-059, LGK974, IWP-L6 and derivatives thereof. In some embodiments, the SHH signaling activator is selected from the group consisting of Smoothened agonist (SAG), SAG analog, SHH, C25-SHH, C24-SHH, purmorphamine, Hg-Ag and/or derivatives thereof.

In some embodiments, the neurons express one or more of the markers selected from the group consisting of glutamate ionotropic receptor NMDA type subunit 1 GRIN1, glutamate decarboxylase 1 GAD1, gamma-aminobutyric acid GABA, tyrosine hydroxylase TH, LIM homeobox transcription factor 1-alpha LMX1A, Forkhead box protein O1 FOXO1, Forkhead box protein A2 FOXA2, Forkhead box protein O4 FOXO4, FOXG1, 2′,3′-cyclic-nucleotide 3′-phosphodiesterase CNP, myelin basic protein MBP, tubulin beta chain 3 TUB3, tubulin beta chain 3 NEUN, solute carrier family 1 member 6 SLC1A6, SST, PV, calbindin, RAX, LHX6, LHX8, DLX1, DLX2, DLX5, DLX6, SOX6, MAFB, NPAS1, ASCL1, SIX6, OLIG2, NKX2.1, NKX2.2, NKX6.2, VGLUT1, MAP2, CTIP2, SATB2, TBR1, DLX2, ASCL1, ChAT, NGFI-B, c-fos, CRF, RAX, POMC, hypocretin, NADPH, NGF, Ach, VAChT, PAX6, EMX2p75, CORIN, TUJ1, NURR1, and/or any combination thereof.

c. Glial Cells

In some embodiments, the neural cells described include glial cells such as, but not limited to, microglia, astrocytes, oligodendrocytes, ependymal cells and Schwann cells, glial precursors, and glial progenitors thereof are produced by differentiating pluripotent stem cells into therapeutically effective glial cells and the like. Differentiation of hypoimmunogenic pluripotent stem cells produces hypoimmunogenic neural cells, such as hypoimmunogenic glial cells.

In some embodiments, glial cells, precursors, and progenitors thereof generated by culturing pluripotent stem cells in medium comprising one or more agents selected from the group consisting of retinoic acid, IL-34, M-CSF, FLT3 ligand, GM-CSF, CCL2, a TGFbeta inhibitor, a BMP signaling inhibitor, a SHH signaling activator, FGF, platelet derived growth factor PDGF, PDGFR-alpha, HGF, IGF1, noggin, SHH, dorsomorphin, noggin, and any combination thereof. In certain instances, the BMP signaling inhibitor is LDN193189, SB431542, or a combination thereof. In some embodiments, the glial cells express NKX2.2, PAX6, SOX10, brain derived neurotrophic factor BDNF, neutrotrophin-3 NT-3, NT-4, EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CX3CR1, P2RY12, IBA-1, TMEM119, CD45, and any combination thereof. Exemplary differentiation medium can include any specific factors and/or small molecules that may facilitate or enable the generation of a glial cell type as recognized by those skilled in the art.

To determine if the cells generated according to the in vitro differentiation protocol display glial cell characteristics and features, the cells can be transplanted into an animal model. In some embodiments, the glial cells are injected into an immunocompromised mouse, e.g., an immunocompromised shiverer mouse. The glial cells are administered to the brain of the mouse and after a pre-selected amount of time the engrafted cells are evaluated. In some instances, the engrafted cells in the brain are visualized by using immunostaining and imaging methods. In some embodiments, it is determined that the glial cells express known glial cell biomarkers.

Useful methods for generating glial cells, precursors, and progenitors thereof from stem cells are found, for example, in U.S. Pat. Nos. 7,579,188; 7,595,194; 8,263,402; 8,206,699; 8,252,586; 9,193,951; 9,862,925; 8,227,247; 9,709,553; US2018/0187148; US2017/0198255; US2017/0183627; US2017/0182097; US2017/253856; US2018/0236004; WO2017/172976; and WO2018/093681. Methods for differentiating pluripotent stem cells are described in, e.g., Kikuchi et al., Nature, 2017, 548, 592-596; Kriks et al., Nature, 2011, 547-551; Doi et al., Stem Cell Reports, 2014, 2, 337-50; Perrier et al., Proc Natl Acad Sci USA, 2004, 101, 12543-12548; Chambers et al., Nat Biotechnol, 2009, 27, 275-280; and Kirkeby et al., Cell Reports, 2012, 1, 703-714.

The efficacy of neural cell transplants for spinal cord injury can be assessed in, for example, a rat model for acutely injured spinal cord, as described by McDonald, et al., Nat. Med., 1999, 5:1410) and Kim, et al., Nature, 2002, 418:50. For instance, successful transplants may show transplant-derived cells present in the lesion 2-5 weeks later, differentiated into astrocytes, oligodendrocytes, and/or neurons, and migrating along the spinal cord from the lesioned end, and an improvement in gait, coordination, and weight-bearing. Specific animal models are selected based on the neural cell type and neurological disease or condition to be treated.

The neural cells can be administered in a manner that permits them to engraft to the intended tissue site and reconstitute or regenerate the functionally deficient area. For instance, neural cells can be transplanted directly into parenchymal or intrathecal sites of the central nervous system, according to the disease being treated. In some embodiments, any of the neural cells described herein including cerebral endothelial cells, neurons, dopaminergic neurons, ependymal cells, astrocytes, microglial cells, oligodendrocytes, and Schwann cells are injected into a patient by way of intravenous, intraspinal, intracerebroventricular, intrathecal, intra-arterial, intramuscular, intraperitoneal, subcutaneous, intramuscular, intra-abdominal, intraocular, retrobulbar and combinations thereof. In some embodiments, the cells are injected or deposited in the form of a bolus injection or continuous infusion. In certain embodiments, the neural cells are administered by injection into the brain, apposite the brain, and combinations thereof. The injection can be made, for example, through a burr hole made in the subject's skull. Suitable sites for administration of the neural cell to the brain include, but are not limited to, the cerebral ventricle, lateral ventricles, cisterna magna, putamen, nucleus basalis, hippocampus cortex, striatum, caudate regions of the brain and combinations thereof.

Additional descriptions of neural cells including dopaminergic neurons for use in the present technology are found in WO2020/018615, the disclosure is herein incorporated by reference in its entirety.

3. Endothelial Cells Differentiated from Hypoimmunogenic Pluripotent Cells

Provided herein are hypoimmunogenic pluripotent cells that are differentiated into various endothelial cell types for subsequent transplantation or engraftment into subjects (e.g., recipients). As will be appreciated by those in the art, the methods for differentiation depend on the desired cell type using known techniques.

In some embodiments, the endothelial cells differentiated from the subject hypoimmunogenic pluripotent cells are administered to a patient, e.g., a human patient in need thereof. The endothelial cells can be administered to a patient suffering from a disease or condition such as, but not limited to, cardiovascular disease, vascular disease, peripheral vascular disease, ischemic disease, myocardial infarction, congestive heart failure, peripheral vascular obstructive disease, stroke, reperfusion injury, limb ischemia, neuropathy (e.g., peripheral neuropathy or diabetic neuropathy), organ failure (e.g., liver failure, kidney failure, and the like), diabetes, rheumatoid arthritis, osteoporosis, vascular injury, tissue injury, hypertension, angina pectoris and myocardial infarction due to coronary artery disease, renal vascular hypertension, renal failure due to renal artery stenosis, claudication of the lower extremities, and the like. In certain embodiments, the patient has suffered from or is suffering from a transient ischemic attack or stroke, which in some cases, may be due to cerebrovascular disease. In some embodiments, the engineered endothelial cells are administered to treat tissue ischemia e.g., as occurs in atherosclerosis, myocardial infarction, and limb ischemia and to repair of injured blood vessels. In some instances, the cells are used in bioengineering of grafts.

For instance, the endothelial cells can be used in cell therapy for the repair of ischemic tissues, formation of blood vessels and heart valves, engineering of artificial vessels, repair of damaged vessels, and inducing the formation of blood vessels in engineered tissues (e.g., prior to transplantation). Additionally, the endothelial cells can be further modified to deliver agents to target and treat tumors.

In many embodiments, provided herein is a method of repair or replacement for tissue in need of vascular cells or vascularization. The method involves administering to a human patient in need of such treatment, a composition containing the isolated endothelial cells to promote vascularization in such tissue. The tissue in need of vascular cells or vascularization can be a cardiac tissue, liver tissue, pancreatic tissue, renal tissue, muscle tissue, neural tissue, bone tissue, among others, which can be a tissue damaged and characterized by excess cell death, a tissue at risk for damage, or an artificially engineered tissue.

In some embodiments, vascular diseases, which may be associated with cardiac diseases or disorders can be treated by administering endothelial cells, such as but not limited to, definitive vascular endothelial cells and endocardial endothelial cells derived as described herein. Such vascular diseases include, but are not limited to, coronary artery disease, cerebrovascular disease, aortic stenosis, aortic aneurysm, peripheral artery disease, atherosclerosis, varicose veins, angiopathy, infarcted area of heart lacking coronary perfusion, non-healing wounds, diabetic or non-diabetic ulcers, or any other disease or disorder in which it is desirable to induce formation of blood vessels.

In certain embodiments, the endothelial cells are used for improving prosthetic implants (e.g., vessels made of synthetic materials such as Dacron and Gortex.) which are used in vascular reconstructive surgery. For example, prosthetic arterial grafts are often used to replace diseased arteries which perfuse vital organs or limbs. In other embodiments, the engineered endothelial cells are used to cover the surface of prosthetic heart valves to decrease the risk of the formation of emboli by making the valve surface less thrombogenic.

The endothelial cells outlined can be transplanted into the patient using well known surgical techniques for grafting tissue and/or isolated cells into a vessel. In some embodiments, the cells are introduced into the patient's heart tissue by injection (e.g., intramyocardial injection, intracoronary injection, trans-endocardial injection, trans-epicardial injection, percutaneous injection), infusion, grafting, and implantation.

Administration (delivery) of the endothelial cells includes, but is not limited to, subcutaneous or parenteral including intravenous, intraarterial (e.g., intracoronary), intramuscular, intraperitoneal, intramyocardial, trans-endocardial, trans-epicardial, intranasal administration as well as intrathecal, and infusion techniques.

As will be appreciated by those in the art, the HIP derivatives are transplanted using techniques known in the art that depend on both the cell type and the ultimate use of these cells. In some embodiments, the cells are transplanted either intravenously or by injection at particular locations in the patient. When transplanted at particular locations, the cells may be suspended in a gel matrix to prevent dispersion while they take hold.

Exemplary endothelial cell types include, but are not limited to, a capillary endothelial cell, vascular endothelial cell, aortic endothelial cell, arterial endothelial cell, venous endothelial cell, renal endothelial cell, brain endothelial cell, liver endothelial cell, and the like.

The endothelial cells outlined herein can express one or more endothelial cell markers. Non-limiting examples of such markers include VE-cadherin (CD 144), ACE (angiotensin-converting enzyme) (CD 143), BNH9/BNF13, CD31, CD34, CD54 (ICAM-1), CD62E (E-Selectin), CD105 (Endoglin), CD146, Endocan (ESM-1), Endoglyx-1, Endomucin, Eotaxin-3, EPAS1 (Endothelial PAS domain protein 1), Factor VIII related antigen, FLI-1, Flk-1 (KDR, VEGFR-2), FLT-1 (VEGFR-1), GATA2, GBP-1 (guanylate-binding protein-1), GRO-alpha, HEX, ICAM-2 (intercellular adhesion molecule 2), LM02, LYVE-1, MRB (magic roundabout), Nucleolin, PAL-E (pathologische anatomie Leiden-endothelium), RTKs, sVCAM-1, TALI, TEM1 (Tumor endothelial marker 1), TEM5 (Tumor endothelial marker 5), TEM7 (Tumor endothelial marker 7), thrombomodulin (TM, CD141), VCAM-1 (vascular cell adhesion molecule-1) (CD106), VEGF, vWF (von Willebrand factor), ZO-1, endothelial cell-selective adhesion molecule (ESAM), CD102, CD93, CD184, CD304, and DLL4.

In some embodiments, the endothelial cells are genetically modified to express an exogenous gene encoding a protein of interest such as but not limited to an enzyme, hormone, receptor, ligand, or drug that is useful for treating a disorder/condition or ameliorating symptoms of the disorder/condition. Standard methods for genetically modifying endothelial cells are described, e.g., in U.S. Pat. No. 5,674,722.

Such endothelial cells can be used to provide constitutive synthesis and delivery of polypeptides or proteins, which are useful in prevention or treatment of disease. In this way, the polypeptide is secreted directly into the bloodstream or other area of the body (e.g., central nervous system) of the individual. In some embodiments, the endothelial cells can be modified to secrete insulin, a blood clotting factor (e.g., Factor VIII or von Willebrand Factor), alpha-1 antitrypsin, adenosine deaminase, tissue plasminogen activator, interleukins (e.g., IL-1, IL-2, IL-3), and the like.

In some embodiments, the endothelial cells can be modified in a way that improves their performance in the context of an implanted graft. Non-limiting illustrative examples include secretion or expression of a thrombolytic agent to prevent intraluminal clot formation, secretion of an inhibitor of smooth muscle proliferation to prevent luminal stenosis due to smooth muscle hypertrophy, and expression and/or secretion of an endothelial cell mitogen or autocrine factor to stimulate endothelial cell proliferation and improve the extent or duration of the endothelial cell lining of the graft lumen.

In some embodiments, the engineered endothelial cells are utilized for delivery of therapeutic levels of a secreted product to a specific organ or limb. For example, a vascular implant lined with endothelial cells engineered (transduced) in vitro can be grafted into a specific organ or limb. The secreted product of the transduced endothelial cells will be delivered in high concentrations to the perfused tissue, thereby achieving a desired effect to a targeted anatomical location.

In other embodiments, the endothelial cells are genetically modified to contain a gene that disrupts or inhibits angiogenesis when expressed by endothelial cells in a vascularizing tumor. In some cases, the endothelial cells can also be genetically modified to express any one of the selectable suicide genes described herein which allows for negative selection of grafted endothelial cells upon completion of tumor treatment.

In some embodiments, endothelial cells described herein are administered to a recipient subject to treat a vascular disorder selected from the group consisting of vascular injury, cardiovascular disease, vascular disease, peripheral vascular disease, ischemic disease, myocardial infarction, congestive heart failure, peripheral vascular obstructive disease, hypertension, ischemic tissue injury, reperfusion injury, limb ischemia, stroke, neuropathy (e.g., peripheral neuropathy or diabetic neuropathy), organ failure (e.g., liver failure, kidney failure, and the like), diabetes, rheumatoid arthritis, osteoporosis, cerebrovascular disease, hypertension, angina pectoris and myocardial infarction due to coronary artery disease, renal vascular hypertension, renal failure due to renal artery stenosis, claudication of the lower extremities, and/or other vascular condition or disease.

In some embodiments, the hypoimmunogenic pluripotent cells are differentiated into endothelial colony forming cells (ECFCs) to form new blood vessels to address peripheral arterial disease. Techniques to differentiate endothelial cells are known. See, e.g., Prasain et al., doi: 10.1038/nbt.3048, incorporated herein by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the method of producing a population of hypoimmunogenic endothelial cells from a population of hypoimmunogenic pluripotent cells by in vitro differentiation comprises: (a) culturing a population of HIP cells in a first culture medium comprising a GSK inhibitor; (b) culturing the population of HIP cells in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of hypoimmunogenic endothelial cells.

In some embodiments, the GSK inhibitor is CHIR-99021, a derivative thereof, or a variant thereof. In some instances, the GSK inhibitor is at a concentration ranging from about 1 mM to about 10 mM. In some embodiments, the ROCK inhibitor is Y-27632, a derivative thereof, or a variant thereof. In some instances, the ROCK inhibitor is at a concentration ranging from about 1 pM to about 20 pM. In some embodiments, the ALK inhibitor is SB-431542, a derivative thereof, or a variant thereof. In some instances, the ALK inhibitor is at a concentration ranging from about 0.5 pM to about 10 pM.

In some embodiments, the first culture medium comprises from 2 pM to about 10 pM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 pM Y-27632 and 1 pM SB-431542. In certain embodiments, the third culture medium further comprises VEGF and bFGF. In particular instances, the first culture medium and/or the second medium is absent of insulin.

The cells provided herein can be cultured on a surface, such as a synthetic surface to support and/or promote differentiation of hypoimmunogenic pluripotent cells into cardiac cells. In some embodiments, the surface comprises a polymer material including, but not limited to, a homopolymer or copolymer of selected one or more acrylate monomers. Non-limiting examples of acrylate monomers and methacrylate monomers include tetra(ethylene glycol) diacrylate, glycerol dimethacrylate, 1,4-butanediol dimethacrylate, poly(ethylene glycol) diacrylate, di(ethylene glycol) dimethacrylate, tetra(ethylene glycol) dimethacrylate, 1,6-hexanediol propoxylate diacrylate, neopentyl glycol diacrylate, trimethylolpropane benzoate diacrylate, trimethylolpropane ethoxylate (1 EO/QH) methyl, tricyclo[5.2.1.0^{2,6}] decane dimethanol diacrylate, neopentyl glycol ethoxylate diacrylate, and trimethylolpropane triacrylate. Acrylate synthesized as known in the art or obtained from a commercial vendor, such as Polysciences, Inc., Sigma Aldrich, Inc. and Sartomer, Inc.

In some embodiments, the endothelial cells may be seeded onto a polymer matrix. In some cases, the polymer matrix is biodegradable. Suitable biodegradable matrices are well known in the art and include collagen-GAG, collagen, fibrin, PLA, PGA, and PLA/PGA copolymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides.

Non-biodegradable polymers may also be used as well. Other non-biodegradable, yet biocompatible polymers include polypyrrole, polyamines, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). The polymer matrix may be formed in any shape, for example, as particles, a sponge, a tube, a sphere, a strand, a coiled strand, a capillary network, a film, a fiber, a mesh, or a sheet. The polymer matrix can be modified to include natural or synthetic extracellular matrix materials and factors.

The polymeric material can be dispersed on the surface of a support material. Useful support materials suitable for culturing cells include a ceramic substance, a glass, a plastic, a polymer or co-polymer, any combinations thereof, or a coating of one material on another. In some instances, a glass includes soda-lime glass, pyrex glass, vycor glass, quartz glass, silicon, or derivatives of these or the like.

In some instances, plastics or polymers including dendritic polymers include poly(vinyl chloride), poly(vinyl alcohol), poly(methyl methacrylate), poly(vinyl acetate-maleic anhydride), poly(dimethylsiloxane) monomethacrylate, cyclic olefin polymers, fluorocarbon polymers, polystyrenes, polypropylene, polyethyleneimine or derivatives of these or the like. In some instances, copolymers include poly(vinyl acetate-co-maleic anhydride), poly(styrene-co-maleic anhydride), poly(ethylene-co-acrylic acid) or derivatives of these or the like.

In some embodiments, the population of hypoimmunogenic endothelial cells is isolated from non-endothelial cells. In some embodiments, the isolated population of hypoimmunogenic endothelial cells are expanded prior to administration. In certain embodiments, the isolated population of hypoimmunogenic endothelial cells are expanded and cryopreserved prior to administration.

Additional descriptions of endothelial cells for use in the methods provided herein are found in WO2020/018615, the disclosure is herein incorporated by reference in its entirety.

4. Thyroid Cells Differentiated from Hypoimmunogenic Pluripotent Cells

In some embodiments, the hypoimmunogenic pluripotent cells are differentiated into thyroid progenitor cells and thyroid follicular organoids that can secrete thyroid hormones to address autoimmune thyroiditis. Techniques to differentiate thyroid cells are known the art. See, e.g., Kurmann et al., Cell Stem Cell, 2015 Nov. 5; 17(5):527-42, incorporated herein by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

5. Hepatocytes Differentiated from Hypoimmunogenic Pluripotent Cells

In some embodiments, the hypoimmunogenic pluripotent cells are differentiated into hepatocytes to address loss of the hepatocyte functioning or cirrhosis of the liver. There are a number of techniques that can be used to differentiate HIP cells into hepatocytes; see for example, Pettinato et al, doi: 10.1038/spre32888, Snykers et al., Methods Mol Biol, 2011 698:305-314, Si-Tayeb et al., Hepatology, 2010, 51:297-305 and Asgari et al., Stem Cell Rev, 2013, 9(4):493-504, all of which are incorporated herein by reference in their entirety and specifically for the methodologies and reagents for differentiation. Differentiation can be assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

6. Pancreatic Islet Cells Differentiated from Hypoimmunogenic Pluripotent Cells

In some embodiments, pancreatic islet cells (also referred to as pancreatic beta cells) are derived from the HIP cells described herein. In some instances, hypoimmunogenic pluripotent cells that are differentiated into various pancreatic islet cell types are transplanted or engrafted into subjects (e.g., recipients). As will be appreciated by those in the art, the methods for differentiation depend on the desired cell type using known techniques. Exemplary pancreatic islet cell types include, but are not limited to, pancreatic islet progenitor cell, immature pancreatic islet cell, mature pancreatic islet cell, and the like. In some embodiments, pancreatic cells described herein are administered to a subject to treat diabetes.

In some embodiments, pancreatic islet cells are derived from the hypoimmunogenic pluripotent cells described herein. Useful method for differentiating pluripotent stem cells into pancreatic islet cells are described, for example, in U.S. Pat. Nos. 9,683,215; 9,157,062; and 8,927,280.

In some embodiments, the pancreatic islet cells produced by the methods as disclosed herein secretes insulin. In some embodiments, a pancreatic islet cell exhibits at least two characteristics of an endogenous pancreatic islet cell, for example, but not limited to, secretion of insulin in response to glucose, and expression of beta cell markers.

Exemplary beta cell markers or beta cell progenitor markers include, but are not limited to, c-peptide, Pdx1, glucose transporter 2 (Glut2), HNF6, VEGF, glucokinase (GCK), prohormone convertase (PC 1/3), Cdcp1, NeuroD, Ngn3, Nkx2.2, Nkx6.1, Nkx6.2, Pax4, Pax6, Ptf1a, Isl1, Sox9, Sox17, and FoxA2.

In some embodiments, the isolated pancreatic islet cells produce insulin in response to an increase in glucose. In various embodiments, the isolated pancreatic islet cells secrete insulin in response to an increase in glucose. In some embodiments, the cells have a distinct morphology such as a cobblestone cell morphology and/or a diameter of about 17 pm to about 25 pm.

In some embodiments, the hypoimmunogenic pluripotent cells are differentiated into beta-like cells or islet organoids for transplantation to address type I diabetes mellitus (T1DM). Cell systems are a promising way to address T1DM, see, e.g., Ellis et al., Nat Rev Gastroenterol Hepatol. 2017 October; 14(10):612-628, incorporated herein by reference. Additionally, Pagliuca et al. (Cell, 2014, 159(2):428-39) reports on the successful differentiation of β-cells from hiPSCs, the contents incorporated herein by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host; Vegas et al., Nat Med, 2016, 22(3):306-11, incorporated herein by reference in its entirety and in particular for the methods and reagents outlined there for the large-scale production of functional human β cells from human pluripotent stem cells.

In some embodiments, the method of producing a population of hypoimmunogenic pancreatic islet cells from a population of hypoimmunogenic pluripotent cells by in vitro differentiation comprises: (a) culturing the population of HIP cells in a first culture medium comprising one or more factors selected from the group consisting insulin-like growth factor, transforming growth factor, FGF, EGF, HGF, SHH, VEGF, transforming growth factor-b superfamily, BMP2, BMP7, a GSK inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of hypoimmune pancreatic islet cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the ALK inhibitor is SB-431542, a derivative thereof, or a variant thereof. In some instances, the ALK inhibitor is at a concentration ranging from about 1 pM to about 10 pM. In some embodiments, the first culture medium and/or second culture medium are absent of animal serum.

In some embodiments, the population of hypoimmunogenic pancreatic islet cells is isolated from non-pancreatic islet cells. In some embodiments, the isolated population of hypoimmunogenic pancreatic islet cells are expanded prior to administration. In certain embodiments, the isolated population of hypoimmunogenic pancreatic islet cells are expanded and cryopreserved prior to administration.

Differentiation is assayed as is known in the art, generally by evaluating the presence of β cell associated or specific markers, including but not limited to, insulin. Differentiation can also be measured functionally, such as measuring glucose metabolism, see generally Muraro et al., Cell Syst. 2016 Oct. 26; 3(4): 385-394.e3, hereby incorporated by reference in its entirety, and specifically for the biomarkers outlined there. Once the beta cells are generated, they can be transplanted (either as a cell suspension or within a gel matrix as discussed herein) into the portal vein/liver, the omentum, the gastrointestinal mucosa, the bone marrow, a muscle, or subcutaneous pouches.

Additional descriptions of pancreatic islet cells including dopaminergic neurons for use in the present technology are found in WO2020/018615, the disclosure is herein incorporated by reference in its entirety.

7. Retinal Pigmented Epithelium (RPE) Cells Differentiated from Hypoimmunogenic Pluripotent Cells

Provided herein are retinal pigmented epithelium (RPE) cells derived from the HIP cells described above. For instance, human RPE cells can be produced by differentiating human HIP cells. In some embodiments, hypoimmunogenic pluripotent cells that are differentiated into various RPE cell types are transplanted or engrafted into subjects (e.g., recipients). As will be appreciated by those in the art, the methods for differentiation depend on the desired cell type using known techniques.

The term “RPE” cells refers to pigmented retinal epithelial cells having a genetic expression profile similar or substantially similar to that of native RPE cells. Such RPE cells derived from pluripotent stem cells may possess the polygonal, planar sheet morphology of native RPE cells when grown to confluence on a planar substrate.

The RPE cells can be implanted into a patient suffering from macular degeneration or a patient having damaged RPE cells. In some embodiments, the patient has age-related macular degeneration (AMD), early AMD, intermediate AMD, late AMD, non-neovascular age-related macular degeneration, dry macular degeneration (dry age-related macular degeneration), wet macular degeneration (wet age-real ted macular degeneration), juvenile macular degeneration (JMD) (e.g., Stargardt disease, Best disease, and juvenile retinoschisis), Leber's Congenital Ameurosis, or retinitis pigmentosa. In other embodiments, the patient suffers from retinal detachment.

Exemplary RPE cell types include, but are not limited to, retinal pigmented epithelium (RPE) cell, RPE progenitor cell, immature RPE cell, mature RPE cell, functional RPE cell, and the like.

Useful methods for differentiating pluripotent stem cells into RPE cells are described in, for example, U.S. Pat. Nos. 9,458,428 and 9,850,463, the disclosures are herein incorporated by reference in their entirety, including the specifications. Additional methods for producing RPE cells from human induced pluripotent stem cells can be found in, for example, Lamba et al., PNAS, 2006, 103(34): 12769-12774; Mellough et al., Stem Cells, 2012, 30(4):673-686; Idelson et al., Cell Stem Cell, 2009, 5(4): 396-408; Rowland et al., Journal of Cellular Physiology, 2012, 227(2):457-466, Buchholz et al., Stem Cells Trans Med, 2013, 2(5): 384-393, and da Cruz et al., Nat Biotech, 2018, 36:328-337.

Human pluripotent stem cells have been differentiated into RPE cells using the techniques outlined in Kamao et al, Stem Cell Reports 2014:2:205-18, hereby incorporated by reference in its entirety and in particular for the methods and reagents outlined there for the differentiation techniques and reagents; see also Mandai et al., N Engl J Med, 2017, 376:1038-1046, the contents herein incorporated in its entirety for techniques for generating sheets of RPE cells and transplantation into patients. Differentiation can be assayed as is known in the art, generally by evaluating the presence of RPE associated and/or specific markers or by measuring functionally. See for example Kamao et al., Stem Cell Reports, 2014, 2(2):205-18, the contents incorporated herein by reference in its entirety and specifically for the markers outlined in the first paragraph of the results section.

In some embodiments, the method of producing a population of hypoimmunogenic retinal pigmented epithelium (RPE) cells from a population of hypoimmunogenic pluripotent cells by in vitro differentiation comprises: (a) culturing the population of hypoimmunogenic pluripotent cells in a first culture medium comprising any one of the factors selected from the group consisting of activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the population of pre-RPE cells in a second culture medium that is different than the first culture medium to produce a population of hypoimmunogenic RPE cells. In some embodiments, the ALK inhibitor is SB-431542, a derivative thereof, or a variant thereof. In some instances, the ALK inhibitor is at a concentration ranging from about 2 mM to about 10 pM. In some embodiments, the ROCK inhibitor is Y-27632, a derivative thereof, or a variant thereof. In some instances, the ROCK inhibitor is at a concentration ranging from about 1 pM to about 10 pM. In some embodiments, the first culture medium and/or second culture medium are absent of animal serum.

Differentiation can be assayed as is known in the art, generally by evaluating the presence of RPE associated and/or specific markers or by measuring functionally. See for example Kamao et al., Stem Cell Reports, 2014, 2(2):205-18, the contents are herein incorporated by reference in its entirety and specifically for the results section.

Additional descriptions of RPE cells for use in the present technology are found in WO2020/018615, the disclosure is herein incorporated by reference in its entirety.

For therapeutic application, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

8. T Lymphocyte Derived from Hypoimmunogenic Pluripotent Cells

Provided herein, T lymphocytes (T cells, including primary T cells) are derived from the HIP cells described herein (e.g., hypoimmunogenic iPSCs). Methods for generating T cells, including CAR-T-cells, from pluripotent stem cells (e.g., iPSC) are described, for example, in Iriguchi et al., Nature Communications 12, 430 (2021); Themeli et al. 16(4):357-366 (2015); Themeli et al., Nature Biotechnology 31:928-933 (2013). T lymphocyte derived hypoimmunogenic cells include, but are not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient.

In some embodiments, the hypoimmunogenic cells do not activate an immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the HIP-derived T cell includes a chimeric antigen receptor (CAR). Any suitable CAR can be included in the HIP-derived T cell, including the CARs described herein. In some embodiments, the HIP-derived T cell includes a polynucleotide encoding a CAR, wherein the polynucleotide is inserted in a genomic locus. In some embodiments, the polynucleotide is inserted into a safe harbor or a target locus. In some embodiments, the polynucleotide is inserted in a B2M, CIITA, TRAC, TRB, PD1 or CTLA4 gene. Any suitable method can be used to insert the CAR into the genomic locus of the hypoimmunogenic cell including the gene editing methods described herein (e.g., a CRISPR/Cas system).

HIP-derived T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

9. NK Cells Derived from Hypoimmunogenic Pluripotent Cells

Provided herein, natural killer (NK) cells are derived from the HIP cells described herein (e.g., hypoimmunogenic iPSCs).

NK cells (also defined as ‘large granular lymphocytes’) represent a cell lineage differentiated from the common lymphoid progenitor (which also gives rise to B lymphocytes and T lymphocytes). Unlike T-cells, NK cells do not naturally comprise CD3 at the plasma membrane. Importantly, NK cells do not express a TCR and typically also lack other antigen-specific cell surface receptors (as well as TCRs and CD3, they also do not express immunoglobulin B-cell receptors, and instead typically express CD16 and CD56). NK cell cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins. On contact with a target cell NK cells release proteins such as perforin, and enzymes such as proteases (granzymes). Perforin can form pores in the cell membrane of a target cell, inducing apoptosis or cell lysis.

There are a number of techniques that can be used to generate NK cells, including CAR-NK-cells, from pluripotent stem cells (e.g., iPSC); see, for example, Zhu et al., Methods Mol Biol. 2019; 2048:107-119; Knorr et al., Stem Cells Transl Med. 2013 2(4):274-83. doi: 10.5966/sctm.2012-0084; Zeng et al., Stem Cell Reports. 2017 Dec. 12; 9(6):1796-1812; Ni et al., Methods Mol Biol. 2013; 1029:33-41; Bernareggi et al., Exp Hematol. 2019 71:13-23; Shankar et al., Stem Cell Res Ther. 2020; 11(1):234, all of which are incorporated herein by reference in their entirety and specifically for the methodologies and reagents for differentiation. Differentiation can be assayed as is known in the art, generally by evaluating the presence of NK cell associated and/or specific markers, including, but not limited to, CD56, KIRs, CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the hypoimmunogenic pluripotent cells are differentiated into hepatocytes to address loss of the hepatocyte functioning or cirrhosis of the liver. There are a number of techniques that can be used to differentiate HIP cells into hepatocytes; see for example, Pettinato et al., doi: 10.1038/spre32888, Snykers et al., Methods Mol Biol., 2011 698:305-314, Si-Tayeb et al., Hepatology, 2010, 51:297-305 and Asgari et al., Stem Cell Rev., 2013, 9(4):493-504, all of which are incorporated herein by reference in their entirety and specifically for the methodologies and reagents for differentiation. Differentiation can be assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

In some embodiments, the NK cells do not activate an immune response in the patient (e.g., recipient upon administration). Provided are methods of treating a disorder by administering a population of NK cells to a subject (e.g., recipient) or patient in need thereof. In some embodiments, the NK cells described herein comprise NK cells engineered (e.g., are modified) to express a chimeric antigen receptor including but not limited to a chimeric antigen receptor described herein. Any suitable CAR can be included in the NK cells, including the CARs described herein. In some embodiments, the NK cell includes a polynucleotide encoding a CAR, wherein the polynucleotide is inserted in a genomic locus. In some embodiments, the polynucleotide is inserted into a safe harbor or a target locus. In some embodiments, the polynucleotide is inserted in a B2M, CIITA, TRAC, TRB, PD1 or CTLA4 gene. Any suitable method can be used to insert the CAR into the genomic locus of the NK cell including the gene editing methods described herein (e.g., a CRISPR/Cas system).

U. Exogenous Polynucleotides

In some embodiments, the hypoimmunogenic cells provided herein are genetically modified to include one or more exogenous polynucleotides inserted into one or more genomic loci of the hypoimmunogenic cell. In some embodiments, the exogenous polynucleotide encodes a protein of interest, e.g., a chimeric antigen receptor. Any suitable method can be used to insert the exogenous polynucleotide into the genomic locus of the hypoimmunogenic cell including the gene editing methods described herein (e.g., a CRISPR/Cas system).

The exogenous polynucleotide can be inserted into any suitable genomic loci of the hypoimmunogenic cell. In some embodiments, the exogenous polynucleotide is inserted into a safe harbor or a target locus as described herein. Suitable safe harbor and target loci include, but are not limited to, a CCR5 gene, a CXCR4 gene, a PPP1R12C (also known as AAVS1) gene, an albumin gene, a SHS231 locus, a CLYBL gene, a Rosa gene (e.g., ROSA26), an F3 gene (also known as CD142), a MICA gene, a MICB gene, a LRP1 gene (also known as CD91), a HMGB1 gene, an ABO gene, a RHD gene, a FUT1 gene, a PDGFRa gene, an OLIG2 gene, a GFAP gene, and a KDM5D gene (also known as HY). In some embodiments, the exogenous polynucleotide is interested into an intron, exon, or coding sequence region of the safe harbor or target gene locus. In some embodiments, the exogenous polynucleotide is inserted into an endogenous gene wherein the insertion causes silencing or reduced expression of the endogenous gene. In some embodiments, the polynucleotide is inserted in a B2M, CIITA, TRAC, TRB, PD1 or CTLA4 gene locus. Exemplary genomic loci for insertion of an exogenous polynucleotide are depicted in Tables 4 and 5.

TABLE 4
Exemplary genomic loci for insertion of exogenous polynucleotides

| | | | | Target region | Also |
|---|---|---|---|---|---|
| Number | species | name | Ensembl ID | for cleavage | known as |
| 1 | human | B2M | ENSG00000166710 | CDS | |
| 2 | human | CIITA | ENSG00000179583 | CDS | |
| 3 | human | TRAC | ENSG00000277734 | CDS | |
| 4 | human | PPP1R12C | ENSG00000125503 | Intron 1 | AAVS1 |
| | | | | and 2 | |
| 5 | human | CLYBL | ENSG00000125246 | Intron 2 | |
| 6 | human | CCR5 | ENSG00000160791 | Exons 1-3, | |
| | | | | introns 1-2, | |
| | | | | and CDS | |
| 7 | human | THUMPD3- | ENSG00000206573 | Intron 1 | ROSA26 |
| | | AS1 | | | |
| 8 | human | Ch-4: | | 500 bp | SHS231 |
| | | 58,976,613 | | window | |
| 9 | human | F3 | ENSG00000117525 | CDS | CD142 |
| 10 | human | MICA | ENSG00000204520 | CDS | |
| 11 | human | MICB | ENSG00000204516 | CDS | |
| 12 | human | LRP1 | ENSG00000123384 | CDS | |
| 13 | human | HMGB1 | ENSG00000189403 | CDS | |
| 14 | human | ABO | ENSG00000175164 | CDS | |
| 15 | human | RHD | ENSG00000187010 | CDS | |
| 16 | human | FUT1 | ENSG00000174951 | CDS | |
| 17 | human | KDM5D | ENSG00000012817 | CDS | HY |

TABLE 5
Non-limiting examples of Cas9 guide RNAs

| | SEQ ID | | | Target | |
|---|---|---|---|---|---|
| Gene | NO: | guide sequence | PAM | site | gRNA cut location |
| ABO | 1 | UCUCUCCAUGUGCAGUAGGA | AGG | Exon | chr9:133,257,541 |
| | | | | 7 | |
| FUT1 | 2 | CUGGAUGUCGGAGGAGUACG | CGG | Exon | chr19:48,750,822 |
| | | | | 4 | |
| RH | 3 | GUCUCCGGAAACUCGAGGUG | AGG | Exon | chr1:25,284,622 |
| | | | | 2 | |
| F3 | 4 | ACAGUGUAGACUUGAUUGAC | GGG | Exon | chr1:94,540,281 |
| (CD142) | | | | 2 | |
| B2M | 5 | CGUGAGUAAACCUGAAUCUU | TGG | Exon | chr15:44,715,434 |
| | | | | 2 | |
| CITTA | 6 | GAUAUUGGCAUAAGCCUCCC | TGG | Exon | chr16:10,895,747 |
| | | | | 3 | |
| TRAC | 7 | AGAGUCUCUCAGCUGGUACA | CGG | Exon | chr14:22,5547,533 |
| | | | | 1 | |

For the Cas9 guides, the spacer sequence for all Cas9 guides is provided in Table 6. with description that the 20nt guide sequence corresponds to a unique guide sequence and can be any of those described herein, including for example those listed in Table 6.

TABLE 6
Cas9 guide RNAs

| | SEQ | |
|---|---|---|
| | ID | |
| Description | NO: | Sequence |
| 20 nt guide | 8 | NNNNNNNNNNNNNNNNNNNN |
| sequence* | | |
| 12 nt crRNA | 9 | GUUUUAGAGCUA |
| repeat sequence | | |
| 4 nt tetraloop | | GAAA |
| sequence | | |
| 64 nt tracrRNA | 10 | UAGCAAGUUAAAAUAAGGCUAGUCCG |
| sequence | | UUAUCAACUUGAAAAAGUGGCACCGA |
| | | GUCGGUGCUUU |
| Exemplary full | 11 | NNNNNNNNNNNNNNNNNNNNGUUUUA |
| sequence | | GAGCUAGAAAUAGCAAGUUAAAAUAA |
| | | GGCUAGUCCGUUAUCAACUUGAAAAA |
| | | GUGGCACCGAGUCGGUGCUUU |

In some embodiments, the hypoimmunogenic cell that includes the exogenous polynucleotide is derived from a hypoimmunogenic pluripotent cell (HIP), for example, as described herein. Such hypoimmunogenic cells include, for example, cardiac cells, neural cells, cerebral endothelial cells, dopaminergic neurons, glial cells, endothelial cells, thyroid cells, pancreatic islet cells (beta cells), retinal pigmented epithelium cells, and T cells. In some embodiments, the hypoimmunogenic cell that includes the exogenous polynucleotide is a pancreatic beta cell, a T cell (e.g., a primary T cell), or a glial progenitor cell.

In some embodiments, the exogenous polynucleotide encodes an exogenous CD47 polypeptide (e.g., a human CD47 polypeptide) and the exogenous polypeptide is inserted into a safe harbor or target gene loci or a safe harbor or target site as disclosed herein or a genomic locus that causes silencing or reduced expression of the endogenous gene. In some embodiments, the polynucleotide is inserted in a B2M, CIITA, TRAC, TRB, PD1 or CTLA4 gene locus. In some embodiments, the gene encoding CD47 is inserted into the specific locus selected from the group consisting of a safe harbor locus, a target locus, a B2M locus, a CIITA locus, a TRAC locus, a TRB locus, a PD1 locus and a CTLA4 locus. In some embodiments, the gene encoding the CAR is inserted into the specific locus selected from the group consisting of a safe harbor locus, a target locus, a B2M locus, a CIITA locus, a TRAC locus and a TRB locus. In some embodiments, the gene encoding CD47 and the gene encoding the CAR are inserted into different loci. In some embodiments, the gene encoding CD47 and the gene encoding the CAR are inserted into the same locus. In some embodiments, the gene encoding CD47 and the gene encoding the CAR are inserted into the B2M locus, the CIITA locus, the TRAC locus, the TRB locus, or the safe harbor or target locus. In some embodiments, the safe harbor or target locus is selected from the group consisting of a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene, locus a MICB gene, locus a LRP1 (CD91) gene locus, a HMGB1 gene locus, an ABO gene locus, ad RHD gene locus, a FUT1 locus, a PDGFRa gene locus, an OLIG2 gene locus, a GFAP gene locus, and a KDM5D gene locus).

In some embodiments, the hypoimmunogenic cell that includes the exogenous polynucleotide is a primary T cell or a T cell derived from a hypoimmunogenic pluripotent cell (e.g., a hypoimmunogenic iPSC). In exemplary embodiments, the exogenous polynucleotide is a chimeric antigen receptor (e.g., any of the CARs described herein). In some embodiments, the exogenous polynucleotide is operably linked to a promoter for expression of the exogenous polynucleotide in the hypoimmunogenic cell.

In some embodiments, the hypoimmunogenic cell the hypoimmunogenic cell that includes the exogenous polynucleotide is a primary T cell or a T cell derived from a hypoimmunogenic pluripotent cell (e.g., a hypoimmunogenic iPSC) and includes a first exogenous polynucleotide that encodes a CAR polypeptide and a second exogenous polynucleotide that encodes a CD47 polypeptide. In some embodiments, the first exogenous polynucleotide and the second exogenous polynucleotide are inserted into the same genomic locus. In some embodiments, the first exogenous polynucleotide and the second exogenous polynucleotide are inserted into different genomic loci. In exemplary embodiments, the hypoimmunogenic cell is a primary T cell or a T cell derived from a hypoimmunogenic pluripotent cell (e.g., an iPSC).

In some embodiments, the hypoimmunogenic cell that includes the exogenous polynucleotide is a primary NK cell or a NK cell derived from a hypoimmunogenic pluripotent cell (e.g., a hypoimmunogenic iPSC). In exemplary embodiments, the exogenous polynucleotide is a chimeric antigen receptor (e.g., any of the CARs described herein). In some embodiments, the exogenous polynucleotide is operably linked to a promoter for expression of the exogenous polynucleotide in the hypoimmunogenic cell. In some embodiments, the hypoimmunogenic cell the hypoimmunogenic cell that includes the exogenous polynucleotide is a primary NK cell or a NK cell derived from a hypoimmunogenic pluripotent cell (e.g., a hypoimmunogenic iPSC) and includes a first exogenous polynucleotide that encodes a CAR polypeptide and a second exogenous polynucleotide that encodes a CD47 polypeptide. In some embodiments, the first exogenous polynucleotide and the second exogenous polynucleotide are inserted into the same genomic locus. In some embodiments, the first exogenous polynucleotide and the second exogenous polynucleotide are inserted into different genomic loci. In exemplary embodiments, the hypoimmunogenic cell is a primary NK cell or a NK cell derived from a hypoimmunogenic pluripotent cell (e.g., an iPSC).

In some embodiments, the hypoimmunogenic cell includes 1, 2, 3, 4, 5, 6, 7, 8, 9, 10 or more different exogenous polynucleotides inserted one or more genomic loci as described herein (e.g., Table 4). In some embodiments, the exogenous polynucleotides are inserted into the same genomic loci. In some embodiments, the exogenous polynucleotides are inserted into different genomic loci.

In some embodiments, the exogenous polynucleotides encode for one of the following factors: DUX4, CD24, CD27, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, Serpinb9, and any of the tolerogenic factors provided herein.

V. Transplantation of Cells

As will be appreciated by those in the art, the cells and derivatives thereof can be transplanted using techniques known in the art that depends on both the cell type and the ultimate use of these cells. In general, the cells described herein can be transplanted either intravenously or by injection at particular locations in the patient. When transplanted at particular locations, the cells may be suspended in a gel matrix to prevent dispersion while they take hold.

W. Immunosuppressive Agents

In some embodiments, an immunosuppressive and/or immunomodulatory agent is not administered to the patient before the first administration of the population of hypoimmunogenic cells. In many embodiments, an immunosuppressive and/or immunomodulatory agent is administered to the patient before the first administration of the population of hypoimmunogenic cells. In some embodiments, an immunosuppressive and/or immunomodulatory agent is administered at least 1, 2, 3, 4, 5, 6, 7, 8, 9, 10, 11, 12, 13, 14 days or more before the first administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first administration of the cells. In particular embodiments, an immunosuppressive and/or immunomodulatory agent is not administered to the patient after the first administration of the cells, or is administered at least 1, 2, 3, 4, 5, 6, 7, 8, 9, 10, 11, 12, 13, 14 days or more after the first administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first administration of the cells. Non-limiting examples of an immunosuppressive and/or immunomodulatory agent include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-a and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-.alpha., IL-4, IL-5, IL-6R, IL-6, IGF, IGFR1, IL-7, IL-8, IL-10, CD11a, or CD58, and antibodies binding to any of their ligands. In some embodiments where an immunosuppressive and/or immunomodulatory agent is administered to the patient before or after the first administration of the cells, the administration is at a lower dosage than would be required for cells with MHC I and/or MHC II expression and without exogenous expression of CD47.

In one embodiment, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents.

In some embodiments, an immunosuppressive and/or immunomodulatory agent is not administered to the patient before the administration of the population of hypoimmunogenic cells. In many embodiments, an immunosuppressive and/or immunomodulatory agent is administered to the patient before the first and/or second administration of the population of hypoimmunogenic cells. In some embodiments, an immunosuppressive and/or immunomodulatory agent is administered at least 1, 2, 3, 4, 5, 6, 7, 8, 9, 10, 11, 12, 13, 14 days or more before the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first and/or second administration of the cells. In particular embodiments, an immunosuppressive and/or immunomodulatory agent is administered at least 1, 2, 3, 4, 5, 6, 7, 8, 9, 10, 11, 12, 13, 14 days or more after the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first and/or second administration of the cells. In some embodiments where an immunosuppressive and/or immunomodulatory agent is administered to the patient before or after the administration of the cells, the administration is at a lower dosage than would be required for cells with MHC I and/or MHC II expression and without exogenous expression of CD47.

### IV. Detailed Embodiments

In one aspect, provided herein is a method comprising administering to a patient a population of hypoimmunogenic cells comprising exogenous CD47 polypeptides and reduced expression of MHC class I and/or class II human leukocyte antigens, wherein the patient is sensitized against one or more alloantigens. In some embodiments, the method is for treating a disorder in the patient.

In some embodiments, the patient is sensitized from a previous pregnancy or a previous allogeneic transplant. In some embodiments, the one or more alloantigens comprise human leukocyte antigens. In some embodiments, the patient exhibits memory B cells and/or memory T cells reactive against the one or more alloantigens. In some embodiments, the allogeneic transplant is selected from the group consisting of an allogeneic cell transplant, an allogeneic blood transfusion, an allogeneic tissue transplant, and an allogeneic organ transplant. In some embodiments, the patient exhibits a reduced or no immune response to the population of hypoimmunogenic cells. In some instances, the patient exhibits an immune response to an allogeneic transplant and exhibits a reduced or no immune response to the population of hypoimmunogenic cells. In some embodiments, the reduced or no immune response is selected from the group consisting of reduced or no systemic immune response, reduced or no adaptive immune response, reduced or no innate immune response, reduced or no T cell response, and reduced or no B cell response to the population of hypoimmunogenic cells.

In some embodiments, the population of the hypoimmunogenic cells is administered at least 1 week or more after the patient is sensitized against one or more alloantigens. In certain embodiments, the population of the hypoimmunogenic cells is administered at least 1 month or more after the patient is sensitized against one or more alloantigens.

In some embodiments, the hypoimmunogenic cells comprise reduced expression of MHC class I and class II human leukocyte antigens. In some embodiments, the hypoimmunogenic cells comprise the exogenous CD47 polypeptides and reduced expression of B2M and/or CIITA. In some embodiments, the hypoimmunogenic cells comprise the exogenous CD47 polypeptides and reduced expression of B2M and CIITA. In some embodiments, the hypoimmunogenic cells further comprise one or more exogenous polypeptides selected from the group consisting of DUX4, CD24, CD46, CD55, CD59, CD200, PD-L1, HLA-E, HLA-G, IDO1, FasL, IL-35, IL-39, CCL21, CCL22, Mfge8, Serpin B9, and/or a combination thereof. In some embodiments, the hypoimmunogenic cells further comprise reduced expression levels of CD142.

In some embodiments, the hypoimmunogenic cells are differentiated cells derived from pluripotent stem cells. In some embodiments, the pluripotent stem cells comprise induced pluripotent stem cells. In some embodiments, the differentiated cells are selected from the group consisting of cardiac cells, neural cells, endothelial cells, T cells, B cells, pancreatic islet cells, retinal pigmented epithelium cells, hepatocytes, thyroid cells, skin cells, blood cells (e.g., plasma cells or platelets), and epithelial cells.

In some embodiments, the hypoimmunogenic cells comprise cells derived from primary T cells. In some embodiments, the cells derived from primary T cells are derived from a pool of T cells comprising primary T cells from one or more (e.g., two or more, three or more, four or more, five or more, ten or more, twenty or more, fifty or more, or one hundred or more) subjects different from the patient.

In some embodiments, the cells derived from primary T cells comprise a chimeric antigen receptor. In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments of a CAR, the antigen binding domain is selected from the group consisting of: (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the antigen binding domain of the CAR is selected from the group consisting of an antibody, an antigen-binding portion thereof, an scFv, and a Fab. In some embodiments, the antigen binding domain binds to CD19 or BCMA.

In some embodiments, the transmembrane domain of the CAR comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments, the signaling domain(s) of the CAR comprises a costimulatory domain(s). In some embodiments, the costimulatory domains comprise two costimulatory domains that are not the same. In some embodiments, the costimulatory domain(s) enhances cytokine production, CAR-T cell proliferation, and/or CAR-T cell persistence during T cell activation.

For a fourth generation CAR comprising a domain which upon successful signaling of the CAR induces expression of a cytokine gene, in some embodiments, the cytokine gene is an endogenous or exogenous cytokine gene to the hypoimmunogenic cells. In some embodiments, the cytokine gene encodes a pro-inflammatory cytokine. In some embodiments, the pro-inflammatory cytokine is selected from the group consisting of IL-1, IL-2, IL-9, IL-12, IL 18, TNF, IFN-gamma, and a functional fragment thereof.

In some embodiments of a fourth generation CAR, the domain which upon successful signaling of the CAR induces expression of the cytokine gene comprises a transcription factor or functional domain or fragment thereof.

In some embodiments of the cells derived from primary T cells, the CAR comprises a CD3 zeta (CD3ζ) domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof, and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof, and (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof, (iii) a 4-1BB domain, or a CD134 domain, or functional variant thereof, and (iv) a cytokine or costimulatory ligand transgene. In certain embodiments, the CAR comprises a (i) an anti-CD19 scFv; (ii) a CD8α hinge and transmembrane domain or functional variant thereof; (iii) a 4-1BB costimulatory domain or functional variant thereof, and (iv) a CD3ζ signaling domain or functional variant thereof.

In some embodiments, the cells derived from primary T cells comprise reduced expression of an endogenous T cell receptor. In particular embodiments, the cells derived from primary T cells comprise reduced expression of cytotoxic T-lymphocyte-associated protein 4 (CTLA4) and/or programmed cell death (PD1). In certain embodiments, the cells derived from primary T cells comprise increased expression of programmed cell death ligand 1 (PD-L1).

In some embodiments of the method, the population of hypoimmunogenic cells elicits a reduced level of immune activation or no immune activation in the patient upon administration. In certain embodiments, the population of hypoimmunogenic cells elicits a reduced level of systemic TH1 activation or no systemic TH1 activation in the patient upon administration. In some embodiments, the population of hypoimmunogenic cells elicits a reduced level of immune activation of peripheral blood mononuclear cells (PBMCs) or no immune activation of PBMCs in the patient upon administration. In particular embodiments, the population of hypoimmunogenic cells elicits a reduced level of donor-specific IgG antibodies or no donor specific IgG antibodies against the hypoimmunogenic cells in the patient upon administration. In some embodiments, the population of hypoimmunogenic cells elicits a reduced level of IgM and IgG antibody production or no IgM and IgG antibody production against the hypoimmunogenic cells in the patient upon administration. In other embodiments, the population of hypoimmunogenic cells elicits a reduced level of cytotoxic T cell killing or no cytotoxic T cell killing of the hypoimmunogenic cells in the patient upon administration. In certain embodiments, the population of hypoimmunogenic cells does not trigger a systemic acute cellular immune response in the patient upon administration.

In some embodiments, the patient is not administered an immunosuppressive agent at least 3 days or more before or after the administration of the population of hypoimmunogenic cells.

In another aspect, provided herein is method comprising administering to a patient a dosing regimen comprising: (a) a first administration comprising a therapeutically effective amount of hypoimmunogenic cells; (b) a recovery period; and (c) a second administration comprising a therapeutically effective amount of hypoimmunogenic cells; wherein the hypoimmunogenic cells comprise exogenous CD47 polypeptides and reduced expression of MHC class I and/or class II human leukocyte antigens, and wherein the patient is sensitized against one or more alloantigens. In some embodiments, the method is useful for treating a disorder in the patient.

In some embodiments, the patient is sensitized from a previous pregnancy or a previous allogeneic transplant. In some embodiments, the one or more alloantigens comprise human leukocyte antigens. In some embodiments, the patient exhibits memory B cells and/or memory T cells reactive against the one or more alloantigens. In some embodiments, the allogeneic transplant is selected from the group consisting of an allogeneic cell transplant, an allogeneic blood transfusion, an allogeneic tissue transplant, and an allogeneic organ transplant.

In some embodiments, the patient exhibits a reduced or no immune response to the population of hypoimmunogenic cells. In some instances, the reduced or no immune response is selected from the group consisting of reduced or no systemic immune response, reduced or no adaptive immune response, reduced or no innate immune response, reduced or no T cell response, and reduced or no B cell response to the population of hypoimmunogenic cells.

In some embodiments, the first administration of hypoimmunogenic cells occurs at least 1 week or more after the patient is sensitized against one or more alloantigens. In some embodiments, the first administration of hypoimmunogenic cells occurs at least 1 month or more after the patient is sensitized against one or more alloantigens.

In some embodiments, the hypoimmunogenic cells further comprise reduced expression of MHC class I and II human leukocyte antigens. In some embodiments, the hypoimmunogenic cells express the exogenous CD47 polypeptide and reduced expression of B2M and/or CIITA. In some embodiments, the hypoimmunogenic cells express the exogenous CD47 polypeptide and reduced expression of B2M and CIITA. In some embodiments, the hypoimmunogenic cells further comprise one or more exogenous polypeptides selected from the group consisting of DUX4, CD24, CD46, CD55, CD59, CD200, PD-L1, HLA-E, HLA-G, IDO1, FasL, IL-35, IL-39, CCL21, CCL22, Mfge8, Serpin B9, and a combination thereof. In some embodiments, the hypoimmunogenic cells further comprise reduced expression levels of CD142.

In some embodiments, the hypoimmunogenic cells are differentiated cells derived from pluripotent stem cells. In certain embodiments, the pluripotent stem cells comprise induced pluripotent stem cells. In many embodiments, the differentiated cells are selected from the group consisting of cardiac cells, neural cells, endothelial cells, T cells, B cells, pancreatic islet cells, retinal pigmented epithelium cells, hepatocytes, thyroid cells, skin cells, blood cells (e.g., plasma cells or platelets), and epithelial cells.

In some embodiments, the hypoimmunogenic cells comprise cells derived from primary T cells. In certain embodiments, the cells derived from primary T cells are derived from a pool of T cells comprising primary T cells from one or more (e.g., two or more, three or more, four or more, five or more, ten or more, twenty or more, fifty or more, or one hundred or more) subjects different from the patient. In some embodiments, the cells derived from primary T cells comprise a chimeric antigen receptor.

In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain is selected from the group consisting of: (a) an antigen binding domain targets an antigen characteristic of a neoplastic cell; (b) an antigen binding domain that targets an antigen characteristic of a T cell, (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell. In some embodiments, the antigen binding domain is selected from the group consisting of an antibody, an antigen-binding portion thereof, an scFv, and a Fab. In certain embodiments, the antigen binding domain binds to CD19 or BCMA.

In some embodiments, the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments, the signaling domain(s) comprises a costimulatory domain(s). In some embodiments, the costimulatory domains comprise two costimulatory domains that are not the same. In some embodiments, the costimulatory domain(s) enhances cytokine production, CAR-T cell proliferation, and/or CAR-T cell persistence during T cell activation.

In some embodiments of a fourth generation CAR, successful signaling of the CAR induces expression of a cytokine gene. In some embodiments, the cytokine gene is an endogenous or exogenous cytokine gene to the hypoimmunogenic cells. In some embodiments, the cytokine gene encodes a pro-inflammatory cytokine. In some embodiments, the pro-inflammatory cytokine is selected from the group consisting of IL-1, IL-2, IL-9, IL-12, IL 18, TNF, IFN-gamma, and a functional fragment thereof. In some embodiments of a fourth generation CAR, the domain which upon successful signaling of the CAR induces expression of the cytokine gene comprises a transcription factor or functional domain or fragment thereof.

In some embodiments, the CAR comprises a CD3 zeta (CD3ζ) domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof, and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof; and (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof, (iii) a 4-1BB domain, or a CD134 domain, or functional variant thereof, and (iv) a cytokine or costimulatory ligand transgene. In some embodiments, the CAR comprises a (i) an anti-CD19 scFv; (ii) a CD8α hinge and transmembrane domain or functional variant thereof (iii) a 4-1BB costimulatory domain or functional variant thereof and (iv) a CD3ζ signaling domain or functional variant thereof.

In some embodiments, the cells derived from primary T cells comprise reduced expression of an endogenous T cell receptor. In some embodiments, the cells derived from primary T cells comprise reduced expression of cytotoxic T-lymphocyte-associated protein 4 (CTLA4) and/or programmed cell death (PD1). In some embodiments, the cells derived from primary T cells comprise increased expression of programmed cell death ligand 1 (PD-L1).

In some embodiments, the recovery period comprises at least 1 month or more (e.g., at least 1 month, 2 months, 3 months, 4 months, or more). In some embodiments, the recovery period comprises at least 2 months or more (e.g., at least 2 months, 3 months, 4 months, or more).

In some embodiments, the second administration of cells is initiated when the hypoimmunogenic cells from the first administration are no longer detectable in the patient.

In some embodiments, upon the first and/or second administrations (e.g., upon the first administration or the second administration or both the first and second administrations), the hypoimmunogenic cells elicit a reduced level of immune activation or no immune activation in the patient. In some embodiments, upon the first and/or second administrations, the hypoimmunogenic cells elicit a reduced level of systemic TH1 activation or no systemic TH1 activation in the patient. In some embodiments, upon the first and/or second administrations, the hypoimmunogenic cells elicit a reduced level of immune activation of peripheral blood mononuclear cells (PBMCs) or no immune activation of PBMCs in the patient. In some embodiments, upon the first and/or second administrations, the hypoimmunogenic cells elicit a reduced level of donor-specific IgG antibodies or no donor-specific IgG antibodies against the hypoimmunogenic cells in the patient. In some embodiments, upon the first and/or second administrations, the hypoimmunogenic cells elicit a reduced level of IgM and IgG antibody production or no IgM and IgG antibody production against the hypoimmunogenic cells in the patient. In some embodiments, upon the first and/or second administrations, the hypoimmunogenic cells elicit a reduced level of cytotoxic T cell killing or no cytotoxic T cell killing of the hypoimmunogenic cells in the patient.

In some embodiments, the patient is not administered an immunosuppressive agent at least 3 days or more before or after the first administration of the hypoimmunogenic cells. In some embodiments, the patient is not administered an immunosuppressive agent at least 3 days or more before or after the second administration of the hypoimmunogenic cells. In certain embodiments, the patient is not administered an immunosuppressive agent during the recovery period.

In some embodiments, method described further comprises administering the dosing regimen at least twice. In certain instances, the dosing regimen is administered at least 2 times (e.g., at least 2, 3, 4, or more times) to a patient who is sensitized against one or more alloantigens.

Provided here is the use of a population of hypoimmunogenic cells comprising exogenous CD47 polypeptides and reduced expression of MHC class I and/or class II human leukocyte antigens for treatment of a disorder in a patient, wherein the patient is sensitized against one or more alloantigens.

Provided here is the use of a population of hypoimmunogenic cells comprising exogenous CD47 polypeptides and reduced expression of MHC class I and class II human leukocyte antigens for treatment of a disorder in a patient, wherein the patient is sensitized against one or more alloantigens.

Provided here is the use of a population of hypoimmunogenic cells comprising exogenous CD47 polypeptides and reduced levels of B2M and CIITA polypeptides for treatment of a disorder in a patient, wherein the patient is sensitized against one or more alloantigens.

Provided here is the use of a population of hypoimmunogenic cells comprising exogenous CD47 polypeptides, a genomic modification of the B2M gene, and a genomic modification of the CIITA gene for treatment of a disorder in a patient, wherein the patient is sensitized against one or more alloantigens.

In some embodiments of the uses of the population of cells, the one or more alloantigens comprise human leukocyte antigens. In some embodiments, the patient exhibits memory B cells and/or memory T cells reactive against the one or more alloantigens.

In some embodiments of the uses described, the patient is sensitized from a previous pregnancy or a previous allogeneic transplant. In some embodiments, the allogeneic transplant is selected from the group consisting of an allogeneic cell transplant, an allogeneic blood transfusion, an allogeneic tissue transplant, and an allogeneic organ transplant.

In some embodiments, the patient exhibits a reduced or no immune response to the population of hypoimmunogenic cells. In certain embodiments, the reduced or no immune response is selected from the group consisting of reduced or no systemic immune response, reduced or no adaptive immune response, reduced or no innate immune response, reduced or no T cell response, and reduced or no B cell response to the population of hypoimmunogenic cells.

In some embodiments, the hypoimmunogenic cells further comprise one or more exogenous polypeptides selected from the group consisting of DUX4, CD24, CD46, CD55, CD59, CD200, PD-L1, HLA-E, HLA-G, IDO1, FasL, IL-35, IL-39, CCL21, CCL22, Mfge8, Serpin B9, and a combination thereof. In certain embodiments, the hypoimmunogenic cells further comprise a genomic modification of the CD142 gene.

In some embodiments, the population of hypoimmunogenic cells comprises differentiated cells derived from pluripotent stem cells. In some embodiments, the pluripotent stem cells comprise induced pluripotent stem cells. In some embodiments, the differentiated cells are selected from the group consisting of cardiac cells, neural cells, endothelial cells, T cells, B cells, pancreatic islet cells, retinal pigmented epithelium cells, hepatocytes, thyroid cells, skin cells, blood cells (e.g., plasma cells or platelets), and epithelial cells.

In some embodiments, the population of hypoimmunogenic cells comprises cells derived from primary T cells. In some embodiments, the cells derived from primary T cells are derived from a pool of T cells comprising primary T cells from one or more (e.g., two or more, three or more, four or more, five or more, ten or more, twenty or more, fifty or more, or one hundred or more) subjects different from the patient. In some embodiments, the cells derived from primary T cells comprise a chimeric antigen receptor (CAR).

In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain is selected from the group consisting of: (a) an antigen binding domain targets an antigen characteristic of a neoplastic cell; (b) an antigen binding domain that targets an antigen characteristic of a T cell, (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments of a CAR, the antigen binding domain is selected from the group consisting of an antibody, an antigen-binding portion thereof, an scFv, and a Fab. In some embodiments, the antigen binding domain binds to CD19 or BCMA.

In some embodiments of a CAR, the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments of a CAR, the signaling domain(s) comprises a costimulatory domain(s). In some embodiments, the costimulatory domains comprise two costimulatory domains that are not the same. In some embodiments, the costimulatory domain(s) enhances cytokine production, CAR-T cell proliferation, and/or CAR-T cell persistence during T cell activation.

As described of a fourth generation CAR, successful signaling of the CAR induces expression of a cytokine gene. In some embodiments, the cytokine gene is an endogenous or exogenous cytokine gene to the hypoimmunogenic cells. In some embodiments, the cytokine gene encodes a pro-inflammatory cytokine. In some embodiments, the pro-inflammatory cytokine is selected from the group consisting of IL-1, IL-2, IL-9, IL-12, IL 18, TNF, IFN-gamma, and a functional fragment thereof. In some embodiments of a fourth generation CAR, the domain which upon successful signaling of the CAR induces expression of the cytokine gene comprises a transcription factor or functional domain or fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8α hinge and transmembrane domain or functional variant thereof (iii) a 4-1BB costimulatory domain or functional variant thereof and (iv) a CD3ζ signaling domain or functional variant thereof.

In some embodiments, the cells derived from primary T cells comprise reduced expression of an endogenous T cell receptor. In some embodiments, the cells derived from primary T cells comprise reduced expression of cytotoxic T-lymphocyte-associated protein 4 (CTLA4) and/or programmed cell death (PD1). In some embodiments, the cells derived from primary T cells comprise increased expression of programmed cell death ligand 1 (PD-L1).

In one aspect, provided herein is a method comprising administering to a patient a population of hypoimmunogenic cells comprising exogenous CD47 polypeptides and reduced expression of MHC class I and/or class II human leukocyte antigens, wherein the patient had previously received an allogeneic transplant.

In some embodiments, the allogeneic transplant is selected from the group consisting of an allogeneic cell transplant, an allogeneic blood transfusion, an allogeneic tissue transplant, and an allogeneic organ transplant. In some embodiments, the patient exhibits memory B cells and/or memory T cells reactive against one or more alloantigens. In some embodiments, the one or more alloantigens comprise human leukocyte antigens.

In some embodiments, the patient exhibits a reduced or no immune response to the population of hypoimmunogenic cells. In some embodiments, the reduced or no immune response is selected from the group consisting of reduced or no systemic immune response, reduced or no adaptive immune response, reduced or no innate immune response, reduced or no T cell response, and reduced or no B cell response to the population of hypoimmunogenic cells.

In some embodiments, the population of the hypoimmunogenic cells is administered at least 1 week or more after the patient had received the allogeneic transplant. In particular embodiments, the population of the hypoimmunogenic cells is administered at least 1 month or more after the patient had received the allogeneic transplant.

In some embodiments, the hypoimmunogenic cells comprise reduced expression of MHC class I and class II human leukocyte antigens. In some embodiments, the hypoimmunogenic cells comprise the exogenous CD47 polypeptides and reduced expression of B2M and/or CIITA. In some embodiments, the hypoimmunogenic cells comprise the exogenous CD47 polypeptides and reduced expression of B2M and CIITA. In some embodiments, the hypoimmunogenic cells further comprise one or more exogenous polypeptides selected from the group consisting of DUX4, CD24, CD46, CD55, CD59, CD200, PD-L1, HLA-E, HLA-G, IDO1, FasL, IL-35, IL-39, CCL21, CCL22, Mfge8, Serpin B9, and a combination thereof. In some embodiments, the hypoimmunogenic cells further comprise reduced expression levels of CD142.

In some embodiments, the hypoimmunogenic cells are differentiated cells derived from pluripotent stem cells. In some embodiments, the pluripotent stem cells comprise induced pluripotent stem cells. In some embodiments, the differentiated cells are selected from the group consisting of cardiac cells, neural cells, endothelial cells, T cells, B cells, pancreatic islet cells, retinal pigmented epithelium cells, hepatocytes, thyroid cells, skin cells, blood cells (e.g., plasma cells or platelets), and epithelial cells.

In some embodiments, the hypoimmunogenic cells comprise cells derived from primary T cells. In some embodiments, the cells derived from primary T cells are derived from a pool of T cells comprising primary T cells from one or more (e.g., two or more, three or more, four or more, five or more, ten or more, twenty or more, fifty or more, or one hundred or more) subjects different from the patient.

In some embodiments, the cells derived from primary T cells comprise a chimeric antigen receptor. In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain is selected from the group consisting of: (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell. In some embodiments, the antigen binding domain is selected from the group consisting of an antibody, an antigen-binding portion thereof, an scFv, and a Fab. In some embodiments, the antigen binding domain binds to CD19 or BCMA.

In some embodiments, the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments, the signaling domain(s) comprises a costimulatory domain(s). In some embodiments, the costimulatory domains comprise two costimulatory domains that are not the same. In some embodiments, the costimulatory domain(s) enhances cytokine production, CAR-T cell proliferation, and/or CAR-T cell persistence during T cell activation.

In some embodiments of a fourth generation CAR-That induces expression of a cytokine gene, the cytokine gene is an endogenous or exogenous cytokine gene to the hypoimmunogenic cells. In some embodiments, the cytokine gene encodes a pro-inflammatory cytokine. In some embodiments, the pro-inflammatory cytokine is selected from the group consisting of IL-1, IL-2, IL-9, IL-12, IL 18, TNF, IFN-gamma, and a functional fragment thereof.

In some embodiments of a fourth generation CAR, the domain which upon successful signaling of the CAR induces expression of the cytokine gene comprises a transcription factor or functional domain or fragment thereof.

In some embodiments, the CAR of the cells derived from primary T cells comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8α hinge and transmembrane domain or functional variant thereof; (iii) a 4-1BB costimulatory domain or functional variant thereof, and (iv) a CD3ζ signaling domain or functional variant thereof.

In some embodiments, the cells derived from primary T cells comprise reduced expression of an endogenous T cell receptor. In some embodiments, the cells derived from primary T cells comprise reduced expression of cytotoxic T-lymphocyte-associated protein 4 (CTLA4) and/or programmed cell death (PD1). In some embodiments, the cells derived from primary T cells comprise increased expression of programmed cell death ligand 1 (PD-L1).

In some embodiments, the population of hypoimmunogenic cells elicits a reduced level of immune activation or no immune activation in the patient upon administration. In some embodiments, the population of hypoimmunogenic cells elicits a reduced level of systemic TH1 activation or no systemic TH1 activation in the patient upon administration. In some embodiments, the population of hypoimmunogenic cells elicits a reduced level of immune activation of peripheral blood mononuclear cells (PBMCs) or no immune activation of PBMCs in the patient upon administration. In some embodiments, the population of hypoimmunogenic cells elicits a reduced level of donor-specific IgG antibodies or no donor specific IgG antibodies against the hypoimmunogenic cells in the patient upon administration. In some embodiments, the population of hypoimmunogenic cells elicits a reduced level of IgM and IgG antibody production or no IgM and IgG antibody production against the hypoimmunogenic cells in the patient upon administration. In some embodiments, the population of hypoimmunogenic cells elicits a reduced level of cytotoxic T cell killing or no cytotoxic T cell killing of the hypoimmunogenic cells in the patient upon administration. In some embodiments, the population of hypoimmunogenic cells does not trigger a systemic acute cellular immune response in the patient upon administration.

In some embodiments, the patient is not administered an immunosuppressive agent at least 3 days or more before or after the administration of the population of hypoimmunogenic cells.

In another aspect, provided is a method comprising administering to a patient a population of hypoimmunogenic cells comprising exogenous CD47 polypeptides and reduced expression of MHC class I and/or class II human leukocyte antigens, wherein the patient had previously exhibited alloimmunization in pregnancy. In some embodiments, the alloimmunization in pregnancy is hemolytic disease of the fetus and newborn (HDFN), neonatal alloimmune neutropenia (NAN) or fetal and neonatal alloimmune thrombocytopenia (FNAIT). In some embodiments, the method described is useful for treating a disorder in the patient.

In some embodiments, the patient exhibits a reduced or no immune response to the population of hypoimmunogenic cells. In some embodiments, the reduced or no immune response is selected from the group consisting of reduced or no systemic immune response, reduced or no adaptive immune response, reduced or no innate immune response, reduced or no T cell response, and reduced or no B cell response to the population of hypoimmunogenic cells.

In many embodiments, the hypoimmunogenic cells comprise reduced expression of MHC class I and class II human leukocyte antigens. In some embodiments, the hypoimmunogenic cells comprise the exogenous CD47 polypeptides and reduced expression of B2M and/or CIITA. In some embodiments, the hypoimmunogenic cells comprise the exogenous CD47 polypeptides and reduced expression of B2M and CIITA. In particular embodiments, the hypoimmunogenic cells further comprise one or more exogenous polypeptides selected from the group consisting of DUX4, CD24, CD46, CD55, CD59, CD200, PD-L1, HLA-E, HLA-G, IDO1, FasL, IL-35, IL-39, CCL21, CCL22, Mfge8, Serpin B9, and a combination thereof. In certain embodiments, the hypoimmunogenic cells further comprise reduced expression levels of CD142.

In some embodiments, the hypoimmunogenic cells are differentiated cells derived from pluripotent stem cells. In certain embodiments, the pluripotent stem cells comprise induced pluripotent stem cells.

In many embodiments, the differentiated cells are selected from the group consisting of cardiac cells, neural cells, endothelial cells, T cells, B cells, pancreatic islet cells, retinal pigmented epithelium cells, hepatocytes, thyroid cells, skin cells, blood cells (e.g., plasma cells or platelets), and epithelial cells.

In some embodiments, the hypoimmunogenic cells comprise cells derived from primary T cells. In certain embodiments, the cells derived from primary T cells are derived from a pool of T cells comprising primary T cells from one or more (e.g., two or more, three or more, four or more, five or more, ten or more, twenty or more, fifty or more, or one hundred or more) subjects different from the patient.

In some embodiments, the cells derived from primary T cells comprise a chimeric antigen receptor. In some embodiments, the chimeric antigen receptor (CAR) is selected from the group consisting of: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the antigen binding domain is selected from the group consisting of (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell. In some embodiments, the antigen binding domain is selected from the group consisting of an antibody, an antigen-binding portion thereof, an scFv, and a Fab. In some embodiments, the antigen binding domain binds to CD19 or BCMA.

In some embodiments, the transmembrane domain comprises one selected from the group consisting of a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments, the signaling domain(s) comprises a costimulatory domain(s). In some embodiments, the costimulatory domains comprise two costimulatory domains that are not the same. In some embodiments, the costimulatory domain(s) enhances cytokine production, CAR-T cell proliferation, and/or CAR-T cell persistence during T cell activation.

For fourth generation CARs that induce expression of a cytokine gene, in some embodiments, the cytokine gene is an endogenous or exogenous cytokine gene to the hypoimmunogenic cells. In some embodiments, the cytokine gene encodes a pro-inflammatory cytokine. In some embodiments, the pro-inflammatory cytokine is selected from the group consisting of IL-1, IL-2, IL-9, IL-12, IL 18, TNF, IFN-gamma, and a functional fragment thereof. In some embodiments, the domain of the CAR which upon successful signaling of the CAR induces expression of the cytokine gene comprises a transcription factor or functional domain or fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain or functional variant thereof.

In some embodiments, the cells derived from primary T cells comprise reduced expression of an endogenous T cell receptor. In some embodiments, the cells derived from primary T cells comprise reduced expression of cytotoxic T-lymphocyte-associated protein 4 (CTLA4) and/or programmed cell death (PD1). In some embodiments, the cells derived from primary T cells comprise increased expression of programmed cell death ligand 1 (PD-L1).

In some embodiments, the population of hypoimmunogenic cells elicits a reduced level of immune activation or no immune activation in the patient upon administration. In some embodiments, the population of hypoimmunogenic cells elicits a reduced level of systemic TH1 activation or no systemic TH1 activation in the patient upon administration. In some embodiments, the population of hypoimmunogenic cells elicits a reduced level of immune activation of peripheral blood mononuclear cells (PBMCs) or no immune activation of PBMCs in the patient upon administration. In some embodiments, the population of hypoimmunogenic cells elicits a reduced level of donor-specific IgG antibodies or no donor specific IgG antibodies against the hypoimmunogenic cells in the patient upon administration. In some embodiments, the population of hypoimmunogenic cells elicits a reduced level of IgM and IgG antibody production or no IgM and IgG antibody production against the hypoimmunogenic cells in the patient upon administration. In some embodiments, the population of hypoimmunogenic cells elicits a reduced level of cytotoxic T cell killing or no cytotoxic T cell killing of the hypoimmunogenic cells in the patient upon administration. In some embodiments, the population of hypoimmunogenic cells does not trigger a systemic acute cellular immune response in the patient upon administration.

In some embodiments, the patient is not administered an immunosuppressive agent at least 3 days or more before or after the administration of the population of hypoimmunogenic cells.

In one aspect, provided herein is a method of treating a sensitized patient having a cellular deficiency comprising administering to the patient a population of cells differentiated from stem cells comprising one or more hypoimmunogenic modifications.

In another aspect, provided herein is a method of treating a sensitized patient who is a candidate for a cellular therapy comprising administering to the patient a population of cells differentiated from stem cells comprising one or more hypoimmunogenic modifications.

In one aspect, provided herein is a method comprising administering to a patient who is a candidate for a cellular therapy a population of cells differentiated from stem cells comprising one or more hypoimmunogenic modifications, wherein the patient received a previous treatment for a condition or disease.

In one aspect, provided herein is a method of treating a sensitized patient who is a candidate for a cellular therapy comprising administering to the patient a population of cells differentiated from stem cells comprising one or more hypoimmunogenic modifications, wherein the patient is not administered an immunosuppressive agent before, during, or after the administration of the population of cells.

In one aspect, provided herein is a method of treating a patient having at least a partial organ failure in need thereof comprising administering to the patient a population of cells differentiated from stem cells comprising one or more hypoimmunogenic modifications prior to administering at least a partial organ transplant to the patient.

In another aspect, provided herein is a method of administering a tissue or organ transplant to a patient in need thereof comprising administering to the patient a population of cells differentiated from stem cells comprising one or more hypoimmunogenic modifications prior to administering the tissue or organ transplant.

In some embodiments, the patient is a sensitized patient. In certain embodiments, the patient is sensitized from a previous pregnancy or a previous transplant. In certain embodiments, the previous transplant is selected from the group consisting of a cell transplant, a blood transfusion, a tissue transplant, and an organ transplant. In some embodiments, the previous transplant is an allogeneic transplant.

In some embodiments, the previous transplant is a transplant selected from the group consisting of a chimera of human origin, a modified non-human autologous cell, a modified autologous cell, an autologous tissue, and an autologous organ. In some embodiments, the patient is sensitized against one or more alloantigens or one or more autologous antigens. In certain embodiments, the patient exhibits memory B cells and/or memory T cells reactive against the one or more alloantigens or one or more autologous antigens.

In some embodiments, the patient has an allergy. In certain embodiments, the allergy is an allergy selected from the group consisting of a hay fever, a food allergy, an insect allergy, a drug allergy, and atopic dermatitis.

In certain embodiments, the population of cells comprises cells that express exogenous CD47 polypeptides and have reduced expression of B2M and/or CIITA. In some embodiments, the population of cells is selected from the group consisting of cardiac cells, neural cells, endothelial cells, T cells, B cells, pancreatic islet cells, retinal pigmented epithelium cells, hepatocytes, thyroid cells, skin cells, blood cells, plasma cells, platelets, renal cells, epithelial cells, and chimeric antigen receptor (CAR) T cells.

In some embodiments, the patient exhibits a reduced or no immune response to the population of cells. In some embodiments, the reduced immune response is compared to the immune response in a patient or control subject administered a “wild-type” population of cells. In some embodiments, the reduced or no immune response to the population of cells response exhibited is selected from the group consisting of reduced or no systemic immune response, reduced or no adaptive immune response, reduced or no innate immune response, reduced or no T cell response, and reduced or no B cell response. In exemplary embodiments, the patient exhibits: a) a reduced level of systemic TH1 activation or no systemic TH1 activation upon administering the population of cells; b) a reduced level of immune activation of peripheral blood mononuclear cells (PBMCs) or no immune activation of PBMCs upon administering the population of cells; c) a reduced level of donor-specific IgG antibodies or no donor specific IgG antibodies against the population of cells upon administering the population of cells; d) a reduced level of IgM and IgG antibody production or no IgM and IgG antibody production against the population of cells upon administering the population of cells; and/or e) a reduced level of cytotoxic T cell killing or no cytotoxic T cell killing of the population of cells upon administering the population of cells.

In certain embodiments, the patient is not administered an immunosuppressive agent before the administration of the population of cells. In some embodiments, the population of cells is administered at least 1 day, at least 2 days, at least 3 days, at least 4 days, at least 5, days, at least 6 days, at least 1 week, or at least 1 month or more after the patient is sensitized.

In some embodiments, the stem cells are pluripotent stem cells. In certain embodiments, the pluripotent stem cells are induced pluripotent stem cells.

In some embodiments, the cellular deficiency is associated with a neurodegenerative disease or the cellular therapy is for the treatment of a neurodegenerative disease. In certain embodiments, the neurodegenerative disease is selected from the group consisting of leukodystrophy, Huntington's disease, Parkinson's disease, multiple sclerosis, transverse myelitis, and Pelizaeus-Merzbacher disease (PMD). In some embodiments, the population of cells comprises cells selected from the group consisting of glial progenitor cells, oligodendrocytes, astrocytes, and dopaminergic neurons. In certain embodiments, the dopaminergic neurons are selected from the group consisting of neural stem cells, neural progenitor cells, immature dopaminergic neurons, and mature dopaminergic neurons.

In some embodiments, the cellular deficiency is associated with diabetes or the cellular therapy is for the treatment of diabetes. In certain embodiments, the population of cells is a population of pancreatic islet cells, including pancreatic beta islet cells. In some embodiments, the pancreatic islet cells are selected from the group consisting of a pancreatic islet progenitor cell, an immature pancreatic islet cell, and a mature pancreatic islet cell.

In certain embodiments, the cellular deficiency is associated with a cardiovascular condition or disease or the cellular therapy is for the treatment of a cardiovascular condition or disease. In some embodiments, the population of cells is a population of cardiomyocytes.

In some embodiments, the cellular deficiency is associated with a vascular condition or disease or the cellular therapy is for the treatment of a vascular condition or disease. In some embodiments, the population of cells is a population of endothelial cells.

In some embodiments, the cellular deficiency is associated with autoimmune thyroiditis or the cellular therapy is for the treatment of autoimmune thyroiditis. In some embodiments, the population of cells is a population of thyroid progenitor cells.

In certain embodiments, the cellular deficiency is associated with a liver disease or the cellular therapy is for the treatment of liver disease. In some embodiments, the liver disease comprises cirrhosis of the liver.

In some embodiments, the population of cells is a population of hepatocytes or hepatic progenitor cells. In certain embodiments, the cellular deficiency is associated with a corneal disease or the cellular therapy is for the treatment of corneal disease. In some embodiments, the corneal disease is Fuchs dystrophy or congenital hereditary endothelial dystrophy. In some embodiments, the population of cells is a population of corneal endothelial progenitor cells or corneal endothelial cells.

In some embodiments, the cellular deficiency is associated with a kidney disease or the cellular therapy is for the treatment of a kidney disease. In some embodiments, the population of cells is a population of renal precursor cells or renal cells.

In certain embodiments, the cellular therapy is for the treatment of a cancer. In some embodiments, the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer. In some embodiments, the population of cells is a population of chimeric antigen receptor (CAR) T-cells.

In some embodiments, the previous treatment did not comprise the population of cells. In certain embodiments, the population of cells is administered for the treatment of the same condition or disease as the previous treatment. In some embodiments, the population of cells exhibits an enhanced therapeutic effect for the treatment of the condition or disease in the patient as compared to the previous treatment. In certain embodiments, the population of cells exhibits a longer therapeutic effect for the treatment of the condition or disease in the patient as compared to the previous treatment. In some embodiments, the population of cells is administered for the treatment of a different condition or disease as the previous treatment. In some embodiments, the previous treatment is therapeutically ineffective. In some embodiments, the patient developed an immune reaction against the previous treatment.

In some embodiments, the previous treatment comprises administering a population of therapeutic cells comprising a suicide gene safety switch system, and the immune reaction occurs in response to activation of the suicide gene safety switch system.

In some embodiments, the previous treatment comprises a mechanically assisted treatment. In exemplary embodiments, the mechanically assisted treatment comprises hemodialysis or a ventricle assist device.

In some embodiments, the tissue and/or organ transplant or partial organ transplant is selected from the group consisting of a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, a bone transplant, a partial lung transplant, a partial kidney transplant, a partial liver transplant, a partial pancreas transplant, a partial intestine transplant, and/or a partial cornea transplant. In some embodiments, the population of cells is administered for treatment of a cellular deficiency in a tissue or organ selected from the group consisting of heart, lung, kidney, liver, pancreas, intestine, stomach, cornea, bone marrow, blood vessel, heart valve, and/or bone.

In some embodiments, the tissue or organ transplant is an allograft transplant. In certain embodiments, the tissue or organ transplant is an autograft transplant. In some embodiments, the population of cells is administered for the treatment of a cellular deficiency in a tissue or organ and the tissue or organ transplant is for the replacement of the same tissue or organ.

In certain embodiments, the population of cells is administered for the treatment of a cellular deficiency in a tissue or organ and the tissue or organ transplant is for the replacement of a different tissue or organ. In some embodiments, the organ transplant is a kidney transplant and the population of cells is a population of pancreatic beta islet cells. In exemplary embodiments, the patient has diabetes.

In another aspect, provided herein is a method comprising administering to a patient a population of hypoimmunogenic cells. In this method, the hypoimmunogenic cells each comprise: a) an exogenous polynucleotide inserted into a genomic locus comprising a safe harbor locus, a target locus, a B2M gene locus, a CIITA gene locus, a TRAC gene locus, or a TRB gene locus; b) exogenous CD47 polypeptides; and c) reduced expression of MHC class I and/or class II human leukocyte antigens.

In one aspect, provided herein is a method comprising administering to a patient a dosing regimen. In this method, the dosing regimen comprises a) a first administration comprising a therapeutically effective amount of hypoimmunogenic cells; b) a recovery period; and c) a second administration comprising a therapeutically effective amount of hypoimmunogenic cells; wherein the hypoimmunogenic cells each comprise an exogenous polynucleotide inserted into a genomic locus comprising a B2M gene locus, a CIITA gene locus, a TRAC gene locus, or a TRB gene locus, and wherein the hypoimmunogenic cells each comprise exogenous CD47 polypeptides and reduced expression of MHC class I and/or class II human leukocyte antigens.

In one aspect, provided herein is the use of a population of hypoimmunogenic cells for treatment of a disease in a patient, wherein the hypoimmunogenic cells each comprise an exogenous polynucleotide inserted into a genomic locus comprising a safe harbor locus, a target locus, a B2M gene locus, a CIITA gene locus, a TRAC gene locus, or a TRB gene locus; and wherein the hypoimmunogenic cells each comprise exogenous CD47 polypeptides and reduced expression of MHC class I and/or class II human leukocyte antigens.

In one aspect, provided herein is a method comprising administering to a patient a population of hypoimmunogenic cells. In this method, the hypoimmunogenic cells each comprise: a) an exogenous polynucleotide inserted into a genomic locus comprising a safe harbor locus, a target locus, a B2M gene locus, a CIITA gene locus, a TRAC gene locus, or a TRB gene locus; b) exogenous CD47 polypeptides; and c) reduced expression of MHC class I and/or class II human leukocyte antigens, wherein the patient had previously received an allogeneic transplant.

In one aspect, provided herein is a method of treating a patient who is a candidate for a cellular therapy comprising administering to a patient a population of hypoimmunogenic cells. In this method, the hypoimmunogenic cells each comprise: a) an exogenous polynucleotide inserted into a genomic locus comprising a safe harbor locus, a target locus, a B2M gene locus, a CIITA gene locus, a TRAC gene locus, or a TRB gene locus; b) exogenous CD47 polypeptides; and c) reduced expression of MHC class I and/or class II human leukocyte antigens.

In one aspect, provided herein is a method comprising administering to a patient who is a candidate for a cellular therapy a population of hypoimmunogenic cells. In this method, the hypoimmunogenic cells each comprise: a) an exogenous polynucleotide inserted into a genomic locus comprising a safe harbor locus, a target locus, a B2M gene locus, a CIITA gene locus, a TRAC gene locus, or a TRB gene locus; b) exogenous CD47 polypeptides; and c) reduced expression of MHC class I and/or class II human leukocyte antigens, wherein the patient received a previous treatment for a condition or disease.

In one aspect, provided herein is a method of treating a patient who is a candidate for a cellular therapy comprising administering to the patient a population of hypoimmunogenic cells, wherein the hypoimmunogenic cells each comprise: a) an exogenous polynucleotide inserted into a genomic locus comprising a B2M gene locus, a CIITA gene locus, a TRAC gene locus, or a TRB gene locus; b) exogenous CD47 polypeptides; and c) reduced expression of MHC class I and/or class II human leukocyte antigens, wherein the patient is not administered an immunosuppressive agent before, during, or after the administration of the population of cells.

In another aspect, provided herein is a method of treating a patient having at least a partial organ failure in need thereof comprising administering to the patient a population of hypoimmunogenic cells. In this method, the hypoimmunogenic cells each comprise: a) an exogenous polynucleotide inserted into a genomic locus comprising a safe harbor locus, a target locus, a B2M gene locus, a CIITA gene locus, a TRAC gene locus, or a TRB gene locus; b) exogenous CD47 polypeptides; and c) reduced expression of MHC class I and/or class II human leukocyte antigens, wherein the population of hypoimmunogenic cells are administered prior to administering at least a partial organ transplant to the patient.

In yet another aspect, provided herein is a method of administering a tissue or organ transplant to a patient in need thereof comprising administering to the patient a population of hypoimmunogenic cells. In this method, the hypoimmunogenic cells each comprise: a) an exogenous polynucleotide inserted into a genomic locus comprising a safe harbor locus, a target locus, a B2M gene locus, a CIITA gene locus, a TRAC gene locus, or a TRB gene locus; and b) exogenous CD47 polypeptides, wherein the population of hypoimmunogenic cells are administered prior to administering the tissue or organ transplant.

In another aspect, provided herein is a method of administering to a patient a population of hypoimmunogenic cells. In this method, the hypoimmunogenic cells each comprise: a) a genetic modification comprising an exogenous polynucleotide encoding a chimeric antigen receptor (CAR) inserted into a genomic locus comprising a safe harbor locus, a target locus, a B2M gene locus, a CIITA gene locus, a TRAC gene locus, or a TRB gene locus; b) exogenous CD47 polypeptides; and c) reduced expression of MHC class I and/or class II human leukocyte antigens.

In another aspect, provided herein is a method of treating a cancer in need of a patient in need thereof comprising administering to the patient a population of hypoimmunogenic cells. The hypoimmunogenic cells each comprise: a) an exogenous polynucleotide encoding a chimeric antigen receptor (CAR) inserted into a genomic locus comprising a safe harbor locus, a target locus, a B2M gene locus, a CIITA gene locus, a TRAC gene locus, or a TRB gene locus; b) exogenous CD47 polypeptides; and c) reduced expression of MHC class I and/or class II human leukocyte antigens.

In some embodiments, the hypoimmunogenic cells comprise an additional exogenous polynucleotide encoding for the exogenous CD47 polypeptides. In certain embodiments, the additional exogenous polynucleotide is i) located at a different genomic locus the genomic locus in (a); or ii) located at the same genomic locus as the genomic locus in (a).

In another aspect, provided herein is method comprising administering to a patient a population of hypoimmunogenic cells. In this method, the hypoimmunogenic cells each comprise: a) a first exogenous polynucleotide encoding a chimeric antigen receptor (CAR) inserted into a first genomic locus; and b) a second exogenous polynucleotide encoding a CD47 polypeptide inserted into a second genomic locus, wherein the hypoimmunogenic cells exhibit reduced expression of MHC class I and/or class II human leukocyte antigens, wherein the first and second genomic loci are each a safe harbor locus, a target locus, a B2M gene locus, a CIITA gene locus, a TRAC gene locus, or a TRB gene locus.

In one aspect, provided herein is a method of treating a cancer in need of a patient in need thereof comprising administering to the patient a population of hypoimmunogenic cells. In this method, the hypoimmunogenic cells each comprise: a) a first exogenous polynucleotide encoding a chimeric antigen receptor (CAR) inserted into a first genomic locus; and b) a second exogenous polynucleotide encoding a CD47 polypeptide inserted into a second genomic locus, wherein the hypoimmunogenic cells exhibit reduced expression of MHC class I and/or class II human leukocyte antigens, wherein the first and second genomic loci are each a safe harbor locus, a target locus, a B2M gene locus, a CIITA gene locus, a TRAC gene locus, or a TRB gene locus.

In some embodiments, the first and second genomic loci are the same. In certain embodiments, the first and second genomic loci are different. In some embodiments, the hypoimmunogenic cells each further comprise a third exogenous polynucleotide inserted into a third genomic locus. In some embodiments, the third genomic locus is the same as the first or second genomic loci. In some embodiments, the third genomic locus is different from the first and/or second genomic loci.

In some embodiments, the safe harbor or target locus is selected from the group consisting of: a CCR5 gene locus, a CXCR4 gene locus, a PPP1R12C (also known as AAVS1) gene, an albumin gene locus, a SHS231 locus, a CLYBL gene locus, a ROSA26 gene locus, a CD142 gene locus, a MICA gene locus, a MICB gene locus, a LRP1 gene locus, a HMGB1 gene locus, an ABO gene locus, a RHD gene locus, a FUT1 gene locus, a PDGFRa gene locus, an OLIG2 gene locus, a GFAP gene locus, and a KDM5D gene locus. In certain embodiments, the CCR5 gene locus is exon 1-3, intron 1-2 or a coding sequence (CDS) of the CCR5 gene. In some embodiments, the PPP1R12C gene locus is intron 1 or intron 2 of the PPP1R12C gene. In some embodiments, the CLYBL gene locus is intron 2 of the CLYBL gene. In certain embodiments, the ROSA26 gene locus is intron 1 of the ROSA26 gene. In some embodiments, the target harbor locus is a SHS231 locus. In some embodiments, the CD142 gene locus is a CDS of the CD142 gene. In certain embodiments, the MICA gene locus is a CDS of the MICA gene. In some embodiments, the MICB gene locus is a CDS of the MICB gene. In some embodiments, the B2M gene locus is a CDS of the B2M gene. In exemplary embodiments, CIITA gene locus is a CDS of the CIITA gene. In certain embodiments, the TRAC gene locus is a CDS of the TRAC gene. In some embodiments, the TRB gene locus is a CDS of the TRB gene.

In certain embodiments, the exogenous polynucleotide is operably linked to a promoter.

In some embodiments, the hypoimmunogenic cells are differentiated cells derived from pluripotent stem cells. In some embodiments, the pluripotent stem cells comprise induced pluripotent stem cells.

In certain embodiments, the differentiated cells are selected from the group consisting of: pancreatic beta islet cells, glial progenitor cells, cardiac cells, neural cells, endothelial cells, B cells, retinal pigmented epithelium cells, hepatocytes, thyroid cells, skin cells, blood cells (e.g., plasma cells or platelets), and epithelial cells. In some embodiments, the differentiated cells are T cells.

In some embodiments, the hypoimmunogenic cells are derived from primary T cells. In certain embodiments, the hypoimmunogenic cells are T cells derived from pluripotent stem cells. In some embodiments, the hypoimmunogenic cells are derived from primary T cells. In some embodiments, the exogenous polynucleotide encodes a chimeric antigen receptor (CAR).

In exemplary embodiments, the chimeric antigen receptor (CAR) is selected from the group consisting of: a) a first generation CAR comprising at least one antigen binding domain, a transmembrane domain, and a signaling domain; b) a second generation CAR comprising at least one antigen binding domain, a transmembrane domain, and at least two signaling domains, c) a third generation CAR comprising at least one antigen binding domain, a transmembrane domain, and at least three signaling domains; and d) a fourth generation CAR comprising at least one antigen binding domain, a transmembrane domain, three or four signaling domains, and a domain which upon successful signaling of the CAR induces expression of a cytokine gene.

In some embodiments, the at least one antigen binding domain is selected from the group consisting of a) an antigen binding domain that targets an antigen characteristic of a neoplastic cell; b) an antigen binding domain that targets an antigen characteristic of a T cell; c) an antigen binding domain targets an antigen characteristic of an autoimmune or inflammatory disorder; d) an antigen binding domain that targets an antigen characteristic of senescent cells; e) an antigen binding domain that targets an antigen characteristic of an infectious disease; and f) an antigen binding domain that binds to a cell surface antigen of a cell.

In certain embodiments, the at least one antigen binding domain is selected from the group consisting of an antibody, an antigen-binding portion thereof, an scFv, and a Fab. In some embodiments, the CAR is a bispecific CAR comprising two antigen binding domains that bind two different antigens. In some embodiments, the at least one antigen binding domain(s) binds to an antigen selected from the group consisting of CD19, CD22, and BCMA. In certain embodiments, the bispecific CAR binds to CD19 and CD22.

In some embodiments, the transmembrane domain of the CAR comprises a transmembrane region selected from the group consisting of a transmembrane region from TCRα, TCRβ, TCRζ, CD3ε, CD3γ, CD3δ, CD3 CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In certain embodiments, the signaling domain(s) of the CAR comprises a costimulatory domain(s). In certain embodiments, the costimulatory domains comprise two costimulatory domains that are not the same. In some embodiments, the costimulatory domain(s) enhances cytokine production, CAR-T cell proliferation, and/or CAR-T cell persistence during T cell activation. In some embodiments, the cytokine gene is an endogenous or exogenous cytokine gene to the hypoimmunogenic cells. In some embodiments, the cytokine gene encodes a pro-inflammatory cytokine. In some embodiments, the pro-inflammatory cytokine is selected from the group consisting of IL-1, IL-2, IL-9, IL-12, IL 18, TNF, IFN-gamma, and a functional fragment thereof. In certain embodiments, the domain which upon successful signaling of the CAR induces expression of the cytokine gene comprises a transcription factor or functional domain or fragment thereof.

In some embodiments, the CAR comprises a CD3 zeta (CD3ζ) domain or an immunoreceptor tyrosine-based activation motif (ITAM), or functional variant thereof. In certain embodiments, the CAR comprises (i) a CD3 zeta domain, or an immunoreceptor tyrosine-based activation motif (ITAM), or functional variant thereof, and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof; and (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof, (iii) a 4-1BB domain, or a CD134 domain, or functional variant thereof and (iv) a cytokine or costimulatory ligand transgene. In certain embodiments, the CAR comprises a (i) an anti-CD19 scFv; (ii) a CD8α hinge and transmembrane domain or functional variant thereof (iii) a 4-1BB costimulatory domain or functional variant thereof and (iv) a CD3ζ signaling domain or functional variant thereof.

In some embodiments, the hypoimmunogenic cells comprise reduced expression of an endogenous T cell receptor. In some embodiments, the hypoimmunogenic cells comprise reduced expression of cytotoxic T-lymphocyte-associated protein 4 (CTLA4) and/or programmed cell death (PD1). In certain embodiments, the hypoimmunogenic cells comprise increased expression of programmed cell death ligand 1 (PD-L1).

In some embodiments, the patient is sensitized against one or more alloantigens. In some embodiments, the patient is sensitized from a previous pregnancy or a previous allogeneic transplant. In certain embodiments, the one or more alloantigens comprise human leukocyte antigens.

In some embodiments, the patient exhibits memory B cells and/or memory T cells reactive against the one or more alloantigens. In certain embodiments, the allogeneic transplant is selected from the group consisting of an allogeneic cell transplant, an allogeneic blood transfusion, an allogeneic tissue transplant, and an allogeneic organ transplant.

In some embodiments, the patient exhibits a reduced or no immune response to the population of cells. In certain embodiments, the reduced or no immune response to the population of cells response exhibited is selected from the group consisting of reduced or no systemic immune response, reduced or no adaptive immune response, reduced or no innate immune response, reduced or no T cell response, and reduced or no B cell response.

In some embodiments, the patient exhibits: a) a reduced level of systemic TH1 activation or no systemic TH1 activation upon administering the population of cells; b) a reduced level of immune activation of peripheral blood mononuclear cells (PBMCs) or no immune activation of PBMCs upon administering the population of cells; c) a reduced level of donor-specific IgG antibodies or no donor specific IgG antibodies against the population of cells upon administering the population of cells; d) a reduced level of IgM and IgG antibody production or no IgM and IgG antibody production against the population of cells upon administering the population of cells; and/or e) a reduced level of cytotoxic T cell killing or no cytotoxic T cell killing of the population of cells upon administering the population of cells.

In some embodiments, the disorder is a cancer or the cellular therapy is for the treatment of a cancer. In some embodiments, the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

### V. Examples

### Example 1: Human B2M^{indel/indel}, CIITA^{indel/indel}, CD47tg Induced Pluripotent Stem Cells in a Xenogeneic Transplantation Study

To study the effects of decreasing MHC I and MHC II expression and increasing CD47 expression for cell transplants, human B2M^{indel/indel}, CIITA^{indel/indel}, CD47tg induced pluripotent stem cells (HIP cells) were transplanted into rhesus monkey (non-human primate or NHP) recipients (xenogeneic transplantation).

Study design and administration. Eight NHPs (F/M, 2-3 kg, 12-36 months of age) were randomized into two groups (n=4) for blinded administration of either wild-type or HIP cells. Under an IACUC-approved protocol, each NHP was administered four subcutaneous injections of ˜10⁷ human wild-type or HIP cells into the back. Characteristics of the human wild-type iPSCs and human HIP iPSCs are shown in FIG. 14. Blood was drawn for analysis prior to injection (“pre-Tx” or day 0) at days 7, 13, 25 and so forth following injection. Both the wild-type and HIP cells also transgenically expressed firefly luciferase for bioluminescence imaging (BLI), and cell survival was monitored by BLI. The study design and results are shown in FIGS. 1A-1F, 2A, 2B, 3A, 3B, 4A-4C, 5A-5C, 6A-6C, and 7A-7C.

No systemic immune responses were observed in the NHPs receiving xenogeneic HIP cells following the initial injection, in contrast to the NHPs injected with wild-type cells, which showed increases in T cell activation, IgM and IgG levels, and donor-specific IgM and IgG. To determine whether HIP cells could be re-administered with a similar lack of immune activation, the NHPs were re-injected with the same cell type (wild-type or HIP) as the second injection between day 118 and day 123 following the initial injections. As before, blood was drawn for analysis prior to re-injection (“pre-Tx or day 0) and at days 7 and 13 thereafter (125 and 131 days after first injection, respectively), and cell survival was monitored by BLI. Remarkably, no systemic immune response was observed in the animals re-injected with xenogeneic HIP cells, whereas the animals re-injected with wild-type cells showed systemic immune activation. Although no systemic immune activation was seen in the animals administered the HIP cells, the cells did not survive over a 13-day period (BLI<5% of initial) on the initial or second dose, apparently due to local xenogeneic responses as well as responses against the vehicle (Matrigel). These results indicate that HIP cells can evade immune recognition and activation on multiple doses.

To determine whether the HIP cells could evade pre-formed immune responses, the four NHPs that were initially administered two doses of wild-type cells were transplanted with HIP cells and vice versa (crossover administration). The HIP or wild-type cells were injected subcutaneously into the animals between day 118 and day 123 following the second injection (day 241 following the initial injection). As before, blood was drawn for analysis 48 days prior to re-injection and at days 7 and 13 thereafter (248 and 254 days after first injection, respectively), and cell survival was monitored by BLI.

T cell activation. T cell activation in animals administered wild-type and HIP human iPSC was measured by Elispot assays. For uni-directional Elispot assays, recipient PBMCs were isolated from rhesus macaques 48 days before and 7 and 13 days after the third injection (crossover administration). T cells were purified from the PBMCs by CD3 MACS-sorting (Miltenyi) and were used as responder cells. Donor cells (wild-type or HIP cells) were mitomycin-treated (50 μg/mL for 30 minutes, Sigma) and used as stimulator cells. 1×10⁵ stimulator cells were incubated with 5×10⁵ recipient responder T-cells for 36 hours and IFN-γ spot frequencies were enumerated using an Elispot plate reader. For the animals administered wild-type cells after two previous injections of HIP cells, Elispot activity observed was highest at day 7 following crossover injection (FIGS. 1A-1F). These results are indicative of systemic TH1 activation and acute cellular immune response after injection of wild-type cells, with no immune suppression by previous injection of HIP cells. By contrast, the animals injected HIP cells after two previous injections of wild-type cells (crossover injection) had Elispot activity comparable to naïve TH1 cells at day 0, indicating no systemic TH1 activation or cellular immune response to the modified cells, even in animals with pre-formed immune responses against the wild-type xenogeneic cells (FIGS. 1A-1F).

Donor-specific antibody activity. Production of donor-specific antibodies by the animals on crossover injection with wild-type and HIP cells was also assayed. Sera from recipient monkeys were de-complemented by heating to 56° C. for 30 minutes. Equal amounts of sera and wild-type or HIP cell suspensions (5×10⁶ cells/mL) were incubated for 45 minutes at 4° C. Cells were labelled with FITC-conjugated goat anti-IgM (BD Bioscience) or anti-IgG and analyzed by flow cytometry (BD Bioscience).

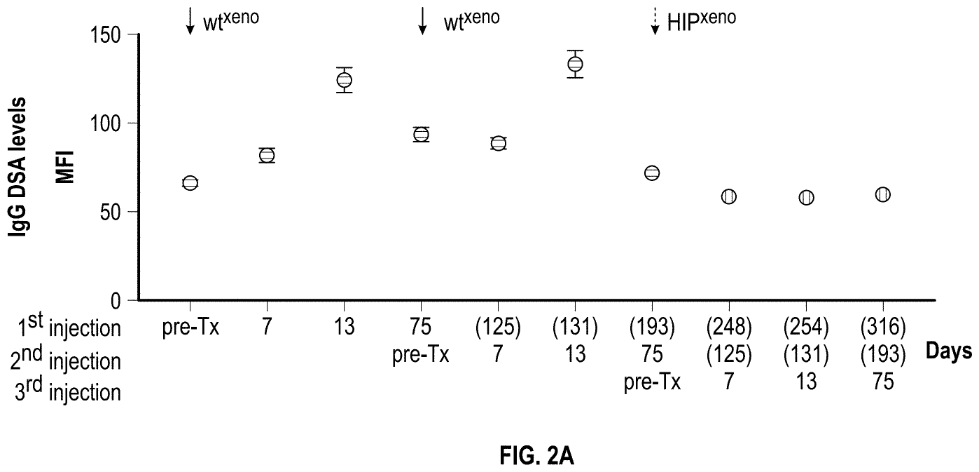

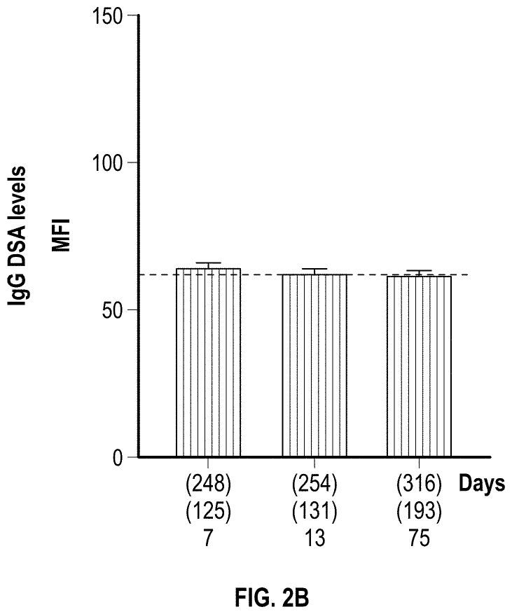

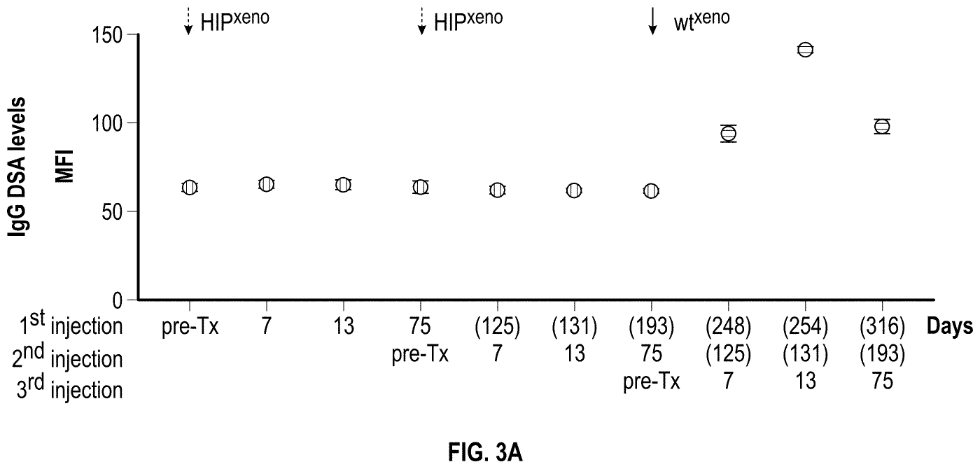

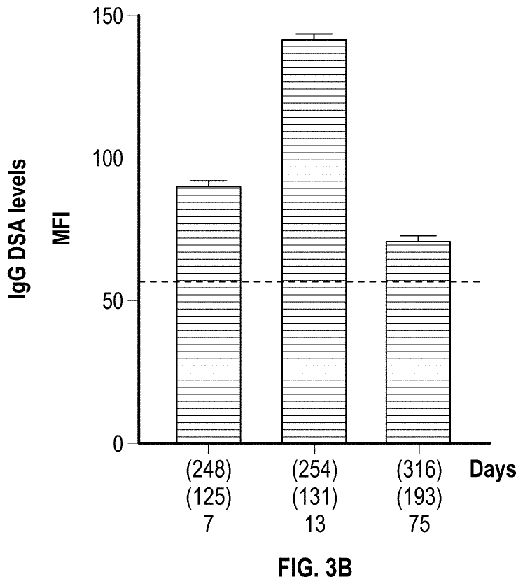

An increase in donor-specific reactivity above pre-injection levels was observed at days 7 and 13 following crossover injection of wild-type cells in animals previously administered HIP cells, with IgM decreasing from day 7 to 13, consistent with isotype switching (data not shown). By contrast, no donor-specific IgM binding was observed in animals administered HIP cells that had previously received two injections of wild-type cells (data not shown). An increase in donor-specific reactivity was observed at day 13 following crossover injection of wild-type cells in animals previously administered HIP cells, with IgG increasing from day 7 to day 13, and then decreasing from day 13 to 75, consistent with isotype switching (FIG. 3A-3B). By contrast, no donor-specific IgG binding was observed in animals administered HIP cells that had previously received two injections of wild-type cells (FIGS. 2A and 2B) at days 7, 13, and 75.

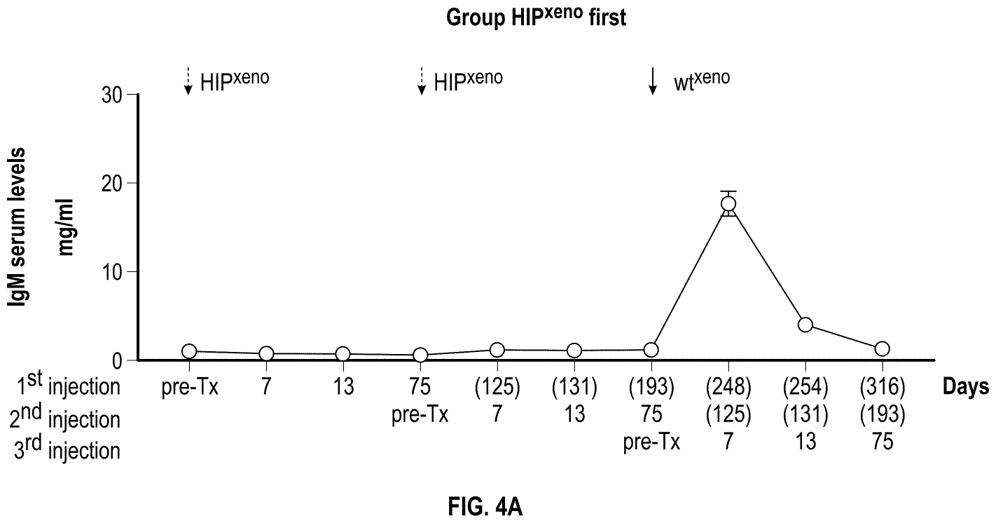

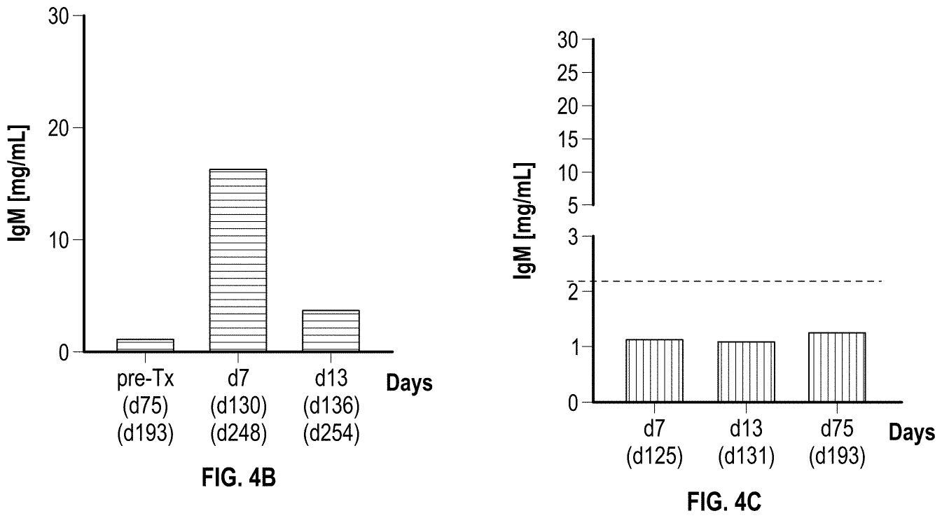

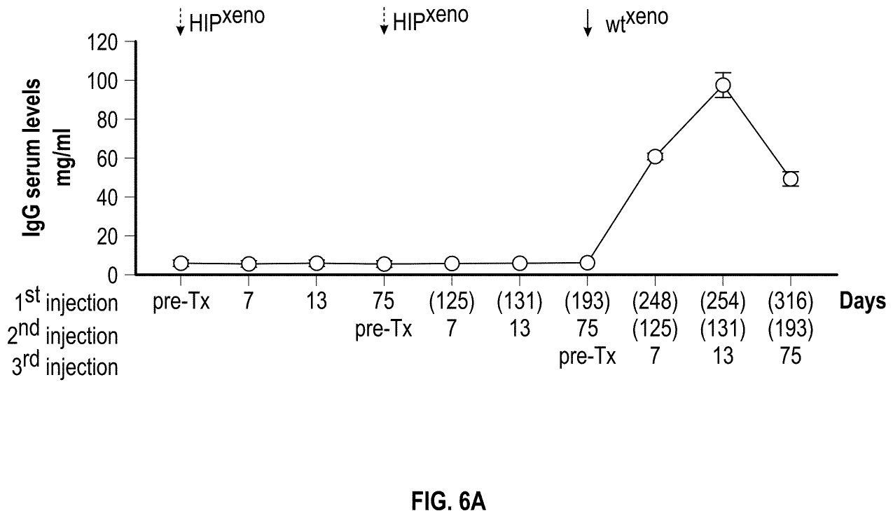

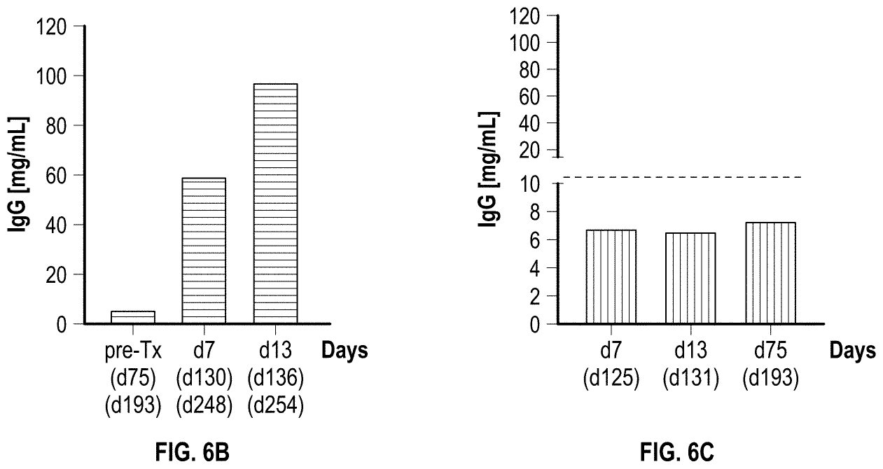

Bulk antibody production. Total antibody production in the animals receiving crossover injections of wild-type or HIP cells was also assayed using IgM and IgG ELISA kits (Abcam). After the removal of unbound proteins by washing, anti-IgM or anti-IgG antibodies conjugated with horseradish peroxidase (HRP), are added. These enzyme-labeled antibodies form complexes with the previously bound IgM or IgG. The enzyme bound to the immunosorbent is assayed by the addition of a chromogenic substrate, 3,3′,5,5′-tetramethyl-benzidine (TMB). In the animals crossover administered wild-type cells following two administrations of HIP cells, a sharp increase in total IgM and IgG was observed, with the greatest IgM production observed at day 7 and greatest IgG production observed at day 13, indicative of isotype switching (FIGS. 4A-4C and 6A-6C).

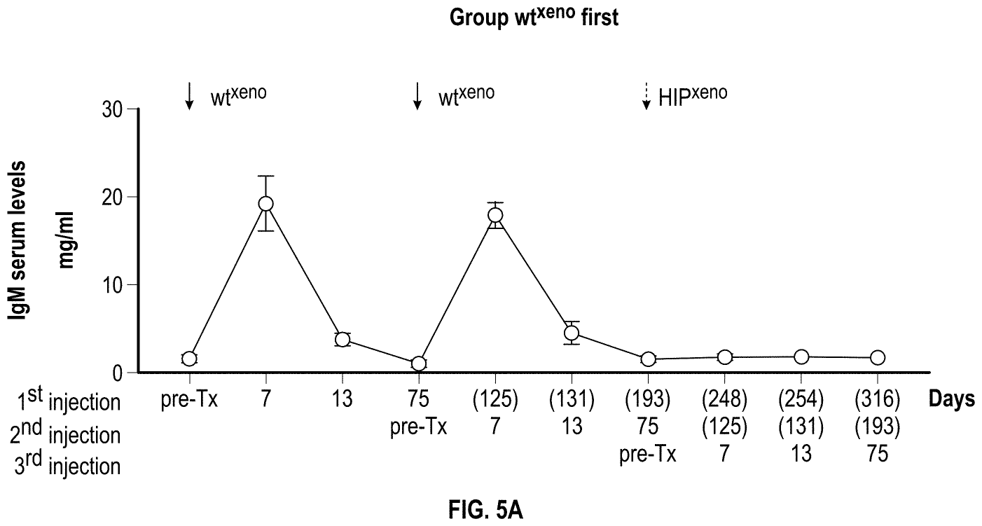

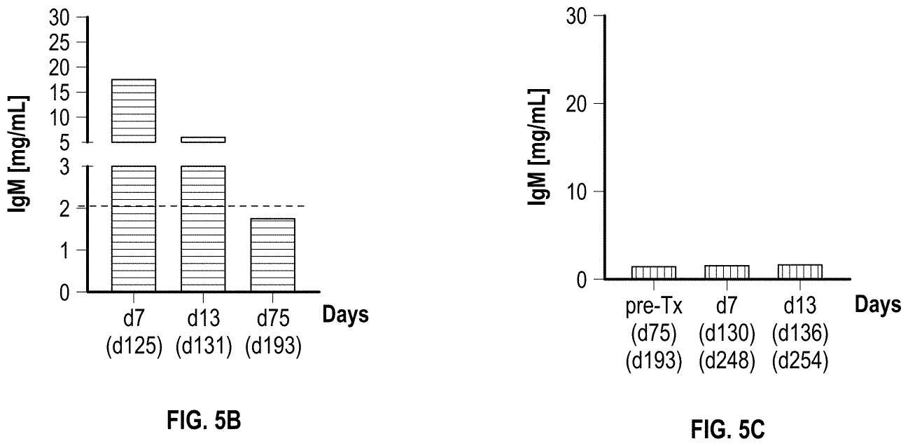

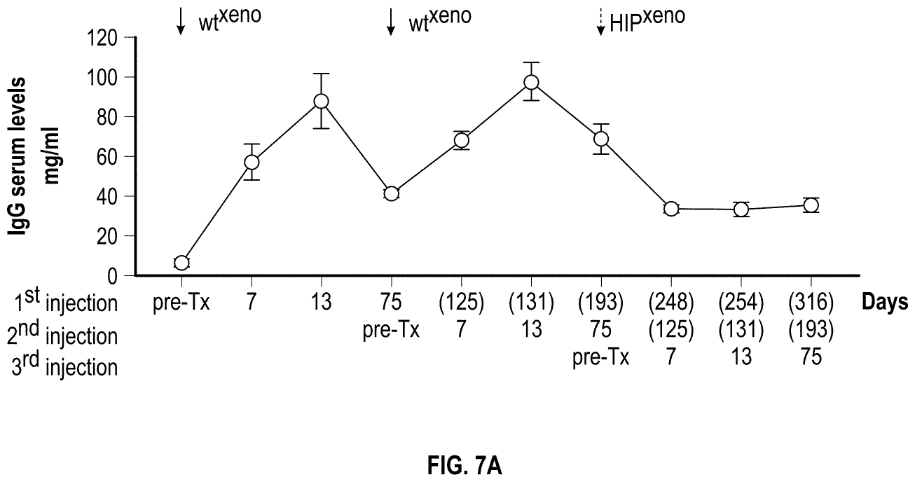

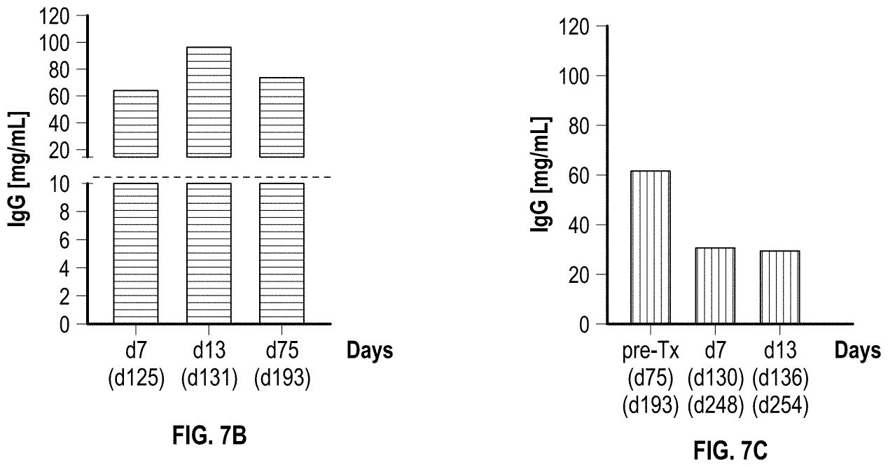

Strikingly, no increase in total IgM or IgG was observed at any time point in the animals crossover administered HIP cells following two injections of wild-type cells (FIGS. 5A-5C and 7A-7C).

Some IgG was observed prior to HIP injection, likely residual production from the previous wild-type administration (FIGS. 7A-5C). Together, these results indicate a near-total lack of humoral immune response to the HIP cells.

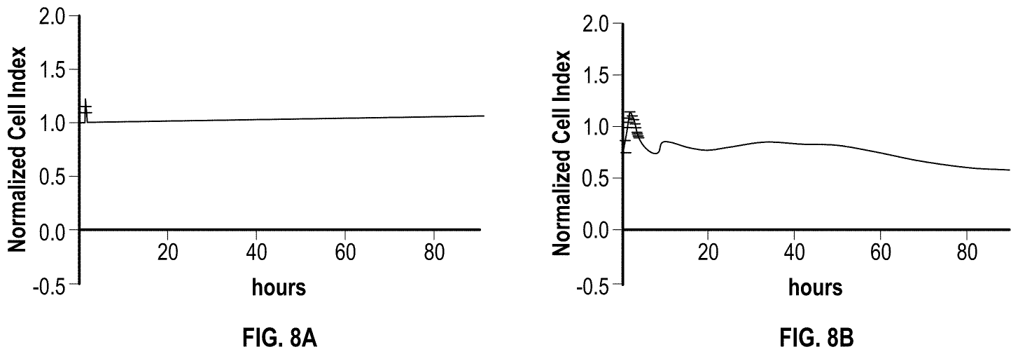

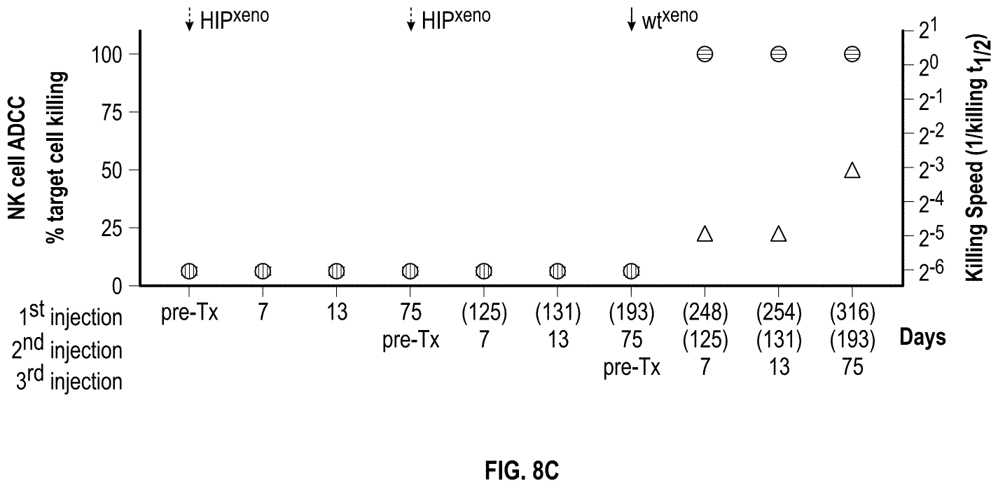

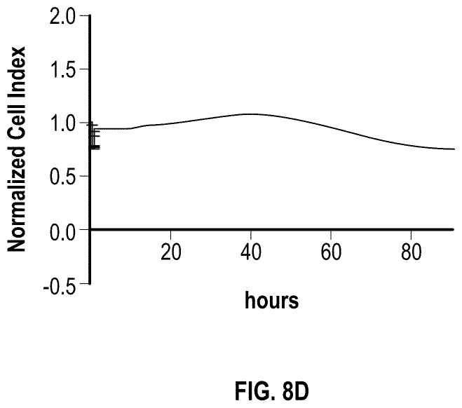

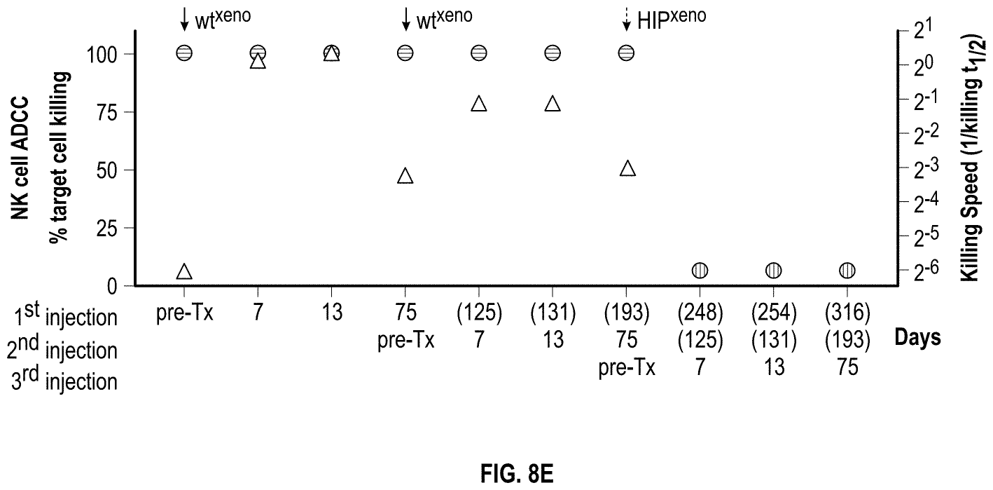

NK cell killing. Systemic innate immunity by NK cells was also assayed in the animals crossover injected wild-type or HIP cells. NK cell killing assays were performed on the XCELLIGENCE MP platform (ACEA BioSciences). 96-well E-plates (ACEA BioSciences) were coated with collagen (Sigma-Aldrich) and 4×10⁵ wild-type or HIP cells were plated in 100n1 cell specific media. After the Cell Index value reached 0.7, rhesus NK cells isolated from the treated animals were added with an E:T ratio of 1:1 with or without 1 ng/ml rhesus IL-2 (MyBiosource, San Diego, Calif.). As a killing control, cells were treated with 2% TRITON X100. No killing was observed by stimulated or unstimulated NK cells on wild-type or HIP cells, indicating that CD47 expression on the HIP cells was effective to protect from NK cells and macrophages in the absence of HLA I and HLA II. (see Deuse et al., 2019, Nat. Biotechnol., 37:252-258). As shown in FIGS. 8A-8c, no NK cell killing was observed following administration of the first dose of HIP cells into wild-type NHP (FIG. 8C) nor with the re-dose of HIP cells into the wild-type NHP (FIG. 8c). Lack of NK cell killing was also observed after crossover injection of the HIP cells into wild-type NHPs having pre-existing immunity despite the HLA I/HLA II (e.g., MHC edits) to the HIP cells (FIGS. 8D AND 8E).

Survival of transplanted cells. Although no systemic immune response was observed for animals crossover administered human HIP cells, the cells did not survive likely due to local xenogeneic responses. For the prior wild-type and HIP injections, histopathology analysis performed on cell plugs removed from the animals showed neutrophil infiltration or fibrin (as indicator that neutrophils have been in the area) as well as signs of foreign body reaction and hypersensitivity reaction type IV against the vehicle, indicative of a xenogeneic response against the human cells and allergic reaction to the vehicle, respectively. The allergic and foreign body reaction against the vehicle were confirmed by an additional control monkey injected with only vehicle (no cells), which demonstrated similar histopathological features.

This example demonstrates that HIP cells can be administered to subjects that have preexisting systemic allogeneic immune responses without provoking a new systemic immune response.

### Example 2: Human B2M^{indel/indel}, CIITA^{indel/indel} CD47tg Induced Pluripotent Stem Cells (iPSCs) and Wildtype iPSCs in Allogeneic Transplantation Crossover Studies

This example describes allogeneic transplantation crossover studies that compare the effects of transplantation of human B2M^{indel/indel}, CIITA^{indel/indel}, CD47tg induced pluripotent stem cells (HIP iPSCs) and wildtype iPSCs into rhesus monkey (non-human primate or NHP) recipients. In one set of crossover studies, wildtype iPSCs were transplanted subcutaneously (s.c,) into the back of the recipient animal, and after about 6 weeks HIP iPSCs were transplanted s.c. in a neighbor location. In a second set of crossover studies, HIP iPSCs were transplanted s.c. into the back of the recipient animal, and after about 6 weeks wildtype iPSCs were transplanted s.c., into a neighbor location. The presence of the engrafted cells and their progeny were monitored.

The data show that the HIP iPSCs are not detected by the immune system of the sensitized NHP recipients (NHP recipients who were initially transplanted with wildtype iPSCs) and thus, avoid immune rejection. The engrafted HIP iPSCs evaded recipient immune responses even though the recipients possess a functional immune system. In addition, NHP recipients who were initially transplanted with HIP iPSCs had an immune response to the subsequently transplanted wildtype iPSCs.

A. Methods

Gene editing of human iPSCs overexpressing rhesus CD47. Human iPSCs B2M^{indel/indel}, CIITA^{indel/} rhesus CD47 tg cells (also referred to as HIP iPSCs or HIP cells) were cultured using standard human iPSC cell culture methods recognized by those skilled in the art. Characteristics of the rhesus wild-type iPSCs and rhesus HIP iPSCs are shown in FIG. 15.

Rhesus iPSC cell culture. Rhesus iPSCs were cultured using standard rhesus iPSC cell culture methods recognized by those skilled in the art.

Luciferase transduction of hIPSCs. hiPSCs (i.e., HIP iPSCs and wildtype iPSCs) were infected with lentiviral particles expressing a luciferase II gene under expression control by a constitutively active promoter (i.e., CAG promotor) Luciferase expression by the infected cells was confirmed using a standard, commercially available luciferase assay.

iPSC preparation for transplantation into non-human primates. iPSCs were resuspend in standard culture media including a pro-survival cocktail (i.e., a cocktail including a caspase inhibitor, BcL-xL, IGF-1, pinacidil and cyclosporine A). Cells were loaded into syringes for the injection.

Intramuscular iPSC injection in rhesus macaques. Animals were sedated with an intramuscular (IM) injection of a rapid-acting anesthetic (i.e., a combination of tiletamine and zolazepan), preferably not in the leg receiving the cell implant. Once anesthetized, both legs of the animal were shaved at the catheter and cell implant sites. Blood samples were taken via percutaneous venipuncture from the femoral vein. A catheter was placed into the saphenous vein (preferably not in the leg receiving cell implant). The area of cell implantation, i.e., the anterior surface of the thigh, or quadricep muscle, was surgically scrubbed using alternating chlorhexidine gluconate/ethanol scrubs, ultimately finishing with chlorhexidine gluconate.

An incision was made through the skin over the middle anterior side of the quadricep muscle of the animals. The quadricep muscle isolated by pinching and the iPSCs were injected in a starburst pattern such that the injected cells were injected in a plurality of locations within the pattern. The incision was closed with a suture and the injection area was marked for future reference.

Luciferin was injected into the recipient animal via pre-placed intravenous catheter for luciferin infusion. Once the animal's vitals such as heartrate returned to normal, the injection area was imaged by way of bioluminescence imaging (BLI). Cell survival was monitored by BLI. The quantitative bioluminescence imaging at a is represented as BLI images and BLI signals over time.

B. Transplantation of HIP iPSCs

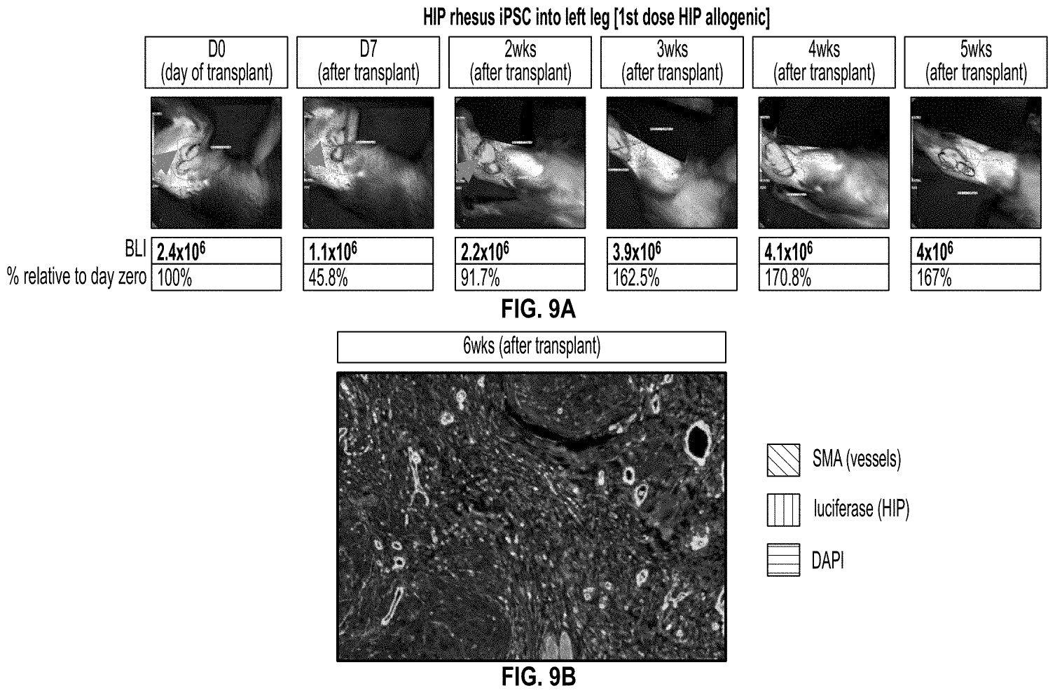

As shown in FIG. 9A, allogeneic HIP rhesus iPSCs were transplanted into the left leg of a rhesus recipient. Such HIP cells did not elicit an immune response in the recipient. The engrafted cells were detected at the injection site for at least 6 weeks after transplantation. FIG. 9B shows immunohistochemical staining of the left leg engrafted with HIP iPSCs at 6 weeks after transplantation. FIG. 9B shows staining of smooth muscle actin (SMA) which represents vessels, and luciferase which shows the transplanted HIP iPSCs.

Also, FIG. 13C shows BLI images of a similar study for monitoring the presence of transplanted allogeneic HIP rhesus iPSCs in the left leg of an allogeneic rhesus recipient. The transplanted cells and progeny thereof were found in the injection site for at least 9 weeks after the initial transplantation. The HIP iPSCs did not elicit a significant immune response in the rhesus recipient, as the cells persisted for at least 9 weeks after transplantation.

C. Crossover Studies: Administration of Wildtype iPSCs Followed by HIP iPSCs in the Same NHP

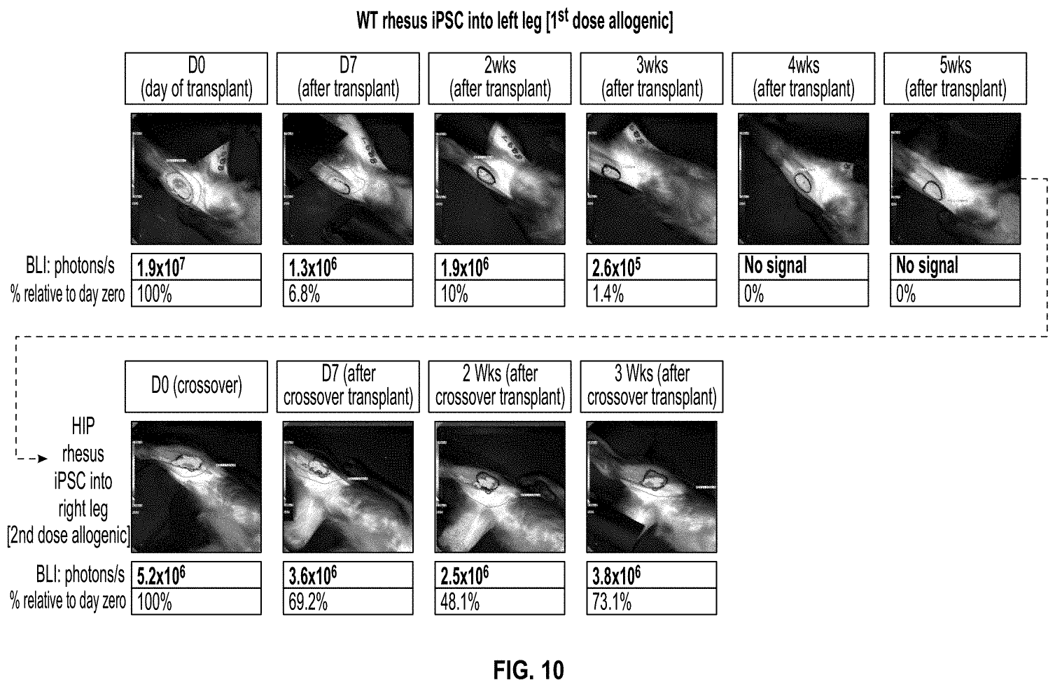

In a crossover study of wildtype iPSCs to HIP iPSCs, allogeneic rhesus wildtype iPSCs were transplanted into the left leg of a rhesus recipient. The population of transplanted rhesus wildtype iPSCs was substantially reduced by day 7 after transplantation (100% to 6.8%; FIG. 10). At 2 weeks after transplantation, only 10% of the transplanted population were detected and at 3 weeks after transplantation, merely 1.4% of the population remained. No transplanted cells were found at the injection site at 4 weeks and 5 weeks after transplantation. As such, the rhesis recipient appeared to be sensitized. In the crossover arm of the study, allogeneic HIP rhesus iPSCs were injected into the right leg of the sensitized rhesus recipient at 5 weeks after the initial wildtype iPSC transplant (also referred to as day 0 (d0) crossover).

At day 0 of crossover transplantation, the transplanted allogeneic HIP rhesus iPSCs were detectable at the injection site (FIG. 10, bottom row). At day 7 (d7) of crossover transplantation, 69.2% of the transplanted HIP iPSCs were detected. Also, 2 weeks after crossover transplantation, 48.1% of the cells remained. As such, the recipient animal elicited an immune response to the wildtype iPSCs in the initial arm of the study, and the HIP iPSCs persisted in the sensitized recipient animal in the crossover arm.

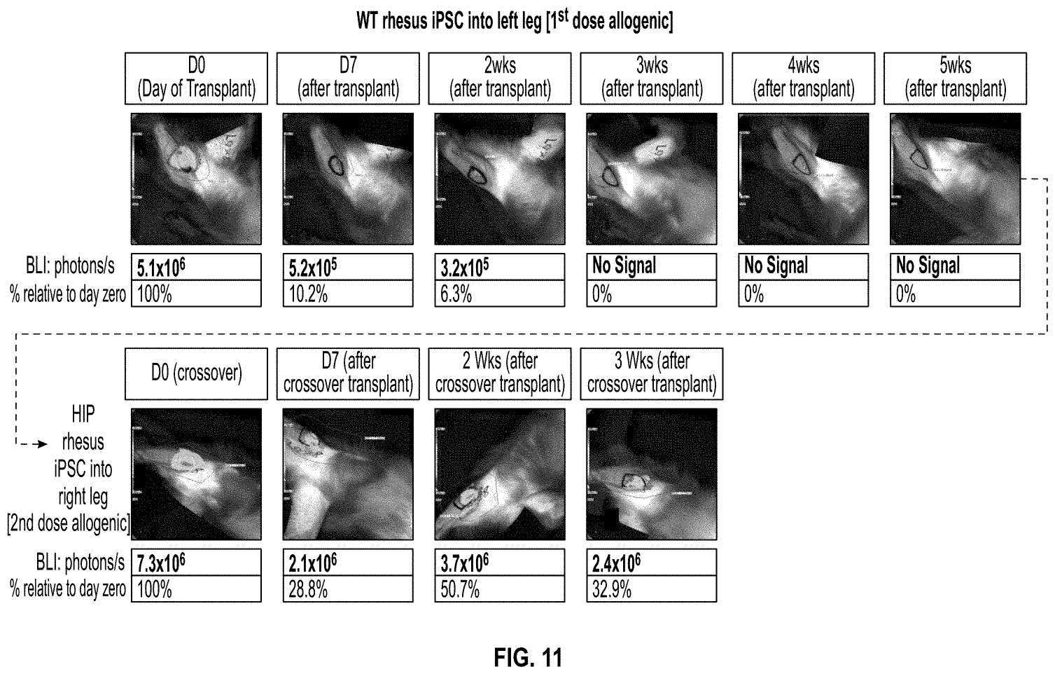

FIG. 11 shows results from another crossover study of wildtype iPSCs to HIP iPSCs. The transplanted rhesus wildtype iPSCs elicited an immune response in the naïve recipient. Specifically, only 10.2% of the transplanted wildtype iPSCs were detected at d7 after transplantation. At 5 weeks after the initial transplantation of the rhesus wildtype iPSCs (also referred to as d0 of crossover transplantation), HIP rhesus iPSCs were transplanted into the right leg of the now sensitized rhesus recipient. The transplanted HIP iPSCs were detected at the injection site (FIG. 11, bottom row). At day 7 after crossover transplantation, 28.8% of the transplanted cells and progeny thereof were located in the injection site. At 3 weeks after crossover transplantation, the population detected was about 32.9% of the transplanted HIP iPSCs.

D. Crossover Studies: Administration of HIP iPSCs Followed by Wildtype iPSCs in the Same NHP

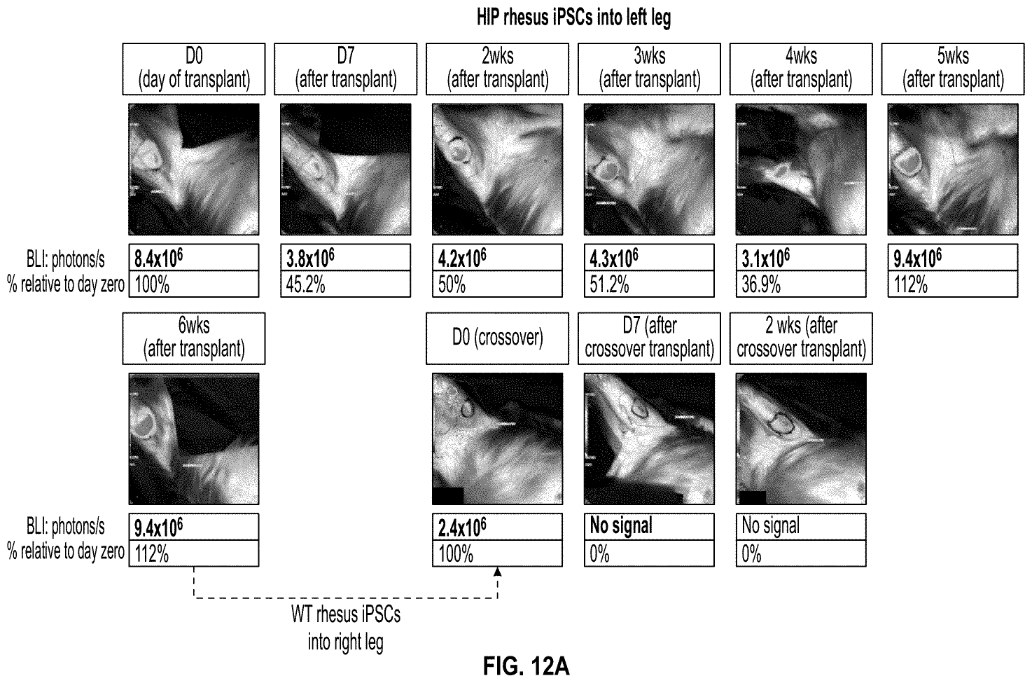

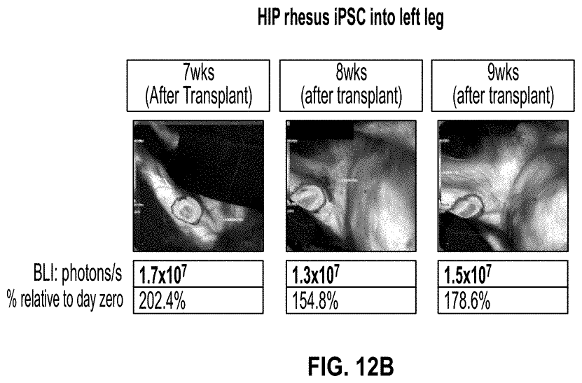

In a crossover study of HIP iPSCs to wildtype iPSCs, allogeneic HIP iPSCs were transplanted into the left leg of a rhesus recipient (FIG. 12). The transplanted HIP iPSCs and progeny thereof were detectable in the injection site for at least 9 weeks after transplantation. At 5 weeks after transplant, there were about 112% of the initial transplanted HIP iPSCs population and progeny thereof, and at 7 weeks, 202.4% of the HIP iPSCs and progeny thereof were present. At 8 weeks and 9 weeks, 154.8% and 178.6% of the HIP iPSCs and progeny thereof were present, respectively. The HIP iPSCs were found in the recipient for at least 9 weeks after the initial transplant.

At 6 weeks after the initial transplantation of the HIP iPSCs s (also referred to as day 0 of crossover transplantation), allogeneic rhesus wildtype iPSCs were transplanted into the right leg of the rhesus recipient. The transplanted wildtype iPSCs were detected at the injection site (FIG. 12, bottom row). At day 7 after crossover transplantation, none of the transplanted cells and progeny thereof were located in the injection site. No luciferase signal was detected. In contrast, at 7 weeks after the initial transplant of HIP iPSCs, there were about 202.4% of the initial transplanted HIP iPSCs population and progeny thereof in the left leg of the rhesus recipient.

The results from the series of crossover studies described above show that HIP iPSCs are able to hide from the immune system of sensitized NHP recipients (NHP recipients who were initially transplanted with wildtype iPSCs) and thus, the HIP iPSCs can avoid immune rejection. In addition, recipients who were initially transplanted with HIP iPSCs generated an immune response to the subsequently transplanted wildtype iPSCs. See, for instance, FIGS. 13A and 13B. The engrafted HIP iPSCs evaded immune responses even though the recipients possessed a functional immune system.

### Example 3: Expression of Exogenous CD47 in Human B2M^{Indel/Indel}, CIITA^{INDEL/INDEL}, CD47tg Induced Pluripotent Stem Cells (iPSCs) Using Safe Harbor Sites

This example describes studies to characterize the expression of exogenous CD47 expression in human B2M^{indel/indel}, CIITA, CD47tg induced pluripotent stem cells (iPSCs), wherein a polynucleotide encoding an exogenous CD47 is inserted into a safe harbor site in the iPSC.

B2M^{indel/indel}, CIITA^{indel/indel} induced pluripotent stem cells (iPSCs) were generated using standard CRISPR/Cas9 gene editing techniques. HDR donor plasmids encoding human CD47, in expression cassettes driven by the CAG or the EF1a promoters and flanked by 1 kb homology arms for three safe harbor sites (AAVS1, CLYBL, or CCR5) were introduced into the B2M^{indel/indel}, CIITA^{indel/indel} iPSCs.

Target integration of the CD47 at the safe harbor sites were achieved using standard CRISPR/Cas9 gene editing techniques to mediate homology directed repair. The following bulk-edited lines were generated:
CAG-CD47_AAVS1
CAG-CD47_CLYBL
CAG-CD47_CCR5
EF1α-CD47_AAVS1
EF1α-CD47_CLYBL
EF1α-CD47_CCR5.

Single cell cloning from the bulk-edited lines were carried out. Clones were assessed for copy number and plasmid insertion, and PCR genotyping was performed to verify the correct location of the integration into the safe harbor site using standard techniques. Clones that passed the genomic assessment were expanded and clonal selection assays were performed to narrow down to 2 or 3 clones for each safe harbor site. Assessment of CD47 expression in the B2M^{indel/indel}, CIITA^{indel/indel}, CD47tg clones were carried out using flow cytometry.

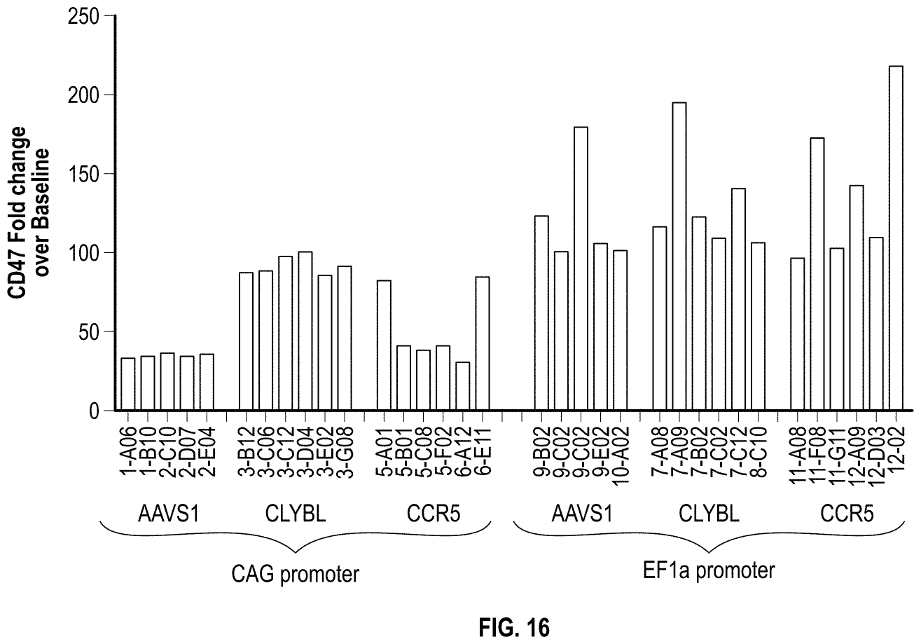

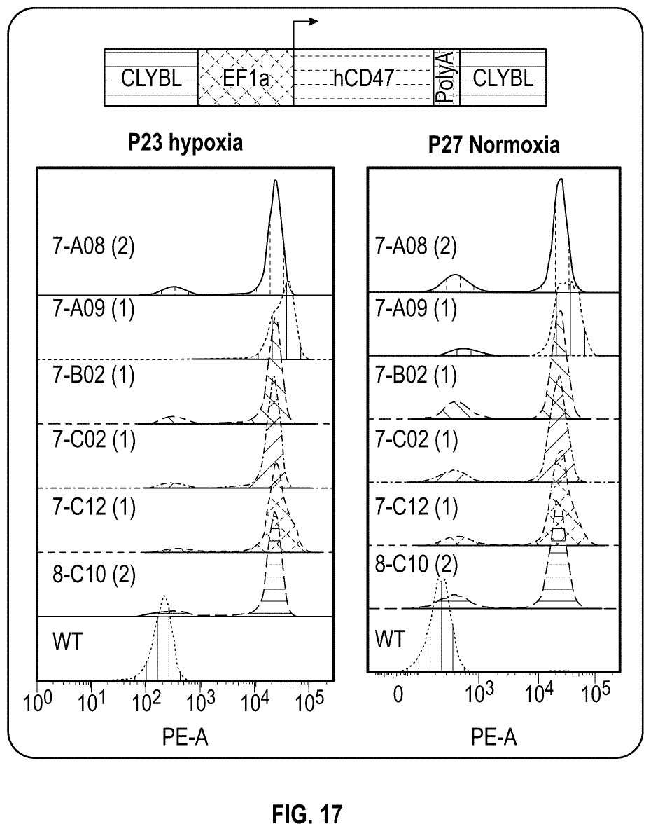

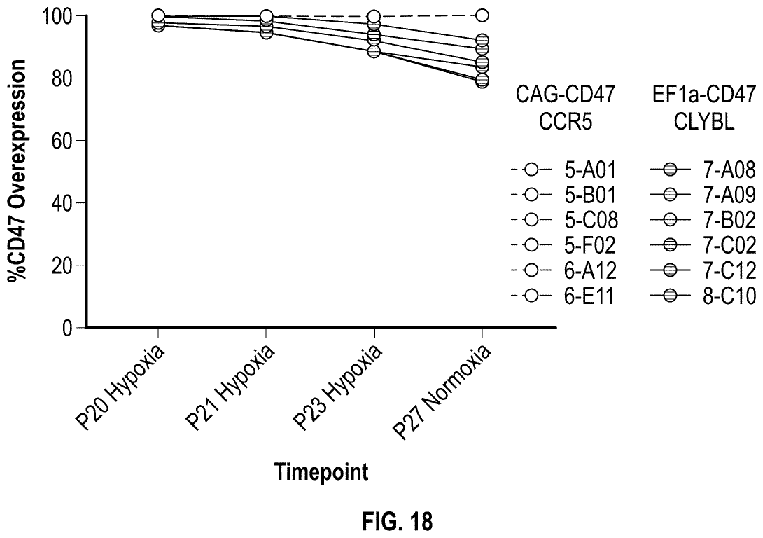

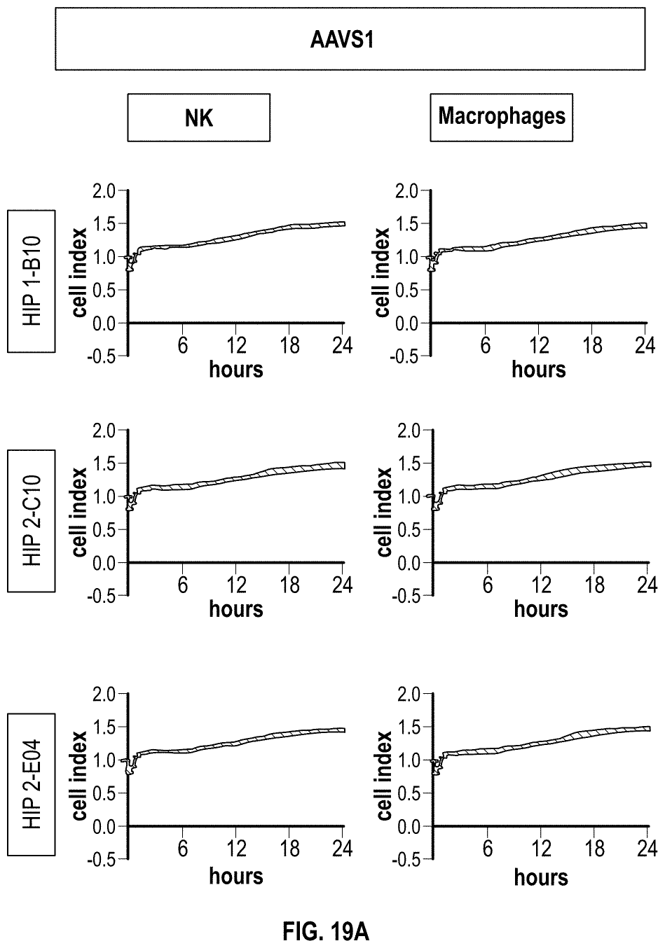

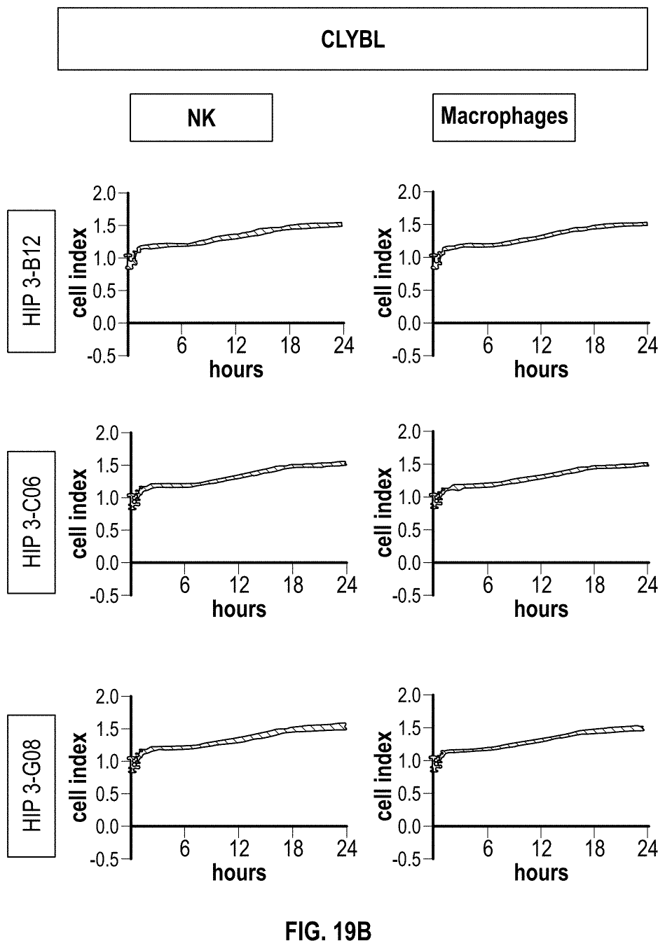

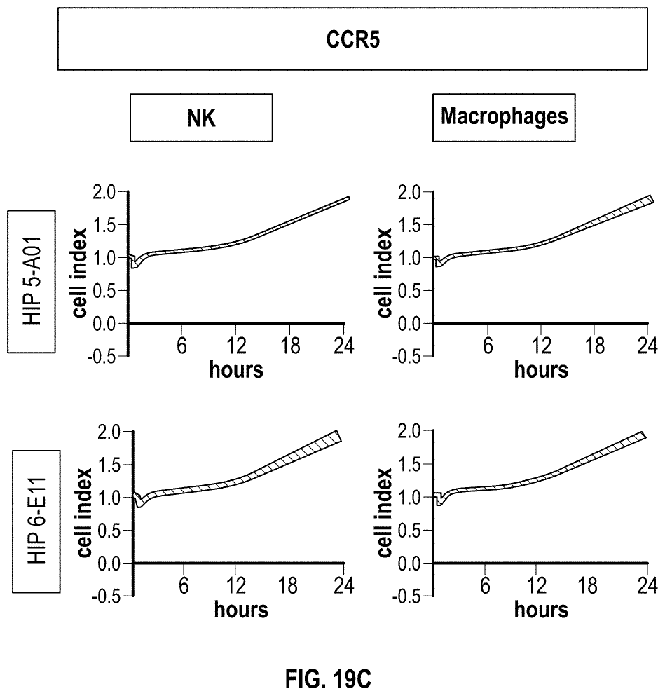

As shown in FIG. 16, B2M^{indel/indel}, CIITA^{indel/indel}, CD47tg where the CD47 transgene is inserted into each of the three harbor sites exhibited enhanced CD47 expression at ˜30-200 fold over endogenous levels. CD47 was also observed to be stably expressed by the CAG promoter from several safe-harbor sites in iPSCs (see FIGS. 17 and 18). Protection of the B2M^{indel/indel}, CIITA^{indel/indel}, CD47tg iPSCs from systemic innate immunity was further assessed using the methods described above. As shown in FIG. 19, B2M^{indel/indel}, CIITA^{indel/indel}, CD47tg iPSCs that include a CD47 transgene inserted into a safe harbor site stably expressed CD47 as sufficient levels to protect from NK cell and macrophage killing.

All headings and section designations are used for clarity and reference purposes only and are not to be considered limiting in any way. For example, those of skill in the art will appreciate the usefulness of combining various aspects from different headings and sections as appropriate according to the spirit and scope of the technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method of treating a patient in need thereof comprising administering a population of engineered cells, wherein the engineered red cells comprise a first exogenous polynucleotide encoding CD47 and
(I) one or more of:
a. reduced expression of major histocompatibility complex (MHC) class I and/or class II human leukocyte antigens;
b. reduced expression of MHC class I and class II human leukocyte antigens;
c. reduced expression of beta-2-microglobulin (B2M) and/or MHC class II transactivator (CIITA); and/or
d. reduced expression of B2M and CIITA;
wherein the reduced expression is due to a modification and the reduced expression is relative to a cell of the same cell type that does not comprise the modification;
(II) wherein the patient is a sensitized patient, wherein the patient:
i. is sensitized against one or more alloantigens;
ii. is sensitized against one or more autologous antigens;
iii. is sensitized from a previous transplant;
iv. is sensitized from a previous pregnancy;
v. received a previous treatment for a condition or disease; and/or
vi. is a tissue or organ transplant patient, and the engineered cells are administered prior to, concurrent with, and/or after administering the tissue or organ transplant.

2. A method of treating a patient in need thereof comprising administering a population of pancreatic islet cells, cardiac progenitor cells, or glial progenitor cells, wherein the pancreatic islet cells, cardiac progenitor cells, or glial progenitor cells, comprise a first exogenous polynucleotide encoding CD47 and
(I) one or more of:
a. reduced expression of major histocompatibility complex (MHC) class I and/or class II human leukocyte antigens;
b. reduced expression of MHC class I and class II human leukocyte antigens;
c. reduced expression of beta-2-microglobulin (B2M) and/or MHC class II transactivator (CIITA); and/or
d. reduced expression of B2M and CIITA;
wherein the reduced expression is due to a modification and the reduced expression is relative to a cell of the same cell type that does not comprise the modification;
(II) wherein:
a. the patient is not a sensitized patient; or
b. the patient is a sensitized patient, wherein the patient:
i. is sensitized against one or more alloantigens;
ii. is sensitized against one or more autologous antigens;
iii. is sensitized from a previous transplant;
iv. is sensitized from a previous pregnancy;
v. received a previous treatment for a condition or disease; and/or
vi. is a tissue or organ patient, and the pancreatic islet cells, cardiac progenitor cells, or glial progenitor cells, are administered prior to administering the tissue or organ transplant.

3.-4. (canceled)

5. The method of claim 1, wherein the patient is a sensitized patient and wherein the patient exhibits memory B cells and/or memory T cells reactive against the one or more alloantigens or one or more autologous antigens, optionally wherein the one or more alloantigens comprise human leukocyte antigens.

6. (canceled)

7. The method of claim 1, wherein the patient is a sensitized patient who is sensitized from a previous transplant, wherein:
a. the previous transplant is selected from the group consisting of a cell transplant, a blood transfusion, a tissue transplant, and an organ transplant, optionally the previous transplant is an allogeneic transplant; or
b. the previous transplant is a transplant selected from the group consisting of a chimera of human origin, a modified non-human autologous cell, a modified autologous cell, an autologous tissue, and an autologous organ, optionally the previous transplant is an autologous transplant.

8. The method of claim 1, wherein the patient is a sensitized patient who is sensitized from a previous pregnancy and wherein the patient had previously exhibited alloimmunization in pregnancy, optionally wherein the alloimmunization in pregnancy is hemolytic disease of the fetus and newborn (HDFN), neonatal alloimmune neutropenia (NAN) or fetal and neonatal alloimmune thrombocytopenia (FNAIT).

9. The method of claim 1, wherein the patient is a sensitized patient who is sensitized from a previous treatment for a condition or disease, wherein the condition or disease is different from or the same as the disease or condition for which the patient is being treated.

10. The method of claim 1, wherein the patient received a previous treatment for a condition or disease, wherein the previous treatment did not comprise the population of cells, and wherein:
a. the population of cells is administered for the treatment of the same condition or disease as the previous treatment;
b. the population of cells exhibits an enhanced therapeutic effect for the treatment of the condition or disease in the patient as compared to the previous treatment;
c. the population of cells exhibits a longer therapeutic effect for the treatment of the condition or disease in the patient as compared to the previous treatment;
d. the previous treatment was therapeutically effective
e. the previous treatment was therapeutically ineffective;
f. the patient developed an immune reaction against the previous treatment; and/or
g. the population of cells is administered for the treatment of a different condition or disease as the previous treatment.

11.-13. (canceled)

14. The method of claim 1, wherein the patient has an allergy, optionally wherein the allergy is an allergy selected from the group consisting of a hay fever, a food allergy, an insect allergy, a drug allergy, and atopic dermatitis.

15.-18. (canceled)

19. The method of claim 1, wherein the cells are differentiated from stem cells, optionally wherein the stem cells are mesenchymal stem cells, embryonic stem cells, pluripotent stem cells, or induced pluripotent stem cells.

20.-22. (canceled)

23. The method of claim 1, wherein the cells are selected from the group consisting of cardiac cells, cardiac progenitor cells, neural cells, glial progenitor cells, endothelial cells, T cells, B cells, pancreatic islet cells, retinal pigmented epithelium cells, hepatocytes, thyroid cells, skin cells, blood cells, plasma cells, platelets, renal cells, epithelial cells, chimeric antigen receptor (CAR) T cells, NK cells, and CAR-NK cells.

24. The method of claim 1, wherein the cells are derived from primary cells, optionally wherein the primary cells are primary T cells, primary beta cells, or primary retinal pigment epithelial cells.

25.-26. (canceled)

27. The method of claim 1, wherein the cells comprise a second exogenous polynucleotide encoding a chimeric antigen receptor (CAR).

28.-53. (canceled)

54. The method of claim 24, wherein the cells derived from primary T cells comprise reduced expression of one or more of:
a. an endogenous T cell receptor;
b. cytotoxic T-lymphocyte-associated protein 4 (CTLA4);
c. programmed cell death (PD1); and
d. programmed cell death ligand 1 (PD-L1), wherein the reduced expression is due to a modification and the reduced expression is relative to a cell of the same cell type that does not comprise the modification.

55. The method of claim 54, wherein the cells derived from primary T cells comprised reduced expression of TRAC.

56. The method of claim 23, wherein the cells are T cells derived from induced pluripotent stem cells that comprise reduced expression of one or more of:
a. an endogenous T cell receptor;
b. cytotoxic T-lymphocyte-associated protein 4 (CTLA4);
c. programmed cell death (PD1); and
d. programmed cell death ligand 1 (PD-L1).

57. The method of claim 56, wherein the cells are T cells derived from induced pluripotent stem cells that comprise reduced expression of TRAC and TRB.

58.-62. (canceled)

63. The method of claim 1, wherein the patient exhibits no immune response upon administration of the population of cells, optionally wherein the no immune response upon administration of the population of cells is selected from the group consisting of no systemic immune response, no adaptive immune response, no innate immune response, no T cell response, no B cell response, and no systemic acute cellular immune response.

64. (canceled)

65. The method of claim 63, wherein the patient exhibits one or more of:
a. no systemic TH1 activation upon administering the population of cells;
b. no immune activation of peripheral blood mononuclear cells (PBMCs) upon administering the population of cells;
c. no donor specific IgG antibodies against the population of cells upon administering the population of cells;
d. no IgM and IgG antibody production against the population of cells upon administering the population of cells; and
e. no cytotoxic T cell killing of the population of cells upon administering the population of cells.

66.-73. (canceled)

74. The method of claim 1, wherein the population of cells is administered for treatment of a cellular deficiency or as a cellular therapy for the treatment of a condition or disease in a tissue or organ selected from the group consisting of heart, lung, kidney, liver, pancreas, intestine, stomach, cornea, bone marrow, blood vessel, heart valve, brain, spinal cord, and bone, wherein:
a. the cellular deficiency is associated with a neurodegenerative disease or the cellular therapy is for the treatment of a neurodegenerative disease;
b. the cellular deficiency is associated with a liver disease or the cellular therapy is for the treatment of liver disease;
c. the cellular deficiency is associated with a corneal disease or the cellular therapy is for the treatment of corneal disease;
d. the cellular deficiency is associated with a cardiovascular condition or disease or the cellular therapy is for the treatment of a cardiovascular condition or disease;
e. the cellular deficiency is associated with diabetes or the cellular therapy is for the treatment of diabetes;
f. the cellular deficiency is associated with a vascular condition or disease or the cellular therapy is for the treatment of a vascular condition or disease;
g. the cellular deficiency is associated with autoimmune thyroiditis or the cellular therapy is for the treatment of autoimmune thyroiditis; or
h. the cellular deficiency is associated with a kidney disease or the cellular therapy is for the treatment of a kidney disease.

75. The method of claim 74, wherein:
a. the neurodegenerative disease is selected from the group consisting of leukodystrophy, Huntington's disease, Parkinson's disease, multiple sclerosis, transverse myelitis, and Pelizaeus-Merzbacher disease (PMD);
b. the liver disease comprises cirrhosis of the liver;
c. the corneal disease is Fuchs dystrophy or congenital hereditary endothelial dystrophy; or
d. the cardiovascular disease is myocardial infarction or congestive heart failure.

76. The method of claim 74, wherein the population of cells comprises:
a. cells selected from the group consisting of glial progenitor cells, oligodendrocytes, astrocytes, and dopaminergic neurons, optionally wherein the dopaminergic neurons are selected from the group consisting of neural stem cells, neural progenitor cells, immature dopaminergic neurons, and mature dopaminergic neurons;
b. hepatocytes or hepatic progenitor cells;
c. corneal endothelial progenitor cells or corneal endothelial cells;
d. cardiomyocytes or cardiac progenitor cells;
e. pancreatic islet cells, including pancreatic beta islet cells, optionally wherein the pancreatic islet cells are selected from the group consisting of a pancreatic islet progenitor cell, an immature pancreatic islet cell, and a mature pancreatic islet cell;
f. endothelial cells;
g. thyroid progenitor cells; or
h. renal precursor cells or renal cells.

77. The method of claim 1, wherein the population of cells is administered for the treatment of cancer, optionally wherein the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

78. (canceled)

79. The method of claim 1, wherein the patient is receiving a tissue or organ transplant, optionally wherein the tissue or organ transplant or partial organ transplant is selected from the group consisting of a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, a bone transplant, a partial lung transplant, a partial kidney transplant, a partial liver transplant, a partial pancreas transplant, a partial intestine transplant, and a partial cornea transplant.

80.-174. (canceled)

175. A method of treating a patient in need thereof comprising administering a population of hypoimmunogenic cells, wherein the hypoimmunogenic cells comprise a first exogenous polynucleotide encoding CD47, a second exogenous polynucleotide encoding a CAR and
(I) one or more of:
a. reduced expression of major histocompatibility complex (MHC) class I and/or class II human leukocyte antigens;
b. reduced expression of MHC class I and class II human leukocyte antigens;
c. reduced expression of beta-2-microglobulin (B2M) and/or MHC class II transactivator (CIITA); and/or
d. reduced expression of B2M and CIITA;
wherein the reduced expression is due to a modification and the reduced expression is relative to a cell of the same cell type that does not comprise the modification;
(II) wherein:
a. the patient is not a sensitized patient; or
b. the patient is a sensitized patient, wherein the patient:
i. is sensitized against one or more alloantigens;
ii. is sensitized against one or more autologous antigens;
iii. is sensitized from a previous transplant;
iv. is sensitized from a previous pregnancy;
v. received a previous treatment for a condition or disease; and/or
vi. is a tissue or organ patient, and the hypoimmunogenic cells are administered prior to administering the tissue or organ transplant.

176.-311. (canceled)


SEQUENCE LISTING (14 sequences)

SEQ ID NO: 1 - RNA, 20 nt, Artificial Sequence
  synthetic guide sequence for ABO
ucucuccaugugcaguagga

SEQ ID NO: 2 - RNA, 20 nt, Artificial Sequence
  synthetic guide sequence for FUT1
cuggaugucggaggaguacg

SEQ ID NO: 3 - RNA, 20 nt, Artificial Sequence
  synthetic guide sequence for RH
gucuccggaaacucgaggug

SEQ ID NO: 4 - RNA, 20 nt, Artificial Sequence
  synthetic guide sequence for F3 (CD142)
acaguguagacuugauugac

SEQ ID NO: 5 - RNA, 20 nt, Artificial Sequence
  synthetic guide sequence for B2M
cgugaguaaaccugaaucuu

SEQ ID NO: 6 - RNA, 20 nt, Artificial Sequence
  synthetic guide sequence for CIITA
gauauuggcauaagccuccc

SEQ ID NO: 7 - RNA, 20 nt, Artificial Sequence
  synthetic guide sequence for TRAC
agagucucucagcugguaca

SEQ ID NO: 8 - RNA, 20 nt, Artificial Sequence
  synthetic guide sequence 20-mer
  misc_feature: n can be any ribonucleotide base
  misc_feature (1)..(20): n is a, c, g, or u
nnnnnnnnnnnnnnnnnnnn

SEQ ID NO: 9 - RNA, 12 nt, Artificial Sequence
  synthetic 12 nt crRNA repeat sequence
guuuuagagcua

SEQ ID NO: 10 - skipped

SEQ ID NO: 11 - RNA, 99 nt, Artificial Sequence
  synthetic guide sequence
  misc_feature: n can be any ribonucleotide base
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuu

SEQ ID NO: 12 - protein, 305 aa, Artificial Sequence
  synthetic human CD47 polypeptide
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 13 - protein, 323 aa, Artificial Sequence
  synthetic human CD47 polypeptide with signal sequence
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 14 - protein, 18 aa, Artificial Sequence
  synthetic Whitlow linker
GSTSGSGKPGSGEGSTKG